SEQUENCE LISTING (159 sequences)

SEQ ID NO: 1 - protein, 188 aa, Homo sapiens [sequence omitted]

SEQ ID NO: 2 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 3 - protein, 208 aa, Homo sapiens
MIIKHFFGTVLVLLASTTIFSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 4 - protein, 207 aa, Homo sapiens
MSTKPDMIQKCLWLEILMGIFIAGTLSLDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCLEEDKNENEDMKEMKENEMKPSEARVPQLSSLELRRYFHRIDNFLKEKKYSDCAWEIVRVEIRRCLYYFYKFTALFRRK

SEQ ID NO: 5 - protein, 195 aa, Homo sapiens
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 6 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 7 - protein, 200 aa, Homo sapiens
MAAAWTVVLVTLVLGLAVAGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 8 - protein, 134 aa, Homo sapiens
ATGVHSEVQLVQSGAEVKKPGESLKISCKGSGYSFTSYWIGWVRQMPGKGLEWMGVIYPGDSDTRYSPSFQGQVTISADKSISTAYLQWSSLKASDTAMYYCARTQWGYNYGSHFFYMDVWGKGTTVTVSSAST

SEQ ID NO: 9 - protein, 117 aa, Homo sapiens
EVQLVQSGGGVVQPGKSLRLSCAASGFAFSSYAMHWVRQAPGKGLEWVAVISYDGSNKYYADSVKGRFTISRDNSKNTLYLQMNSLRAEDTAVYYCARDRSYYLDYWGQGTLVTVSS

SEQ ID NO: 10 - protein, 120 aa, Homo sapiens
EVQLVESGAEVVKPGASVKMSCKASGYPFTSYNIHWIKQTPGQGLEWIGAIYPGNGDTSYTQKFKVKATLTSDKSSSTAYMQLSSLTSEDSAVYFCARYGNYPSYAMDYWGQGTSVTVSS

SEQ ID NO: 11 - protein, 129 aa, Homo sapiens
QVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSS

SEQ ID NO: 12 - protein, 130 aa, Homo sapiens [sequence omitted]

SEQ ID NO: 13 - protein, 123 aa, Homo sapiens
QVQLVESGGGVAQPGRSLRLSCAASGFSFSRHGMHWVRQAPGKGLEWVAGIWFDGTNDYYTDSVKGRFTISRDNSRSTLYLDINSLRAEDTAVYYCAREDPHLLIATLDLWGLGTLVTVSSAS

SEQ ID NO: 14 - protein, 121 aa, Homo sapiens
SQVQLQQSGPGLVKPSQTLSLTCGISGDSVSSKSAAWNWIRQSPSRGLEWLGRTYYRSKWHNDYAVSVKSRITINPDTSKNQFSLQLNSVTPEDTAVYYCARGQMGALDVWGQGTTVTVSS

SEQ ID NO: 15 - protein, 123 aa, Homo sapiens
EVQVLESGGGLVQPGGSLRLSCAASGFRFSSYAMSWVRQAPGKGLEWVSGISGTGENTYYADSVKGRFTISRDNSKNTLYVQMNSLRAEDTAVYYCAKDAILGSGHPWYFHVWGRGTLVTVSS

SEQ ID NO: 16 - protein, 123 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGYFGSGSLYGIDVWGQGTTVTVSS

SEQ ID NO: 17 - protein, 123 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKTDIKWGATNYGMDVWGQGTTVTVSS

SEQ ID NO: 18 - protein, 131 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKEIGGFDFRSGSQRSYYYYGVDVWGQGTTVTVSS

SEQ ID NO: 19 - protein, 120 aa, Homo sapiens
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAIYMDEAWAFEIWGQGTMVTVSS

SEQ ID NO: 20 - protein, 121 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARHLYRYGYRNYFDYWGQGTLVTVSS

SEQ ID NO: 21 - protein, 131 aa, Homo sapiens
ATGSWAQSVLTQPPSASGTPGQRVTISCSGSSSNIGSDHVYWYQQLPGTAPKLFIYRNNQRPSGVPDRFSGSKSGTSASLAISGLRSEDEADYYCAAWDASLSGYVFGTGTKVTVLGQPKANPTVTLFPPS

SEQ ID NO: 22 - protein, 113 aa, Homo sapiens
ETTLTQSPATLSLSPGERATLSCRASQSVRSNLAWYQQKPGQAPRPLIYDASTRATGIPDRFSGSGSGTDFTLTISRLEPEDFAVYYCQQRSNWPPTFGQGTKVEVKSGLVPR

SEQ ID NO: 23 - protein, 111 aa, Homo sapiens
DIVMTQSPASLTVSLGQRATISCRASKSVSASGYNYLHWYQQRPGQPPKLLIYLAFNLESGVPARFNGSGSGTDFTLNIHPVEEEDAATYYCQHSRDLPFTFGSGTKLEIK

SEQ ID NO: 24 - protein, 111 aa, Homo sapiens
DIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPTFGQGTKVEIK

SEQ ID NO: 25 - protein, 108 aa, Homo sapiens
DIQLTQSPSFLSASVGDRVTITCRASQDISSYLAWYQQKPGNAPKLLIYAASLLQSGVPSRFSAFGSGTEFTLTISSLQPEDFATYYCQHLKSYPLFTFGPGTKVDIK

SEQ ID NO: 26 - protein, 108 aa, Homo sapiens
DIQVTQSPSSLSAFVGDRVTITCRASQGLTSFINWYQQKPGRAPKLLISSASSLQRGVPSRFTASGSGTHFTLTISSLQPEDFATYYCQQSYGTPALAFGGGTKVDIK

SEQ ID NO: 27 - protein, 110 aa, Homo sapiens
QSVLTQPPSASGTPGQRVTISCSGSSSNIGSYYVYWYQQFPGTAPKLLIYGNNQRPSGVPDRFSGSKSGTSASLAITGLQAEDEADYYCQSYDSSLSGVIFGGGTKLTVL

SEQ ID NO: 28 - protein, 108 aa, Homo sapiens
SYVLTQPPSVSVAPGQTARMTCGGNNIGSESVHWFQQKPGQAPVLVVYDDSDRPSGIPERFSGSNSGNTATLTISRVEAGDEADYYCQVWDSSSDHAVFGGGTQLTVL

SEQ ID NO: 29 - protein, 110 aa, Homo sapiens
SYVLTQPPSVSVAPGQTARITCGGNNIGSKSVHWYQQKPGQAPVLVVYDDSDRPSGIPERFSGSNSGNTATLTISRVEAGDEADYYCQVWDSNHDHPGVVFGGGTKLTVL

SEQ ID NO: 30 - protein, 109 aa, Homo sapiens
SYVLTQPPSVSVAPGQTARITCGGNNIGSKSVHWYQQKPGQAPVLVVYDDSDRPSGIPERFSGSNSGNTATLTISRVEAGDEADYYCQVWDGTRDHLVVFGGGTKLTVL

SEQ ID NO: 31 - protein, 110 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYSSSPPGYTFGQGTKLEIK

SEQ ID NO: 32 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYTISLFTFGQGTKLEIK

SEQ ID NO: 33 - protein, 107 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 34 - protein, 805 aa, Homo sapiens [sequence omitted]

SEQ ID NO: 35 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 36 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 37 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 38 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 39 - protein, 492 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 40 - protein, 352 aa, Homo sapiens [sequence omitted]

SEQ ID NO: 41 - protein, 372 aa, Homo sapiens
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 42 - protein, 404 aa, Homo sapiens
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 43 - protein, 399 aa, Homo sapiens
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 44 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 45 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 46 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 47 - protein, 153 aa, Homo sapiens [sequence omitted]

SEQ ID NO: 48 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 49 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 50 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 51 - skipped

SEQ ID NO: 52 - protein, 150 aa, Vesiculovirus sp.
IGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDI

SEQ ID NO: 53 - protein, 130 aa, Vesiculovirus sp.
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNL

SEQ ID NO: 54 - protein, 115 aa, Vesiculovirus sp.
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTT

SEQ ID NO: 55 - protein, 100 aa, Vesiculovirus sp.
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFING

SEQ ID NO: 56 - protein, 84 aa, Vesiculovirus sp.
IQADGWMCHASKWVTTCDFRWYGPKYITHSIRSFTPSVEQCKESIEQTKQGTWLNPGFPPQSCGYATVTDAEAVIVQVTPHHVL

SEQ ID NO: 57 - protein, 27 aa, unidentified
GYIPEAPRDGQAYVRKDGEWVLLSTFL

SEQ ID NO: 58 - protein, 33 aa, unidentified
RMKQLEDKVEELLSKQYHLENEVARLKKLVGER

SEQ ID NO: 59 - protein, 33 aa, unidentified
RMKQLEDKVEELLSKNYHLENEVARLKKLVGER

SEQ ID NO: 60 - protein, 40 aa, Alphainfluenzavirus sp.
MNPNQKIITIGSICLVVGLISLILQIGNIISIWISHSIQT

SEQ ID NO: 61 - protein, 75 aa, Alphainfluenzavirus sp.
MNPNQKIITIGSICMVTGIVSLMLQIGNMISIWVSHSIHTGNQHQSEPISNTNFLTEKAVASVKLAGNSSLCPIN

SEQ ID NO: 62 - protein, 68 aa, Dengue virus
KLCIEAKISNTTTDSRCPTQGEATLVEEQDTNFVCRRTFVDRGHGNGCGLFGKGSLITCAKFKCVTKL

SEQ ID NO: 63 - protein, 83 aa, Dengue virus
IELLKTEVTNPAVLRKLCIEAKISNTTTDSRCPTQGEATLVEEQDTNFVCRRTFVDRGHGNGCGLFGKGSLITCAKFKCVTKL

SEQ ID NO: 64 - protein, 83 aa, Dengue virus
KLCIEAKISNTTTDSRCPTQGEATLVEEQDTNFVCRRTFVDRGHGNGCGLFGKGSLITCAKFKCVTKLEGKIVQYENLKYSVI

SEQ ID NO: 65 - protein, 59 aa, Dengue virus
EAKISNTTTDSRCPTQGEATLVEEQDTNFVCRRTFVDRGHGNGCGLFGKGSLITCAKFK

SEQ ID NO: 66 - protein, 22 aa, Vesiculovirus sp.
IASFFFIIGLIIGLFLVLRVGI

SEQ ID NO: 67 - protein, 37 aa, Vesiculovirus sp.
PIELVEGWFSSWKSSIASFFFIIGLIIGLFLVLRVGI

SEQ ID NO: 68 - protein, 53 aa, Vesiculovirus sp.
DDESLFFGDTGLSKNPIELVEGWFSSWKSSIASFFFIIGLIIGLFLVLRVGIH

SEQ ID NO: 69 - protein, 81 aa, Vesiculovirus sp.
GMLDSDLHLSSKAQVFEHPHIQDAASQLPDDESLFFGDTGLSKNPIELVEGWFSSWKSSIASFFFIIGLIIGLFLVLRVGI

SEQ ID NO: 70 - protein, 25 aa, Vesiculovirus sp.
HLCIKLKHTKKRQIYTDIEMNRLGK

SEQ ID NO: 71 - protein, 23 aa, Alphainfluenzavirus sp.
IITIGSVCMTIGMANLILQIGNI

SEQ ID NO: 72 - protein, 23 aa, Alphainfluenzavirus sp.
LAIYSTVASSLVLVVSLGAISFW

SEQ ID NO: 73 - protein, 52 aa, Dengue virus
AYGVLFSGVSWTMKIGIGILLTWLGLNSRSTSLSMTCIAVGMVTLYLGVMVQ

SEQ ID NO: 74 - protein, 21 aa, Lentivirus sp.
FIMIVGGLVGLRIVFAVLSIV

SEQ ID NO: 75 - protein, 5 aa, Homo sapiens
SYWIG

SEQ ID NO: 76 - protein, 17 aa, Homo sapiens
VIYPGDSDTRYSPSFQG

SEQ ID NO: 77 - protein, 16 aa, Homo sapiens
TQWGYNYGSHFFYMDV

SEQ ID NO: 78 - protein, 5 aa, Homo sapiens
SYAMH

SEQ ID NO: 79 - protein, 17 aa, Homo sapiens
VISYDGSNKYYADSVKG

SEQ ID NO: 80 - protein, 8 aa, Homo sapiens
DRSYYLDY

SEQ ID NO: 81 - protein, 5 aa, Homo sapiens
SYNIH

SEQ ID NO: 82 - protein, 17 aa, Homo sapiens
AIYPGNGDTSYTQKFKV

SEQ ID NO: 83 - protein, 11 aa, Homo sapiens
YGNYPSYAMDY

SEQ ID NO: 84 - protein, 5 aa, Homo sapiens
TYAMH

SEQ ID NO: 85 - protein, 17 aa, Homo sapiens
VISYDANYKYYADSVKG

SEQ ID NO: 86 - protein, 20 aa, Homo sapiens
DSQLRSLLYFEWLSQGYFDY

SEQ ID NO: 87 - protein, 5 aa, Homo sapiens
DYGMS

SEQ ID NO: 88 - protein, 17 aa, Homo sapiens
GLNWNGDITAFTDSVKG

SEQ ID NO: 89 - protein, 21 aa, Homo sapiens
VRTWGDYTTGEEIINSWYFDL

SEQ ID NO: 90 - protein, 5 aa, Homo sapiens
RHGMH

SEQ ID NO: 91 - protein, 17 aa, Homo sapiens
GIWFDGTNDYYTDSVKG

SEQ ID NO: 92 - protein, 12 aa, Homo sapiens
EDPHLLIATLDL

SEQ ID NO: 93 - protein, 7 aa, Homo sapiens
SKSAAWN

SEQ ID NO: 94 - protein, 18 aa, Homo sapiens
RTYYRSKWHNDYAVSVKS

SEQ ID NO: 95 - protein, 8 aa, Homo sapiens
GQMGALDV

SEQ ID NO: 96 - protein, 5 aa, Homo sapiens
SYAMS

SEQ ID NO: 97 - protein, 17 aa, Homo sapiens
GISGTGENTYYADSVKG

SEQ ID NO: 98 - protein, 14 aa, Homo sapiens
DAILGSGHPWYFHV

SEQ ID NO: 99 - protein, 5 aa, Homo sapiens
SYAMS

SEQ ID NO: 100 - protein, 17 aa, Homo sapiens
AISGSGGSTYYADSVKG

SEQ ID NO: 101 - protein, 14 aa, Homo sapiens
DGYFGSGSLYGIDV

SEQ ID NO: 102 - protein, 5 aa, Homo sapiens
SYGMH

SEQ ID NO: 103 - protein, 17 aa, Homo sapiens
VISYDGSNKYYADSVKG

SEQ ID NO: 104 - protein, 14 aa, Homo sapiens
TDIKWGATNYGMDV

SEQ ID NO: 105 - protein, 5 aa, Homo sapiens
SYGMH

SEQ ID NO: 106 - protein, 17 aa, Homo sapiens
VISYDGSNKYYADSVKG

SEQ ID NO: 107 - protein, 22 aa, Homo sapiens
EIGGFDFRSGSQRSYYYYGVDV

SEQ ID NO: 108 - protein, 7 aa, Homo sapiens
SGDYYWS

SEQ ID NO: 109 - protein, 16 aa, Homo sapiens
YIYYSGSTYYNPSLKS

SEQ ID NO: 110 - protein, 10 aa, Homo sapiens
YMDEAWAFEI

SEQ ID NO: 111 - protein, 5 aa, Homo sapiens
SYYWS

SEQ ID NO: 112 - protein, 16 aa, Homo sapiens
YIYYSGSTNYNPSLKS

SEQ ID NO: 113 - protein, 13 aa, Homo sapiens
HLYRYGYRNYFDY

SEQ ID NO: 114 - protein, 13 aa, Homo sapiens
SGSSSNIGSDHVY

SEQ ID NO: 115 - protein, 7 aa, Homo sapiens
RNNQRPS

SEQ ID NO: 116 - protein, 11 aa, Homo sapiens
AAWDASLSGYV

SEQ ID NO: 117 - protein, 11 aa, Homo sapiens
RASQSVRSNLA

SEQ ID NO: 118 - protein, 7 aa, Homo sapiens
DASTRAT

SEQ ID NO: 119 - protein, 9 aa, Homo sapiens
QQRSNWPPT

SEQ ID NO: 120 - protein, 15 aa, Homo sapiens
RASKSVSASGYNYLH

SEQ ID NO: 121 - protein, 7 aa, Homo sapiens
LAFNLES

SEQ ID NO: 122 - protein, 9 aa, Homo sapiens
QHSRDLPFT

SEQ ID NO: 123 - protein, 15 aa, Homo sapiens
KSSQSVTFNYKNYLA

SEQ ID NO: 124 - protein, 7 aa, Homo sapiens
WASTRES

SEQ ID NO: 125 - protein, 9 aa, Homo sapiens
QQHYRTPPT

SEQ ID NO: 126 - protein, 11 aa, Homo sapiens
RASQDISSYLA

SEQ ID NO: 127 - protein, 7 aa, Homo sapiens
AASLLQS

SEQ ID NO: 128 - protein, 10 aa, Homo sapiens
QHLKSYPLFT

SEQ ID NO: 129 - protein, 11 aa, Homo sapiens
RASQGLTSFIN

SEQ ID NO: 130 - protein, 7 aa, Homo sapiens
SASSLQR

SEQ ID NO: 131 - protein, 10 aa, Homo sapiens
QQSYGTPALA

SEQ ID NO: 132 - protein, 13 aa, Homo sapiens
SGSSSNIGSYYVY

SEQ ID NO: 133 - protein, 7 aa, Homo sapiens
GNNQRPS

SEQ ID NO: 134 - protein, 11 aa, Homo sapiens
QSYDSSLSGVI

SEQ ID NO: 135 - protein, 11 aa, Homo sapiens
GGNNIGSESVH

SEQ ID NO: 136 - protein, 7 aa, Homo sapiens
DDSDRPS

SEQ ID NO: 137 - protein, 11 aa, Homo sapiens
QVWDSSSDHAV

SEQ ID NO: 138 - protein, 11 aa, Homo sapiens
GGNNIGSKSVH

SEQ ID NO: 139 - protein, 7 aa, Homo sapiens
DDSDRPS

SEQ ID NO: 140 - protein, 13 aa, Homo sapiens
QVWDSNHDHPGVV

SEQ ID NO: 141 - protein, 11 aa, Homo sapiens
GGNNIGSKSVH

SEQ ID NO: 142 - protein, 7 aa, Homo sapiens
DDSDRPS

SEQ ID NO: 143 - protein, 12 aa, Homo sapiens
QVWDGTRDHLVV

SEQ ID NO: 144 - protein, 12 aa, Homo sapiens
RASQSVSSSYLA

SEQ ID NO: 145 - protein, 7 aa, Homo sapiens
GASSRAT

SEQ ID NO: 146 - protein, 11 aa, Homo sapiens
QQYSSSPPGYT

SEQ ID NO: 147 - protein, 11 aa, Homo sapiens
RASQSISSYLN

SEQ ID NO: 148 - protein, 7 aa, Homo sapiens
AASSLQS

SEQ ID NO: 149 - protein, 10 aa, Homo sapiens
QQSYTISLFT

SEQ ID NO: 150 - protein, 11 aa, Homo sapiens
RASQGISSWLA

SEQ ID NO: 151 - protein, 7 aa, Homo sapiens
AASSLQS

SEQ ID NO: 152 - protein, 9 aa, Homo sapiens
QQANSFPLT

SEQ ID NO: 153 - protein, 294 aa, synthetic construct
MALTFALLVALLVLSCKSSCSVGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIEQKLISEEDL

SEQ ID NO: 154 - protein, 425 aa, synthetic construct
MALTFALLVALLVLSCKSSCSVGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 155 - protein, 293 aa, synthetic construct
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIEQKLISEEDL

SEQ ID NO: 156 - protein, 424 aa, synthetic construct
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 157 - protein, 272 aa, synthetic construct
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIEQKLISEEDL

SEQ ID NO: 158 - protein, 403 aa, synthetic construct
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 159 - protein, 6 aa, synthetic construct
HHHHHH

(19) United States
(12) Patent Application Publication
(10) Pub. No.: US 2024/0398902 A1
(43) Pub. Date: Dec. 5, 2024

(54) MULTIVALENT INTERFERON PARTICLES COMPOSITIONS AND METHODS OF USE

(71) Applicant: Achelois BioPharma, Inc., Redwood City, CA (US)

(72) Inventors: Michael CHEN, Palo Alto, CA (US); Chang-Zheng CHEN, Palo Alto, CA (US)

(21) Appl. No.: 18/690,910

(22) PCT Filed: Sep. 12, 2022
(86) PCT No.: PCT/US2022/043205

§ 371 Date: Mar. 11, 2024

Related U.S. Application Data
(60) Provisional application No. 63/243,676, filed on Sep. 13, 2021.

(51) Int. Cl. A61K 38/21; A61K 9/16; A61K 47/68

(52) U.S. Cl. CPC A61K 38/217; A61K 9/1658; A61K 38/21; A61K 38/212; A61K 38/215; A61K 47/6813; A61K 47/6841; A61K 47/6855; A61K 47/6857; A61K 47/6859; A61K 47/6861; A61K 47/6863; A61K 47/6865; A61K 47/6867; A61K 47/6869

(57) ABSTRACT

Disclosed herein are multivalent particles that comprise multiple copies of an interferon (IFN) fusion proteins, wherein fusion protein comprises interferon polypeptide fused to a transmembrane domain and expressed on the surface of the multivalent particle. It also discloses composition and uses of the multivalent particles for treating diseases, disorders, and infections.

### CROSS-REFERENCE

This application claims the benefit of U.S. Provisional Application No. 63/243,676 filed Sep. 13, 2021, which is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Disclosed herein is a multivalent particle comprising a first fusion protein that comprises an interferon (IFN) polypeptide and a transmembrane polypeptide wherein the first fusion protein is expressed on a surface of the multivalent particle. In some embodiments, the multivalent particle further comprises a second fusion protein. In some embodiments, the second fusion protein comprises a transmembrane polypeptide and an IFN polypeptide that has less than 100% sequence identity to the IFN polypeptide of the first fusion protein and wherein the second fusion protein is expressed on the surface of the multivalent particle. In some embodiments, the IFN polypeptide of the first fusion protein comprises a human IFN polypeptide sequence. In some embodiments, the IFN polypeptide of the second fusion protein comprises a human IFN polypeptide sequence. In some embodiments, the IFN polypeptide of the first fusion protein comprises a Type I IFN, Type II IFN, or a Type III IFN. In some embodiments, the Type I IFN comprises IFN-alpha, IFN-beta, IFN-epsilon, IFN-kappa, or IFN-omega. In some embodiments, the Type II IFN comprises IFN-gamma. In some embodiments, the Type III IFN comprises IFN-lambda. In some embodiments, the IFN polypeptide of the second fusion protein comprises a Type I IFN, Type II IFN, or a Type III IFN. In some embodiments, the Type I IFN comprises IFN-alpha, IFN-beta, IFN-epsilon, IFN-kappa, or IFN-omega. In some embodiments, the Type II IFN comprises IFN-gamma. In some embodiments, the Type III IFN comprises IFN-lambda. In some embodiments, the IFN polypeptide of the first fusion protein comprises an amino acid sequence with at least 90% sequence identity to any one of SEQ ID NOs: 1-7. In some embodiments, the IFN polypeptide of the second fusion protein comprises an amino acid sequence with at least 90% sequence identity to any one of SEQ ID NOs: 1-7. In some embodiments, the second fusion protein comprises a transmembrane polypeptide and a homing polypeptide that targets the multivalent particle to a target cell or a target protein wherein the second fusion protein is expressed on the surface of the multivalent particle. In some embodiments, the target cell comprises a cancer cell. In some embodiments, the cancer cell is from a cancer comprising melanoma, leukemia, lymphoma, multiple myeloma, liver cancer, pancreatic cancer, lung cancer, breast cancer, prostate cancer, brain cancer, colorectal cancer, bladder cancer, kidney cancer, cervical cancer, ovarian cancer, esophageal cancer, mesothelioma, gastric cancer, and sarcoma. In some embodiments, the target protein comprises a viral surface protein from hepatitis B virus (HBV), SARS CoV-2, SARS CoV-1, MERS CoV, Influenza, respiratory syncytial virus, HIV, or measles. In some embodiments, the target protein comprises a viral spike protein. In some embodiments, the homing polypeptide comprises an antibody that binds specifically to an antigen on the target cell or the target viral protein. In some embodiments, the antigen on the target cell comprises an antigen listed in Table 2. In some embodiments, the antibody comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc. In some embodiments, the antibody comprises an amino acid sequence from at least one complementarity determining region of BG10-19, 80R, 7D10, FI6, 1E01, H015, 2H5, ADRI-2F3, H004, H009, H007, H019, or H020. In some embodiments, the antibody comprises a multi-specific antibody. In some embodiments, the homing polypeptide comprises a mammalian receptor that has binding specificity to the target viral protein. In some embodiments, the mammalian receptor comprises NTCP, ACE2, TMPRSS2, DPP4, CD4, HVEM, PD-1, CCR5, CXCR5, CD209, or CLEC4M. In some embodiments, the homing polypeptide comprises a mammalian ligand that recognizes a tumor-associated receptor. In some embodiments, the mammalian ligand comprises EGF, VEGF, TGFbeta, IL-4, IL-11, IGF1, IL-6, or RGD peptide.

In some embodiments, the first fusion protein is monomeric. In some embodiments, the first fusion protein comprises an oligomerization domain. In some embodiments, the second fusion protein is monomeric. In some embodiments, the second fusion protein comprises an oligomerization domain. In some embodiments, the oligomerization domain of the first fusion protein comprises a dimerization domain. In some embodiments, the dimerization domain comprises a leucine zipper dimerization domain. In some embodiments, the oligomerization domain of the first fusion protein comprises a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain comprises a tetramerization domain. In some embodiments, the tetramerization domain comprises an influenza neuraminidase stem domain. In some embodiments, the oligomerization domain of the second fusion protein comprises a dimerization domain. In some embodiments, the dimerization domain comprises a leucine zipper dimerization domain. In some embodiments, the oligomerization domain of the second fusion protein comprises a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain of the second fusion protein comprises a tetramerization domain. In some embodiments, the tetramerization domain comprises an influenza neuraminidase stem domain. In some embodiments, the oligomerization domain of the first fusion protein comprises an amino acid sequence that has at least 90% sequence identity to an amino acid sequence of any one of SEQ ID NOs: 52-65. In some embodiments, the oligomerization domain of the second fusion protein comprises an amino acid sequence that has at least 90% sequence identity to an amino acid sequence of anyone of SEQ ID NOs: 52-65.

In some embodiments, the first fusion protein comprises a signal peptide. In some embodiments, domains of the first fusion protein are arranged from the N-terminus to the C-terminus in the following orders: (a) signal peptide, interferon polypeptide, oligomerization domain, and transmembrane polypeptide; (b) signal peptide, interferon polypeptide, transmembrane polypeptide, and oligomerization domain; or (c) signal peptide, oligomerization domain, display peptide, and transmembrane polypeptide. In some embodiments, the first fusion protein further comprises a cytosolic domain. In some embodiments, domains of the first fusion protein are arranged from the N-terminus to the C-terminus in the following orders: (a) signal peptide, interferon polypeptide, oligomerization domain, transmembrane polypeptide, and cytosolic domain; (b) signal peptide, interferon polypeptide, transmembrane polypeptide, oligomerization domain, and cytosolic domain; or (c) signal peptide, oligomerization domain, interferon polypeptide, transmembrane polypeptide, and cytosolic domain. In some embodiments, the second fusion protein comprises a signal peptide. In some embodiments, domains of the second fusion protein are arranged from the N-terminus to the C-terminus in the following orders: (a) signal peptide, interferon polypeptide, oligomerization domain, and transmembrane polypeptide; (b) signal peptide, interferon polypeptide, transmembrane polypeptide, and oligomerization domain; or (c) signal peptide, oligomerization domain, interferon polypeptide, and transmembrane polypeptide. In some embodiments, the second fusion protein further comprises a cytosolic domain. In some embodiments, domains of the second fusion protein are arranged from the N-terminus to the C-terminus in the following orders: (a) signal peptide, interferon polypeptide, oligomerization domain, transmembrane polypeptide, and cytosolic domain; (b) signal peptide, interferon polypeptide, transmembrane polypeptide, oligomerization domain, and cytosolic domain; or (c) signal peptide, oligomerization domain, interferon polypeptide, transmembrane polypeptide, and cytosolic domain. In some embodiments, the second fusion protein comprises a signal peptide and an oligomerization domain. In some embodiments, domains of the second fusion protein are arranged from the N-terminus to the C-terminus in the following orders: (a) signal peptide, homing polypeptide, oligomerization domain, and transmembrane polypeptide; (b) signal peptide, homing polypeptide, transmembrane polypeptide, and oligomerization domain; or (c) signal peptide, oligomerization domain, homing polypeptide, and transmembrane polypeptide. In some embodiments, the second fusion protein further comprises a cytosolic domain. In some embodiments, domains of the second fusion protein are arranged from the N-terminus to the C-terminus in the following orders: (a) signal peptide, homing polypeptide, oligomerization domain, transmembrane polypeptide, and cytosolic domain; (b) signal peptide, homing polypeptide, transmembrane polypeptide, oligomerization domain, and cytosolic domain; or (c) signal peptide, oligomerization domain, homing polypeptide, transmembrane polypeptide, and cytosolic domain.

In some embodiments, the transmembrane polypeptide of the first fusion protein anchors the first fusion protein to a lipid bilayer of the multivalent particle. In some embodiments, the transmembrane polypeptide of the second fusion protein anchors the second fusion protein to a lipid bilayer of the multivalent particle. In some embodiments, the transmembrane polypeptide of the second fusion protein anchors the second fusion protein to a lipid bilayer of the multivalent particle. In some embodiments, the transmembrane polypeptide of the first fusion protein comprises a transmembrane domain of a Vesicular Stomatitis virus glycoprotein (VSV-G). In some embodiments, the transmembrane polypeptide of the first fusion protein comprises a cytosolic domain of a Vesicular Stomatitis virus glycoprotein (VSV-G). In some embodiments, the transmembrane polypeptide of the first fusion protein comprises a transmembrane domain of Influenza Neuraminidase (NA). In some embodiments, the transmembrane polypeptide of the first fusion protein comprises a transmembrane domain of influenza Hemagglutinin (HA). In some embodiments, the transmembrane polypeptide of the first fusion protein comprises a transmembrane domain of HIV surface glycoprotein GP120 or GP41. In some embodiments, the transmembrane polypeptide of the first fusion protein comprises a transmembrane domain of Dengue E Protein. In some embodiments, the transmembrane polypeptide of the first fusion protein comprises a transmembrane domain of measles virus surface glycoprotein hemagglutinin (H) protein. In some embodiments, the transmembrane polypeptide of the first fusion protein comprises an amino acid sequence with at least about 90% sequence identity to any one of SEQ ID NOs: 66-74. In some embodiments, the transmembrane polypeptide of the second fusion protein comprises a transmembrane domain of a Vesicular Stomatitis virus glycoprotein (VSV-G). In some embodiments, the transmembrane polypeptide of the second fusion protein comprises a cytosolic domain of a Vesicular Stomatitis virus glycoprotein (VSV-G). In some embodiments, the transmembrane polypeptide of the second fusion protein comprises a transmembrane domain of Influenza Neuraminidase (NA). In some embodiments, the transmembrane polypeptide of the second fusion protein comprises a transmembrane domain of influenza Hemagglutinin (HA). In some embodiments, the transmembrane polypeptide of the second fusion protein comprises a transmembrane domain of HIV surface glycoprotein GP120 or GP41. In some embodiments, the transmembrane polypeptide of the second fusion protein comprises a transmembrane domain of Dengue E Protein. In some embodiments, the transmembrane polypeptide of the second fusion protein comprises a transmembrane domain of measles virus surface glycoprotein hemagglutinin (H) protein. In some embodiments, the transmembrane polypeptide of the second fusion protein comprises an amino acid sequence with at least about 90% sequence identity to any one of SEQ ID NOs: 66-74. In some embodiments, the transmembrane polypeptide of the second fusion protein comprises a transmembrane domain of a Vesicular Stomatitis virus glycoprotein (VSV-G). In some embodiments, the transmembrane polypeptide of the second fusion protein comprises a cytosolic domain of a Vesicular Stomatitis virus glycoprotein (VSV-G). In some embodiments, the transmembrane polypeptide of the second fusion protein comprises a transmembrane domain of Influenza Neuraminidase (NA). In some embodiments, the transmembrane polypeptide of the second fusion protein comprises a transmembrane domain of influenza Hemagglutinin (HA). In some embodiments, the transmembrane polypeptide of the second fusion protein comprises a transmembrane domain of HIV surface glycoprotein GP120 or GP41. In some embodiments, the transmembrane polypeptide of the second fusion protein comprises a transmembrane domain of Dengue E Protein. In some embodiments, the transmembrane polypeptide of the second fusion protein comprises a transmembrane domain of measles virus surface glycoprotein hemagglutinin (H) protein. In some embodiments, the transmembrane polypeptide of the second fusion protein comprises an amino acid sequence with at least about 90% sequence identity to any one of SEQ ID NOs: 66-74.

In some embodiments, the multivalent particle is synthetic. In some embodiments, the multivalent particle is recombinant. In some embodiments, the multivalent particle comprises an enveloped particle. In some embodiments, the multivalent particle comprises a lentiviral particle. In some embodiments, the multivalent particle does not comprise viral genetic material. In some embodiments, the multivalent particle comprises a lipid bilayer. In some embodiments, the multivalent particle comprises a virus. In some embodiments, the multivalent particle comprises a replication incompetent virus. In some embodiments, the multivalent particle comprises a replication competent virus. In some embodiments, the multivalent particle comprises a viral-like particle. In some embodiments, the multivalent particle comprises an extracellular vesicle. In some embodiments, the extracellular vesicle comprises an ectosome. In some embodiments, the extracellular vesicle comprises an exosome. In some embodiments, the first fusion protein is expressed at a valency of about 10 copies on the surface of the multivalent particle. In some embodiments, the first fusion protein is expressed at a valency of about 10 to 15 copies on the surface of the multivalent particle. In some embodiments, the first fusion protein is expressed at a valency of at least about 25 copies on the surface of the multivalent particle. In some embodiments, the first fusion protein is expressed at a valency of at least about 50 copies on the surface of the multivalent particle. In some embodiments, the first fusion protein is expressed at a valency of at least about 100 copies on the surface of the multivalent particle. In some embodiments, the first fusion protein is expressed at a valency of at least about 200 copies on the surface of the multivalent particle. In some embodiments, the first fusion protein is expressed at a valency of at least about 400 copies on the surface of the multivalent particle. In some embodiments, the first fusion protein is expressed at a valency of at least about 600 copies on the surface of the multivalent particle. In some embodiments, the first fusion protein is expressed at a valency of at least about 1000 copies on the surface of the multivalent particle. In some embodiments, the second fusion protein is expressed at a valency of about 10 copies on the surface of the multivalent particle. In some embodiments, the second fusion protein is expressed at a valency of about 10 to 15 copies on the surface of the multivalent particle. In some embodiments, the second fusion protein is expressed at a valency of at least about 25 copies on the surface of the multivalent particle. In some embodiments, the second fusion protein is expressed at a valency of at least about 50 copies on the surface of the multivalent particle. In some embodiments, the second fusion protein is expressed at a valency of at least about 100 copies on the surface of the multivalent particle. In some embodiments, the second fusion protein is expressed at a valency of at least about 200 copies on the surface of the multivalent particle. In some embodiments, the second fusion protein is expressed at a valency of at least about 400 copies on the surface of the multivalent particle. In some embodiments, the second fusion protein is expressed at a valency of at least about 600 copies on the surface of the multivalent particle. In some embodiments, the second fusion protein is expressed at a valency of at least about 1000 copies on the surface of the multivalent particle. In some embodiments, the multivalent particle comprises a fluorophore expressed on a surface of the multivalent particle. In some embodiments, the fluorophore is conjugated to a membrane-intercalating polypeptide.

In some embodiments, the antibody comprises a CDR-H1 according to SEQ ID NO: 75, a CDR-H2 according to SEQ ID NO: 76, a CDR-H3 according to SEQ ID NO: 77, a CDR-L1 according to SEQ ID NO: 114, a CDR-L2 according to SEQ ID NO: 115, and a CDR-L3 according to SEQ ID NO: 116. In some embodiments, the antibody comprises a CDR-H1 according to SEQ ID NO: 78, a CDR-H2 according to SEQ ID NO: 79, a CDR-H3 according to SEQ ID NO: 80, a CDR-L1 according to SEQ ID NO: 117, a CDR-L2 according to SEQ ID NO: 118, and a CDR-L3 according to SEQ ID NO: 119. In some embodiments, the antibody comprises a CDR-H1 according to SEQ ID NO: 81, a CDR-H2 according to SEQ ID NO: 82, a CDR-H3 according to SEQ ID NO: 83, a CDR-L1 according to SEQ ID NO: 120, a CDR-L2 according to SEQ ID NO: 121, and a CDR-L3 according to SEQ ID NO: 122. In some embodiments, the antibody comprises a CDR-III according to SEQ ID NO: 84, a CDR-H2 according to SEQ ID NO: 85, a CDR-H3 according to SEQ ID NO: 86, a CDR-L1 according to SEQ ID NO: 123, a CDR-L2 according to SEQ ID NO: 124, and a CDR-L3 according to SEQ ID NO: 125. In some embodiments, the antibody comprises a CDR-III according to SEQ ID NO: 87, a CDR-H2 according to SEQ ID NO: 88, a CDR-H3 according to SEQ ID NO: 89, a CDR-L1 according to SEQ ID NO: 126, a CDR-L2 according to SEQ ID NO: 127, and a CDR-L3 according to SEQ ID NO: 128. In some embodiments, the antibody comprises a CDR-III according to SEQ ID NO: 90, a CDR-H2 according to SEQ ID NO: 91, a CDR-H3 according to SEQ ID NO: 92, a CDR-L1 according to SEQ ID NO: 129, a CDR-L2 according to SEQ ID NO: 130, and a CDR-L3 according to SEQ ID NO: 131. In some embodiments, the antibody comprises a CDR-H1 according to SEQ ID NO: 93, a CDR-H2 according to SEQ ID NO: 94, a CDR-H3 according to SEQ ID NO: 95, a CDR-L1 according to SEQ ID NO: 132, a CDR-L2 according to SEQ ID NO: 133, and a CDR-L3 according to SEQ ID NO: 134. In some embodiments, the antibody comprises a CDR-H1 according to SEQ ID NO: 96, a CDR-H2 according to SEQ ID NO: 97, a CDR-H3 according to SEQ ID NO: 98, a CDR-L1 according to SEQ ID NO: 135, a CDR-L2 according to SEQ ID NO: 136, and a CDR-L3 according to SEQ ID NO: 137. In some embodiments, the antibody comprises a CDR-H1 according to SEQ ID NO: 99, a CDR-H2 according to SEQ ID NO: 100, a CDR-H3 according to SEQ ID NO: 101, a CDR-L1 according to SEQ ID NO: 138, a CDR-L2 according to SEQ ID NO: 139, and a CDR-L3 according to SEQ ID NO: 140. In some embodiments, the antibody comprises a CDR-H1 according to SEQ ID NO: 102, a CDR-H2 according to SEQ ID NO: 103, a CDR-H3 according to SEQ ID NO: 104, a CDR-L1 according to SEQ ID NO: 141, a CDR-L2 according to SEQ ID NO: 142, and a CDR-L3 according to SEQ ID NO: 143. In some embodiments, the antibody comprises a CDR-H1 according to SEQ ID NO: 105, a CDR-H2 according to SEQ ID NO: 106, a CDR-H3 according to SEQ ID NO: 107, a CDR-L1 according to SEQ ID NO: 144, a CDR-L2 according to SEQ ID NO: 145, and a CDR-L3 according to SEQ ID NO: 146. In some embodiments, the antibody comprises a CDR-H1 according to SEQ ID NO: 108, a CDR-H2 according to SEQ ID NO: 109, a CDR-H3 according to SEQ ID NO: 110, a CDR-L1 according to SEQ ID NO: 147, a CDR-L2 according to SEQ ID NO: 148, and a CDR-L3 according to SEQ ID NO: 149. In some embodiments, the antibody comprises a CDR-H1 according to SEQ ID NO: 111, a CDR-H2 according to SEQ ID NO: 112, a CDR-H3 according to SEQ ID NO: 113, a CDR-L1 according to SEQ ID NO: 150, a CDR-L2 according to SEQ ID NO: 151, and a CDR-L3 according to SEQ ID NO: 152. In some embodiments, the antibody comprises: (a) a heavy chain variable region (VH) comprising an amino acid sequence that has at least 90% sequence identity to any one of SEQ ID Nos: 8-20; and (b) a light chain variable region (VL) comprising an amino acid sequence that has at least 90% sequence identity to any one of SEQ ID Nos: 21-33. In some embodiments, the mammalian receptor comprises an amino acid sequence that has at least 90% sequence identity to any one of SEQ ID NOs: 34-43. In some embodiments, the mammalian ligand comprises an amino acid sequence that has at least 90% sequence identity to any one of SEQ ID NOs: 44-51. In some embodiments, (a) the IFN polypeptide of the first fusion protein comprises an amino acid sequence that has at least 90% sequence identity to any one of SEQ ID NOs: 1-7; and (b) the transmembrane polypeptide of the first fusion protein comprises an amino acid sequence that has at least about 90% sequence identity to any one of SEQ ID NOs: 66-74. In some embodiments, (a) the IFN polypeptide of the second fusion protein comprises an amino acid sequence that has at least 90% sequence identity to any one of SEQ ID NOs: 1-7; and (b) the transmembrane polypeptide of the second fusion protein comprises an amino acid sequence that has at least about 90% sequence identity to any one of SEQ ID NOs: 66-74. In some embodiments, (a) the antibody comprises: (i) a heavy chain variable region (VH) comprising an amino acid sequence that has at least 90% sequence identity to any one of SEQ ID Nos: 8-20; and (ii) a light chain variable region (VL) comprising an amino acid sequence that has at least 90% sequence identity to any one of SEQ ID Nos: 21-33; and (b) the transmembrane polypeptide of the second fusion protein comprises an amino acid sequence that has at least about 90% sequence identity to any one of SEQ ID NOs: 66-74. In some embodiments, (a) the mammalian receptor comprises an amino acid sequence that has at least 90% sequence identity to any one of SEQ ID NOs: 34-43; and (b) the transmembrane polypeptide of the second fusion protein comprises an amino acid sequence that has at least about 90% sequence identity to any one of SEQ ID NOs: 66-74. In some embodiments, (a) the mammalian ligand comprises an amino acid sequence that has at least 90% sequence identity to any one of SEQ ID NOs: 44-51; and (b) the transmembrane polypeptide of the second fusion protein comprises an amino acid sequence that has at least about 90% sequence identity to any one of SEQ ID NOs: 66-74. In some embodiments, (a) the IFN polypeptide of the first fusion protein comprises an amino acid sequence that has at least 90% sequence identity to any one of SEQ ID NOs: 1-7; (b) the transmembrane polypeptide of the first fusion protein comprises an amino acid sequence that has at least about 90% sequence identity to any one of SEQ ID NOs: 66-74; and (c) the oligomerization domain of the first fusion protein comprises an amino acid sequence that has at least 90% sequence identity to any one of SEQ ID NOs: 52-65. In some embodiments, the first fusion protein comprises an amino acid sequence that has at least 90% sequence identity to any one of SEQ ID NOs: 153-158. In some embodiments, the second fusion protein comprises an oligomerization domain, wherein: (a) the IFN polypeptide of the second fusion protein comprises an amino acid sequence that has at least 90% sequence identity to any one of SEQ ID NOs: 1-7; (b) the transmembrane polypeptide of the second fusion protein comprises an amino acid sequence that has at least about 90% sequence identity to any one of SEQ ID NOs: 66-74; and (c) the oligomerization domain of the second fusion protein comprises an amino acid sequence that has at least 90% sequence identity to any one of SEQ ID NOs: 52-65. In some embodiments, the second fusion protein comprises an oligomerization domain, wherein: (a) the antibody comprises: (i) a heavy chain variable region (VH) comprising an amino acid sequence that has at least 90% sequence identity to any one of SEQ ID Nos: 8-20; and (ii) a light chain variable region (VL) comprising an amino acid sequence that has at least 90% sequence identity to any one of SEQ ID Nos: 21-33; (b) the transmembrane polypeptide of the second fusion protein comprises an amino acid sequence that has at least about 90% sequence identity to any one of SEQ ID NOs: 66-74; and (c) the oligomerization domain of the second fusion protein comprises an amino acid sequence that has at least 90% sequence identity to any one of SEQ ID NOs: 52-65. In some embodiments, the second fusion protein comprises an oligomerization domain, wherein: (a) the mammalian receptor comprises an amino acid sequence that has at least 90% sequence identity to any one of SEQ ID NOs: 34-43; (b) the transmembrane polypeptide of the second fusion protein comprises an amino acid sequence that has at least about 90% sequence identity to any one of SEQ ID NOs: 66-74; and (c) the oligomerization domain of the second fusion protein comprises an amino acid sequence that has at least 90% sequence identity to any one of SEQ ID NOs: 52-65. In some embodiments, the second fusion protein comprises an oligomerization domain, wherein: (a) the mammalian ligand comprises an amino acid sequence that has at least 90% sequence identity to any one of SEQ ID NOs: 44-51; (b) the transmembrane polypeptide of the second fusion protein comprises an amino acid sequence that has at least about 90% sequence identity to any one of SEQ ID NOs: 66-74; and (c) the oligomerization domain of the second fusion protein comprises an amino acid sequence that has at least 90% sequence identity to any one of SEQ ID NOs: 52-65.

Disclosed herein is a method of treating a disease in a subject in need thereof comprising expressing an IFN on a surface of a multivalent particle, wherein the multivalent particle has a binding affinity to an IFN receptor that is higher than the binding affinity of a soluble version of the IFN to the IFN receptor.

Disclosed herein is a composition comprising a multivalent interferon particle (IFN-MVP) that comprises an enveloped particle displaying at least 10 copies of an interferon (IFN) on a surface of the IFN-MVP. Disclosed herein is a composition comprising a guided IFN-MVP wherein the guided IFN-MVP comprises an enveloped particle that co-displays at least 10 copies of an IFN and at least 10 copies of a homing polypeptide on a surface of the IFN-MVP. Disclosed herein is a composition comprising an antibody-guided IFN-MVP wherein the antibody-guided IFN-MVP comprises an enveloped particle that co-displays at least 10 copies of an IFN and at least 10 copies of an antibody on a surface of the IFN-MVP wherein the antibody binds specifically to an antigen on a target cell or a target viral protein. Disclosed herein is a composition comprising a receptor-guided IFN-MVP wherein the receptor-guided IFN-MVP comprises an enveloped particle that co-displays at least 10 copies of an IFN and at least 10 copies of a receptor on a surface of the IFN-MVP wherein the receptor binds specifically to a target ligand. Disclosed herein is a composition comprising a ligand-guided IFN-MVP wherein the ligand-guided IFN-MVP comprises an enveloped particle that co-displays at least 10 copies of an IFN and at least 10 copies of a ligand on a surface of the IFN-MVP wherein the ligand binds specifically to a target receptor.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates the design of monomeric interferon (IFN)-multivalent particles (MVPs).

FIG. 1B illustrates the design of trimeric IFN-MVPs.

FIG. 2A illustrates the production of monomeric IFN viral-like particles (VLP) with viral genome.

FIG. 2B illustrates the production of monomeric IFN-VLPs without viral genome.

FIG. 2C illustrates the production of monomeric IFN-extracellular vesicles (EV).

FIG. 3A illustrates the production of trimeric IFN-VLPs with viral genome.

FIG. 3B illustrates the production of trimeric IFN-VLPs without viral genome.

FIG. 3C illustrates the production of trimeric IFN-EVs.

FIG. 4A illustrates the design of an oligomeric IFN-MVP.

FIG. 4B illustrates the design of a mixed oligomeric IFN-MVP co-displaying distinct IFNs.

FIG. 5A illustrates the production of mixed monomeric and trimeric combinatorial IFN-VLPs with viral genome.

FIG. 5B illustrates the production of mixed monomeric and trimeric combinatorial IFN-VLPs without viral genome.

FIG. 5C illustrates the production of mixed monomeric and trimeric combinatorial IFN-EVs.

FIG. 6 illustrates the design of a mixed oligomeric, antibody-guided IFN-MVP.

FIG. 7A illustrates the mechanism of dual-action, antibody-guided IFN-MVPs co-displaying IFNs and antibodies specific for viral surface proteins.

FIG. 7B illustrates the mechanism of dual-action, antibody-guided IFN-MVPs co-displaying IFNs and antibodies specific for tumor antigens.

FIG. 8 illustrates dual action of antiviral antibody-guided IFN-MVPs that can modulate immune responses in infected cells and directly neutralize virions.

FIG. 9A illustrates the production of mixed monomeric and trimeric antibody-guided IFN-VLPs with viral genome.

FIG. 9B illustrates the production of mixed monomeric and trimeric antibody-guided IFN-VLPs without viral genome.

FIG. 9C illustrates the production of mixed monomeric and trimeric antibody-guided IFN-EVs.

FIG. 10 illustrates the design of a mixed oligomeric, receptor-guided IFN-MVP.

FIG. 11A illustrates the mechanism of dual-action, receptor-guided IFN-MVPs co-displaying IFNs and receptors specific for viral surface proteins.

FIG. 11B illustrates the mechanism of dual-action, receptor-guided IFN-MVPs co-displaying IFNs and receptors specific for tumor antigens.

FIG. 12 illustrates dual action of antiviral receptor-guided IFN-MVPs that can modulate immune responses in infected cells and directly neutralize virions.

FIG. 13A illustrates the production of mixed monomeric and trimeric receptor-guided IFN-VLPs with viral genome.

FIG. 13B illustrates the production of mixed monomeric and trimeric receptor-guided IFN-VLPs without viral genome.

FIG. 13C illustrates the production of mixed monomeric and trimeric receptor-guided IFN-EVs.

FIG. 14A illustrates western blot analysis of trimeric IFN-MVPs.

FIG. 14B illustrates western blot analysis of monomeric IFN-MVPs.

FIG. 14C illustrates quantitative western blot analysis of trimeric IFN-MVPs.

FIG. 14D illustrates quantitative western blot analysis of monomeric IFN-MVPs.

FIG. 15A illustrates IFNα reporter signaling pathway in HEK-Blue 293 reporter cells.

FIG. 15B illustrates in vitro reporter assay titration curves for monomeric and trimeric IFNα-MVPs with soluble IFNα as positive control and IFN γ as negative control.

FIG. 16A illustrates IFNβ reporter signaling pathway in HEK-Blue 293 reporter cells.

FIG. 16B illustrates in vitro reporter assay titration curves for monomeric and trimeric IFNβ-MVPs with soluble IFNβ as positive control and IFNγ as negative control.

FIG. 17A illustrates IFNγ reporter signaling pathway in HEK-Blue 293 reporter cells.

FIG. 17B illustrates in vitro reporter assay titration curves for monomeric and trimeric IFNγ-MVPs with soluble IFNγ as positive control and IFNα as negative control.

FIG. 18A illustrates the effects of IFNα-MVPs on MHC I expression in S293 cells.

FIG. 18B illustrates the effects of IFNβ-MVPs on MHC I expression in S293 cells.

FIG. 18C illustrates the effects of IFNγ-MVPs on MHC I expression in S293 cells.

FIG. 19A illustrates the antiviral effects of various IFN-MVPs on HBV infection in vitro.

FIG. 19B illustrates in vitro inhibition of HBV replication by IFNα-MVPs.

FIG. 19C illustrates in vitro inhibition of HBV replication by IFNβ-MVPs.

FIG. 19D illustrates in vitro inhibition of HBV replication by IFNγ-MVPs.

FIG. 20 illustrates a method to determine antigen-specific binding by antibody-guided IFN-MVPs.

FIG. 21 illustrates a method to determine antigen-specific binding by receptor-guided IFN-MVPs.

FIG. 22 illustrates a method to determine antigen-specific modulation of MHC I expression by antibody-guided IFN-MVPs.

FIG. 23 illustrates a method to determine antigen-specific modulation of MHC I expression by receptor-guided IFN-MVPs.

### DETAILED DESCRIPTION OF THE INVENTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January;27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8;309 (3): 657-70, (“Aho” numbering scheme); and Whitelegg N R and Rees A R, “WAM: an improved algorithm for modelling antibodies on the WEB,” Protein Eng. 2000 December; 13 (12): 819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

### Multivalent Interferon Particles (IFN-MVPs)

Interferons (IFN) are a category of cytokines with immune-modulating, antiproliferative, and antiviral functions. Named for their ability to directly interfere with viral replication, IFNs induce the expression of numerous “interferon stimulated genes” (ISGs) within target cells and suppress viral protein translation or early viral infection steps, such as endosomal fusion. IFNs also stimulate the expression of chemokines to recruit lymphocytes to the site of a viral infection, and augment viral antigen presentation via upregulation of major histocompatibility complex (MHC) I expression. IFNs also activate several subsets of immune cells, including stimulating cytotoxic activity in natural killer (NK) cells, priming dendritic cells for cross-presentation to CD8 T cells and stimulating macrophages to secrete a number of inflammatory cytokines. In cancer settings, IFNs delay the cell cycle and induce tumor cell apoptosis to slow tumor growth while stimulating presentation of tumor-associated antigens (TAAs) via MHC I expression, allowing for immune surveillance and clearance of tumor cells. IFNs can also promote tumor cell growth, immune evasion and metastasis. The wide array of antiviral and antiproliferative functions mediated by IFNs make them attractive drug candidates for the treatment of numerous cancers and viruses.

IFNs have found a variety of clinical uses in the treatment of pathogenic viruses and cancer. IFN-α has been used as an initial treatment for a variety of leukemias and solid tumors, and pegylated IFN-α is currently an approved treatment for chronic hepatitis B virus (HBV) infection. However, in both oncogenic and viral therapeutic settings, IFN therapies suffer from limited patient response and strong non-specific immunological side effects. For example, in treating chronic HBV infection, PEG-IFN-α can result in seroconversion towards key viral antigens in around 20% of patients who tolerate the treatment, and serum HBV DNA clearance in an even lower proportion of patients. Meanwhile, patients undergoing IFN therapies regularly experience flu-like symptoms and exacerbated autoimmune conditions. As a result, IFN treatments are limited to short term regimens for most patients.

Disclosed herein are multivalent IFN particles (IFN-MVPs) with properties distinct from soluble IFNs. Multivalent IFN particles can utilize an avidity effect to form strong interactions with cognate cell surface receptors, thus eliciting stronger IFN signals at a significantly lower concentration than the corresponding soluble IFNs. In some embodiments, IFN-MVPs can be programmed to target specific tissue or cell-types to enhance specificity of antiviral and antiproliferative functions. Disclosed herein are compositions that comprise DNA or mRNA that encode the proteins that assemble into IFN-MVPs.

IFN-MVPs can be recombinant or synthetic particles displaying interferons in various oligomeric patterns. The recombinant IFN-MVPs are genetically encoded vesicles, for example viral-like particles (VLPs), exosomes, or ectosomes displaying many copies of IFN's fused to special display carrier peptides. Multivalent particles, which can be programmed to display combinations of distinct types of IFNs in various oligomeric conformations, are designed to form multivalent interactions with cognate receptors on target cells and to achieve stronger signaling at a relatively lower concentrations than soluble IFNs. Further, multivalent particles can be programmed to target specific cell types to mitigate the systemic immunological side effects of soluble IFNs. For example, disclosed herein are receptor-guided IFN multivalent particles and antibody-guided IFN multivalent particles co-displaying cellular receptors or antibodies in various oligomeric conformations alongside IFNs. The displayed receptors or antibodies allow the targeted IFN-multivalent particles to bind preferentially to cells displaying the corresponding ligands or antibody target, thus restricting the immunological effects of IFN-multivalent particles signaling to designated target cells, such as tumor cells expressing tumor-associated antigens (TAAs), infected cells displaying viral surface proteins, or immune cells expressing specific receptors or ligands. The combination of multivalent IFNs and targeting molecules displayed on mixed, guided IFN multivalent particles reduces the concentration of IFN-required for stimulation and mitigates non-specific IFN signaling while enhancing the antiviral and antiproliferative effects of IFNs on selected subsets of target cells. Thus, the mixed receptor-guided and antibody-guided IFN multivalent particles of the present disclosure can serve as IFN-based therapeutics for a broad set of diseases, while minimizing the typical immune toxicity of traditional IFN therapies.

Described herein, in some embodiments, are multivalent particles comprising a first fusion protein that comprises an interferon (IFN) polypeptide and a transmembrane polypeptide. In some embodiments, the first fusion protein is expressed on a surface of the multivalent particle. In some embodiments, the first fusion protein is expressed at a valency of at least about 10 copies on a surface of the multivalent particle.

In some embodiments, the multivalent particle further comprises a second fusion protein. In some embodiments, the second fusion protein comprises a transmembrane polypeptide and an IFN polypeptide that has less than 100% sequence identity to the IFN polypeptide of the first fusion protein. In some embodiments, the second fusion protein is expressed on a surface of the multivalent particle. In some embodiments, the second fusion protein is expressed at a valency of at least about 10 copies on a surface of the multivalent particle.

In some embodiments, the IFN polypeptide of the first fusion protein comprises a Type I IFN, Type II IFN, or a Type III IFN. In some embodiments, the IFN polypeptide of the second fusion protein comprises a Type I IFN, Type II IFN, or a Type III IFN. In some embodiments, the Type I IFN comprises IFN-alpha, IFN-beta, IFN-epsilon, IFN-kappa, or IFN-omega. In some embodiments, the Type II IFN comprises IFN-gamma. In some embodiments, the Type III IFN comprises IFN-lambda. In some embodiments, the IFN polypeptide of the first fusion protein comprises a human IFN polypeptide. In some embodiments, the IFN polypeptide of the second fusion protein comprises a human IFN polypeptide.

In some embodiments, the IFN polypeptide of the first fusion protein comprises an amino acid sequence with at least 75%, 80%, 85%, 90%, 95%, 96%, 97%, 98%, 99%, 100% sequence identity to any one of SEQ ID NOs: 1-7 listed in Table 1. In some embodiments, the IFN polypeptide of the second fusion protein comprises an amino acid sequence with at least 75%, 80%, 85%, 90%, 95%, 96%, 97%, 98%, 99%, 100% sequence identity sequence identity to any one of SEQ ID NOs: 1-7 listed in Table 1.

TABLE 1
Exemplary Human IFN Polypeptide Sequences

| | | SEQ ID |
|---|---|---|
| Name | Amino Acid Sequence | NO: |
| IFN alpha-2 | MALTFALLVALLVLSCKSSCSVGCDLPQ | 1 |
| | THSLGSRRTLMLLAQMRKISLFSCLKDR | |
| | HDFGFPQEEFGNQFQKAETIPVLHEMIQ | |
| | QIFNLFSTKDSSAAWDETLLDKFYTELY | |
| | QQLNDLEACVIQGVGVTETPLMKEDSIL | |
| | AVRKYFQRITLYLKEKKYSPCAWEVVR | |
| | AEIMRSFSLSTNLQESLRSKE | |
| IFN beta | MTNKCLLQIALLLCFSTTALSMSYNLLG | 2 |
| | FLQRSSNFQCQKLLWQLNGRLEYCLKD | |
| | RMNFDIPEEIKQLQQFQKEDAALTIYEM | |
| | LQNIFAIFRQDSSSTGWNETIVENLLANV | |
| | YHQINHLKTVLEEKLEKEDFTRGKLMSS | |
| | LHLKRYYGRILHYLKAKEYSHCAWTIV | |
| | RVEILRNFYFINRLTGYLRN | |
| IFN epsilon | MIIKHFFGTVLVLLASTTIFSLDLKLIIFQ | 3 |
| | QRQVNQESLKLLNKLQTLSIQQCLPHRK | |
| | NFLLPQKSLSPQQYQKGHTLAILHEMLQ | |
| | QIFSLFRANISLDGWEENHTEKFLIQLHQ | |
| | QLEYLEALMGLEAEKLSGTLGSDNLRLQ | |
| | VKMYFRRIHDYLENQDYSTCAWAIVQV | |
| | EISRCLFFVFSLTEKLSKQGRPLNDMKQE | |
| | LTTEFRSPR | |
| IFN kappa | MSTKPDMIQKCLWLEILMGIFIAGTLSLD | 4 |
| | CNLLNVHLRRVTWQNLRHLSSMSNSFP | |
| | VECLRENIAFELPQEFLQYTQPMKRDIK | |
| | KAFYEMSLQAFNIFSQHTFKYWKERHL | |
| | KQIQIGLDQQAEYLNQCLEEDKNENED | |
| | MKEMKENEMKPSEARVPQLSSLELRRY | |
| | FHRIDNFLKEKKYSDCAWEIVR VEIRRC | |
| | LYYFYKFTALFRRK | |
| IFN omega-1 | MALLFPLLAALVMTSYSPVGSLGCDLPQ | 5 |
| | NHGLLSRNTLVLLHQMRRISPFLCLKDR | |
| | RDFRFPQEMVKGSQLQKAHVMSVLHE | |
| | MLQQIFSLFHTERSSAAWNMTLLDQLHT | |
| | GLHQQLQHLETCLLQVVGEGESAGAISS | |
| | PALTLRRYFQGIRVYLKEKKYSDCAWE | |
| | VVRMEIMKSLFLSTNMQERLRSKDRDL | |
| | GSS | |
| IFN-gamma | MKYTSYILAFQLCIVLGSLGCYCQDPYV | 6 |
| | KEAENLKKYFNAGHSDVADNGTLFLGIL | |
| | KNWKEESDRKIMQSQIVSFYFKLFKNFK | |
| | DDQSIQKSVETIKEDMNVKFFNSNKKKR | |
| | DDFEKLTNYSVTDLNVQRKAIHELIQVM | |
| | AELSPAAKTGKRKRSQMLFRGRRASQ | |
| IFN lambda-1 | MAAAWTVVLVTLVLGLAVAGPVPTSKP | 7 |
| | TTTGKGCHIGRFKSLSPQELASFKKARD | |
| | ALEESLKLKNWSCSSPVFPGNWDLRLLQ | |
| | VRERPVALEAELALTLKVLEAAAGPALE | |
| | DVLDQPLHTLHHILSQLQACIQPQPTAGP | |
| | RPRGRLHHWLHRLQEAPKKESAGCLEA | |
| | SVTFNLFRLLTRDLKYVADGNLCLRTST | |
| | HPEST | |

In some embodiments, the IFN polypeptide of the first fusion protein comprises an amino acid sequence with at least 75% sequence identity to any one of SEQ ID NOs: 1-7. In some embodiments, the IFN polypeptide of the first fusion protein comprises an amino acid sequence with at least 76% sequence identity to any one of SEQ ID NOs: 1-7. In some embodiments, the IFN polypeptide of the first fusion protein comprises an amino acid sequence with at least 77% sequence identity to any one of SEQ ID NOs: 1-7. In some embodiments, the IFN polypeptide of the first fusion protein comprises an amino acid sequence with at least 78% sequence identity to any one of SEQ ID NOs: 1-7. In some embodiments, the IFN polypeptide of the first fusion protein comprises an amino acid sequence with at least 79% sequence identity to any one of SEQ ID NOs: 1-7. In some embodiments, the IFN polypeptide of the first fusion protein comprises an amino acid sequence with at least 80% sequence identity to any one of SEQ ID NOs: 1-7. In some embodiments, the IFN polypeptide of the first fusion protein comprises an amino acid sequence with at least 81% sequence identity to any one of SEQ ID NOs: 1-7. In some embodiments, the IFN polypeptide of the first fusion protein comprises an amino acid sequence with at least 82% sequence identity to any one of SEQ ID NOs: 1-7. In some embodiments, the IFN polypeptide of the first fusion protein comprises an amino acid sequence with at least 83% sequence identity to any one of SEQ ID NOs: 1-7. In some embodiments, the IFN polypeptide of the first fusion protein comprises an amino acid sequence with at least 84% sequence identity to any one of SEQ ID NOs: 1-7. In some embodiments, the IFN polypeptide of the first fusion protein comprises an amino acid sequence with at least 85% sequence identity to any one of SEQ ID NOs: 1-7. In some embodiments, the IFN polypeptide of the first fusion protein comprises an amino acid sequence with at least 86% sequence identity to any one of SEQ ID NOs: 1-7. In some embodiments, the IFN polypeptide of the first fusion protein comprises an amino acid sequence with at least 87% sequence identity to any one of SEQ ID NOs: 1-7. In some embodiments, the IFN polypeptide of the first fusion protein comprises an amino acid sequence with at least 88% sequence identity to any one of SEQ ID NOs: 1-7. In some embodiments, the IFN polypeptide of the first fusion protein comprises an amino acid sequence with at least 89% sequence identity to any one of SEQ ID NOs: 1-7. In some embodiments, the IFN polypeptide of the first fusion protein comprises an amino acid sequence with at least 90% sequence identity to any one of SEQ ID NOs: 1-7. In some embodiments, the IFN polypeptide of the first fusion protein comprises an amino acid sequence with at least 91% sequence identity to any one of SEQ ID NOs: 1-7. In some embodiments, the IFN polypeptide of the first fusion protein comprises an amino acid sequence with at least 92% sequence identity to any one of SEQ ID NOs: 1-7. In some embodiments, the IFN polypeptide of the first fusion protein comprises an amino acid sequence with at least 93% sequence identity to any one of SEQ ID NOs: 1-7. In some embodiments, the IFN polypeptide of the first fusion protein comprises an amino acid sequence with at least 94% sequence identity to any one of SEQ ID NOs: 1-7. In some embodiments, the IFN polypeptide of the first fusion protein comprises an amino acid sequence with at least 95% sequence identity to any one of SEQ ID NOs: 1-7. In some embodiments, the IFN polypeptide of the first fusion protein comprises an amino acid sequence with at least 96% sequence identity to any one of SEQ ID NOs: 1-7. In some embodiments, the IFN polypeptide of the first fusion protein comprises an amino acid sequence with at least 97% sequence identity to any one of SEQ ID NOs: 1-7. In some embodiments, the IFN polypeptide of the first fusion protein comprises an amino acid sequence with at least 98% sequence identity to any one of SEQ ID NOs: 1-7. In some embodiments, the IFN polypeptide of the first fusion protein comprises an amino acid sequence with at least 99% sequence identity to any one of SEQ ID NOs: 1-7. In some embodiments, the IFN polypeptide of the first fusion protein comprises an amino acid sequence according to any one of SEQ ID NOs: 1-7.

In some embodiments, the IFN polypeptide of the second fusion protein comprises an amino acid sequence with at least 75% sequence identity to any one of SEQ ID NOs: 1-7. In some embodiments, the IFN polypeptide of the second fusion protein comprises an amino acid sequence with at least 76% sequence identity to any one of SEQ ID NOs: 1-7. In some embodiments, the IFN polypeptide of the second fusion protein comprises an amino acid sequence with at least 77% sequence identity to any one of SEQ ID NOs: 1-7. In some embodiments, the IFN polypeptide of the second fusion protein comprises an amino acid sequence with at least 78% sequence identity to any one of SEQ ID NOs: 1-7. In some embodiments, the IFN polypeptide of the second fusion protein comprises an amino acid sequence with at least 79% sequence identity to any one of SEQ ID NOs: 1-7. In some embodiments, the IFN polypeptide of the second fusion protein comprises an amino acid sequence with at least 80% sequence identity to any one of SEQ ID NOs: 1-7. In some embodiments, the IFN polypeptide of the second fusion protein comprises an amino acid sequence with at least 81% sequence identity to any one of SEQ ID NOs: 1-7. In some embodiments, the IFN polypeptide of the second fusion protein comprises an amino acid sequence with at least 82% sequence identity to any one of SEQ ID NOs: 1-7. In some embodiments, the IFN polypeptide of the second fusion protein comprises an amino acid sequence with at least 83% sequence identity to any one of SEQ ID NOs: 1-7. In some embodiments, the IFN polypeptide of the second fusion protein comprises an amino acid sequence with at least 84% sequence identity to any one of SEQ ID NOs: 1-7. In some embodiments, the IFN polypeptide of the second fusion protein comprises an amino acid sequence with at least 85% sequence identity to any one of SEQ ID NOs: 1-7. In some embodiments, the IFN polypeptide of the second fusion protein comprises an amino acid sequence with at least 86% sequence identity to any one of SEQ ID NOs: 1-7. In some embodiments, the IFN polypeptide of the second fusion protein comprises an amino acid sequence with at least 87% sequence identity to any one of SEQ ID NOs: 1-7. In some embodiments, the IFN polypeptide of the second fusion protein comprises an amino acid sequence with at least 88% sequence identity to any one of SEQ ID NOs: 1-7. In some embodiments, the IFN polypeptide of the second fusion protein comprises an amino acid sequence with at least 89% sequence identity to any one of SEQ ID NOs: 1-7. In some embodiments, the IFN polypeptide of the second fusion protein comprises an amino acid sequence with at least 90% sequence identity to any one of SEQ ID NOs: 1-7. In some embodiments, the IFN polypeptide of the second fusion protein comprises an amino acid sequence with at least 91% sequence identity to any one of SEQ ID NOs: 1-7. In some embodiments, the IFN polypeptide of the second fusion protein comprises an amino acid sequence with at least 92% sequence identity to any one of SEQ ID NOs: 1-7. In some embodiments, the IFN polypeptide of the second fusion protein comprises an amino acid sequence with at least 93% sequence identity to any one of SEQ ID NOs: 1-7. In some embodiments, the IFN polypeptide of the second fusion protein comprises an amino acid sequence with at least 94% sequence identity to any one of SEQ ID NOs: 1-7. In some embodiments, the IFN polypeptide of the second fusion protein comprises an amino acid sequence with at least 95% sequence identity to any one of SEQ ID NOs: 1-7. In some embodiments, the IFN polypeptide of the second fusion protein comprises an amino acid sequence with at least 96% sequence identity to any one of SEQ ID NOs: 1-7. In some embodiments, the IFN polypeptide of the second fusion protein comprises an amino acid sequence with at least 97% sequence identity to any one of SEQ ID NOs: 1-7. In some embodiments, the IFN polypeptide of the second fusion protein comprises an amino acid sequence with at least 98% sequence identity to any one of SEQ ID NOs: 1-7. In some embodiments, the IFN polypeptide of the second fusion protein comprises an amino acid sequence with at least 99% sequence identity to any one of SEQ ID NOs: 1-7. In some embodiments, the IFN polypeptide of the second fusion protein comprises an amino acid sequence according to any one of SEQ ID NOs: 1-7.

In some embodiments, the second fusion protein comprises a transmembrane polypeptide and a homing polypeptide that targets the multivalent particle to a target cell. In some embodiments, the second fusion protein comprises a transmembrane polypeptide and a homing polypeptide that targets the multivalent particle to a target protein. In some embodiments, the second fusion protein is expressed on a surface of the multivalent particle. In some embodiments, the second fusion protein is expressed at a valency of at least about 10 copies on a surface of the multivalent particle. In some embodiments, the target cell comprises a cancer cell. In some embodiments, the cancer cell is from a cancer selected from a group comprising melanoma, leukemia, lymphoma, multiple myeloma, liver cancer, pancreatic cancer, lung cancer, breast cancer, prostate cancer, brain cancer, colorectal cancer, bladder cancer, kidney cancer, cervical cancer, ovarian cancer, esophageal cancer, mesothelioma, gastric cancer, and sarcoma.

In some embodiments, the target protein comprises a viral surface protein. In some embodiments, the viral surface protein is from hepatitis B virus (HBV), SARS CoV-2, SARS CoV-1, MERS CoV, Influenza, respiratory syncytial virus, HIV, or measles. In some embodiments, the target protein comprises a viral spike protein. In some embodiments, the homing polypeptide comprises an antibody that binds specifically to an antigen on the target cell or the target protein. In some embodiments, the antigen on the target cell comprises an antigen listed in Table 2. In some embodiments, the target protein comprises the receptor binding domain (RBD) of the spike protein of SARS CoV-1. In some embodiments, the target protein comprises the receptor binding domain (RBD) of the spike protein of SARS CoV-2. In some embodiments, the target protein comprises the N-terminal domain (NTD) of the spike protein of MERS CoV. In some embodiments, the target protein comprises the Influenza Hemagglutinin (HA) stem. In some embodiments, the target protein comprises the Influenza Neuraminidase enzyme active site. In some embodiments, the target protein comprises the small hepatitis B surface antigen (S-HBsAg) of the HBV. In some embodiments, the target protein comprises the medium hepatitis B surface antigen (M-HBsAg) of the HBV. In some embodiments, the target protein comprises the large hepatitis B surface antigen (L-HBsAg) of the HBV. In some embodiments, the target protein comprises the Pre-S1 domain of L-HBsAg of the HBV.

TABLE 2
Exemplary Cancer Types and Antigens

| Cancer Type | Antigen |
|---|---|
| Melanoma | Tyrosinase |
| Melanoma | MARTI |
| Melanoma | gp 100 |
| Melanoma | NY-ESO-1 |
| Leukemia/Lymphoma | WT1 |
| Leukemia/Lymphoma | Minor Histocompatibility Antigens |
| Leukemia/Lymphoma | CD19 |
| Leukemia/Lymphoma | CD20 |
| Leukemia/Lymphoma | CD22 |
| Leukemia/Lymphoma | CD33 |
| Leukemia/Lymphoma | CD38 |
| Multiple Myeloma | BCMA |
| Multiple Myeloma | NY-ESO-1 |
| Liver Cancer | AFP |
| Liver Cancer | Glypican-3 |
| Pancreatic Cancer | Mesothelin |
| Pancreatic Cancer | CA19-9 |
| Pancreatic Cancer | CEA |
| Pancreatic Cancer | EpCAM |
| Lung Cancer | EGFR |
| Lung Cancer | NY-ESO-1 |
| Lung Cancer | Mesothelin |
| Lung Cancer | MAGE-A1/3/4 |
| Lung Cancer | DLL-3 |
| Lung Cancer | PSCA |
| Lung Cancer | Carcinoembryonic Antigen (CEA) |
| Lung Cancer | squamous cell carcinoma antigen (SCC) |
| Lung Cancer | neuron-specific enolase (NSE) |
| Lung Cancer | cytokeratin 19 fragment (CYFRA) |
| Lung Cancer | pro-gastrin-releasing peptide (proGRP) |
| Breast Cancer | HER2 |
| Breast Cancer | CEA |
| Breast Cancer | cMet |
| Breast Cancer | MUC1 |
| Breast Cancer | Mesothelin |
| Breast Cancer | EGFR |
| Breast Cancer | CD32 |
| Prostate Cancer | PSMA |
| Prostate Cancer | EpCAM |
| Prostate Cancer | Prostate-specific antigen (PSA) |
| Brain Cancer | EGFRvIII |
| Brain Cancer | GD2 |
| Brain Cancer | EGFR806 |
| Brain Cancer | IL-13Rα2 |
| Brain Cancer | CD171 |
| Brain Cancer | EpHA2 |
| Colorectal Cancer | EpCAM |
| Colorectal Cancer | Claudin 18.2 |
| Bladder Cancer | MAGE-A3 |
| Bladder Cancer | HER2 |
| Bladder Cancer | ROR2 |
| Bladder Cancer | PSMA |
| Bladder Cancer | FRA |
| Kidney Cancer | VEGFRE2 |
| Kidney Cancer | CD70 |
| Cervical Cancer | Mesothelin |
| Ovarian Cancer | MUC16 |
| Ovarian Cancer | Mesothelin |
| Ovarian Cancer | Alpha Folate Receptor |
| Ovarian Cancer | HER2 |
| Ovarian Cancer | CD70 |
| Esophageal Cancer | EpHA2 |
| Esophageal Cancer | Guanylyl Cyclase C (GUCY2C) |
| Mesothelioma | Mesothelin |
| Gastric Cancer | EpCAM |
| Sarcoma | NY-ESO-1 |

In some embodiments, the antibody comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc. In some embodiments, the antibody comprises an amino acid sequence from at least one complementarity determining region of BG10-19, 80R, 7D10, FI6, 1E01, H015, 2H5, ADRI-2F3, H004, H009, H007, H019, or H020. In some embodiments, the antibody comprises a multi-specific antibody. In some embodiments, the antibody comprises a bispecific antibody. In some embodiments, the antibody comprises a tri-specific antibody. In some embodiments, the antibody comprises a heavy domain variable region (VH) according to any one of the sequences in Table 3A. In some embodiments, the antibody comprises a light domain variable region (VL) according to any one of the sequences in Table 3A. In some embodiments, the antibody comprises a complementarity determining region (CDR) of any one of the sequences in Table 3B. In some embodiments, the CDR sequences (CDR-H1, CDR-H2, CDR-H3 and CDR-L1, CDR-L2, CDR-L3) are determined according to any of the definitions described above.

In some embodiments, the antibody comprises a heavy chain complementarity determining region 1 (CDR-H1) according to any one of SEQ ID NOs: 75, 78, 81, 84, 87, 90, 93, 96, 99, 102, 105, 108, and 111. In some embodiments, the antibody comprises a heavy chain complementarity determining region 2 (CDR-H2) according to any one of SEQ ID NOs: 76, 79, 82, 85, 88, 91, 94, 97, 100, 103, 106, 109, and 112. In some embodiments, the antibody comprises a heavy chain complementarity determining region 3 (CDR-H3) according to any one of SEQ ID NOs: 77, 80, 83, 86, 89, 92, 95, 98, 101, 104, 107, 110, and 113. In some embodiments, the antibody comprises a light chain complementarity determining region 1 (CDR-L1) according to any one of SEQ ID NOs: 114, 117, 120, 123, 126, 129, 132, 135, 138, 141, 144, 147, and 150. In some embodiments, the antibody comprises a light chain complementarity determining region 2 (CDR-L2) according to any one of SEQ ID NOs: 115, 118, 121, 124, 127, 130, 133, 136, 139, 142, 145, 148, and 151. In some embodiments, the antibody comprises a light chain complementarity determining region 4 (CDR-L3) according to any one of SEQ ID NOs: 116, 119, 122, 125, 128, 131, 134, 137, 140, 143, 146, 149, and 152. In some embodiments, the antibody comprises a CDR-H1 according to SEQ ID NO: 75, a CDR-H2 according to SEQ ID NO: 76, a CDR-H3 according to SEQ ID NO: 77, a CDR-L1 according to SEQ ID NO: 114, a CDR-L2 according to SEQ ID NO: 115, and a CDR-L3 according to SEQ ID NO: 116. In some embodiments, the antibody comprises a CDR-H1 according to SEQ ID NO: 78, a CDR-H2 according to SEQ ID NO: 79, a CDR-H3 according to SEQ ID NO: 80, a CDR-L1 according to SEQ ID NO: 117, a CDR-L2 according to SEQ ID NO: 118, and a CDR-L3 according to SEQ ID NO: 119. In some embodiments, the antibody comprises a CDR-H1 according to SEQ ID NO: 81, a CDR-H2 according to SEQ ID NO: 82, a CDR-H3 according to SEQ ID NO: 83, a CDR-L1 according to SEQ ID NO: 120, a CDR-L2 according to SEQ ID NO: 121, and a CDR-L3 according to SEQ ID NO: 122. In some embodiments, the antibody comprises a CDR-H1 according to SEQ ID NO: 84, a CDR-H2 according to SEQ ID NO: 85, a CDR-H3 according to SEQ ID NO: 86, a CDR-L1 according to SEQ ID NO: 123, a CDR-L2 according to SEQ ID NO: 124, and a CDR-L3 according to SEQ ID NO: 125. In some embodiments, the antibody comprises a CDR-H1 according to SEQ ID NO: 87, a CDR-H2 according to SEQ ID NO: 88, a CDR-H3 according to SEQ ID NO: 89, a CDR-L1 according to SEQ ID NO: 126, a CDR-L2 according to SEQ ID NO: 127, and a CDR-L3 according to SEQ ID NO: 128. In some embodiments, the antibody comprises a CDR-H1 according to SEQ ID NO: 90, a CDR-H2 according to SEQ ID NO: 91, a CDR-H3 according to SEQ ID NO: 92, a CDR-L1 according to SEQ ID NO: 129, a CDR-L2 according to SEQ ID NO: 130, and a CDR-L3 according to SEQ ID NO: 131. In some embodiments, the antibody comprises a CDR-H1 according to SEQ ID NO: 93, a CDR-H2 according to SEQ ID NO: 94, a CDR-H3 according to SEQ ID NO: 95, a CDR-L1 according to SEQ ID NO: 132, a CDR-L2 according to SEQ ID NO: 133, and a CDR-L3 according to SEQ ID NO: 134. In some embodiments, the antibody comprises a CDR-H1 according to SEQ ID NO: 96, a CDR-H2 according to SEQ ID NO: 97, a CDR-H3 according to SEQ ID NO: 98, a CDR-L1 according to SEQ ID NO: 135, a CDR-L2 according to SEQ ID NO: 136, and a CDR-L3 according to SEQ ID NO: 137. In some embodiments, the antibody comprises a CDR-H1 according to SEQ ID NO: 99, a CDR-H2 according to SEQ ID NO: 100, a CDR-H3 according to SEQ ID NO: 101, a CDR-L1 according to SEQ ID NO: 138, a CDR-L2 according to SEQ ID NO: 139, and a CDR-L3 according to SEQ ID NO: 140. In some embodiments, the antibody comprises a CDR-H1 according to SEQ ID NO: 102, a CDR-H2 according to SEQ ID NO: 103, a CDR-H3 according to SEQ ID NO: 104, a CDR-L1 according to SEQ ID NO: 141, a CDR-L2 according to SEQ ID NO: 142, and a CDR-L3 according to SEQ ID NO: 143. In some embodiments, the antibody comprises a CDR-H1 according to SEQ ID NO: 105, a CDR-H2 according to SEQ ID NO: 106, a CDR-H3 according to SEQ ID NO: 107, a CDR-L1 according to SEQ ID NO: 144, a CDR-L2 according to SEQ ID NO: 145, and a CDR-L3 according to SEQ ID NO: 146. In some embodiments, the antibody comprises a CDR-H1 according to SEQ ID NO: 108, a CDR-H2 according to SEQ ID NO: 109, a CDR-H3 according to SEQ ID NO: 110, a CDR-L1 according to SEQ ID NO: 147, a CDR-L2 according to SEQ ID NO: 148, and a CDR-L3 according to SEQ ID NO: 149. In some embodiments, the antibody comprises a CDR-H1 according to SEQ ID NO: 111, a CDR-H2 according to SEQ ID NO: 112, a CDR-H3 according to SEQ ID NO: 113, a CDR-L1 according to SEQ ID NO: 150, a CDR-L2 according to SEQ ID NO: 151, and a CDR-L3 according to SEQ ID NO: 152.

In some embodiments, the antibody comprises a VH comprising an amino acid sequence with at least 75%, 80%, 85%, 90%, 95%, 96%, 97%, 98%, 99%, 100% sequence identity to any one of SEQ ID NOs: 8-20. In some embodiments, the antibody comprises a VL comprising an amino acid sequence with at least 75%, 80%, 85%, 90%, 95%, 96%, 97%, 98%, 99%, 100% sequence identity to any one of SEQ ID NOs: 21-33. In some embodiments, the antibody comprises a VH comprising an amino acid sequence with at least 75% sequence identity to any one of SEQ ID NOs: 8-20 and a VL comprising an amino acid sequence with at least 75% sequence identity to any one of SEQ ID NOs: 21-33. In some embodiments, the antibody comprises a VH comprising an amino acid sequence with at least 76% sequence identity to any one of SEQ ID NOs: 8-20 and a VL comprising an amino acid sequence with at least 76% sequence identity to any one of SEQ ID NOs: 21-33. In some embodiments, the antibody comprises a VH comprising an amino acid sequence with at least 77% sequence identity to any one of SEQ ID NOs: 8-20 and a VL comprising an amino acid sequence with at least 77% sequence identity to any one of SEQ ID NOs: 21-33. In some embodiments, the antibody comprises a VH comprising an amino acid sequence with at least 78% sequence identity to any one of SEQ ID NOs: 8-20 and a VL comprising an amino acid sequence with at least 78% sequence identity to any one of SEQ ID NOs: 21-33. In some embodiments, the antibody comprises a VH comprising an amino acid sequence with at least 79% sequence identity to any one of SEQ ID NOs: 8-20 and a VL comprising an amino acid sequence with at least 79% sequence identity to any one of SEQ ID NOs: 21-33. In some embodiments, the antibody comprises a VH comprising an amino acid sequence with at least 80% sequence identity to any one of SEQ ID NOs: 8-20 and a VL comprising an amino acid sequence with at least 80% sequence identity to any one of SEQ ID NOs: 21-33. In some embodiments, the antibody comprises a VH comprising an amino acid sequence with at least 81% sequence identity to any one of SEQ ID NOs: 8-20 and a VL comprising an amino acid sequence with at least 81% sequence identity to any one of SEQ ID NOs: 21-33. In some embodiments, the antibody comprises a VH comprising an amino acid sequence with at least 82% sequence identity to any one of SEQ ID NOs: 8-20 and a VL comprising an amino acid sequence with at least 82% sequence identity to any one of SEQ ID NOs: 21-33. In some embodiments, the antibody comprises a VH comprising an amino acid sequence with at least 83% sequence identity to any one of SEQ ID NOs: 8-20 and a VL comprising an amino acid sequence with at least 83% sequence identity to any one of SEQ ID NOs: 21-33. In some embodiments, the antibody comprises a VH comprising an amino acid sequence with at least 84% sequence identity to any one of SEQ ID NOs: 8-20 and a VL comprising an amino acid sequence with at least 84% sequence identity to any one of SEQ ID NOs: 21-33. In some embodiments, the antibody comprises a VH comprising an amino acid sequence with at least 85% sequence identity to any one of SEQ ID NOs: 8-20 and a VL comprising an amino acid sequence with at least 85% sequence identity to any one of SEQ ID NOs: 21-33. In some embodiments, the antibody comprises a VH comprising an amino acid sequence with at least 86% sequence identity to any one of SEQ ID NOs: 8-20 and a VL comprising an amino acid sequence with at least 86% sequence identity to any one of SEQ ID NOs: 21-33. In some embodiments, the antibody comprises a VH comprising an amino acid sequence with at least 87% sequence identity to any one of SEQ ID NOs: 8-20 and a VL comprising an amino acid sequence with at least 87% sequence identity to any one of SEQ ID NOs: 21-33. In some embodiments, the antibody comprises a VH comprising an amino acid sequence with at least 88% sequence identity to any one of SEQ ID NOs: 8-20 and a VL comprising an amino acid sequence with at least 88% sequence identity to any one of SEQ ID NOs: 21-33. In some embodiments, the antibody comprises a VH comprising an amino acid sequence with at least 89% sequence identity to any one of SEQ ID NOs: 8-20 and a VL comprising an amino acid sequence with at least 89% sequence identity to any one of SEQ ID NOs: 21-33. In some embodiments, the antibody comprises a VH comprising an amino acid sequence with at least 90% sequence identity to any one of SEQ ID NOs: 8-20 and a VL comprising an amino acid sequence with at least 90% sequence identity to any one of SEQ ID NOs: 21-33. In some embodiments, the antibody comprises a VH comprising an amino acid sequence with at least 91% sequence identity to any one of SEQ ID NOs: 8-20 and a VL comprising an amino acid sequence with at least 91% sequence identity to any one of SEQ ID NOs: 21-33. In some embodiments, the antibody comprises a VH comprising an amino acid sequence with at least 92% sequence identity to any one of SEQ ID NOs: 8-20 and a VL comprising an amino acid sequence with at least 92% sequence identity to any one of SEQ ID NOs: 21-33. In some embodiments, the antibody comprises a VH comprising an amino acid sequence with at least 93% sequence identity to any one of SEQ ID NOs: 8-20 and a VL comprising an amino acid sequence with at least 93% sequence identity to any one of SEQ ID NOs: 21-33. In some embodiments, the antibody comprises a VH comprising an amino acid sequence with at least 94% sequence identity to any one of SEQ ID NOs: 8-20 and a VL comprising an amino acid sequence with at least 94% sequence identity to any one of SEQ ID NOs: 21-33. In some embodiments, the antibody comprises a VH comprising an amino acid sequence with at least 95% sequence identity to any one of SEQ ID NOs: 8-20 and a VL comprising an amino acid sequence with at least 95% sequence identity to any one of SEQ ID NOs: 21-33. In some embodiments, the antibody comprises a VH comprising an amino acid sequence with at least 96% sequence identity to any one of SEQ ID NOs: 8-20 and a VL comprising an amino acid sequence with at least 96% sequence identity to any one of SEQ ID NOs: 21-33. In some embodiments, the antibody comprises a VH comprising an amino acid sequence with at least 97% sequence identity to any one of SEQ ID NOs: 8-20 and a VL comprising an amino acid sequence with at least 97% sequence identity to any one of SEQ ID NOs: 21-33. In some embodiments, the antibody comprises a VH comprising an amino acid sequence with at least 98% sequence identity to any one of SEQ ID NOs: 8-20 and a VL comprising an amino acid sequence with at least 98% sequence identity to any one of SEQ ID NOs: 21-33. In some embodiments, the antibody comprises a VH comprising an amino acid sequence with at least 99% sequence identity to any one of SEQ ID NOs: 8-20 and a VL comprising an amino acid sequence with at least 99% sequence identity to any one of SEQ ID NOs: 21-33.

In some embodiments, the antibody comprises a VH comprising an amino acid sequence of any one of SEQ ID NOs: 8-20 and a VL comprising an amino acid sequence of any one of SEQ ID NOs: 21-33. In some embodiments, the VH domain comprises the amino acid sequence according to SEQ ID NO: 8, and the VL domain comprises the amino acid sequence according to SEQ ID NO: 21. In some embodiments, the VH domain comprises the amino acid sequence according to SEQ ID NO: 9, and the VL domain comprises the amino acid sequence according to SEQ ID NO: 22. In some embodiments, the VH domain comprises the amino acid sequence according to SEQ ID NO: 10, and the VL domain comprises the amino acid sequence according to SEQ ID NO: 23. In some embodiments, the VH domain comprises the amino acid sequence according to SEQ ID NO: 11, and the VL domain comprises the amino acid sequence according to SEQ ID NO: 24. In some embodiments, the VH domain comprises the amino acid sequence according to SEQ ID NO: 12, and the VL domain comprises the amino acid sequence according to SEQ ID NO: 25. In some embodiments, the VH domain comprises the amino acid sequence according to SEQ ID NO: 13, and the VL domain comprises the amino acid sequence according to SEQ ID NO: 26. In some embodiments, the VH domain comprises the amino acid sequence according to SEQ ID NO: 14, and the VL domain comprises the amino acid sequence according to SEQ ID NO: 27. In some embodiments, the VH domain comprises the amino acid sequence according to SEQ ID NO: 15, and the VL domain comprises the amino acid sequence according to SEQ ID NO: 28. In some embodiments, the VH domain comprises the amino acid sequence according to SEQ ID NO: 16, and the VL domain comprises the amino acid sequence according to SEQ ID NO: 29. In some embodiments, the VH domain comprises the amino acid sequence according to SEQ ID NO: 17, and the VL domain comprises the amino acid sequence according to SEQ ID NO: 30. In some embodiments, the VH domain comprises the amino acid sequence according to SEQ ID NO: 18, and the VL domain comprises the amino acid sequence according to SEQ ID NO: 31. In some embodiments, the VH domain comprises the amino acid sequence according to SEQ ID NO: 19, and the VL domain comprises the amino acid sequence according to SEQ ID NO: 32. In some embodiments, the VH domain comprises the amino acid sequence according to SEQ ID NO: 20, and the VL domain comprises the amino acid sequence according to SEQ ID NO: 33.

TABLE 3A
Exemplary Antibody Sequences

| | | | SEQ | | SEQ | |
|---|---|---|---|---|---|---|
| | | | ID | VH Amino Acid | ID | VL Amino Acid |
| Name | Modality | Target | NO | Sequence | NO | Sequence |
| BG10- | Virus; | Spike RBD | 8 | ATGVHSEVQLVQSGA | 21 | ATGSWAQSVLTQPPS |
| 19 | SARS | | | EVKKPGESLKISCKGS | | ASGTPGQRVTISCSGS |
| | CoV-2 | | | GYSFTSYWIGWVRQ | | SSNIGSDHVYWYQQ |
| | | | | MPGKGLEWMGVIYP | | LPGTAPKLFIYRNNQ |
| | | | | GDSDTRYSPSFQGQV | | RPSGVPDRFSGSKSG |
| | | | | TISADKSISTAYLQWS | | TSASLAISGLRSEDEA |
| | | | | SLKASDTAMYYCART | | DYYCAAWDASLSGY |
| | | | | QWGYNYGSHFFYMD | | VFGTGTKVTVLGQP |
| | | | | VWGKGTTVTVSSAST | | KANPTVTLFPPS |
| 80R | Virus; | Spike RBD | 9 | EVQLVQSGGGVVQPG | 22 | ETTLTQSPATLSLSPG |
| | SARS | | | KSLRLSCAASGFAFSS | | ERATLSCRASQSVRS |
| | CoV-1 | | | YAMHWVRQAPGKGL | | NLAWYQQKPGQAPR |
| | | | | EWVAVISYDGSNKYY | | PLIYDASTRATGIPDR |
| | | | | ADSVKGRFTISRDNSK | | FSGSGSGTDFTLTISR |
| | | | | NTLYLQMNSLRAEDT | | LEPEDFAVYYCQQRS |
| | | | | AVYYCARDRSYYLD | | NWPPTFGQGTKVEV |
| | | | | YWGQGTLVTVSS | | KSGLVPR |
| 7D10 | Virus; | Spike NTD | 10 | EVQLVESGAEVVKPG | 23 | DIVMTQSPASLTVSL |
| | MERS | | | ASVKMSCKASGYPFT | | GQRATISCRASKSVS |
| | CoV | | | SYNIHWIKQTPGQGLE | | ASGYNYLHWYQQRP |
| | | | | WIGAIYPGNGDTSYT | | GQPPKLLIYLAFNLES |
| | | | | QKFKVKATLTSDKSS | | GVPARFNGSGSGTDF |
| | | | | STAYMQLSSLTSEDSA | | TLNIHPVEEEDAATY |
| | | | | VYFCARYGNYPSYA | | YCQHSRDLPFTFGSG |
| | | | | MDYWGQGTSVTVSS | | TKLEIK |
| FI6 | Virus; | Hemagglutinin | 11 | QVQLVESGGGVVQPG | 24 | DIVMTQSPDSLAVSL |
| | Influenza | stem | | RSLRLSCAASGFTFST | | GERATINCKSSQSVT |
| | | | | YAMHWVRQAPGKGL | | FNYKNYLAWYQQKP |
| | | | | EWVAVISYDANYKY | | GQPPKLLIYWASTRE |
| | | | | YADSVKGRFTISRDNS | | SGVPDRFSGSGSGTD |
| | | | | KNTLYLQMNSLRAED | | FTLTISSLQAEDVAV |
| | | | | TAVYYCAKDSQLRSL | | YYCQQHYRTPPTFGQ |
| | | | | LYFEWLSQGYFDYW | | GTKVEIK |
| | | | | GQGTLVTVSS | | |
| 1E01 | Virus; | Neuraminidase | 12 | EVQLVESGGRVVRPG | 25 | DIQLTQSPSFLSASVG |
| | Influenza | Active Site | | GSLRLSCAASGFTFDD | | DRVTITCRASQDISSY |
| | | | | YGMSWVRQPPGKGL | | LAWYQQKPGNAPKL |
| | | | | EFVSGLNWNGDITAF | | LIYAASLLQSGVPSRF |
| | | | | TDSVKGRFTISRDNVK | | SAFGSGTEFTLTISSL |
| | | | | SSLYLQMNSLRADDT | | QPEDFATYYCQHLKS |
| | | | | AFYYCARVRTWGDY | | YPLFTFGPGTKVDIK |
| | | | | TTGEEIINSWYFDLWG | | |
| | | | | RGTLVTVSS | | |
| H015 | Virus; | S HBsAg | 13 | QVQLVESGGGVAQPG | 26 | DIQVTQSPSSLSAFVG |
| | HBV | | | RSLRLSCAASGFSFSR | | DRVTITCRASQGLTS |
| | | | | HGMHWVRQAPGKGL | | FINWYQQKPGRAPKL |
| | | | | EWVAGIWFDGTNDY | | LISSASSLQRGVPSRF |
| | | | | YTDSVKGRFTISRDNS | | TASGSGTHFTLTISSL |
| | | | | RSTLYLDINSLRAEDT | | QPEDFATYYCQQSY |
| | | | | AVYYCAREDPHLLIA | | GTPALAFGGGTKVDI |
| | | | | TLDLWGLGTLVTVSS | | K |
| | | | | AS | | |
| 2H5 | Virus; | L PreS1 | 14 | SQVQLQQSGPGLVKP | 27 | QSVLTQPPSASGTPG |
| | HBV | | | SQTLSLTCGISGDSVS | | QRVTISCSGSSSNIGS |
| | | | | SKSAAWNWIRQSPSR | | YYVYWYQQFPGTAP |
| | | | | GLEWLGRTYYRSKW | | KLLIYGNNQRPSGVP |
| | | | | HNDYAVSVKSRITINP | | DRFSGSKSGTSASLAI |
| | | | | DTSKNQFSLQLNSVTP | | TGLQAEDEADYYCQ |
| | | | | EDTAVYYCARGQMG | | SYDSSLSGVIFGGGT |
| | | | | ALDVWGQGTTVTVSS | | KLTVL |
| ADRI- | Virus; | S HBsAg | 15 | EVQVLESGGGLVQPG | 28 | SYVLTQPPSVSVAPG |
| 2F3 | HBV | | | GSLRLSCAASGFRESS | | QTARMTCGGNNIGSE |
| | | | | YAMSWVRQAPGKGL | | SVHWFQQKPGQAPV |
| | | | | EWVSGISGTGENTYY | | LVVYDDSDRPSGIPE |
| | | | | ADSVKGRFTISRDNSK | | RFSGSNSGNTATLTIS |
| | | | | NTLYVQMNSLRAEDT | | RVEAGDEADYYCQV |
| | | | | AVYYCAKDAILGSGH | | WDSSSDHAVFGGGT |
| | | | | PWYFHVWGRGTLVT | | QLTVL |
| | | | | VSS | | |
| H004 | Virus; | S HBsAg | 16 | EVQLVESGGGLVQPG | 29 | SYVLTQPPSVSVAPG |
| | HBV | | | GSLRLSCAASGFTFSS | | QTARITCGGNNIGSK |
| | | | | YAMSWVRQAPGKGL | | SVHWYQQKPGQAPV |
| | | | | EWVSAISGSGGSTYY | | LVVYDDSDRPSGIPE |
| | | | | ADSVKGRFTISRDNSK | | RFSGSNSGNTATLTIS |
| | | | | NTLYLQMNSLRAEDT | | RVEAGDEADYYCQV |
| | | | | AVYYCAKDGYFGSGS | | WDSNHDHPGVVFGG |
| | | | | LYGIDVWGQGTTVTV | | GTKLTVL |
| | | | | SS | | |
| H009 | Virus; | S HBsAg | 17 | QVQLVESGGGVVQPG | 30 | SYVLTQPPSVSVAPG |
| | HBV | | | RSLRLSCAASGFTFSS | | QTARITCGGNNIGSK |
| | | | | YGMHWVRQAPGKGL | | SVHWYQQKPGQAPV |
| | | | | EWVAVISYDGSNKYY | | LVVYDDSDRPSGIPE |
| | | | | ADSVKGRFTISRDNSK | | RFSGSNSGNTATLTIS |
| | | | | NTLYLQMNSLRAEDT | | RVEAGDEADYYCQV |
| | | | | AVYYCAKTDIKWGA | | WDGTRDHLVVFGGG |
| | | | | TNYGMDVWGQGTTV | | TKLTVL |
| | | | | TVSS | | |
| H007 | Virus; | S HBsAg | 18 | QVQLVESGGGVVQPG | 31 | EIVLTQSPGTLSLSPG |
| | HBV | | | RSLRLSCAASGFTFSS | | ERATLSCRASQSVSS |
| | | | | YGMHWVRQAPGKGL | | SYLAWYQQKPGQAP |
| | | | | EWVAVISYDGSNKYY | | RLLIYGASSRATGIPD |
| | | | | ADSVKGRFTISRDNSK | | RFSGSGSGTDFTLTIS |
| | | | | NTLYLQMNSLRAEDT | | RLEPEDFAVYYCQQ |
| | | | | AVYYCAKEIGGFDFR | | YSSSPPGYTFGQGTK |
| | | | | SGSQRSYYYYGVDV | | LEIK |
| | | | | WGQGTTVTVSS | | |
| H019 | Virus; | S HBsAg | 19 | QVQLQESGPGLVKPS | 32 | DIQMTQSPSSLSASV |
| | HBV | | | QTLSLTCTVSGGSISS | | GDRVTITCRASQSISS |
| | | | | GDYYWSWIRQPPGKG | | YLNWYQQKPGKAPK |
| | | | | LEWIGYIYYSGSTYYN | | LLIYAASSLQSGVPSR |
| | | | | PSLKSRVTISVDTSKN | | FSGSGSGTDFTLTISS |
| | | | | QFSLKLSSVTAADTA | | LQPEDFATYYCQQSY |
| | | | | VYYCAIYMDEAWAF | | TISLFTFGQGTKLEIK |
| | | | | EIWGQGTMVTVSS | | |
| H020 | Virus; | S HBsAg | 20 | QVQLQESGPGLVKPS | 33 | DIQMTQSPSSVSASV |
| | HBV | | | ETLSLTCTVSGGSISSY | | GDRVTITCRASQGISS |
| | | | | YWSWIRQPPGKGLEW | | WLAWYQQKPGKAP |
| | | | | IGYIYYSGSTNYNPSL | | KLLIYAASSLQSGVPS |
| | | | | KSRVTISVDTSKNQFS | | RFSGSGSGTDFTLTIS |
| | | | | LKLSSVTAADTAVYY | | SLQPEDFATYYCQQA |
| | | | | CARHLYRYGYRNYFD | | NSFPLTFGGGTKVEI |
| | | | | YWGQGTLVTVSS | | K |

TABLE 3B
Exemplary Complementarity Determining Region (CDR*) Sequences
Heavy Chain Complementarity Determining Region (CDR-H) Sequences

| | | SEQ | | SEQ | | SEQ |
|---|---|---|---|---|---|---|
| | | ID | | ID | | ID |
| Name | CDR-H1 | NO: | CDR-H2 | NO: | CDR-H3 | NO: |
| BG10- | SYWIG | 75 | VIYPGDSDTRYSPSF | 76 | TQWGYNYGSHFFY | 77 |
| 19 | | | QG | | MDV | |
| 80R | SYAMH | 78 | VISYDGSNKYYADS | 79 | DRSYYLDY | 80 |
| | | | VKG | | | |
| 7D10 | SYNIH | 81 | AIYPGNGDTSYTQKF | 82 | YGNYPSYAMDY | 83 |
| | | | KV | | | |
| FI6 | TYAMH | 84 | VISYDANYKYYADS | 85 | DSQLRSLLYFEWLS | 86 |
| | | | VKG | | QGYFDY | |
| 1E01 | DYGMS | 87 | GLNWNGDITAFTDS | 88 | VRTWGDYTTGEEII | 89 |
| | | | VKG | | NSWYFDL | |
| H015 | RHGMH | 90 | GIWFDGTNDYYTDS | 91 | EDPHLLIATLDL | 92 |
| | | | VKG | | | |
| 2H5 | SKSAAWN | 93 | RTYYRSKWHNDYA | 94 | GQMGALDV | 95 |
| | | | VSVKS | | | |
| ADRI- | SYAMS | 96 | GISGTGENTYYADS | 97 | DAILGSGHPWYFHV | 98 |
| 2F3 | | | VKG | | | |
| H004 | SYAMS | 99 | AISGSGGSTYYADSV | 100 | DGYFGSGSLYGIDV | 101 |
| | | | KG | | | |
| H009 | SYGMH | 102 | VISYDGSNKYYADS | 103 | TDIKWGATNYGMD | 104 |
| | | | VKG | | V | |
| H007 | SYGMH | 105 | VISYDGSNKYYADS | 106 | EIGGFDFRSGSQRSY | 107 |
| | | | VKG | | YYYGVDV | |
| H019 | SGDYYWS | 108 | YIYYSGSTYYNPSLK | 109 | YMDEAWAFEI | 110 |
| | | | S | | | |
| H020 | SYYWS | 111 | YIYYSGSTNYNPSLK | 112 | HLYRYGYRNYFDY | 113 |
| | | | S | | | |

Light Chain Complementarity Determining Region (CDR-H) Sequences

| | | SEQ | | SEQ | | SEQ |
|---|---|---|---|---|---|---|
| | | ID | | ID | | ID |
| Name | CDR-L1 | NO: | CDR-L2 | NO: | CDR-L3 | NO: |
| BG10- | SGSSSNIGS | 114 | RNNQRPS | 115 | AAWDASLSGYV | 116 |
| 19 | DHVY | | | | | |
| 80R | RASQSVRSN | 117 | DASTRAT | 118 | QQRSNWPPT | 119 |
| | LA | | | | | |
| 7D10 | RASKSVSAS | 120 | LAFNLES | 121 | QHSRDLPFT | 122 |
| | GYNYLH | | | | | |
| FI6 | KSSQSVTFN | 123 | WASTRES | 124 | QQHYRTPPT | 125 |
| | YKNYLA | | | | | |
| 1E01 | RASQDISSY | 126 | AASLLQS | 127 | QHLKSYPLFT | 128 |
| | LA | | | | | |
| H015 | RASQGLTSF | 129 | SASSLQR | 130 | QQSYGTPALA | 131 |
| | IN | | | | | |
| 2H5 | SGSSSNIGS | 132 | GNNQRPS | 133 | QSYDSSLSGVI | 134 |
| | YYVY | | | | | |
| ADRI- | GGNNIGSES | 135 | DDSDRPS | 136 | QVWDSSSDHAV | 137 |
| 2F3 | VH | | | | | |
| H004 | GGNNIGSKS | 138 | DDSDRPS | 139 | QVWDSNHDHPGVV | 140 |
| | VH | | | | | |
| H009 | GGNNIGSKS | 141 | DDSDRPS | 142 | QVWDGTRDHLVV | 143 |
| | VH | | | | | |
| H007 | RASQSVSSS | 144 | GASSRAT | 145 | QQYSSSPPGYT | 146 |
| | YLA | | | | | |
| H019 | RASQSISSY | 147 | AASSLqS | 148 | QQSYTISLFT | 149 |
| | LN | | | | | |
| H020 | RASQGISSW | 150 | AASSLqS | 151 | QQANSFPLT | 152 |
| | LA | | | | | |

* The CDR sequences listed in Table 3B are defined according to the Kabat numbering scheme.

In some embodiments, the homing polypeptide comprises a receptor that has binding specificity for the target protein. In some embodiments, the homing polypeptide comprises a receptor that has binding specificity for the target viral protein. In some embodiments, the homing polypeptide comprises a mammalian receptor that has binding specificity for the target viral protein. In some embodiments, the receptor comprises Sodium taurocholate cotransporting polypeptide (NTCP), angiotensin-converting enzyme 2 (ACE2), transmembrane serine protease 2 (TMPRSS2), dipeptidyl peptidase-4 (DPP4), cluster of differentiation 4 (CD4), herpes virus entry mediator (HVEM), programmed cell death ligand 1 (PD-1), C—C motif chemokine receptor 5 (CCR5), C—X—C chemokine receptor type 4 (CXCR4), C—X—C chemokine receptor type 5 (CXCR5), cluster of differentiation 209 (CD209), or C-type lectin domain family 4 member M (CLEC4M). In some embodiments, the homing polypeptide comprises a ligand that recognizes a tumor-associated receptor. In some embodiments, the homing polypeptide comprises a mammalian ligand that recognizes a tumor-associated receptor. In some embodiments, the ligand comprises epidermal growth factor (EGF), vascular endothelial growth factor (VEGF), transforming growth factor beta (TGF-β), interleukin 4 (IL-4), interleukin 11 (IL-11), insulin like growth factor 1 (IGF1), interleukin 6 (IL-6), or arginine-glycine-aspartic acid (RGD) peptide. In some embodiments, the homing polypeptide comprises an amino acid sequence with at least 75%, 80%, 85%, 90%, 95%, 96%, 97%, 98%, 99%, 100% sequence identity to any one of SEQ ID NOs: 34-51.

TABLE 4
Exemplary Mammalian Receptor and Ligand Polypeptide Sequences

| | | | SEQ |
|---|---|---|---|
| | | | ID |
| Name | Category | Amino Acid Sequence | NO: |
| ACE2 | receptor | MSSSSWLLLSLVAVTAAQSTIEEQAKTFLDKFNHEAEDLF | 34 |
| | | YQSSLASWNYNTNITEENVQNMNNAGDKWSAFLKEQSTL | |
| | | AQMYPLQEIQNLTVKLQLQALQQNGSSVLSEDKSKRINTI | |
| | | LNTMSTIYSTGKVCNPDNPQECLLLEPGLNEIMANSLDYN | |
| | | ERLWAWESWRSEVGKQLRPLYEEYVVLKNEMARANHYE | |
| | | DYGDYWRGDYEVNGVDGYDYSRGQLIEDVEHTFEEIKPL | |
| | | YEHLHAYVRAKLMNAYPSYISPIGCLPAHLLGDMWGRFW | |
| | | TNLYSLTVPFGQKPNIDVTDAMVDQAWDAQRIFKEAEKF | |
| | | FVSVGLPNMTQGFWENSMLTDPGNVQKAVCHPTAWDLG | |
| | | KGDFRILMCTKVTMDDFLTAHHEMGHIQYDMAYAAQPF | |
| | | LLRNGANEGFHEAVGEIMSLSAATPKHLKSIGLLSPDFQED | |
| | | NETEINFLLKQALTIVGTLPFTYMLEKWRWMVFKGEIPKD | |
| | | QWMKKWWEMKREIVGVVEPVPHDETYCDPASLFHVSND | |
| | | YSFIRYYTRTLYQFQFQEALCQAAKHEGPLHKCDISNSTEA | |
| | | GQKLFNMLRLGKSEPWTLALENVVGAKNMNVRPLLNYF | |
| | | EPLFTWLKDQNKNSFVGWSTDWSPYADQSIKVRISLKSAL | |
| | | GDKAYEWNDNEMYLFRSSVAYAMRQYFLKVKNQMILFG | |
| | | EEDVRVANLKPRISFNFFVTAPKNVSDIIPRTEVEKAIRMSR | |
| | | SRINDAFRLNDNSLEFLGIQPTLGPPNQPPVSIWLIVFGVVM | |
| | | GVIVVGIVILIFTGIRDRKKKNKARSGENPYASIDISKGENN | |
| | | PGFQNTDDVQTSF | |
| DPP4 | receptor | MKTPWKVLLGLLGAAALVTIITVPVVLLNKGTDDATADS | 35 |
| | | RKTYTLTDYLKNTYRLKLYSLRWISDHEYLYKQENNILVF | |
| | | NAEYGNSSVFLENSTFDEFGHSINDYSISPDGQFILLEYNYV | |
| | | KQWRHSYTASYDIYDLNKRQLITEERIPNNTQWVTWSPV | |
| | | GHKLAYVWNNDIYVKIEPNLPSYRITWTGKEDIIYNGITD | |
| | | WVYEEEVFSAYSALWWSPNGTFLAYAQFNDTEVPLIEYSF | |
| | | YSDESLQYPKTVRVPYPKAGAVNPTVKFFVVNTDSLSSVT | |
| | | NATSIQITAPASMLIGDHYLCDVTWATQERISLQWLRRIQN | |
| | | YSVMDICDYDESSGRWNCLVARQHIEMSTTGWVGRFRPS | |
| | | EPHFTLDGNSFYKIISNEEGYRHICYFQIDKKDCTFITKGTW | |
| | | EVIGIEALTSDYLYYISNEYKGMPGGRNLYKIQLSDYTKVT | |
| | | CLSCELNPERCQYYSVSFSKEAKYYQLRCSGPGLPLYTLH | |
| | | SSVNDKGLRVLEDNSALDKMLQNVQMPSKKLDFIILNETK | |
| | | FWYQMILPPHFDKSKKYPLLLDVYAGPCSQKADTVFRLN | |
| | | WATYLASTENIIVASFDGRGSGYQGDKIMHAINRRLGTFE | |
| | | VEDQIEAARQFSKMGFVDNKRIAIWGWSYGGYVTSMVLG | |
| | | SGSGVFKCGIAVAPVSRWEYYDSVYTERYMGLPTPEDNL | |
| | | DHYRNSTVMSRAENFKQVEYLLIHGTADDNVHFQQSAQI | |
| | | SKALVDVGVDFQAMWYTDEDHGIASSTAHQHIYTHMSHF | |
| | | IKQCFSLP | |
| CD4 | receptor | MNRGVPFRHLLLVLQLALLPAATQGKKVVLGKKGDTVEL | 36 |
| | | TCTASQKKSIQFHWKNSNQIKILGNQGSFLTKGPSKLNDR | |
| | | ADSRRSLWDQGNFPLIIKNLKIEDSDTYICEVEDQKEEVQL | |
| | | LVFGLTANSDTHLLQGQSLTLTLESPPGSSPSVQCRSPRGK | |
| | | NIQGGKTLSVSQLELQDSGTWTCTVLQNQKKVEFKIDIVV | |
| | | LAFQKASSIVYKKEGEQVEFSFPLAFTVEKLTGSGELWWQ | |
| | | AERASSSKSWITFDLKNKEVSVKRVTQDPKLQMGKKLPL | |
| | | HLTLPQALPQYAGSGNLTLALEAKTGKLHQEVNLVVMRA | |
| | | TQLQKNLTCEVWGPTSPKLMLSLKLENKEAKVSKREKAV | |
| | | WVLNPEAGMWQCLLSDSGQVLLESNIKVLPTWSTPVQPM | |
| | | ALIVLGGVAGLLLFIGLGIFFCVRCRHRRRQAERMSQIKRL | |
| | | LSEKKTCQCPHRFQKTCSPI | |
| HVEM | receptor | MEPPGDWGPPPWRSTPKTDVLRLVLYLTFLGAPCYAPALP | 37 |
| | | SCKEDEYPVGSECCPKCSPGYRVKEACGELTGTVCEPCPP | |
| | | GTYIAHLNGLSKCLQCQMCDPAMGLRASRNCSRTENAVC | |
| | | GCSPGHFCIVQDGDHCAACRAYATSSPGQRVQKGGTESQ | |
| | | DTLCQNCPPGTFSPNGTLEECQHQTKCSWLVTKAGAGTSS | |
| | | SHWVWWFLSGSLVIVIVCSTVGLIICVKRRKPRGDVVKVI | |
| | | VSVQRKRQEAEGEATVIEALQAPPDVTTVAVEETIPSFTGR | |
| | | SPNH | |
| PD-1 | receptor | MQIPQAPWPVVWAVLQLGWRPGWFLDSPDRPWNPPTFSP | 38 |
| | | ALLVVTEGDNATFTCSFSNTSESFVLNWYRMSPSNQTDKL | |
| | | AAFPEDRSQPGQDCRFRVTQLPNGRDFHMSVVRARRNDS | |
| | | GTYLCGAISLAPKAQIKESLRAELRVTERRAEVPTAHPSPS | |
| | | PRPAGQFQTLVVGVVGGLLGSLVLLVWVLAVICSRAARG | |
| | | TIGARRTGQPLKEDPSAVPVFSVDYGELDFQWREKTPEPP | |
| | | VPCVPEQTEYATIVFPSGMGTSSPARRGSADGPRSAQPLRP | |
| | | EDGHCSWPL | |
| TMPR | receptor | MALNSGSPPAIGPYYENHGYQPENPYPAQPTVVPTVYEVH | 39 |
| SS2 | | PAQYYPSPVPQYAPRVLTQASNPVVCTQPKSPSGTVCTSK | |
| | | TKKALCITLTLGTFLVGAALAAGLLWKFMGSKCSNSGIEC | |
| | | DSSGTCINPSNWCDGVSHCPGGEDENRCVRLYGPNFILQV | |
| | | YSSQRKSWHPVCQDDWNENYGRAACRDMGYKNNFYSS | |
| | | QGIVDDSGSTSFMKLNTSAGNVDIYKKLYHSDACSSKAVV | |
| | | SLRCIACGVNLNSSRQSRIVGGESALPGAWPWQVSLHVQN | |
| | | VHVCGGSIITPEWIVTAAHCVEKPLNNPWHWTAFAGILRQ | |
| | | SFMFYGAGYQVEKVISHPNYDSKTKNNDIALMKLQKPLTF | |
| | | NDLVKPVCLPNPGMMLQPEQLCWISGWGATEEKGKTSEV | |
| | | LNAAKVLLIETQRCNSRYVYDNLITPAMICAGFLQGNVDS | |
| | | CQGDSGGPLVTSKNNIWWLIGDTSWGSGCAKAYRPGVYG | |
| | | NVMVFTDWIYRQMRADG | |
| CCR5 | receptor | MDYQVSSPIYDINYYTSEPCQKINVKQIAARLLPPLYSLVFI | 40 |
| | | FGFVGNMLVILILINCKRLKSMTDIYLLNLAISDLFFLLTVP | |
| | | FWAHYAAAQWDFGNTMCQLLTGLYFIGFFSGIFFIILLTID | |
| | | RYLAVVHAVFALKARTVTFGVVTSVITWVVAVFASLPGII | |
| | | FTRSQKEGLHYTCSSHFPYSQYQFWKNFQTLKIVILGLVLP | |
| | | LLVMVICYSGILKTLLRCRNEKKRHRAVRLIFTIMIVYFLF | |
| | | WAPYNIVLLLNTFQEFFGLNNCSSSNRLDQAMQVTETLG | |
| | | MTHCCINPIIYAFVGEKFRNYLLVFFQKHIAKRFCKCCSIFQ | |
| | | QEAPERASSVYTRSTGEQEISVGL | |
| CXCR5 | receptor | MNYPLTLEMDLENLEDLFWELDRLDNYNDTSLVENHLCP | 41 |
| | | ATEGPLMASFKAVFVPVAYSLIFLLGVIGNVLVLVILERHR | |
| | | QTRSSTETFLFHLAVADLLLVFILPFAVAEGSVGWVLGTFL | |
| | | CKTVIALHKVNFYCSSLLLACIAVDRYLAIVHAVHAYRHR | |
| | | RLLSIHITCGTIWLVGFLLALPEILFAKVSQGHHNNSLPRCT | |
| | | FSQENQAETHAWFTSRFLYHVAGFLLPMLVMGWCYVGV | |
| | | VHRLRQAQRRPQRQKAVRVAILVTSIFFLCWSPYHIVIFLD | |
| | | TLARLKAVDNTCKLNGSLPVAITMCEFLGLAHCCLNPML | |
| | | YTFAGVKFRSDLSRLLTKLGCTGPASLCQLFPSWRRSSLSE | |
| | | SENATSLTTF | |
| CD209 | receptor | MSDSKEPRLQQLGLLEEEQLRGLGFRQTRGYKSLAGCLG | 42 |
| | | HGPLVLQLLSFTLLAGLLVQVSKVPSSISQEQSRQDAIYQN | |
| | | LTQLKAAVGELSEKSKLQEIYQELTQLKAAVGELPEKSKL | |
| | | QEIYQELTRLKAAVGELPEKSKLQEIYQELTWLKAAVGEL | |
| | | PEKSKMQEIYQELTRLKAAVGELPEKSKQQEIYQELTRLK | |
| | | AAVGELPEKSKQQEIYQELTRLKAAVGELPEKSKQQEIYQ | |
| | | ELTQLKAAVERLCHPCPWEWTFFQGNCYFMSNSQRNWH | |
| | | DSITACKEVGAQLVVIKSAEEQNFLQLQSSRSNRFTWMGL | |
| | | SDLNQEGTWQWVDGSPLLPSFKQYWNRGEPNNVGEEDC | |
| | | AEFSGNGWNDDKCNLAKFWICKKSAASCSRDEEQFLSPA | |
| | | PATPNPPPA | |
| CLEC | receptor | MSDSKEPRVQQLGLLEEDPTTSGIRLFPRDFQFQQIHGHKS | 43 |
| 4M | | STGCLGHGALVLQLLSFMLLAGVLVAILVQVSKVPSSLSQ | |
| | | EQSEQDAIYQNLTQLKAAVGELSEKSKLQEIYQELTQLKA | |
| | | AVGELPEKSKLQEIYQELTRLKAAVGELPEKSKLQEIYQEL | |
| | | TRLKAAVGELPEKSKLQEIYQELTRLKAAVGELPEKSKLQ | |
| | | EIYQELTELKAAVGELPEKSKLQEIYQELTQLKAAVGELPD | |
| | | QSKQQQIYQELTDLKTAFERLCRHCPKDWTFFQGNCYFM | |
| | | SNSQRNWHDSVTACQEVRAQLVVIKTAEEQNFLQLQTSR | |
| | | SNRFSWMGLSDLNQEGTWQWVDGSPLSPSFQRYWNSGEP | |
| | | NNSGNEDCAEFSGSGWNDNRCDVDNYWICKKPAACFRD | |
| | | E | |
| EGF | ligand | MLLTLIILLPVVSKFSFVSLSAPQHWSCPEGTLAGNGNSTC | 44 |
| | | VGPAPFLIFSHGNSIFRIDTEGTNYEQLVVDAGVSVIMDFH | |
| | | YNEKRIYWVDLERQLLQRVFLNGSRQERVCNIEKNVSGM | |
| | | AINWINEEVIWSNQQEGIITVTDMKGNNSHILLSALKYPAN | |
| | | VAVDPVERFIFWSSEVAGSLYRADLDGVGVKALLETSEKI | |
| | | TAVSLDVLDKRLFWIQYNREGSNSLICSCDYDGGSVHISK | |
| | | HPTQHNLFAMSLFGDRIFYSTWKMKTIWIANKHTGKDMV | |
| | | RINLHSSFVPLGELKVVHPLAQPKAEDDTWEPEQKLCKLR | |
| | | KGNCSSTVCGQDLQSHLCMCAEGYALSRDRKYCEDVNEC | |
| | | AFWNHGCTLGCKNTPGSYYCTCPVGFVLLPDGKRCHQLV | |
| | | SCPRNVSECSHDCVLTSEGPLCFCPEGSVLERDGKTCSGCS | |
| | | SPDNGGCSQLCVPLSPVSWECDCFPGYDLQLDEKSCAASG | |
| | | PQPFLLFANSQDIRHMHFDGTDYGTLLSQQMGMVYALDH | |
| | | DPVENKIYFAHTALKWIERANMDGSQRERLIEEGVDVPEG | |
| | | LAVDWIGRRFYWTDRGKSLIGRSDLNGKRSKIITKENISQP | |
| | | RGIAVHPMAKRLFWTDTGINPRIESSSLQGLGRLVIASSDLI | |
| | | WPSGITIDFLTDKLYWCDAKQSVIEMANLDGSKRRRLTQN | |
| | | DVGHPFAVAVFEDYVWFSDWAMPSVMRVNKRTGKDRV | |
| | | RLQGSMLKPSSLVVVHPLAKPGADPCLYQNGGCEHICKK | |
| | | RLGTAWCSCREGFMKASDGKTCLALDGHQLLAGGEVDL | |
| | | KNQVTPLDILSKTRVSEDNITESQHMLVAEIMVSDQDDCA | |
| | | PVGCSMYARCISEGEDATCQCLKGFAGDGKLCSDIDECEM | |
| | | GVPVCPPASSKCINTEGGYVCRCSEGYQGDGIHCLDIDEC | |
| | | QLGEHSCGENASCTNTEGGYTCMCAGRLSEPGLICPDSTP | |
| | | PPHLREDDHHYSVRNSDSECPLSHDGYCLHDGVCMYIEAL | |
| | | DKYACNCVVGYIGERCQYRDLKWWELRHAGHGQQQKVI | |
| | | VVAVCVVVLVMLLLLSLWGAHYYRTQKLLSKNPKNPYE | |
| | | ESSRDVRSRRPADTEDGMSSCPQPWFVVIKEHQDLKNGG | |
| | | QPVAGEDGQAADGSMQPTSWRQEPQLCGMGTEQGCWIP | |
| | | VSSDKGSCPQVMERSFHMPSYGTQTLEGGVEKPHSLLSAN | |
| | | PLWQQRALDPPHQMELTQ | |
| VEGF | ligand | MNFLLSWVHWSLALLLYLHHAKWSQAAPMAEGGGQNH | 45 |
| | | HEVVKFMDVYQRSYCHPIETLVDIFQEYPDEIEYIFKPSCV | |
| | | PLMRCGGCCNDEGLECVPTEESNITMQIMRIKPHQGQHIG | |
| | | EMSFLQHNKCECRPKKDRARQEKKSVRGKGKGQKRKRK | |
| | | KSRYKSWSVYVGARCCLMPWSLPGPHPCGPCSERRKHLF | |
| | | VQDPQTCKCSCKNTDSRCKARQLELNERTCRCDKPRR | |
| TGF- | ligand | MPPSGLRLLLLLLPLLWLLVLTPGRPAAGLSTCKTIDMELV | 46 |
| β1 | | KRKRIEAIRGQILSKLRLASPPSQGEVPPGPLPEAVLALYNS | |
| | | TRDRVAGESAEPEPEPEADYYAKEVTRVLMVETHNEIYD | |
| | | KFKQSTHSIYMFFNTSELREAVPEPVLLSRAELRLLRLKLK | |
| | | VEQHVELYQKYSNNSWRYLSNRLLAPSDSPEWLSFDVTG | |
| | | VVRQWLSRGGEIEGFRLSAHCSCDSRDNTLQVDINGFTTG | |
| | | RRGDLATIHGMNRPFLLLMATPLERAQHLQSSRHRRALDT | |
| | | NYCFSSTEKNCCVRQLYIDFRKDLGWKWIHEPKGYHANF | |
| | | CLGPCPYIWSLDTQYSKVLALYNQHNPGASAAPCCVPQA | |
| | | LEPLPIVYYVGRKPKVEQLSNMIVRSCKCS | |
| IL-4 | ligand | MGLTSQLLPPLFFLLACAGNFVHGHKCDITLQEIIKTLNSL | 47 |
| | | TEQKTLCTELTVTDIFAASKNTTEKETFCRAATVLRQFYSH | |
| | | HEKDTRCLGATAQQFHRHKQLIRFLKRLDRNLWGLAGLN | |
| | | SCPVKEANQSTLENFLERLKTIMREKYSKCSS | |
| IL-11 | ligand | MNCVCRLVLVVLSLWPDTAVAPGPPPGPPRVSPDPRAELD | 48 |
| | | STVLLTRSLLADTRQLAAQLRDKFPADGDHNLDSLPTLAM | |
| | | SAGALGALQLPGVLTRLRADLLSYLRHVQWLRRAGGSSL | |
| | | KTLEPELGTLQARLDRLLRRLQLLMSRLALPQPPPDPPAPP | |
| | | LAPPSSAWGGIRAAHAILGGLHLTLDWAVRGLLLLKTRL | |
| IGF1 | ligand | MGKISSLPTQLFKCCFCDFLKVKMHTMSSSHLFYLALCLL | 49 |
| | | TFTSSATAGPETLCGAELVDALQFVCGDRGFYFNKPTGYG | |
| | | SSSRRAPQTGIVDECCFRSCDLRRLEMYCAPLKPAKSARS | |
| | | VRAQRHTDMPKTQKYQPPSTNKNTKSQRRKGWPKTHPG | |
| | | GEQKEGTEASLQIRGKKKEQRREIGSRNAECRGKKGK | |
| IL-6 | ligand | MNSFSTSAFGPVAFSLGLLLVLPAAFPAPVPPGEDSKDVA | 50 |
| | | APHRQPLTSSERIDKQIRYILDGISALRKETCNKSNMCESSK | |
| | | EALAENNLNLPKMAEKDGCFQSGFNEETCLVKIITGLLEFE | |
| | | VYLEYLQNRFESSEEQARAVQMSTKVLIQFLOKKAKNLD | |
| | | AITTPDPTTNASLLTKLQAQNQWLQDMTTHLILRSFKEFL | |
| | | QSSLRALRQM | |
| RGD | ligand | RGD | 51 |
| peptide | | | |

In some embodiments, the homing polypeptide comprises an amino acid sequence with at least 75% sequence identity to any one of SEQ ID NOs: 34-51. In some embodiments, the homing polypeptide comprises an amino acid sequence with at least 76% sequence identity to any one of SEQ ID NOs: 34-51. In some embodiments, the homing polypeptide comprises an amino acid sequence with at least 77% sequence identity to any one of SEQ ID NOs: 34-51. In some embodiments, the homing polypeptide comprises an amino acid sequence with at least 78% sequence identity to any one of SEQ ID NOs: 34-51. In some embodiments, the homing polypeptide comprises an amino acid sequence with at least 79% sequence identity to any one of SEQ ID NOs: 34-51. In some embodiments, the homing polypeptide comprises an amino acid sequence with at least 80% sequence identity to any one of SEQ ID NOs: 34-51. In some embodiments, the homing polypeptide comprises an amino acid sequence with at least 81% sequence identity to any one of SEQ ID NOs: 34-51. In some embodiments, the homing polypeptide comprises an amino acid sequence with at least 82% sequence identity to any one of SEQ ID NOs: 34-51. In some embodiments, the homing polypeptide comprises an amino acid sequence with at least 83% sequence identity to any one of SEQ ID NOs: 34-51. In some embodiments, the homing polypeptide comprises an amino acid sequence with at least 84% sequence identity to any one of SEQ ID NOs: 34-51. In some embodiments, the homing polypeptide comprises an amino acid sequence with at least 85% sequence identity to any one of SEQ ID NOs: 34-51. In some embodiments, the homing polypeptide comprises an amino acid sequence with at least 86% sequence identity to any one of SEQ ID NOs: 34-51. In some embodiments, the homing polypeptide comprises an amino acid sequence with at least 87% sequence identity to any one of SEQ ID NOs: 34-51. In some embodiments, the homing polypeptide comprises an amino acid sequence with at least 88% sequence identity to any one of SEQ ID NOs: 34-51. In some embodiments, the homing polypeptide comprises an amino acid sequence with at least 89% sequence identity to any one of SEQ ID NOs: 34-51. In some embodiments, the homing polypeptide comprises an amino acid sequence with at least 90% sequence identity to any one of SEQ ID NOs: 34-51. In some embodiments, the homing polypeptide comprises an amino acid sequence with at least 91% sequence identity to any one of SEQ ID NOs: 34-51. In some embodiments, the homing polypeptide comprises an amino acid sequence with at least 92% sequence identity to any one of SEQ ID NOs: 34-51. In some embodiments, the homing polypeptide comprises an amino acid sequence with at least 93% sequence identity to any one of SEQ ID NOs: 34-51. In some embodiments, the homing polypeptide comprises an amino acid sequence with at least 94% sequence identity to any one of SEQ ID NOs: 34-51. In some embodiments, the homing polypeptide comprises an amino acid sequence with at least 95% sequence identity to any one of SEQ ID NOs: 34-51. In some embodiments, the homing polypeptide comprises an amino acid sequence with at least 96% sequence identity to any one of SEQ ID NOs: 34-51. In some embodiments, the homing polypeptide comprises an amino acid sequence with at least 97% sequence identity to any one of SEQ ID NOs: 34-51. In some embodiments, the homing polypeptide comprises an amino acid sequence with at least 98% sequence identity to any one of SEQ ID NOs: 34-51. In some embodiments, the homing polypeptide comprises an amino acid sequence with at least 99% sequence identity to any one of SEQ ID NOs: 34-51. In some embodiments, the homing polypeptide comprises an amino acid sequence according to any one of SEQ ID NOs: 34-51.

### Oligomerization Domain

In some embodiments, the first fusion protein is monomeric. In some embodiments, the first fusion protein comprises an oligomerization domain. In some embodiments, the second fusion protein is monomeric. In some embodiments, the second fusion protein comprises an oligomerization domain.

In some embodiments, the oligomerization domain of the first fusion protein comprises a dimerization domain. In some embodiments, the oligomerization domain of the second fusion protein comprises a dimerization domain. In some embodiments, the dimerization domain comprises a leucine zipper dimerization domain. In some embodiments, the oligomerization domain of the first fusion protein comprises a trimerization domain. In some embodiments, the oligomerization domain of the second fusion protein comprises a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain of the first fusion protein comprises a tetramerization domain. In some embodiments, the oligomerization domain of the second fusion protein comprises a tetramerization domain. In some embodiments, the tetramerization domain comprises an influenza neuraminidase stem domain.

TABLE 5
Exemplary Oligomerization Domain Sequences

| Oligomerization | | | |
|---|---|---|---|
| Domain | Oligomer | Amino Acid Sequence | SEQ ID NO. |
| D4 Variation 1 | Trimer | IGTALQVKMPKSHKAIQADGWMCHASK | 52 |
| | | WVTTCDFRWYGPKYITHSIRSFTPSVEQ | |
| | | CKESIEQTKQGTWLNPGFPPQSCGYATV | |
| | | TD7AEAVIVQVTPHHVLVDEYTGEWVD | |
| | | SQFIN8GKCSNYICPTVHNSTTWHSDYK | |
| | | VKGLCDSNLISMDI | |
| D4 Variation 2 | Trimer | IQADGWMCHASKWVTTCDFRWYGPKY | 53 |
| | | ITHSIRSFTPSVEQCKESIEQTKQGTWLNP | |
| | | GFPPQSCGYATVIDAEAVIVQVTPHHVL | |
| | | VDEYTGEWVDSQFINGKCSNYICPTVHN | |
| | | STTWHSDYKVKGLCDSNL | |
| D4 Variation 3 | Trimer | IQADGWMCHASKWVTTCDFRWYGPKY | 54 |
| | | ITHSIRSFTPSVEQCKESIEQTKQGTWLNP | |
| | | GFPPQSCGYATVIDAEAVIVQVTPHHVL | |
| | | VDEYTGEWVDSQFINGKCSNYICPTVHN | |
| | | STT | |
| D4 Variation 4 | Trimer | IQADGWMCHASKWVTTCDFRWYGPKY | 55 |
| | | ITHSIRSFTPSVEQCKESIEQTKQGTWLNP | |
| | | GFPPQSCGYATVIDAEAVIVQVTPHHVL | |
| | | VDEYTGEWVDSQFING | |
| D4 Variation 5 | Trimer | IQADGWMCHASKWVTTCDFRWYGPKY | 56 |
| | | ITHSIRSFTPSVEQCKESIEQTKQGTWLNP | |
| | | GFPPQSCGYATVTDAEAVIVQVTPHHVL | |
| Foldon | Trimer | GYIPEAPRDGQAYVRKDGEWVLLSTFL | 57 |
| Leucine Zipper | Dimer | RMKQLEDKVEELLSKQYHLENEVARLK | 58 |
| V1 | | KLVGER | |
| Leucine Zipper | Dimer | RMKQLEDKVEELLSKNYHLENEVARLK | 59 |
| V2 | | KLVGER | |
| Neuraminidase | Tetramer | MNPNQKIITIGSICLVVGLISLILQIGNIISI | 60 |
| Stem V1 | | WISHSIQT | |
| Neuraminidase | Tetramer | MNPNQKIITIGSICMVTGIVSLMLQIGNM | 61 |
| Stem V2 | | ISIWVSHSIHTGNQHQSEPISNTNFLTEKA | |
| | | VASVKLAGNSSLCPIN | |
| Dengue E Fusion | Trimer | KLCIEAKISNTTTDSRCPTQGEATLVEEQ | 62 |
| V1 | | DTNFVCRRTFVDRGHGNGCGLFGKGSLI | |
| | | TCAKFKCVTKL | |
| Dengue E Fusion | Trimer | IELLKTEVTNPAVLRKLCIEAKISNTTTDS | 63 |
| V2 | | RCPTQGEATLVEEQDTNFVCRRTFVDRG | |
| | | HGNGCGLFGKGSLITCAKFKCVTKL | |
| Dengue E Fusion | Trimer | KLCIEAKISNTTTDSRCPTQGEATLVEEQ | 64 |
| V3 | | DTNFVCRRTFVDRGHGNGCGLFGKGSLI | |
| | | TCAKFKCVTKLEGKIVQYENLKYSVI | |
| Dengue E Fusion | Trimer | EAKISNTTTDSRCPTQGEATLVEEQDTNF | 65 |
| V4 | | VCRRTFVDRGHGNGCGLFGKGSLITCAK | |
| | | FK | |

In some embodiments, the oligomerization domain of the first or second fusion protein comprises an amino acid sequence disclosed in Table 5, or an amino acid sequence that is substantially identical to an amino acid sequence in Table 5 (e.g., 75%, 80%, 85%, 90%, 95%, 96%, 97%, 98%, 99% sequence identity). In some instances, the oligomerization domain of the first or second fusion protein comprises an amino acid sequence comprising at least a portion having at least or about 10, 20, 30, 40, 50, 60, 70, 80, 90, 100, 110, 120, 130 amino acid sequences of any sequence according to Table 5.

In some embodiments, the oligomerization domain of the first fusion protein comprises an amino acid sequence that has at least 75% sequence identity to an amino acid sequence according to any one of SEQ ID NOs: 52-65. In some embodiments, the oligomerization domain of the first fusion protein comprises an amino acid sequence that has at least 76% sequence identity to an amino acid sequence according to any one of SEQ ID NOs: 52-65. In some embodiments, the oligomerization domain of the first fusion protein comprises an amino acid sequence that has at least 77% sequence identity to an amino acid sequence according to any one of SEQ ID NOs: 52-65. In some embodiments, the oligomerization domain of the first fusion protein comprises an amino acid sequence that has at least 78% sequence identity to an amino acid sequence according to any one of SEQ ID NOs: 52-65. In some embodiments, the oligomerization domain of the first fusion protein comprises an amino acid sequence that has at least 79% sequence identity to an amino acid sequence according to any one of SEQ ID NOs: 52-65. In some embodiments, the oligomerization domain of the first fusion protein comprises an amino acid sequence that has at least 80% sequence identity to an amino acid sequence according to any one of SEQ ID NOs: 52-65. In some embodiments, the oligomerization domain of the first fusion protein comprises an amino acid sequence that has at least 81% sequence identity to an amino acid sequence according to any one of SEQ ID NOs: 52-65. In some embodiments, the oligomerization domain of the first fusion protein comprises an amino acid sequence that has at least 82% sequence identity to an amino acid sequence according to any one of SEQ ID NOs: 52-65. In some embodiments, the oligomerization domain of the first fusion protein comprises an amino acid sequence that has at least 83% sequence identity to an amino acid sequence according to any one of SEQ ID NOs: 52-65. In some embodiments, the oligomerization domain of the first fusion protein comprises an amino acid sequence that has at least 84% sequence identity to an amino acid sequence according to any one of SEQ ID NOs: 52-65. In some embodiments, the oligomerization domain of the first fusion protein comprises an amino acid sequence that has at least 85% sequence identity to an amino acid sequence according to any one of SEQ ID NOs: 52-65. In some embodiments, the oligomerization domain of the first fusion protein comprises an amino acid sequence that has at least 86% sequence identity to an amino acid sequence according to any one of SEQ ID NOs: 52-65. In some embodiments, the oligomerization domain of the first fusion protein comprises an amino acid sequence that has at least 87% sequence identity to an amino acid sequence according to any one of SEQ ID NOs: 52-65. In some embodiments, the oligomerization domain of the first fusion protein comprises an amino acid sequence that has at least 88% sequence identity to an amino acid sequence according to any one of SEQ ID NOs: 52-65. In some embodiments, the oligomerization domain of the first fusion protein comprises an amino acid sequence that has at least 89% sequence identity to an amino acid sequence according to any one of SEQ ID NOs: 52-65. In some embodiments, the oligomerization domain of the first fusion protein comprises an amino acid sequence that has at least 90% sequence identity to an amino acid sequence according to any one of SEQ ID NOs: 52-65. In some embodiments, the oligomerization domain of the first fusion protein comprises an amino acid sequence that has at least 91% sequence identity to an amino acid sequence according to any one of SEQ ID NOs: 52-65. In some embodiments, the oligomerization domain of the first fusion protein comprises an amino acid sequence that has at least 92% sequence identity to an amino acid sequence according to any one of SEQ ID NOs: 52-65. In some embodiments, the oligomerization domain of the first fusion protein comprises an amino acid sequence that has at least 93% sequence identity to an amino acid sequence according to any one of SEQ ID NOs: 52-65. In some embodiments, the oligomerization domain of the first fusion protein comprises an amino acid sequence that has at least 94% sequence identity to an amino acid sequence according to any one of SEQ ID NOs: 52-65. In some embodiments, the oligomerization domain of the first fusion protein comprises an amino acid sequence that has at least 95% sequence identity to an amino acid sequence according to any one of SEQ ID NOs: 52-65. In some embodiments, the oligomerization domain of the first fusion protein comprises an amino acid sequence that has at least 96% sequence identity to an amino acid sequence according to any one of SEQ ID NOs: 52-65. In some embodiments, the oligomerization domain of the first fusion protein comprises an amino acid sequence that has at least 97% sequence identity to an amino acid sequence according to any one of SEQ ID NOs: 52-65. In some embodiments, the oligomerization domain of the first fusion protein comprises an amino acid sequence that has at least 98% sequence identity to an amino acid sequence according to any one of SEQ ID NOs: 52-65. In some embodiments, the oligomerization domain of the first fusion protein comprises an amino acid sequence that has at least 99% sequence identity to an amino acid sequence according to any one of SEQ ID NOs: 52-65. In some embodiments, the oligomerization domain of the first fusion protein comprises an amino acid sequence according to any one of SEQ ID NOs: 52-65.

In some embodiments, the oligomerization domain of the second fusion protein comprises an amino acid sequence that has at least 75% sequence identity to an amino acid sequence according to any one of SEQ ID NOs: 52-65. In some embodiments, the oligomerization domain of the second fusion protein comprises an amino acid sequence that has at least 76% sequence identity to an amino acid sequence according to any one of SEQ ID NOs: 52-65. In some embodiments, the oligomerization domain of the second fusion protein comprises an amino acid sequence that has at least 77% sequence identity to an amino acid sequence according to any one of SEQ ID NOs: 52-65. In some embodiments, the oligomerization domain of the second fusion protein comprises an amino acid sequence that has at least 78% sequence identity to an amino acid sequence according to any one of SEQ ID NOs: 52-65. In some embodiments, the oligomerization domain of the second fusion protein comprises an amino acid sequence that has at least 79% sequence identity to an amino acid sequence according to any one of SEQ ID NOs: 52-65. In some embodiments, the oligomerization domain of the second fusion protein comprises an amino acid sequence that has at least 80% sequence identity to an amino acid sequence according to any one of SEQ ID NOs: 52-65. In some embodiments, the oligomerization domain of the second fusion protein comprises an amino acid sequence that has at least 81% sequence identity to an amino acid sequence according to any one of SEQ ID NOs: 52-65. In some embodiments, the oligomerization domain of the second fusion protein comprises an amino acid sequence that has at least 82% sequence identity to an amino acid sequence according to any one of SEQ ID NOs: 52-65. In some embodiments, the oligomerization domain of the second fusion protein comprises an amino acid sequence that has at least 83% sequence identity to an amino acid sequence according to any one of SEQ ID NOs: 52-65. In some embodiments, the oligomerization domain of the second fusion protein comprises an amino acid sequence that has at least 84% sequence identity to an amino acid sequence according to any one of SEQ ID NOs: 52-65. In some embodiments, the oligomerization domain of the second fusion protein comprises an amino acid sequence that has at least 85% sequence identity to an amino acid sequence according to any one of SEQ ID NOs: 52-65. In some embodiments, the oligomerization domain of the second fusion protein comprises an amino acid sequence that has at least 86% sequence identity to an amino acid sequence according to any one of SEQ ID NOs: 52-65. In some embodiments, the oligomerization domain of the second fusion protein comprises an amino acid sequence that has at least 87% sequence identity to an amino acid sequence according to any one of SEQ ID NOs: 52-65. In some embodiments, the oligomerization domain of the second fusion protein comprises an amino acid sequence that has at least 88% sequence identity to an amino acid sequence according to any one of SEQ ID NOs: 52-65. In some embodiments, the oligomerization domain of the second fusion protein comprises an amino acid sequence that has at least 89% sequence identity to an amino acid sequence according to any one of SEQ ID NOs: 52-65. In some embodiments, the oligomerization domain of the second fusion protein comprises an amino acid sequence that has at least 90% sequence identity to an amino acid sequence according to any one of SEQ ID NOs: 52-65. In some embodiments, the oligomerization domain of the second fusion protein comprises an amino acid sequence that has at least 91% sequence identity to an amino acid sequence according to any one of SEQ ID NOs: 52-65. In some embodiments, the oligomerization domain of the second fusion protein comprises an amino acid sequence that has at least 92% sequence identity to an amino acid sequence according to any one of SEQ ID NOs: 52-65. In some embodiments, the oligomerization domain of the second fusion protein comprises an amino acid sequence that has at least 93% sequence identity to an amino acid sequence according to any one of SEQ ID NOs: 52-65. In some embodiments, the oligomerization domain of the second fusion protein comprises an amino acid sequence that has at least 94% sequence identity to an amino acid sequence according to any one of SEQ ID NOs: 52-65. In some embodiments, the oligomerization domain of the second fusion protein comprises an amino acid sequence that has at least 95% sequence identity to an amino acid sequence according to any one of SEQ ID NOs: 52-65. In some embodiments, the oligomerization domain of the second fusion protein comprises an amino acid sequence that has at least 96% sequence identity to an amino acid sequence according to any one of SEQ ID NOs: 52-65. In some embodiments, the oligomerization domain of the second fusion protein comprises an amino acid sequence that has at least 97% sequence identity to an amino acid sequence according to any one of SEQ ID NOs: 52-65. In some embodiments, the oligomerization domain of the second fusion protein comprises an amino acid sequence that has at least 98% sequence identity to an amino acid sequence according to any one of SEQ ID NOs: 52-65. In some embodiments, the oligomerization domain of the second fusion protein comprises an amino acid sequence that has at least 99% sequence identity to an amino acid sequence according to any one of SEQ ID NOs: 52-65. In some embodiments, the oligomerization domain of the second fusion protein comprises an amino acid sequence according to any one of SEQ ID NOs: 52-65.

In some embodiments, the first fusion protein comprises a signal peptide. In some embodiments, domains of the first fusion protein are arranged from the N-terminus to the C-terminus in the following orders: (a) signal peptide, interferon polypeptide, oligomerization domain, and transmembrane polypeptide; (b) signal peptide, interferon polypeptide, transmembrane polypeptide, and oligomerization domain; or (c) signal peptide, oligomerization domain, display peptide, and transmembrane polypeptide. In some embodiments, the first fusion protein further comprises a cytosolic domain. In some embodiments, domains of the first fusion protein are arranged from the N-terminus to the C-terminus in the following orders: (a) signal peptide, interferon polypeptide, oligomerization domain, transmembrane polypeptide, and cytosolic domain; (b) signal peptide, interferon polypeptide, transmembrane polypeptide, oligomerization domain, and cytosolic domain; or (c) signal peptide, oligomerization domain, interferon polypeptide, transmembrane polypeptide, and cytosolic domain.

In some embodiments, the second fusion protein comprises a signal peptide. In some embodiments, the second fusion protein comprises the IFN polypeptide that has less than 100% sequence identity to the IFN polypeptide of the first fusion protein. In some embodiments, domains of the second fusion protein are arranged from the N-terminus to the C-terminus in the following orders: (a) signal peptide, interferon polypeptide, oligomerization domain, and transmembrane polypeptide; (b) signal peptide, interferon polypeptide, transmembrane polypeptide, and oligomerization domain; or (c) signal peptide, oligomerization domain, interferon polypeptide, and transmembrane polypeptide. In some embodiments, the second fusion protein further comprises a cytosolic domain. In some embodiments, domains of the second fusion protein are arranged from the N-terminus to the C-terminus in the following orders: (a) signal peptide, interferon polypeptide, oligomerization domain, transmembrane polypeptide, and cytosolic domain; (b) signal peptide, interferon polypeptide, transmembrane polypeptide, oligomerization domain, and cytosolic domain; or (c) signal peptide, oligomerization domain, interferon polypeptide, transmembrane polypeptide, and cytosolic domain.

In some embodiments, the second fusion protein comprises a signal peptide. In some embodiments, the second fusion protein comprises the homing polypeptide. In some embodiments, domains of the second fusion protein are arranged from the N-terminus to the C-terminus in the following orders: (a) signal peptide, homing polypeptide, oligomerization domain, and transmembrane polypeptide; (b) signal peptide, homing polypeptide, transmembrane polypeptide, and oligomerization domain; or (c) signal peptide, oligomerization domain, homing polypeptide, and transmembrane polypeptide. In some embodiments, the second fusion protein further comprises a cytosolic domain. In some embodiments, domains of the second fusion protein are arranged from the N-terminus to the C-terminus in the following orders: (a) signal peptide, homing polypeptide, oligomerization domain, transmembrane polypeptide, and cytosolic domain; (b) signal peptide, homing polypeptide, transmembrane polypeptide, oligomerization domain, and cytosolic domain; or (c) signal peptide, oligomerization domain, homing peptide, transmembrane polypeptide, and cytosolic domain.

### Transmembrane Domain

In some embodiments, the transmembrane polypeptide of the first fusion protein anchors the first fusion protein to a bilayer of the multivalent particle. In some embodiments, the transmembrane polypeptide of the first fusion protein anchors the first fusion protein to a lipid bilayer of the multivalent particle. In some embodiments, the transmembrane polypeptide of the second fusion protein anchors the second fusion protein to a bilayer of the multivalent particle. In some embodiments, the transmembrane polypeptide of the second fusion protein anchors the second fusion protein to a lipid bilayer of the multivalent particle.

In some embodiments, the transmembrane polypeptide of the first fusion protein comprises the transmembrane domain of a Vesicular Stomatitis virus glycoprotein (VSV-G). In some embodiments, the transmembrane polypeptide of the first fusion protein comprises the cytosolic region of a Vesicular Stomatitis virus glycoprotein (VSV-G). In some embodiments, the transmembrane polypeptide of the first fusion protein comprises the transmembrane domain of influenza Neuraminidase (NA). In some embodiments, the transmembrane polypeptide of the first fusion protein comprises the transmembrane domain of influenza Hemagglutinin (HA). In some embodiments, the transmembrane polypeptide of the first fusion protein comprises the transmembrane domain of Dengue E Protein. In some embodiments, the transmembrane polypeptide of the first fusion protein comprises the transmembrane domain of HIV surface glycoprotein GP120 or GP41. In some embodiments, the transmembrane polypeptide of the first fusion protein comprises the transmembrane domain of measles virus surface glycoprotein hemagglutinin (H) protein.

In some embodiments, the transmembrane polypeptide of the first fusion protein comprises the transmembrane domain of a Vesicular Stomatitis virus glycoprotein (VSV-G). In some embodiments, the transmembrane polypeptide of the first fusion protein comprises the transmembrane domain and cytosolic domain of a Vesicular Stomatitis virus glycoprotein (VSV-G). In some embodiments, the transmembrane polypeptide of the first fusion protein comprises the transmembrane domain of a Dengue E protein. In some embodiments, the transmembrane polypeptide of the first fusion protein comprises the transmembrane domain and cytosolic domain of a Dengue E protein. In some embodiments, the transmembrane polypeptide of the first fusion protein comprises the transmembrane domain of influenza Hemagglutinin (HA). In some embodiments, the transmembrane polypeptide of the first fusion protein comprises the transmembrane domain and cytosolic domain of influenza Hemagglutinin (HA). In some embodiments, the transmembrane polypeptide of the first fusion protein comprises the transmembrane domain of HIV surface glycoprotein GP120 or GP41. In some embodiments, the transmembrane polypeptide of the first fusion protein comprises the transmembrane domain and cytosolic domain of HIV surface glycoprotein GP120 or GP41. In some embodiments, the transmembrane domain of the first fusion protein comprises the transmembrane polypeptide of measles virus surface glycoprotein hemagglutinin (H) protein. In some embodiments, the transmembrane polypeptide of the first fusion protein comprises the transmembrane domain and cytosolic domain of measles virus surface glycoprotein hemagglutinin (H) protein. In some embodiments, the transmembrane polypeptide of the first fusion protein comprises the transmembrane domain of influenza Neuraminidase (NA). In some embodiments, the transmembrane polypeptide of the first fusion protein comprises the transmembrane domain and cytosolic domain of influenza Neuraminidase (NA).

In some embodiments, the transmembrane polypeptide of the second fusion protein comprises the transmembrane domain of a Vesicular Stomatitis virus glycoprotein (VSV-G). In some embodiments, the transmembrane polypeptide of the second fusion protein comprises the cytosolic region of a Vesicular Stomatitis virus glycoprotein (VSV-G). In some embodiments, the transmembrane polypeptide of the second fusion protein comprises the transmembrane domain of influenza Neuraminidase (NA). In some embodiments, the transmembrane polypeptide of the second fusion protein comprises the transmembrane domain of influenza Hemagglutinin (HA). In some embodiments, the transmembrane polypeptide of the second fusion protein comprises the transmembrane domain of Dengue E Protein. In some embodiments, the transmembrane polypeptide of the second fusion protein comprises the transmembrane domain of HIV surface glycoprotein GP120 or GP41. In some embodiments, the transmembrane polypeptide of the second fusion protein comprises the transmembrane domain of measles virus surface glycoprotein hemagglutinin (H) protein.

In some embodiments, the transmembrane polypeptide of the second fusion protein comprises the transmembrane domain of a Vesicular Stomatitis virus glycoprotein (VSV-G). In some embodiments, the transmembrane polypeptide of the second fusion protein comprises the transmembrane domain and cytosolic domain of a Vesicular Stomatitis virus glycoprotein (VSV-G). In some embodiments, the transmembrane polypeptide of the second fusion protein comprises the transmembrane domain of a Dengue E protein. In some embodiments, the transmembrane polypeptide of the second fusion protein comprises the transmembrane domain and cytosolic domain of a Dengue E protein. In some embodiments, the transmembrane polypeptide of the second fusion protein comprises the transmembrane domain of influenza Hemagglutinin (HA). In some embodiments, the transmembrane polypeptide of the second fusion protein comprises the transmembrane domain and cytosolic domain of influenza Hemagglutinin (HA). In some embodiments, the transmembrane polypeptide of the second fusion protein comprises the transmembrane domain of HIV surface glycoprotein GP120 or GP41. In some embodiments, the transmembrane polypeptide of the second fusion protein comprises the transmembrane domain and cytosolic domain of HIV surface glycoprotein GP120 or GP41. In some embodiments, the transmembrane domain of the second fusion protein comprises the transmembrane polypeptide of measles virus surface glycoprotein hemagglutinin (H) protein. In some embodiments, the transmembrane polypeptide of the second fusion protein comprises the transmembrane domain and cytosolic domain of measles virus surface glycoprotein hemagglutinin (H) protein. In some embodiments, the transmembrane polypeptide of the second fusion protein comprises the transmembrane domain of influenza Neuraminidase (NA). In some embodiments, the transmembrane polypeptide of the second fusion protein comprises the transmembrane domain and cytosolic domain of influenza Neuraminidase (NA).

TABLE 6
Exemplary Transmembrane Domain Sequences

| | | SEQ |
|---|---|---|
| | | ID |
| Domain | Amino Acid Sequence | NO: |
| VSV-G | IASFFFIIGLIIGLFLVLRVGI | 66 |
| Transmembrane | | |
| (TM) V1 | | |
| VSV-G | PIELVEGWFSSWKSSIASFFFIIGLIIG | 67 |
| Transmembrane | LFLVLRVGI | |
| (TM) V2 | | |
| VSV-G | DDESLFFGDTGLSKNPIELVEGWFSSWK | 68 |
| Transmembrane | SSIASFFFIIGLIIGLFLVLRVGIH | |
| (TM) V3 | | |
| VSV-G | GMLDSDLHLSSKAQVFEHPHIQDAASQL | 69 |
| Transmembrane | PDDESLFFGDTGLSKNPIELVEGWFSSW | |
| (TM) V4 | KSSIASFFFIIGLIIGLFLVLRVGI | |
| VSV-G | HLCIKLKHTKKRQIYTDIEMNRLGK | 70 |
| Cytosolic Tail | | |
| (CT) | | |
| Influenza | IITIGSVCMTIGMANLILQIGNI | 71 |
| Neuraminidase | | |
| TM (NA) | | |
| Influenza | LAIYSTVASSLVLVVSLGAISFW | 72 |
| Hemagglutinin | | |
| TM (HA) | | |
| Dengue E | AYGVLFSGVSWTMKIGIGILLTWLGLNS | 73 |
| Protein TM | RSTSLSMTCIAVGMVTLYLGVMVQ | |
| HIV gp TM | FIMIVGGLVGLRIVFAVLSIV | 74 |

In some embodiments, the transmembrane domain of the first fusion protein comprises an amino acid sequence disclosed in Table 6, or an amino acid sequence that is substantially identical to an amino acid sequence in Table 6 (e.g. 75%, 80%, 85%, 90%, 95%, 96%, 97%, 98%, 99% sequence identity). In some instances, the transmembrane domain of the first fusion protein comprises an amino acid sequence comprising at least a portion having at least or about 10, 20, 30, 40, 50, 60, 70, 80, amino acid sequences of any sequence according to Table 6. In some embodiments, the transmembrane domain of the second fusion protein comprises an amino acid sequence disclosed in Table 6, or an amino acid sequence that is substantially identical to an amino acid sequence in Table 6 (e.g. 75%, 80%, 85%, 90%, 95%, 96%, 97%, 98%, 99% sequence identity). In some instances, the transmembrane domain of the second fusion protein comprises an amino acid sequence comprising at least a portion having at least or about 10, 20, 30, 40, 50, 60, 70, 80, amino acid sequences of any sequence according to Table 6.

In some embodiments, the transmembrane domain of the first fusion protein comprises an amino acid sequence with at least 75% sequence identity to any one of SEQ ID NOs: 66-74. In some embodiments, the transmembrane domain of the first fusion protein comprises an amino acid sequence with at least 76% sequence identity to any one of SEQ ID NOs: 66-74. In some embodiments, the transmembrane domain of the first fusion protein comprises an amino acid sequence with at least 77% sequence identity to any one of SEQ ID NOs: 66-74. In some embodiments, the transmembrane domain of the first fusion protein comprises an amino acid sequence with at least 78% sequence identity to any one of SEQ ID NOs: 66-74. In some embodiments, the transmembrane domain of the first fusion protein comprises an amino acid sequence with at least 79% sequence identity to any one of SEQ ID NOs: 66-74. In some embodiments, the transmembrane domain of the first fusion protein comprises an amino acid sequence with at least 80% sequence identity to any one of SEQ ID NOs: 66-74. In some embodiments, the transmembrane domain of the first fusion protein comprises an amino acid sequence with at least 81% sequence identity to any one of SEQ ID NOs: 66-74. In some embodiments, the transmembrane domain of the first fusion protein comprises an amino acid sequence with at least 82% sequence identity to any one of SEQ ID NOs: 66-74. In some embodiments, the transmembrane domain of the first fusion protein comprises an amino acid sequence with at least 83% sequence identity to any one of SEQ ID NOs: 66-74. In some embodiments, the transmembrane domain of the first fusion protein comprises an amino acid sequence with at least 84% sequence identity to any one of SEQ ID NOs: 66-74. In some embodiments, the transmembrane domain of the first fusion protein comprises an amino acid sequence with at least 85% sequence identity to any one of SEQ ID NOs: 66-74. In some embodiments, the transmembrane domain of the first fusion protein comprises an amino acid sequence with at least 86% sequence identity to any one of SEQ ID NOs: 66-74. In some embodiments, the transmembrane domain of the first fusion protein comprises an amino acid sequence with at least 87% sequence identity to any one of SEQ ID NOs: 66-74. In some embodiments, the transmembrane domain of the first fusion protein comprises an amino acid sequence with at least 88% sequence identity to any one of SEQ ID NOs: 66-74. In some embodiments, the transmembrane domain of the first fusion protein comprises an amino acid sequence with at least 89% sequence identity to any one of SEQ ID NOs: 66-74. In some embodiments, the transmembrane domain of the first fusion protein comprises an amino acid sequence with at least 90% sequence identity to any one of SEQ ID NOs: 66-74. In some embodiments, the transmembrane domain of the first fusion protein comprises an amino acid sequence with at least 91% sequence identity to any one of SEQ ID NOs: 66-74. In some embodiments, the transmembrane domain of the first fusion protein comprises an amino acid sequence with at least 92% sequence identity to any one of SEQ ID NOs: 66-74. In some embodiments, the transmembrane domain of the first fusion protein comprises an amino acid sequence with at least 93% sequence identity to any one of SEQ ID NOs: 66-74. In some embodiments, the transmembrane domain of the first fusion protein comprises an amino acid sequence with at least 94% sequence identity to any one of SEQ ID NOs: 66-74. In some embodiments, the transmembrane domain of the first fusion protein comprises an amino acid sequence with at least 95% sequence identity to any one of SEQ ID NOs: 66-74. In some embodiments, the transmembrane domain of the first fusion protein comprises an amino acid sequence with at least 96% sequence identity to any one of SEQ ID NOs: 66-74. In some embodiments, the transmembrane domain of the first fusion protein comprises an amino acid sequence with at least 97% sequence identity to any one of SEQ ID NOs: 66-74. In some embodiments, the transmembrane domain of the first fusion protein comprises an amino acid sequence with at least 98% sequence identity to any one of SEQ ID NOs: 66-74. In some embodiments, the transmembrane domain of the first fusion protein comprises an amino acid sequence with at least 99% sequence identity to any one of SEQ ID NOs: 66-74. In some embodiments, the transmembrane domain of the first fusion protein comprises an amino acid sequence according to any one of SEQ ID NOs: 66-74.

In some embodiments, the transmembrane domain of the second fusion protein comprises an amino acid sequence with at least 75% sequence identity to any one of SEQ ID NOs: 66-74. In some embodiments, the transmembrane domain of the second fusion protein comprises an amino acid sequence with at least 76% sequence identity to any one of SEQ ID NOs: 66-74. In some embodiments, the transmembrane domain of the second fusion protein comprises an amino acid sequence with at least 77% sequence identity to any one of SEQ ID NOs: 66-74. In some embodiments, the transmembrane domain of the second fusion protein comprises an amino acid sequence with at least 78% sequence identity to any one of SEQ ID NOs: 66-74. In some embodiments, the transmembrane domain of the second fusion protein comprises an amino acid sequence with at least 79% sequence identity to any one of SEQ ID NOs: 66-74. In some embodiments, the transmembrane domain of the second fusion protein comprises an amino acid sequence with at least 80% sequence identity to any one of SEQ ID NOs: 66-74. In some embodiments, the transmembrane domain of the second fusion protein comprises an amino acid sequence with at least 81% sequence identity to any one of SEQ ID NOs: 66-74. In some embodiments, the transmembrane domain of the second fusion protein comprises an amino acid sequence with at least 82% sequence identity to any one of SEQ ID NOs: 66-74. In some embodiments, the transmembrane domain of the second fusion protein comprises an amino acid sequence with at least 83% sequence identity to any one of SEQ ID NOs: 66-74. In some embodiments, the transmembrane domain of the second fusion protein comprises an amino acid sequence with at least 84% sequence identity to any one of SEQ ID NOs: 66-74. In some embodiments, the transmembrane domain of the second fusion protein comprises an amino acid sequence with at least 85% sequence identity to any one of SEQ ID NOs: 66-74. In some embodiments, the transmembrane domain of the second fusion protein comprises an amino acid sequence with at least 86% sequence identity to any one of SEQ ID NOs: 66-74. In some embodiments, the transmembrane domain of the second fusion protein comprises an amino acid sequence with at least 87% sequence identity to any one of SEQ ID NOs: 66-74. In some embodiments, the transmembrane domain of the second fusion protein comprises an amino acid sequence with at least 88% sequence identity to any one of SEQ ID NOs: 66-74. In some embodiments, the transmembrane domain of the second fusion protein comprises an amino acid sequence with at least 89% sequence identity to any one of SEQ ID NOs: 66-74. In some embodiments, the transmembrane domain of the second fusion protein comprises an amino acid sequence with at least 90% sequence identity to any one of SEQ ID NOs: 66-74. In some embodiments, the transmembrane domain of the second fusion protein comprises an amino acid sequence with at least 91% sequence identity to any one of SEQ ID NOs: 66-74. In some embodiments, the transmembrane domain of the second fusion protein comprises an amino acid sequence with at least 92% sequence identity to any one of SEQ ID NOs: 66-74. In some embodiments, the transmembrane domain of the second fusion protein comprises an amino acid sequence with at least 93% sequence identity to any one of SEQ ID NOs: 66-74. In some embodiments, the transmembrane domain of the second fusion protein comprises an amino acid sequence with at least 94% sequence identity to any one of SEQ ID NOs: 66-74. In some embodiments, the transmembrane domain of the second fusion protein comprises an amino acid sequence with at least 95% sequence identity to any one of SEQ ID NOs: 66-74. In some embodiments, the transmembrane domain of the second fusion protein comprises an amino acid sequence with at least 96% sequence identity to any one of SEQ ID NOs: 66-74. In some embodiments, the transmembrane domain of the second fusion protein comprises an amino acid sequence with at least 97% sequence identity to any one of SEQ ID NOs: 66-74. In some embodiments, the transmembrane domain of the second fusion protein comprises an amino acid sequence with at least 98% sequence identity to any one of SEQ ID NOs: 66-74. In some embodiments, the transmembrane domain of the second fusion protein comprises an amino acid sequence with at least 99% sequence identity to any one of SEQ ID NOs: 66-74. In some embodiments, the transmembrane domain of the second fusion protein comprises an amino acid sequence according to any one of SEQ ID NOs: 66-74.

In some embodiments, the IFN polypeptide of the first fusion protein comprises IFN-alpha, IFN-beta, IFN-epsilon, IFN-kappa, IFN-omega, IFN-gamma, or IFN-lambda, and the transmembrane polypeptide of the first fusion protein comprises a transmembrane domain of a Vesicular Stomatitis virus glycoprotein (VSV-G), a transmembrane domain and a cytosolic domain of VSV-G, a transmembrane domain of Influenza Neuraminidase (NA), a transmembrane domain and a cytosolic domain of Influenza Neuraminidase (NA), a transmembrane domain of influenza Hemagglutinin (HA), a transmembrane domain and a cytosolic domain of influenza Hemagglutinin (HA), a transmembrane domain of HIV surface glycoprotein GP120 or GP41, a transmembrane domain and a cytosolic domain of HIV surface glycoprotein GP120 or GP4, a transmembrane domain of Dengue E Protein, a transmembrane domain and a cytosolic domain of Dengue E Protein, a transmembrane domain of measles virus surface glycoprotein hemagglutinin (H) protein, or a transmembrane domain and a cytosolic domain of measles virus surface glycoprotein hemagglutinin (H) protein. In some embodiments, the IFN polypeptide of the second fusion protein comprises IFN-alpha, IFN-beta, IFN-epsilon, IFN-kappa, IFN-omega, IFN-gamma, or IFN-lambda, and the transmembrane polypeptide of the second fusion protein comprises a transmembrane domain of a Vesicular Stomatitis virus glycoprotein (VSV-G), a transmembrane domain and a cytosolic domain of VSV-G, a transmembrane domain of Influenza Neuraminidase (NA), a transmembrane domain and a cytosolic domain of Influenza Neuraminidase (NA), a transmembrane domain of influenza Hemagglutinin (HA), a transmembrane domain and a cytosolic domain of influenza Hemagglutinin (HA), a transmembrane domain of HIV surface glycoprotein GP120 or GP41, a transmembrane domain and a cytosolic domain of HIV surface glycoprotein GP120 or GP4, a transmembrane domain of Dengue E Protein, a transmembrane domain and a cytosolic domain of Dengue E Protein, a transmembrane domain of measles virus surface glycoprotein hemagglutinin (H) protein, or a transmembrane domain and a cytosolic domain of measles virus surface glycoprotein hemagglutinin (H) protein. In some embodiments, the IFN polypeptide of the first fusion protein comprises a mammalian IFN. In some embodiments, the IFN polypeptide of the first fusion protein comprises a human IFN. In some embodiments, the IFN polypeptide of the second fusion protein comprises a mammalian IFN. In some embodiments, the IFN polypeptide of the second fusion protein comprises a human IFN.

In some embodiments, the IFN polypeptide of the first fusion protein comprises an amino acid sequence with at least 75% sequence identity to any one of SEQ ID NOs: 1-7, and the transmembrane polypeptide of the first fusion protein comprises an amino acid sequence with at least 75% sequence identity to any one of SEQ ID NOs: 66-74. In some embodiments, the IFN polypeptide of the first fusion protein comprises an amino acid sequence with at least 76% sequence identity to any one of SEQ ID NOs: 1-7, and the transmembrane polypeptide of the first fusion protein comprises an amino acid sequence with at least 76% sequence identity to any one of SEQ ID NOs: 66-74. In some embodiments, the IFN polypeptide of the first fusion protein comprises an amino acid sequence with at least 77% sequence identity to any one of SEQ ID NOs: 1-7, and the transmembrane polypeptide of the first fusion protein comprises an amino acid sequence with at least 77% sequence identity to any one of SEQ ID NOs: 66-74. In some embodiments, the IFN polypeptide of the first fusion protein comprises an amino acid sequence with at least 78% sequence identity to any one of SEQ ID NOs: 1-7, and the transmembrane polypeptide of the first fusion protein comprises an amino acid sequence with at least 78% sequence identity to any one of SEQ ID NOs: 66-74. In some embodiments, the IFN polypeptide of the first fusion protein comprises an amino acid sequence with at least 79% sequence identity to any one of SEQ ID NOs: 1-7, and the transmembrane polypeptide of the first fusion protein comprises an amino acid sequence with at least 79% sequence identity to any one of SEQ ID NOs: 66-74. In some embodiments, the IFN polypeptide of the first fusion protein comprises an amino acid sequence with at least 80% sequence identity to any one of SEQ ID NOs: 1-7, and the transmembrane polypeptide of the first fusion protein comprises an amino acid sequence with at least 80% sequence identity to any one of SEQ ID NOs: 66-74. In some embodiments, the IFN polypeptide of the first fusion protein comprises an amino acid sequence with at least 81% sequence identity to any one of SEQ ID NOs: 1-7, and the transmembrane polypeptide of the first fusion protein comprises an amino acid sequence with at least 81% sequence identity to any one of SEQ ID NOs: 66-74. In some embodiments, the IFN polypeptide of the first fusion protein comprises an amino acid sequence with at least 82% sequence identity to any one of SEQ ID NOs: 1-7, and the transmembrane polypeptide of the first fusion protein comprises an amino acid sequence with at least 82% sequence identity to any one of SEQ ID NOs: 66-74. In some embodiments, the IFN polypeptide of the first fusion protein comprises an amino acid sequence with at least 83% sequence identity to any one of SEQ ID NOs: 1-7, and the transmembrane polypeptide of the first fusion protein comprises an amino acid sequence with at least 83% sequence identity to any one of SEQ ID NOs: 66-74. In some embodiments, the IFN polypeptide of the first fusion protein comprises an amino acid sequence with at least 84% sequence identity to any one of SEQ ID NOs: 1-7, and the transmembrane polypeptide of the first fusion protein comprises an amino acid sequence with at least 84% sequence identity to any one of SEQ ID NOs: 66-74. In some embodiments, the IFN polypeptide of the first fusion protein comprises an amino acid sequence with at least 85% sequence identity to any one of SEQ ID NOs: 1-7, and the transmembrane polypeptide of the first fusion protein comprises an amino acid sequence with at least 85% sequence identity to any one of SEQ ID NOs: 66-74. In some embodiments, the IFN polypeptide of the first fusion protein comprises an amino acid sequence with at least 86% sequence identity to any one of SEQ ID NOs: 1-7, and the transmembrane polypeptide of the first fusion protein comprises an amino acid sequence with at least 86% sequence identity to any one of SEQ ID NOs: 66-74. In some embodiments, the IFN polypeptide of the first fusion protein comprises an amino acid sequence with at least 87% sequence identity to any one of SEQ ID NOs: 1-7, and the transmembrane polypeptide of the first fusion protein comprises an amino acid sequence with at least 87% sequence identity to any one of SEQ ID NOs: 66-74. In some embodiments, the IFN polypeptide of the first fusion protein comprises an amino acid sequence with at least 88% sequence identity to any one of SEQ ID NOs: 1-7, and the transmembrane polypeptide of the first fusion protein comprises an amino acid sequence with at least 88% sequence identity to any one of SEQ ID NOs: 66-74. In some embodiments, the IFN polypeptide of the first fusion protein comprises an amino acid sequence with at least 89% sequence identity to any one of SEQ ID NOs: 1-7, and the transmembrane polypeptide of the first fusion protein comprises an amino acid sequence with at least 89% sequence identity to any one of SEQ ID NOs: 66-74. In some embodiments, the IFN polypeptide of the first fusion protein comprises an amino acid sequence with at least 90% sequence identity to any one of SEQ ID NOs: 1-7, and the transmembrane polypeptide of the first fusion protein comprises an amino acid sequence with at least 90% sequence identity to any one of SEQ ID NOs: 66-74. In some embodiments, the IFN polypeptide of the first fusion protein comprises an amino acid sequence with at least 91% sequence identity to any one of SEQ ID NOs: 1-7, and the transmembrane polypeptide of the first fusion protein comprises an amino acid sequence with at least 91% sequence identity to any one of SEQ ID NOs: 66-74. In some embodiments, the IFN polypeptide of the first fusion protein comprises an amino acid sequence with at least 92% sequence identity to any one of SEQ ID NOs: 1-7, and the transmembrane polypeptide of the first fusion protein comprises an amino acid sequence with at least 92% sequence identity to any one of SEQ ID NOs: 66-74. In some embodiments, the IFN polypeptide of the first fusion protein comprises an amino acid sequence with at least 93% sequence identity to any one of SEQ ID NOs: 1-7, and the transmembrane polypeptide of the first fusion protein comprises an amino acid sequence with at least 93% sequence identity to any one of SEQ ID NOs: 66-74. In some embodiments, the IFN polypeptide of the first fusion protein comprises an amino acid sequence with at least 94% sequence identity to any one of SEQ ID NOs: 1-7, and the transmembrane polypeptide of the first fusion protein comprises an amino acid sequence with at least 94% sequence identity to any one of SEQ ID NOs: 66-74. In some embodiments, the IFN polypeptide of the first fusion protein comprises an amino acid sequence with at least 95% sequence identity to any one of SEQ ID NOs: 1-7, and the transmembrane polypeptide of the first fusion protein comprises an amino acid sequence with at least 95% sequence identity to any one of SEQ ID NOs: 66-74. In some embodiments, the IFN polypeptide of the first fusion protein comprises an amino acid sequence with at least 96% sequence identity to any one of SEQ ID NOs: 1-7, and the transmembrane polypeptide of the first fusion protein comprises an amino acid sequence with at least 96% sequence identity to any one of SEQ ID NOs: 66-74. In some embodiments, the IFN polypeptide of the first fusion protein comprises an amino acid sequence with at least 97% sequence identity to any one of SEQ ID NOs: 1-7, and the transmembrane polypeptide of the first fusion protein comprises an amino acid sequence with at least 97% sequence identity to any one of SEQ ID NOs: 66-74. In some embodiments, the IFN polypeptide of the first fusion protein comprises an amino acid sequence with at least 98% sequence identity to any one of SEQ ID NOs: 1-7, and the transmembrane polypeptide of the first fusion protein comprises an amino acid sequence with at least 98% sequence identity to any one of SEQ ID NOs: 66-74. In some embodiments, the IFN polypeptide of the first fusion protein comprises an amino acid sequence with at least 99% sequence identity to any one of SEQ ID NOs: 1-7, and the transmembrane polypeptide of the first fusion protein comprises an amino acid sequence with at least 99% sequence identity to any one of SEQ ID NOs: 66-74. In some embodiments, the IFN polypeptide of the first fusion protein comprises an amino acid sequence according to any one of SEQ ID NOs: 1-7, and the transmembrane polypeptide of the first fusion protein comprises an amino acid sequence according to any one of SEQ ID NOs: 66-74.

In some embodiments, the IFN polypeptide of the second fusion protein comprises an amino acid sequence with at least 75% sequence identity to any one of SEQ ID NOs: 1-7, and the transmembrane polypeptide of the second fusion protein comprises an amino acid sequence with at least 75% sequence identity to any one of SEQ ID NOs: 66-74. In some embodiments, the IFN polypeptide of the second fusion protein comprises an amino acid sequence with at least 76% sequence identity to any one of SEQ ID NOs: 1-7, and the transmembrane polypeptide of the second fusion protein comprises an amino acid sequence with at least 76% sequence identity to any one of SEQ ID NOs: 66-74. In some embodiments, the IFN polypeptide of the second fusion protein comprises an amino acid sequence with at least 77% sequence identity to any one of SEQ ID NOs: 1-7, and the transmembrane polypeptide of the second fusion protein comprises an amino acid sequence with at least 77% sequence identity to any one of SEQ ID NOs: 66-74. In some embodiments, the IFN polypeptide of the second fusion protein comprises an amino acid sequence with at least 78% sequence identity to any one of SEQ ID NOs: 1-7, and the transmembrane polypeptide of the second fusion protein comprises an amino acid sequence with at least 78% sequence identity to any one of SEQ ID NOs: 66-74. In some embodiments, the IFN polypeptide of the second fusion protein comprises an amino acid sequence with at least 79% sequence identity to any one of SEQ ID NOs: 1-7, and the transmembrane polypeptide of the second fusion protein comprises an amino acid sequence with at least 79% sequence identity to any one of SEQ ID NOs: 66-74. In some embodiments, the IFN polypeptide of the second fusion protein comprises an amino acid sequence with at least 80% sequence identity to any one of SEQ ID NOs: 1-7, and the transmembrane polypeptide of the second fusion protein comprises an amino acid sequence with at least 80% sequence identity to any one of SEQ ID NOs: 66-74. In some embodiments, the IFN polypeptide of the second fusion protein comprises an amino acid sequence with at least 81% sequence identity to any one of SEQ ID NOs: 1-7, and the transmembrane polypeptide of the second fusion protein comprises an amino acid sequence with at least 81% sequence identity to any one of SEQ ID NOs: 66-74. In some embodiments, the IFN polypeptide of the second fusion protein comprises an amino acid sequence with at least 82% sequence identity to any one of SEQ ID NOs: 1-7, and the transmembrane polypeptide of the second fusion protein comprises an amino acid sequence with at least 82% sequence identity to any one of SEQ ID NOs: 66-74. In some embodiments, the IFN polypeptide of the second fusion protein comprises an amino acid sequence with at least 83% sequence identity to any one of SEQ ID NOs: 1-7, and the transmembrane polypeptide of the second fusion protein comprises an amino acid sequence with at least 83% sequence identity to any one of SEQ ID NOs: 66-74. In some embodiments, the IFN polypeptide of the second fusion protein comprises an amino acid sequence with at least 84% sequence identity to any one of SEQ ID NOs: 1-7, and the transmembrane polypeptide of the second fusion protein comprises an amino acid sequence with at least 84% sequence identity to any one of SEQ ID NOs: 66-74. In some embodiments, the IFN polypeptide of the second fusion protein comprises an amino acid sequence with at least 85% sequence identity to any one of SEQ ID NOs: 1-7, and the transmembrane polypeptide of the second fusion protein comprises an amino acid sequence with at least 85% sequence identity to any one of SEQ ID NOs: 66-74. In some embodiments, the IFN polypeptide of the second fusion protein comprises an amino acid sequence with at least 86% sequence identity to any one of SEQ ID NOs: 1-7, and the transmembrane polypeptide of the second fusion protein comprises an amino acid sequence with at least 86% sequence identity to any one of SEQ ID NOs: 66-74. In some embodiments, the IFN polypeptide of the second fusion protein comprises an amino acid sequence with at least 87% sequence identity to any one of SEQ ID NOs: 1-7, and the transmembrane polypeptide of the second fusion protein comprises an amino acid sequence with at least 87% sequence identity to any one of SEQ ID NOs: 66-74. In some embodiments, the IFN polypeptide of the second fusion protein comprises an amino acid sequence with at least 88% sequence identity to any one of SEQ ID NOs: 1-7, and the transmembrane polypeptide of the second fusion protein comprises an amino acid sequence with at least 88% sequence identity to any one of SEQ ID NOs: 66-74. In some embodiments, the IFN polypeptide of the second fusion protein comprises an amino acid sequence with at least 89% sequence identity to any one of SEQ ID NOs: 1-7, and the transmembrane polypeptide of the second fusion protein comprises an amino acid sequence with at least 89% sequence identity to any one of SEQ ID NOs: 66-74. In some embodiments, the IFN polypeptide of the second fusion protein comprises an amino acid sequence with at least 90% sequence identity to any one of SEQ ID NOs: 1-7, and the transmembrane polypeptide of the second fusion protein comprises an amino acid sequence with at least 90% sequence identity to any one of SEQ ID NOs: 66-74. In some embodiments, the IFN polypeptide of the second fusion protein comprises an amino acid sequence with at least 91% sequence identity to any one of SEQ ID NOs: 1-7, and the transmembrane polypeptide of the second fusion protein comprises an amino acid sequence with at least 91% sequence identity to any one of SEQ ID NOs: 66-74. In some embodiments, the IFN polypeptide of the second fusion protein comprises an amino acid sequence with at least 92% sequence identity to any one of SEQ ID NOs: 1-7, and the transmembrane polypeptide of the second fusion protein comprises an amino acid sequence with at least 92% sequence identity to any one of SEQ ID NOs: 66-74. In some embodiments, the IFN polypeptide of the second fusion protein comprises an amino acid sequence with at least 93% sequence identity to any one of SEQ ID NOs: 1-7, and the transmembrane polypeptide of the second fusion protein comprises an amino acid sequence with at least 93% sequence identity to any one of SEQ ID NOs: 66-74. In some embodiments, the IFN polypeptide of the second fusion protein comprises an amino acid sequence with at least 94% sequence identity to any one of SEQ ID NOs: 1-7, and the transmembrane polypeptide of the second fusion protein comprises an amino acid sequence with at least 94% sequence identity to any one of SEQ ID NOs: 66-74. In some embodiments, the IFN polypeptide of the second fusion protein comprises an amino acid sequence with at least 95% sequence identity to any one of SEQ ID NOs: 1-7, and the transmembrane polypeptide of the second fusion protein comprises an amino acid sequence with at least 95% sequence identity to any one of SEQ ID NOs: 66-74. In some embodiments, the IFN polypeptide of the second fusion protein comprises an amino acid sequence with at least 96% sequence identity to any one of SEQ ID NOs: 1-7, and the transmembrane polypeptide of the second fusion protein comprises an amino acid sequence with at least 96% sequence identity to any one of SEQ ID NOs: 66-74. In some embodiments, the IFN polypeptide of the second fusion protein comprises an amino acid sequence with at least 97% sequence identity to any one of SEQ ID NOs: 1-7, and the transmembrane polypeptide of the second fusion protein comprises an amino acid sequence with at least 97% sequence identity to any one of SEQ ID NOs: 66-74. In some embodiments, the IFN polypeptide of the second fusion protein comprises an amino acid sequence with at least 98% sequence identity to any one of SEQ ID NOs: 1-7, and the transmembrane polypeptide of the second fusion protein comprises an amino acid sequence with at least 98% sequence identity to any one of SEQ ID NOs: 66-74. In some embodiments, the IFN polypeptide of the second fusion protein comprises an amino acid sequence with at least 99% sequence identity to any one of SEQ ID NOs: 1-7, and the transmembrane polypeptide of the second fusion protein comprises an amino acid sequence with at least 99% sequence identity to any one of SEQ ID NOs: 66-74. In some embodiments, the IFN polypeptide of the second fusion protein comprises an amino acid sequence according to any one of SEQ ID NOs: 1-7, and the transmembrane polypeptide of the second fusion protein comprises an amino acid sequence according to any one of SEQ ID NOs: 66-74.

In some embodiments, the second fusion protein comprises a transmembrane polypeptide and a homing polypeptide that targets the multivalent particle to a target cell or a target protein. In some embodiments, the homing polypeptide comprises an antibody that binds specifically to an antigen on the target cell or the target protein. In some embodiments, the antigen on the target cell comprises an antigen listed in Table 2 and the transmembrane polypeptide of the second fusion protein comprises a transmembrane domain of a Vesicular Stomatitis virus glycoprotein (VSV-G), a transmembrane domain and a cytosolic domain of VSV-G, a transmembrane domain of Influenza Neuraminidase (NA), a transmembrane domain and a cytosolic domain of Influenza Neuraminidase (NA), a transmembrane domain of influenza Hemagglutinin (HA), a transmembrane domain and a cytosolic domain of influenza Hemagglutinin (HA), a transmembrane domain of HIV surface glycoprotein GP120 or GP41, a transmembrane domain and a cytosolic domain of HIV surface glycoprotein GP120 or GP4, a transmembrane domain of Dengue E Protein, a transmembrane domain and a cytosolic domain of Dengue E Protein, a transmembrane domain of measles virus surface glycoprotein hemagglutinin (H) protein, or a transmembrane domain and a cytosolic domain of measles virus surface glycoprotein hemagglutinin (H) protein. In some embodiments, the antibody comprises an amino acid sequence from at least one complementarity determining region of BG10-19, 80R, 7D10, FI6, 1E01, H015, 2H5, ADRI-2F3, H004, H009, H007, H019, or H020, and the transmembrane polypeptide of the second fusion protein comprises a transmembrane domain of a Vesicular Stomatitis virus glycoprotein (VSV-G), a transmembrane domain and a cytosolic domain of VSV-G, a transmembrane domain of Influenza Neuraminidase (NA), a transmembrane domain and a cytosolic domain of Influenza Neuraminidase (NA), a transmembrane domain of influenza Hemagglutinin (HA), a transmembrane domain and a cytosolic domain of influenza Hemagglutinin (HA), a transmembrane domain of HIV surface glycoprotein GP120 or GP41, a transmembrane domain and a cytosolic domain of HIV surface glycoprotein GP120 or GP4, a transmembrane domain of Dengue E Protein, a transmembrane domain and a cytosolic domain of Dengue E Protein, a transmembrane domain of measles virus surface glycoprotein hemagglutinin (H) protein, or a transmembrane domain and a cytosolic domain of measles virus surface glycoprotein hemagglutinin (H) protein.

In some embodiments, the antibody comprises a CDR-H1 according to SEQ ID NO: 75, a CDR-H2 according to SEQ ID NO: 76, a CDR-H3 according to SEQ ID NO: 77, a CDR-L1 according to SEQ ID NO: 114, a CDR-L2 according to SEQ ID NO: 115, and a CDR-L3 according to SEQ ID NO: 116, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 75, a CDR-H2 according to SEQ ID NO: 76, a CDR-H3 according to SEQ ID NO: 77, a CDR-L1 according to SEQ ID NO: 114, a CDR-L2 according to SEQ ID NO: 115, and a CDR-L3 according to SEQ ID NO: 116, and the transmembrane polypeptide of the second fusion protein comprises an amino acid sequence according to any one of SEQ ID NOs: 66-74. In some embodiments, the antibody comprises a CDR-H1 according to SEQ ID NO: 78, a CDR-H2 according to SEQ ID NO: 79, a CDR-H3 according to SEQ ID NO: 80, a CDR-L1 according to SEQ ID NO: 117, a CDR-L2 according to SEQ ID NO: 118, and a CDR-L3 according to SEQ ID NO: 119, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 78, a CDR-H2 according to SEQ ID NO: 79, a CDR-H3 according to SEQ ID NO: 80, a CDR-L1 according to SEQ ID NO: 117, a CDR-L2 according to SEQ ID NO: 118, and a CDR-L3 according to SEQ ID NO: 119, and the transmembrane polypeptide of the second fusion protein comprises an amino acid sequence according to any one of SEQ ID NOs: 66-74. In some embodiments, the antibody comprises a CDR-H1 according to SEQ ID NO: 81, a CDR-H2 according to SEQ ID NO: 82, a CDR-H3 according to SEQ ID NO: 83, a CDR-L1 according to SEQ ID NO: 120, a CDR-L2 according to SEQ ID NO: 121, and a CDR-L3 according to SEQ ID NO: 122, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 81, a CDR-H2 according to SEQ ID NO: 82, a CDR-H3 according to SEQ ID NO: 83, a CDR-L1 according to SEQ ID NO: 120, a CDR-L2 according to SEQ ID NO: 121, and a CDR-L3 according to SEQ ID NO: 122, and the transmembrane polypeptide of the second fusion protein comprises an amino acid sequence according to any one of SEQ ID NOS: 66-74. In some embodiments, the antibody comprises a CDR-H1 according to SEQ ID NO: 84, a CDR-H2 according to SEQ ID NO: 85, a CDR-H3 according to SEQ ID NO: 86, a CDR-L1 according to SEQ ID NO: 123, a CDR-L2 according to SEQ ID NO: 124, and a CDR-L3 according to SEQ ID NO: 125, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 84, a CDR-H2 according to SEQ ID NO: 85, a CDR-H3 according to SEQ ID NO: 86, a CDR-L1 according to SEQ ID NO: 123, a CDR-L2 according to SEQ ID NO: 124, and a CDR-L3 according to SEQ ID NO: 125, and the transmembrane polypeptide of the second fusion protein comprises an amino acid sequence according to any one of SEQ ID NOs: 66-74. In some embodiments, the antibody comprises a CDR-H1 according to SEQ ID NO: 87, a CDR-H2 according to SEQ ID NO: 88, a CDR-H3 according to SEQ ID NO: 89, a CDR-L1 according to SEQ ID NO: 126, a CDR-L2 according to SEQ ID NO: 127, and a CDR-L3 according to SEQ ID NO: 128, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 87, a CDR-H2 according to SEQ ID NO: 88, a CDR-H3 according to SEQ ID NO: 89, a CDR-L1 according to SEQ ID NO: 126, a CDR-L2 according to SEQ ID NO: 127, and a CDR-L3 according to SEQ ID NO: 128, and the transmembrane polypeptide of the second fusion protein comprises an amino acid sequence according to any one of SEQ ID NOs: 66-74. In some embodiments, the antibody comprises a CDR-H1 according to SEQ ID NO: 90, a CDR-H2 according to SEQ ID NO: 91, a CDR-H3 according to SEQ ID NO: 92, a CDR-L1 according to SEQ ID NO: 129, a CDR-L2 according to SEQ ID NO: 130, and a CDR-L3 according to SEQ ID NO: 131, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 90, a CDR-H2 according to SEQ ID NO: 91, a CDR-H3 according to SEQ ID NO: 92, a CDR-L1 according to SEQ ID NO: 129, a CDR-L2 according to SEQ ID NO: 130, and a CDR-L3 according to SEQ ID NO: 131, and the transmembrane polypeptide of the second fusion protein comprises an amino acid sequence according to any one of SEQ ID NOs: 66-74. In some embodiments, the antibody comprises a CDR-H1 according to SEQ ID NO: 93, a CDR-H2 according to SEQ ID NO: 94, a CDR-H3 according to SEQ ID NO: 95, a CDR-L1 according to SEQ ID NO: 132, a CDR-L2 according to SEQ ID NO: 133, and a CDR-L3 according to SEQ ID NO: 134, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 93, a CDR-H2 according to SEQ ID NO: 94, a CDR-H3 according to SEQ ID NO: 95, a CDR-L1 according to SEQ ID NO: 132, a CDR-L2 according to SEQ ID NO: 133, and a CDR-L3 according to SEQ ID NO: 134, and the transmembrane polypeptide of the second fusion protein comprises an amino acid sequence according to any one of SEQ ID NOs: 66-74. In some embodiments, the antibody comprises a CDR-H1 according to SEQ ID NO: 96, a CDR-H2 according to SEQ ID NO: 97, a CDR-H3 according to SEQ ID NO: 98, a CDR-L1 according to SEQ ID NO: 135, a CDR-L2 according to SEQ ID NO: 136, and a CDR-L3 according to SEQ ID NO: 137, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 96, a CDR-H2 according to SEQ ID NO: 97, a CDR-H3 according to SEQ ID NO: 98, a CDR-L1 according to SEQ ID NO: 135, a CDR-L2 according to SEQ ID NO: 136, and a CDR-L3 according to SEQ ID NO: 137, and the transmembrane polypeptide of the second fusion protein comprises an amino acid sequence according to any one of SEQ ID NOs: 66-74. In some embodiments, the antibody comprises a CDR-H1 according to SEQ ID NO: 99, a CDR-H2 according to SEQ ID NO: 100, a CDR-H3 according to SEQ ID NO: 101, a CDR-L1 according to SEQ ID NO: 138, a CDR-L2 according to SEQ ID NO: 139, and a CDR-L3 according to SEQ ID NO: 140, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 99, a CDR-H2 according to SEQ ID NO: 100, a CDR-H3 according to SEQ ID NO: 101, a CDR-L1 according to SEQ ID NO: 138, a CDR-L2 according to SEQ ID NO: 139, and a CDR-L3 according to SEQ ID NO: 140, and the transmembrane polypeptide of the second fusion protein comprises an amino acid sequence according to any one of SEQ ID NOs: 66-74. In some embodiments, the antibody comprises a CDR-H1 according to SEQ ID NO: 102, a CDR-H2 according to SEQ ID NO: 103, a CDR-H3 according to SEQ ID NO: 104, a CDR-L1 according to SEQ ID NO: 141, a CDR-L2 according to SEQ ID NO: 142, and a CDR-L3 according to SEQ ID NO: 143, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 102, a CDR-H2 according to SEQ ID NO: 103, a CDR-H3 according to SEQ ID NO: 104, a CDR-L1 according to SEQ ID NO: 141, a CDR-L2 according to SEQ ID NO: 142, and a CDR-L3 according to SEQ ID NO: 143, and the transmembrane polypeptide of the second fusion protein comprises an amino acid sequence according to any one of SEQ ID NOs: 66-74. In some embodiments, the antibody comprises a CDR-H1 according to SEQ ID NO: 105, a CDR-H2 according to SEQ ID NO: 106, a CDR-H3 according to SEQ ID NO: 107, a CDR-L1 according to SEQ ID NO: 144, a CDR-L2 according to SEQ ID NO: 145, and a CDR-L3 according to SEQ ID NO: 146, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 105, a CDR-H2 according to SEQ ID NO: 106, a CDR-H3 according to SEQ ID NO: 107, a CDR-L1 according to SEQ ID NO: 144, a CDR-L2 according to SEQ ID NO: 145, and a CDR-L3 according to SEQ ID NO: 146, and the transmembrane polypeptide of the second fusion protein comprises an amino acid sequence according to any one of SEQ ID NOs: 66-74. In some embodiments, the antibody comprises a CDR-H1 according to SEQ ID NO: 108, a CDR-H2 according to SEQ ID NO: 109, a CDR-H3 according to SEQ ID NO: 110, a CDR-L1 according to SEQ ID NO: 147, a CDR-L2 according to SEQ ID NO: 148, and a CDR-L3 according to SEQ ID NO: 149, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 108, a CDR-H2 according to SEQ ID NO: 109, a CDR-H3 according to SEQ ID NO: 110, a CDR-L1 according to SEQ ID NO: 147, a CDR-L2 according to SEQ ID NO: 148, and a CDR-L3 according to SEQ ID NO: 149, and the transmembrane polypeptide of the second fusion protein comprises an amino acid sequence according to any one of SEQ ID NOs: 66-74. In some embodiments, the antibody comprises a CDR-H1 according to SEQ ID NO: 111, a CDR-H2 according to SEQ ID NO: 112, a CDR-H3 according to SEQ ID NO: 113, a CDR-L1 according to SEQ ID NO: 150, a CDR-L2 according to SEQ ID NO: 151, and a CDR-L3 according to SEQ ID NO: 152, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74. In some embodiments, the antibody comprises a CDR-H1 according to SEQ ID NO: 111, a CDR-H2 according to SEQ ID NO: 112, a CDR-H3 according to SEQ ID NO: 113, a CDR-L1 according to SEQ ID NO: 150, a CDR-L2 according to SEQ ID NO: 151, and a CDR-L3 according to SEQ ID NO: 152, and the transmembrane polypeptide of the second fusion protein comprises an amino acid sequence according to any one of SEQ ID NOs: 66-74.

In some embodiments, the antibody comprises a heavy chain variable region (VH) comprising an amino acid sequence with at least 75% sequence identity to any one of SEQ ID Nos: 8-20 and a light chain variable region (VL) comprising an amino acid sequence with at least 75% sequence identity to any one of SEQ ID Nos: 21-33, and the transmembrane polypeptide of the second fusion protein comprises an amino acid sequence with at least 75% sequence identity to any one of SEQ ID NOs: 66-74. In some embodiments, the antibody comprises a heavy chain variable region (VH) comprising an amino acid sequence with at least 76% sequence identity to any one of SEQ ID Nos: 8-20 and a light chain variable region (VL) comprising an amino acid sequence with at least 76% sequence identity to any one of SEQ ID Nos: 21-33, and the transmembrane polypeptide of the second fusion protein comprises an amino acid sequence with at least 76% sequence identity to any one of SEQ ID NOs: 66-74. In some embodiments, the antibody comprises a heavy chain variable region (VH) comprising an amino acid sequence with at least 77% sequence identity to any one of SEQ ID Nos: 8-20 and a light chain variable region (VL) comprising an amino acid sequence with at least 77% sequence identity to any one of SEQ ID Nos: 21-33, and the transmembrane polypeptide of the second fusion protein comprises an amino acid sequence with at least 77% sequence identity to any one of SEQ ID NOs: 66-74. In some embodiments, the antibody comprises a heavy chain variable region (VH) comprising an amino acid sequence with at least 78% sequence identity to any one of SEQ ID Nos: 8-20 and a light chain variable region (VL) comprising an amino acid sequence with at least 78% sequence identity to any one of SEQ ID Nos: 21-33, and the transmembrane polypeptide of the second fusion protein comprises an amino acid sequence with at least 78% sequence identity to any one of SEQ ID NOs: 66-74. In some embodiments, the antibody comprises a heavy chain variable region (VH) comprising an amino acid sequence with at least 79% sequence identity to any one of SEQ ID Nos: 8-20 and a light chain variable region (VL) comprising an amino acid sequence with at least 79% sequence identity to any one of SEQ ID Nos: 21-33, and the transmembrane polypeptide of the second fusion protein comprises an amino acid sequence with at least 79% sequence identity to any one of SEQ ID NOs: 66-74. In some embodiments, the antibody comprises a heavy chain variable region (VH) comprising an amino acid sequence with at least 80% sequence identity to any one of SEQ ID Nos: 8-20 and a light chain variable region (VL) comprising an amino acid sequence with at least 80% sequence identity to any one of SEQ ID Nos: 21-33, and the transmembrane polypeptide of the second fusion protein comprises an amino acid sequence with at least 80% sequence identity to any one of SEQ ID NOs: 66-74. In some embodiments, the antibody comprises a heavy chain variable region (VH) comprising an amino acid sequence with at least 81% sequence identity to any one of SEQ ID Nos: 8-20 and a light chain variable region (VL) comprising an amino acid sequence with at least 81% sequence identity to any one of SEQ ID Nos: 21-33, and the transmembrane polypeptide of the second fusion protein comprises an amino acid sequence with at least 81% sequence identity to any one of SEQ ID NOs: 66-74. In some embodiments, the antibody comprises a heavy chain variable region (VH) comprising an amino acid sequence with at least 82% sequence identity to any one of SEQ ID Nos: 8-20 and a light chain variable region (VL) comprising an amino acid sequence with at least 82% sequence identity to any one of SEQ ID Nos: 21-33, and the transmembrane polypeptide of the second fusion protein comprises an amino acid sequence with at least 82% sequence identity to any one of SEQ ID NOs: 66-74. In some embodiments, the antibody comprises a heavy chain variable region (VH) comprising an amino acid sequence with at least 83% sequence identity to any one of SEQ ID Nos: 8-20 and a light chain variable region (VL) comprising an amino acid sequence with at least 83% sequence identity to any one of SEQ ID Nos: 21-33, and the transmembrane polypeptide of the second fusion protein comprises an amino acid sequence with at least 83% sequence identity to any one of SEQ ID NOs: 66-74. In some embodiments, the antibody comprises a heavy chain variable region (VH) comprising an amino acid sequence with at least 84% sequence identity to any one of SEQ ID Nos: 8-20 and a light chain variable region (VL) comprising an amino acid sequence with at least 84% sequence identity to any one of SEQ ID Nos: 21-33, and the transmembrane polypeptide of the second fusion protein comprises an amino acid sequence with at least 84% sequence identity to any one of SEQ ID NOs: 66-74. In some embodiments, the antibody comprises a heavy chain variable region (VH) comprising an amino acid sequence with at least 85% sequence identity to any one of SEQ ID Nos: 8-20 and a light chain variable region (VL) comprising an amino acid sequence with at least 85% sequence identity to any one of SEQ ID Nos: 21-33, and the transmembrane polypeptide of the second fusion protein comprises an amino acid sequence with at least 85% sequence identity to any one of SEQ ID NOs: 66-74. In some embodiments, the antibody comprises a heavy chain variable region (VH) comprising an amino acid sequence with at least 86% sequence identity to any one of SEQ ID Nos: 8-20 and a light chain variable region (VL) comprising an amino acid sequence with at least 86% sequence identity to any one of SEQ ID Nos: 21-33, and the transmembrane polypeptide of the second fusion protein comprises an amino acid sequence with at least 86% sequence identity to any one of SEQ ID NOs: 66-74. In some embodiments, the antibody comprises a heavy chain variable region (VH) comprising an amino acid sequence with at least 87% sequence identity to any one of SEQ ID Nos: 8-20 and a light chain variable region (VL) comprising an amino acid sequence with at least 87% sequence identity to any one of SEQ ID Nos: 21-33, and the transmembrane polypeptide of the second fusion protein comprises an amino acid sequence with at least 87% sequence identity to any one of SEQ ID NOs: 66-74. In some embodiments, the antibody comprises a heavy chain variable region (VH) comprising an amino acid sequence with at least 88% sequence identity to any one of SEQ ID Nos: 8-20 and a light chain variable region (VL) comprising an amino acid sequence with at least 88% sequence identity to any one of SEQ ID Nos: 21-33, and the transmembrane polypeptide of the second fusion protein comprises an amino acid sequence with at least 88% sequence identity to any one of SEQ ID NOs: 66-74. In some embodiments, the antibody comprises a heavy chain variable region (VH) comprising an amino acid sequence with at least 89% sequence identity to any one of SEQ ID Nos: 8-20 and a light chain variable region (VL) comprising an amino acid sequence with at least 89% sequence identity to any one of SEQ ID Nos: 21-33, and the transmembrane polypeptide of the second fusion protein comprises an amino acid sequence with at least 89% sequence identity to any one of SEQ ID NOs: 66-74. In some embodiments, the antibody comprises a heavy chain variable region (VH) comprising an amino acid sequence with at least 90% sequence identity to any one of SEQ ID Nos: 8-20 and a light chain variable region (VL) comprising an amino acid sequence with at least 90% sequence identity to any one of SEQ ID Nos: 21-33, and the transmembrane polypeptide of the second fusion protein comprises an amino acid sequence with at least 90% sequence identity to any one of SEQ ID NOs: 66-74. In some embodiments, the antibody comprises a heavy chain variable region (VH) comprising an amino acid sequence with at least 91% sequence identity to any one of SEQ ID Nos: 8-20 and a light chain variable region (VL) comprising an amino acid sequence with at least 91% sequence identity to any one of SEQ ID Nos: 21-33, and the transmembrane polypeptide of the second fusion protein comprises an amino acid sequence with at least 91% sequence identity to any one of SEQ ID NOs: 66-74. In some embodiments, the antibody comprises a heavy chain variable region (VH) comprising an amino acid sequence with at least 92% sequence identity to any one of SEQ ID Nos: 8-20 and a light chain variable region (VL) comprising an amino acid sequence with at least 92% sequence identity to any one of SEQ ID Nos: 21-33, and the transmembrane polypeptide of the second fusion protein comprises an amino acid sequence with at least 92% sequence identity to any one of SEQ ID NOs: 66-74. In some embodiments, the antibody comprises a heavy chain variable region (VH) comprising an amino acid sequence with at least 93% sequence identity to any one of SEQ ID Nos: 8-20 and a light chain variable region (VL) comprising an amino acid sequence with at least 93% sequence identity to any one of SEQ ID Nos: 21-33, and the transmembrane polypeptide of the second fusion protein comprises an amino acid sequence with at least 93% sequence identity to any one of SEQ ID NOs: 66-74. In some embodiments, the antibody comprises a heavy chain variable region (VH) comprising an amino acid sequence with at least 94% sequence identity to any one of SEQ ID Nos: 8-20 and a light chain variable region (VL) comprising an amino acid sequence with at least 94% sequence identity to any one of SEQ ID Nos: 21-33, and the transmembrane polypeptide of the second fusion protein comprises an amino acid sequence with at least 94% sequence identity to any one of SEQ ID NOs: 66-74. In some embodiments, the antibody comprises a heavy chain variable region (VH) comprising an amino acid sequence with at least 95% sequence identity to any one of SEQ ID Nos: 8-20 and a light chain variable region (VL) comprising an amino acid sequence with at least 95% sequence identity to any one of SEQ ID Nos: 21-33, and the transmembrane polypeptide of the second fusion protein comprises an amino acid sequence with at least 95% sequence identity to any one of SEQ ID NOs: 66-74. In some embodiments, the antibody comprises a heavy chain variable region (VH) comprising an amino acid sequence with at least 96% sequence identity to any one of SEQ ID Nos: 8-20 and a light chain variable region (VL) comprising an amino acid sequence with at least 96% sequence identity to any one of SEQ ID Nos: 21-33, and the transmembrane polypeptide of the second fusion protein comprises an amino acid sequence with at least 96% sequence identity to any one of SEQ ID NOs: 66-74. In some embodiments, the antibody comprises a heavy chain variable region (VH) comprising an amino acid sequence with at least 97% sequence identity to any one of SEQ ID Nos: 8-20 and a light chain variable region (VL) comprising an amino acid sequence with at least 97% sequence identity to any one of SEQ ID Nos: 21-33, and the transmembrane polypeptide of the second fusion protein comprises an amino acid sequence with at least 97% sequence identity to any one of SEQ ID NOs: 66-74. In some embodiments, the antibody comprises a heavy chain variable region (VH) comprising an amino acid sequence with at least 98% sequence identity to any one of SEQ ID Nos: 8-20 and a light chain variable region (VL) comprising an amino acid sequence with at least 98% sequence identity to any one of SEQ ID Nos: 21-33, and the transmembrane polypeptide of the second fusion protein comprises an amino acid sequence with at least 98% sequence identity to any one of SEQ ID NOs: 66-74. In some embodiments, the antibody comprises a heavy chain variable region (VH) comprising an amino acid sequence with at least 99% sequence identity to any one of SEQ ID Nos: 8-20 and a light chain variable region (VL) comprising an amino acid sequence with at least 99% sequence identity to any one of SEQ ID Nos: 21-33, and the transmembrane polypeptide of the second fusion protein comprises an amino acid sequence with at least 99% sequence identity to any one of SEQ ID NOs: 66-74. In some embodiments, the antibody comprises a heavy chain variable region (VH) comprising an amino acid sequence according to any one of SEQ ID Nos: 8-20 and a light chain variable region (VL) comprising an amino acid sequence according to any one of SEQ ID Nos: 21-33, and the transmembrane polypeptide of the second fusion protein comprises an amino acid sequence according to any one of SEQ ID NOs: 66-74.

In some embodiments, the homing polypeptide comprises an amino acid sequence with at least 75% sequence identity to any one of SEQ ID NOs: 34-51, and the transmembrane polypeptide of the second fusion protein comprises an amino acid sequence with at least 75% sequence identity to any one of SEQ ID NOs: 66-74. In some embodiments, the homing polypeptide comprises an amino acid sequence with at least 76% sequence identity to any one of SEQ ID NOs: 34-51, and the transmembrane polypeptide of the second fusion protein comprises an amino acid sequence with at least 76% sequence identity to any one of SEQ ID NOs: 66-74. In some embodiments, the homing polypeptide comprises an amino acid sequence with at least 77% sequence identity to any one of SEQ ID NOs: 34-51, and the transmembrane polypeptide of the second fusion protein comprises an amino acid sequence with at least 77% sequence identity to any one of SEQ ID NOs: 66-74. In some embodiments, the homing polypeptide comprises an amino acid sequence with at least 78% sequence identity to any one of SEQ ID NOs: 34-51, and the transmembrane polypeptide of the second fusion protein comprises an amino acid sequence with at least 78% sequence identity to any one of SEQ ID NOs: 66-74. In some embodiments, the homing polypeptide comprises an amino acid sequence with at least 79% sequence identity to any one of SEQ ID NOs: 34-51, and the transmembrane polypeptide of the second fusion protein comprises an amino acid sequence with at least 79% sequence identity to any one of SEQ ID NOs: 66-74. In some embodiments, the homing polypeptide comprises an amino acid sequence with at least 80% sequence identity to any one of SEQ ID NOs: 34-51, and the transmembrane polypeptide of the second fusion protein comprises an amino acid sequence with at least 80% sequence identity to any one of SEQ ID NOs: 66-74. In some embodiments, the homing polypeptide comprises an amino acid sequence with at least 81% sequence identity to any one of SEQ ID NOs: 34-51, and the transmembrane polypeptide of the second fusion protein comprises an amino acid sequence with at least 81% sequence identity to any one of SEQ ID NOs: 66-74. In some embodiments, the homing polypeptide comprises an amino acid sequence with at least 82% sequence identity to any one of SEQ ID NOs: 34-51, and the transmembrane polypeptide of the second fusion protein comprises an amino acid sequence with at least 82% sequence identity to any one of SEQ ID NOs: 66-74. In some embodiments, the homing polypeptide comprises an amino acid sequence with at least 83% sequence identity to any one of SEQ ID NOs: 34-51, and the transmembrane polypeptide of the second fusion protein comprises an amino acid sequence with at least 83% sequence identity to any one of SEQ ID NOs: 66-74. In some embodiments, the homing polypeptide comprises an amino acid sequence with at least 84% sequence identity to any one of SEQ ID NOs: 34-51, and the transmembrane polypeptide of the second fusion protein comprises an amino acid sequence with at least 84% sequence identity to any one of SEQ ID NOs: 66-74. In some embodiments, the homing polypeptide comprises an amino acid sequence with at least 85% sequence identity to any one of SEQ ID NOs: 34-51, and the transmembrane polypeptide of the second fusion protein comprises an amino acid sequence with at least 85% sequence identity to any one of SEQ ID NOs: 66-74. In some embodiments, the homing polypeptide comprises an amino acid sequence with at least 86% sequence identity to any one of SEQ ID NOs: 34-51, and the transmembrane polypeptide of the second fusion protein comprises an amino acid sequence with at least 86% sequence identity to any one of SEQ ID NOs: 66-74. In some embodiments, the homing polypeptide comprises an amino acid sequence with at least 87% sequence identity to any one of SEQ ID NOs: 34-51, and the transmembrane polypeptide of the second fusion protein comprises an amino acid sequence with at least 87% sequence identity to any one of SEQ ID NOs: 66-74. In some embodiments, the homing polypeptide comprises an amino acid sequence with at least 88% sequence identity to any one of SEQ ID NOs: 34-51, and the transmembrane polypeptide of the second fusion protein comprises an amino acid sequence with at least 88% sequence identity to any one of SEQ ID NOs: 66-74. In some embodiments, the homing polypeptide comprises an amino acid sequence with at least 89% sequence identity to any one of SEQ ID NOs: 34-51, and the transmembrane polypeptide of the second fusion protein comprises an amino acid sequence with at least 89% sequence identity to any one of SEQ ID NOs: 66-74. In some embodiments, the homing polypeptide comprises an amino acid sequence with at least 90% sequence identity to any one of SEQ ID NOs: 34-51, and the transmembrane polypeptide of the second fusion protein comprises an amino acid sequence with at least 90% sequence identity to any one of SEQ ID NOs: 66-74. In some embodiments, the homing polypeptide comprises an amino acid sequence with at least 91% sequence identity to any one of SEQ ID NOs: 34-51, and the transmembrane polypeptide of the second fusion protein comprises an amino acid sequence with at least 91% sequence identity to any one of SEQ ID NOs: 66-74. In some embodiments, the homing polypeptide comprises an amino acid sequence with at least 92% sequence identity to any one of SEQ ID NOs: 34-51, and the transmembrane polypeptide of the second fusion protein comprises an amino acid sequence with at least 92% sequence identity to any one of SEQ ID NOs: 66-74. In some embodiments, the homing polypeptide comprises an amino acid sequence with at least 93% sequence identity to any one of SEQ ID NOs: 34-51, and the transmembrane polypeptide of the second fusion protein comprises an amino acid sequence with at least 93% sequence identity to any one of SEQ ID NOs: 66-74. In some embodiments, the homing polypeptide comprises an amino acid sequence with at least 94% sequence identity to any one of SEQ ID NOs: 34-51, and the transmembrane polypeptide of the second fusion protein comprises an amino acid sequence with at least 94% sequence identity to any one of SEQ ID NOs: 66-74. In some embodiments, the homing polypeptide comprises an amino acid sequence with at least 95% sequence identity to any one of SEQ ID NOs: 34-51, and the transmembrane polypeptide of the second fusion protein comprises an amino acid sequence with at least 95% sequence identity to any one of SEQ ID NOs: 66-74. In some embodiments, the homing polypeptide comprises an amino acid sequence with at least 96% sequence identity to any one of SEQ ID NOs: 34-51, and the transmembrane polypeptide of the second fusion protein comprises an amino acid sequence with at least 96% sequence identity to any one of SEQ ID NOs: 66-74. In some embodiments, the homing polypeptide comprises an amino acid sequence with at least 97% sequence identity to any one of SEQ ID NOs: 34-51, and the transmembrane polypeptide of the second fusion protein comprises an amino acid sequence with at least 97% sequence identity to any one of SEQ ID NOs: 66-74. In some embodiments, the homing polypeptide comprises an amino acid sequence with at least 98% sequence identity to any one of SEQ ID NOs: 34-51, and the transmembrane polypeptide of the second fusion protein comprises an amino acid sequence with at least 98% sequence identity to any one of SEQ ID NOs: 66-74. In some embodiments, the homing polypeptide comprises an amino acid sequence with at least 99% sequence identity to any one of SEQ ID NOs: 34-51, and the transmembrane polypeptide of the second fusion protein comprises an amino acid sequence with at least 99% sequence identity to any one of SEQ ID NOs: 66-74. In some embodiments, the homing polypeptide comprises an amino acid sequence according to any one of SEQ ID NOs: 34-51, and the transmembrane polypeptide of the second fusion protein comprises an amino acid sequence according to any one of SEQ ID NOs: 66-74.

In some embodiments, the IFN polypeptide of the first fusion protein comprises IFN-alpha, IFN-beta, IFN-epsilon, IFN-kappa, IFN-omega, IFN-gamma, or IFN-lambda; the transmembrane polypeptide of the first fusion protein comprises a transmembrane domain of a Vesicular Stomatitis virus glycoprotein (VSV-G), a transmembrane domain and a cytosolic domain of VSV-G, a transmembrane domain of Influenza Neuraminidase (NA), a transmembrane domain and a cytosolic domain of Influenza Neuraminidase (NA), a transmembrane domain of influenza Hemagglutinin (HA), a transmembrane domain and a cytosolic domain of influenza Hemagglutinin (HA), a transmembrane domain of HIV surface glycoprotein GP120 or GP41, a transmembrane domain and a cytosolic domain of HIV surface glycoprotein GP120 or GP4, a transmembrane domain of Dengue E Protein, a transmembrane domain and a cytosolic domain of Dengue E Protein, a transmembrane domain of measles virus surface glycoprotein hemagglutinin (H) protein, or a transmembrane domain and a cytosolic domain of measles virus surface glycoprotein hemagglutinin (H) protein; and the oligomerization domain of the first fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, the IFN polypeptide of the second fusion protein comprises IFN-alpha, IFN-beta, IFN-epsilon, IFN-kappa, IFN-omega, IFN-gamma, or IFN-lambda: the transmembrane polypeptide of the second fusion protein comprises a transmembrane domain of a Vesicular Stomatitis virus glycoprotein (VSV-G), a transmembrane domain and a cytosolic domain of VSV-G, a transmembrane domain of Influenza Neuraminidase (NA), a transmembrane domain and a cytosolic domain of Influenza Neuraminidase (NA), a transmembrane domain of influenza Hemagglutinin (HA), a transmembrane domain and a cytosolic domain of influenza Hemagglutinin (HA), a transmembrane domain of HIV surface glycoprotein GP120 or GP41, a transmembrane domain and a cytosolic domain of HIV surface glycoprotein GP120 or GP4, a transmembrane domain of Dengue E Protein, a transmembrane domain and a cytosolic domain of Dengue E Protein, a transmembrane domain of measles virus surface glycoprotein hemagglutinin (H) protein, or a transmembrane domain and a cytosolic domain of measles virus surface glycoprotein hemagglutinin (H) protein; and the oligomerization domain of the second fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, the homing polypeptide comprises an antibody that binds specifically to an antigen on the target cell or the target protein. Exemplary IFN-MVP fusion protein sequences are listed in Table 7.

TABLE 7
Exemplary IFN-MVP Fusion Protein Sequences

| | | | | | SEQ |
|---|---|---|---|---|---|
| IFN-MVP | IFN¹ | TM² | OD³ | Amino Acid Sequence | ID No: |
| IFNα/VM | Human | VM | None | MALTFALLVALLVLSCKSSCSVGCDLP | 153 |
| | IFNα | | | QTHSLGSRRTLMLLAQMRKISLFSCLKD | |
| | | | | RHDFGFPQEEFGNQFQKAETIPVLHEMI | |
| | | | | QQIFNLFSTKDSSAAWDETLLDKFYTEL | |
| | | | | YQQLNDLEACVIQGVGVTETPLMKEDS | |
| | | | | ILAVRKYFQRITLYLKEKKYSPCAWEV | |
| | | | | VRAEIMRSFSLSTNLQESLRSKERGMLD | |
| | | | | SDLHLSSKAQVFEHPHIQDAASQLPDDE | |
| | | | | SLFFGDTGLSKNPIELVEGWFSSWKSSI | |
| | | | | ASFFFIIGLIIGLFLVLRVGIHLCIKLKHT | |
| | | | | KKRQIEQKLISEEDL | |
| IFNα/D4 | Human | VM | D4 | MALTFALLVALLVLSCKSSCSVGCDLP | 154 |
| | IFNα | | | QTHSLGSRRTLMLLAQMRKISLFSCLKD | |
| | | | | RHDFGFPQEEFGNQFQKAETIPVLHEMI | |
| | | | | QQIFNLFSTKDSSAAWDETLLDKFYTEL | |
| | | | | YQQLNDLEACVIQGVGVTETPLMKEDS | |
| | | | | ILAVRKYFQRITLYLKEKKYSPCAWEV | |
| | | | | VRAEIMRSFSLSTNLQESLRSKEIIQADG | |
| | | | | WMCHASKWVTTCDFRWYGPKYITHSI | |
| | | | | RSFTPSVEQCKESIEQTKQGTWLNPGFP | |
| | | | | PQSCGYATVIDAEAVIVQVTPHHVLVD | |
| | | | | EYTGEWVDSQFINGKCSNYICPTVHNST | |
| | | | | TWHSDYKVKGLCDSNLGMLDSDLHLS | |
| | | | | SKAQVFEHPHIQDAASQLPDDESLFFGD | |
| | | | | TGLSKNPIELVEGWFSSWKSSIASFFFIIG | |
| | | | | LIIGLFLVLRVGIHLCIKLKHTKKRQIYT | |
| | | | | DIEMNRLGK | |
| IFNβ/VM | Human | VM | None | MTNKCLLQIALLLCFSTTALSMSYNLLG | 155 |
| | IFNβ | | | FLQRSSNFQCQKLLWQLNGRLEYCLKD | |
| | | | | RMNFDIPEEIKQLQQFQKEDAALTIYEM | |
| | | | | LQNIFAIFRQDSSSTGWNETIVENLLAN | |
| | | | | VYHQINHLKTVLEEKLEKEDFTRGKLM | |
| | | | | SSLHLKRYYGRILHYLKAKEYSHCAWT | |
| | | | | IVRVEILRNFYFINRLTGYLRNRGMLDS | |
| | | | | DLHLSSKAQVFEHPHIQDAASQLPDDES | |
| | | | | LFFGDTGLSKNPIELVEGWFSSWKSSIA | |
| | | | | SFFFIIGLIIGLFLVLRVGIHLCIKLKHTK | |
| | | | | KRQIEQKLISEEDL | |
| IFNβ/D4 | Human | VM | D4 | MTNKCLLQIALLLCFSTTALSMSYNLLG | 156 |
| | IFNβ | | | FLQRSSNFQCQKLLWQLNGRLEYCLKD | |
| | | | | RMNFDIPEEIKQLQQFQKEDAALTIYEM | |
| | | | | LQNIFAIFRQDSSSTGWNETIVENLLAN | |
| | | | | VYHQINHLKTVLEEKLEKEDFTRGKLM | |
| | | | | SSLHLKRYYGRILHYLKAKEYSHCAWT | |
| | | | | IVRVEILRNFYFINRLTGYLRNIIQADGW | |
| | | | | MCHASKWVTTCDFRWYGPKYITHSIRS | |
| | | | | FTPSVEQCKESIEQTKQGTWLNPGFPPQ | |
| | | | | SCGYATVIDAEAVIVQVTPHHVLVDEY | |
| | | | | TGEWVDSQFINGKCSNYICPTVHNSTT | |
| | | | | WHSDYKVKGLCDSNLGMLDSDLHLSS | |
| | | | | KAQVFEHPHIQDAASQLPDDESLFFGDT | |
| | | | | GLSKNPIELVEGWFSSWKSSIASFFFIIGL | |
| | | | | IIGLFLVLRVGIHLCIKLKHTKKRQIYTDI | |
| | | | | EMNRLGK | |
| IFNγ/VM | Human | VM | None | MKYTSYILAFQLCIVLGSLGCYCQDPYV | 157 |
| | IFNγ | | | KEAENLKKYFNAGHSDVADNGTLFLGI | |
| | | | | LKNWKEESDRKIMQSQIVSFYFKLFKNF | |
| | | | | KDDQSIQKSVETIKEDMNVKFFNSNKK | |
| | | | | KRDDFEKLTNYSVTDLNVQRKAIHELIQ | |
| | | | | VMAELSPAAKTGKRKRSQMLFRGRRA | |
| | | | | SQRGMLDSDLHLSSKAQVFEHPHIQDA | |
| | | | | ASQLPDDESLFFGDTGLSKNPIELVEGW | |
| | | | | FSSWKSSIASFFFIIGLIIGLFLVLRVGIHL | |
| | | | | CIKLKHTKKRQIEQKLISEEDL | |
| IFNγ/D4 | Human | VM | D4 | MKYTSYILAFQLCIVLGSLGCYCQDPYV | 158 |
| | IFNγ | | | KEAENLKKYFNAGHSDVADNGTLFLGI | |
| | | | | LKNWKEESDRKIMQSQIVSFYFKLFKNF | |
| | | | | KDDQSIQKSVETIKEDMNVKFFNSNKK | |
| | | | | KRDDFEKLTNYSVTDLNVQRKAIHELIQ | |
| | | | | VMAELSPAAKTGKRKRSQMLFRGRRA | |
| | | | | SQIIQADGWMCHASKWVTTCDFRWYG | |
| | | | | PKYITHSIRSFTPSVEQCKESIEQTKQGT | |
| | | | | WLNPGFPPQSCGYATVTDAEAVIVQVT | |
| | | | | PHHVLVDEYTGEWVDSQFINGKCSNYI | |
| | | | | CPTVHNSTTWHSDYKVKGLCDSNLGM | |
| | | | | LDSDLHLSSKAQVFEHPHIQDAASQLPD | |
| | | | | DESLFFGDTGLSKNPIELVEGWFSSWKS | |
| | | | | SIASFFFIIGLIIGLFLVLRVGIHLCIKLKH | |
| | | | | TKKRQIYTDIEMNRLGK | |

IFN¹: interferon
TM²: transmembrane domain
OD³: oligomerization domain

In some embodiments, the first fusion protein comprises an amino acid sequence with at least 75% sequence identity to any one of SEQ ID NOs: 153-158. In some embodiments, the first fusion protein comprises an amino acid sequence with at least 76% sequence identity to any one of SEQ ID NOs: 153-158. In some embodiments, the first fusion protein comprises an amino acid sequence with at least 77% sequence identity to any one of SEQ ID NOs: 153-158. In some embodiments, the first fusion protein comprises an amino acid sequence with at least 78% sequence identity to any one of SEQ ID NOs: 153-158. In some embodiments, the first fusion protein comprises an amino acid sequence with at least 79% sequence identity to any one of SEQ ID NOs: 153-158. In some embodiments, the first fusion protein comprises an amino acid sequence with at least 80% sequence identity to any one of SEQ ID NOs: 153-158. In some embodiments, the first fusion protein comprises an amino acid sequence with at least 81% sequence identity to any one of SEQ ID NOs: 153-158. In some embodiments, the first fusion protein comprises an amino acid sequence with at least 82% sequence identity to any one of SEQ ID NOs: 153-158. In some embodiments, the first fusion protein comprises an amino acid sequence with at least 83% sequence identity to any one of SEQ ID NOs: 153-158. In some embodiments, the first fusion protein comprises an amino acid sequence with at least 84% sequence identity to any one of SEQ ID NOs: 153-158. In some embodiments, the first fusion protein comprises an amino acid sequence with at least 85% sequence identity to any one of SEQ ID NOs: 153-158. In some embodiments, the first fusion protein comprises an amino acid sequence with at least 86% sequence identity to any one of SEQ ID NOs: 153-158. In some embodiments, the first fusion protein comprises an amino acid sequence with at least 87% sequence identity to any one of SEQ ID NOs: 153-158. In some embodiments, the first fusion protein comprises an amino acid sequence with at least 88% sequence identity to any one of SEQ ID NOs: 153-158. In some embodiments, the first fusion protein comprises an amino acid sequence with at least 89% sequence identity to any one of SEQ ID NOs: 153-158. In some embodiments, the first fusion protein comprises an amino acid sequence with at least 90% sequence identity to any one of SEQ ID NOs: 153-158. In some embodiments, the first fusion protein comprises an amino acid sequence with at least 91% sequence identity to any one of SEQ ID NOs: 153-158. In some embodiments, the first fusion protein comprises an amino acid sequence with at least 92% sequence identity to any one of SEQ ID NOs: 153-158. In some embodiments, the first fusion protein comprises an amino acid sequence with at least 93% sequence identity to any one of SEQ ID NOs: 153-158. In some embodiments, the first fusion protein comprises an amino acid sequence with at least 94% sequence identity to any one of SEQ ID NOs: 153-158. In some embodiments, the first fusion protein comprises an amino acid sequence with at least 95% sequence identity to any one of SEQ ID NOs: 153-158. In some embodiments, the first fusion protein comprises an amino acid sequence with at least 96% sequence identity to any one of SEQ ID NOs: 153-158. In some embodiments, the first fusion protein comprises an amino acid sequence with at least 97% sequence identity to any one of SEQ ID NOs: 153-158. In some embodiments, the first fusion protein comprises an amino acid sequence with at least 98% sequence identity to any one of SEQ ID NOs: 153-158. In some embodiments, the first fusion protein comprises an amino acid sequence with at least 99% sequence identity to any one of SEQ ID NOs: 153-158. In some embodiments, the first fusion protein comprises an amino acid sequence according to any one of SEQ ID NOs: 153-158.

In some embodiments, the second fusion protein comprises an amino acid sequence with at least 75% sequence identity to any one of SEQ ID NOs: 153-158. In some embodiments, the second fusion protein comprises an amino acid sequence with at least 76% sequence identity to any one of SEQ ID NOs: 153-158. In some embodiments, the second fusion protein comprises an amino acid sequence with at least 77% sequence identity to any one of SEQ ID NOs: 153-158. In some embodiments, the second fusion protein comprises an amino acid sequence with at least 78% sequence identity to any one of SEQ ID NOs: 153-158. In some embodiments, the second fusion protein comprises an amino acid sequence with at least 79% sequence identity to any one of SEQ ID NOs: 153-158. In some embodiments, the second fusion protein comprises an amino acid sequence with at least 80% sequence identity to any one of SEQ ID NOs: 153-158. In some embodiments, the second fusion protein comprises an amino acid sequence with at least 81% sequence identity to any one of SEQ ID NOs: 153-158. In some embodiments, the second fusion protein comprises an amino acid sequence with at least 82% sequence identity to any one of SEQ ID NOs: 153-158. In some embodiments, the second fusion protein comprises an amino acid sequence with at least 83% sequence identity to any one of SEQ ID NOs: 153-158. In some embodiments, the second fusion protein comprises an amino acid sequence with at least 84% sequence identity to any one of SEQ ID NOs: 153-158. In some embodiments, the second fusion protein comprises an amino acid sequence with at least 85% sequence identity to any one of SEQ ID NOs: 153-158. In some embodiments, the second fusion protein comprises an amino acid sequence with at least 86% sequence identity to any one of SEQ ID NOs: 153-158. In some embodiments, the second fusion protein comprises an amino acid sequence with at least 87% sequence identity to any one of SEQ ID NOs: 153-158. In some embodiments, the second fusion protein comprises an amino acid sequence with at least 88% sequence identity to any one of SEQ ID NOs: 153-158. In some embodiments, the second fusion protein comprises an amino acid sequence with at least 89% sequence identity to any one of SEQ ID NOs: 153-158. In some embodiments, the second fusion protein comprises an amino acid sequence with at least 90% sequence identity to any one of SEQ ID NOs: 153-158. In some embodiments, the second fusion protein comprises an amino acid sequence with at least 91% sequence identity to any one of SEQ ID NOs: 153-158. In some embodiments, the second fusion protein comprises an amino acid sequence with at least 92% sequence identity to any one of SEQ ID NOs: 153-158. In some embodiments, the second fusion protein comprises an amino acid sequence with at least 93% sequence identity to any one of SEQ ID NOs: 153-158. In some embodiments, the second fusion protein comprises an amino acid sequence with at least 94% sequence identity to any one of SEQ ID NOs: 153-158. In some embodiments, the second fusion protein comprises an amino acid sequence with at least 95% sequence identity to any one of SEQ ID NOs: 153-158. In some embodiments, the second fusion protein comprises an amino acid sequence with at least 96% sequence identity to any one of SEQ ID NOs: 153-158. In some embodiments, the second fusion protein comprises an amino acid sequence with at least 97% sequence identity to any one of SEQ ID NOs: 153-158. In some embodiments, the second fusion protein comprises an amino acid sequence with at least 98% sequence identity to any one of SEQ ID NOs: 153-158. In some embodiments, the second fusion protein comprises an amino acid sequence with at least 99% sequence identity to any one of SEQ ID NOs: 153-158. In some embodiments, the second fusion protein comprises an amino acid sequence according to any one of SEQ ID NOs: 153-158.

In some embodiments, the IFN polypeptide of the first fusion protein comprises an amino acid sequence with at least 75% sequence identity to any one of SEQ ID NOs: 1-7; the transmembrane polypeptide of the first fusion protein comprises an amino acid sequence with at least 75% sequence identity to any one of SEQ ID NOs: 66-74; and the oligomerization domain of the first fusion protein comprises an amino acid sequence with at least 75% sequence identity to any one of SEQ ID NOs: 52-65. In some embodiments, the IFN polypeptide of the first fusion protein comprises an amino acid sequence with at least 76% sequence identity to any one of SEQ ID NOs: 1-7; the transmembrane polypeptide of the first fusion protein comprises an amino acid sequence with at least 76% sequence identity to any one of SEQ ID NOs: 66-74; and the oligomerization domain of the first fusion protein comprises an amino acid sequence with at least 76% sequence identity to any one of SEQ ID NOs: 52-65. In some embodiments, the IFN polypeptide of the first fusion protein comprises an amino acid sequence with at least 77% sequence identity to any one of SEQ ID NOs: 1-7; the transmembrane polypeptide of the first fusion protein comprises an amino acid sequence with at least 77% sequence identity to any one of SEQ ID NOs: 66-74; and the oligomerization domain of the first fusion protein comprises an amino acid sequence with at least 77% sequence identity to any one of SEQ ID NOs: 52-65. In some embodiments, the IFN polypeptide of the first fusion protein comprises an amino acid sequence with at least 78% sequence identity to any one of SEQ ID NOs: 1-7; the transmembrane polypeptide of the first fusion protein comprises an amino acid sequence with at least 78% sequence identity to any one of SEQ ID NOs: 66-74; and the oligomerization domain of the first fusion protein comprises an amino acid sequence with at least 78% sequence identity to any one of SEQ ID NOs: 52-65. In some embodiments, the IFN polypeptide of the first fusion protein comprises an amino acid sequence with at least 79% sequence identity to any one of SEQ ID NOs: 1-7; the transmembrane polypeptide of the first fusion protein comprises an amino acid sequence with at least 79% sequence identity to any one of SEQ ID NOs: 66-74; and the oligomerization domain of the first fusion protein comprises an amino acid sequence with at least 79% sequence identity to any one of SEQ ID NOs: 52-65. In some embodiments, the IFN polypeptide of the first fusion protein comprises an amino acid sequence with at least 80% sequence identity to any one of SEQ ID NOs: 1-7; the transmembrane polypeptide of the first fusion protein comprises an amino acid sequence with at least 80% sequence identity to any one of SEQ ID NOs: 66-74; and the oligomerization domain of the first fusion protein comprises an amino acid sequence with at least 80% sequence identity to any one of SEQ ID NOs: 52-65. In some embodiments, the IFN polypeptide of the first fusion protein comprises an amino acid sequence with at least 81% sequence identity to any one of SEQ ID NOs: 1-7; the transmembrane polypeptide of the first fusion protein comprises an amino acid sequence with at least 81% sequence identity to any one of SEQ ID NOs: 66-74; and the oligomerization domain of the first fusion protein comprises an amino acid sequence with at least 81% sequence identity to any one of SEQ ID NOs: 52-65. In some embodiments, the IFN polypeptide of the first fusion protein comprises an amino acid sequence with at least 82% sequence identity to any one of SEQ ID NOs: 1-7; the transmembrane polypeptide of the first fusion protein comprises an amino acid sequence with at least 82% sequence identity to any one of SEQ ID NOs: 66-74; and the oligomerization domain of the first fusion protein comprises an amino acid sequence with at least 82% sequence identity to any one of SEQ ID NOs: 52-65. In some embodiments, the IFN polypeptide of the first fusion protein comprises an amino acid sequence with at least 83% sequence identity to any one of SEQ ID NOs: 1-7; the transmembrane polypeptide of the first fusion protein comprises an amino acid sequence with at least 83% sequence identity to any one of SEQ ID NOs: 66-74; and the oligomerization domain of the first fusion protein comprises an amino acid sequence with at least 83% sequence identity to any one of SEQ ID NOs: 52-65. In some embodiments, the IFN polypeptide of the first fusion protein comprises an amino acid sequence with at least 84% sequence identity to any one of SEQ ID NOs: 1-7; the transmembrane polypeptide of the first fusion protein comprises an amino acid sequence with at least 84% sequence identity to any one of SEQ ID NOs: 66-74; and the oligomerization domain of the first fusion protein comprises an amino acid sequence with at least 84% sequence identity to any one of SEQ ID NOs: 52-65. In some embodiments, the IFN polypeptide of the first fusion protein comprises an amino acid sequence with at least 85% sequence identity to any one of SEQ ID NOs: 1-7; the transmembrane polypeptide of the first fusion protein comprises an amino acid sequence with at least 85% sequence identity to any one of SEQ ID NOs: 66-74; and the oligomerization domain of the first fusion protein comprises an amino acid sequence with at least 85% sequence identity to any one of SEQ ID NOs: 52-65. In some embodiments, the IFN polypeptide of the first fusion protein comprises an amino acid sequence with at least 86% sequence identity to any one of SEQ ID NOs: 1-7; the transmembrane polypeptide of the first fusion protein comprises an amino acid sequence with at least 86% sequence identity to any one of SEQ ID NOs: 66-74; and the oligomerization domain of the first fusion protein comprises an amino acid sequence with at least 86% sequence identity to any one of SEQ ID NOs: 52-65. In some embodiments, the IFN polypeptide of the first fusion protein comprises an amino acid sequence with at least 87% sequence identity to any one of SEQ ID NOs: 1-7; the transmembrane polypeptide of the first fusion protein comprises an amino acid sequence with at least 87% sequence identity to any one of SEQ ID NOs: 66-74; and the oligomerization domain of the first fusion protein comprises an amino acid sequence with at least 87% sequence identity to any one of SEQ ID NOs: 52-65. In some embodiments, the IFN polypeptide of the first fusion protein comprises an amino acid sequence with at least 88% sequence identity to any one of SEQ ID NOs: 1-7; the transmembrane polypeptide of the first fusion protein comprises an amino acid sequence with at least 88% sequence identity to any one of SEQ ID NOs: 66-74; and the oligomerization domain of the first fusion protein comprises an amino acid sequence with at least 88% sequence identity to any one of SEQ ID NOs: 52-65. In some embodiments, the IFN polypeptide of the first fusion protein comprises an amino acid sequence with at least 89% sequence identity to any one of SEQ ID NOs: 1-7; the transmembrane polypeptide of the first fusion protein comprises an amino acid sequence with at least 89% sequence identity to any one of SEQ ID NOs: 66-74; and the oligomerization domain of the first fusion protein comprises an amino acid sequence with at least 89% sequence identity to any one of SEQ ID NOs: 52-65. In some embodiments, the IFN polypeptide of the first fusion protein comprises an amino acid sequence with at least 90% sequence identity to any one of SEQ ID NOs: 1-7; the transmembrane polypeptide of the first fusion protein comprises an amino acid sequence with at least 90% sequence identity to any one of SEQ ID NOs: 66-74; and the oligomerization domain of the first fusion protein comprises an amino acid sequence with at least 90% sequence identity to any one of SEQ ID NOs: 52-65. In some embodiments, the IFN polypeptide of the first fusion protein comprises an amino acid sequence with at least 91% sequence identity to any one of SEQ ID NOs: 1-7; the transmembrane polypeptide of the first fusion protein comprises an amino acid sequence with at least 91% sequence identity to any one of SEQ ID NOs: 66-74; and the oligomerization domain of the first fusion protein comprises an amino acid sequence with at least 91% sequence identity to any one of SEQ ID NOs: 52-65. In some embodiments, the IFN polypeptide of the first fusion protein comprises an amino acid sequence with at least 92% sequence identity to any one of SEQ ID NOs: 1-7; the transmembrane polypeptide of the first fusion protein comprises an amino acid sequence with at least 92% sequence identity to any one of SEQ ID NOs: 66-74; and the oligomerization domain of the first fusion protein comprises an amino acid sequence with at least 92% sequence identity to any one of SEQ ID NOs: 52-65. In some embodiments, the IFN polypeptide of the first fusion protein comprises an amino acid sequence with at least 93% sequence identity to any one of SEQ ID NOs: 1-7; the transmembrane polypeptide of the first fusion protein comprises an amino acid sequence with at least 93% sequence identity to any one of SEQ ID NOs: 66-74; and the oligomerization domain of the first fusion protein comprises an amino acid sequence with at least 93% sequence identity to any one of SEQ ID NOs: 52-65. In some embodiments, the IFN polypeptide of the first fusion protein comprises an amino acid sequence with at least 94% sequence identity to any one of SEQ ID NOs: 1-7; the transmembrane polypeptide of the first fusion protein comprises an amino acid sequence with at least 94% sequence identity to any one of SEQ ID NOs: 66-74; and the oligomerization domain of the first fusion protein comprises an amino acid sequence with at least 94% sequence identity to any one of SEQ ID NOs: 52-65. In some embodiments, the IFN polypeptide of the first fusion protein comprises an amino acid sequence with at least 95% sequence identity to any one of SEQ ID NOs: 1-7; the transmembrane polypeptide of the first fusion protein comprises an amino acid sequence with at least 95% sequence identity to any one of SEQ ID NOs: 66-74; and the oligomerization domain of the first fusion protein comprises an amino acid sequence with at least 95% sequence identity to any one of SEQ ID NOs: 52-65. In some embodiments, the IFN polypeptide of the first fusion protein comprises an amino acid sequence with at least 96% sequence identity to any one of SEQ ID NOs: 1-7; the transmembrane polypeptide of the first fusion protein comprises an amino acid sequence with at least 96% sequence identity to any one of SEQ ID NOs: 66-74; and the oligomerization domain of the first fusion protein comprises an amino acid sequence with at least 96% sequence identity to any one of SEQ ID NOs: 52-65. In some embodiments, the IFN polypeptide of the first fusion protein comprises an amino acid sequence with at least 97% sequence identity to any one of SEQ ID NOs: 1-7; the transmembrane polypeptide of the first fusion protein comprises an amino acid sequence with at least 97% sequence identity to any one of SEQ ID NOs: 66-74; and the oligomerization domain of the first fusion protein comprises an amino acid sequence with at least 97% sequence identity to any one of SEQ ID NOs: 52-65. In some embodiments, the IFN polypeptide of the first fusion protein comprises an amino acid sequence with at least 98% sequence identity to any one of SEQ ID NOs: 1-7; the transmembrane polypeptide of the first fusion protein comprises an amino acid sequence with at least 98% sequence identity to any one of SEQ ID NOs: 66-74; and the oligomerization domain of the first fusion protein comprises an amino acid sequence with at least 98% sequence identity to any one of SEQ ID NOs: 52-65. In some embodiments, the IFN polypeptide of the first fusion protein comprises an amino acid sequence with at least 99% sequence identity to any one of SEQ ID NOs: 1-7; the transmembrane polypeptide of the first fusion protein comprises an amino acid sequence with at least 99% sequence identity to any one of SEQ ID NOs: 66-74; and the oligomerization domain of the first fusion protein comprises an amino acid sequence with at least 99% sequence identity to any one of SEQ ID NOs: 52-65. In some embodiments, the IFN polypeptide of the first fusion protein comprises an amino acid sequence according to any one of SEQ ID NOs: 1-7; the transmembrane polypeptide of the first fusion protein comprises an amino acid sequence according to any one of SEQ ID NOs: 66-74; and the oligomerization domain of the first fusion protein comprises an amino acid sequence according to any one of SEQ ID NOs: 52-65.

In some embodiments, the IFN polypeptide of the second fusion protein comprises an amino acid sequence with at least 75% sequence identity to any one of SEQ ID NOs: 1-7; the transmembrane polypeptide of the second fusion protein comprises an amino acid sequence with at least 75% sequence identity to any one of SEQ ID NOs: 66-74; and the oligomerization domain of the second fusion protein comprises an amino acid sequence with at least 75% sequence identity to any one of SEQ ID NOs: 52-65. In some embodiments, the IFN polypeptide of the second fusion protein comprises an amino acid sequence with at least 76% sequence identity to any one of SEQ ID NOs: 1-7; the transmembrane polypeptide of the second fusion protein comprises an amino acid sequence with at least 76% sequence identity to any one of SEQ ID NOs: 66-74; and the oligomerization domain of the second fusion protein comprises an amino acid sequence with at least 76% sequence identity to any one of SEQ ID NOs: 52-65. In some embodiments, the IFN polypeptide of the second fusion protein comprises an amino acid sequence with at least 77% sequence identity to any one of SEQ ID NOs: 1-7; the transmembrane polypeptide of the second fusion protein comprises an amino acid sequence with at least 77% sequence identity to any one of SEQ ID NOs: 66-74; and the oligomerization domain of the second fusion protein comprises an amino acid sequence with at least 77% sequence identity to any one of SEQ ID NOs: 52-65. In some embodiments, the IFN polypeptide of the second fusion protein comprises an amino acid sequence with at least 78% sequence identity to any one of SEQ ID NOs: 1-7; the transmembrane polypeptide of the second fusion protein comprises an amino acid sequence with at least 78% sequence identity to any one of SEQ ID NOs: 66-74; and the oligomerization domain of the second fusion protein comprises an amino acid sequence with at least 78% sequence identity to any one of SEQ ID NOs: 52-65. In some embodiments, the IFN polypeptide of the second fusion protein comprises an amino acid sequence with at least 79% sequence identity to any one of SEQ ID NOs: 1-7; the transmembrane polypeptide of the second fusion protein comprises an amino acid sequence with at least 79% sequence identity to any one of SEQ ID NOs: 66-74; and the oligomerization domain of the second fusion protein comprises an amino acid sequence with at least 79% sequence identity to any one of SEQ ID NOs: 52-65. In some embodiments, the IFN polypeptide of the second fusion protein comprises an amino acid sequence with at least 80% sequence identity to any one of SEQ ID NOs: 1-7; the transmembrane polypeptide of the second fusion protein comprises an amino acid sequence with at least 80% sequence identity to any one of SEQ ID NOs: 66-74; and the oligomerization domain of the second fusion protein comprises an amino acid sequence with at least 80% sequence identity to any one of SEQ ID NOs: 52-65. In some embodiments, the IFN polypeptide of the second fusion protein comprises an amino acid sequence with at least 81% sequence identity to any one of SEQ ID NOs: 1-7; the transmembrane polypeptide of the second fusion protein comprises an amino acid sequence with at least 81% sequence identity to any one of SEQ ID NOs: 66-74; and the oligomerization domain of the second fusion protein comprises an amino acid sequence with at least 81% sequence identity to any one of SEQ ID NOs: 52-65. In some embodiments, the IFN polypeptide of the second fusion protein comprises an amino acid sequence with at least 82% sequence identity to any one of SEQ ID NOs: 1-7; the transmembrane polypeptide of the second fusion protein comprises an amino acid sequence with at least 82% sequence identity to any one of SEQ ID NOs: 66-74; and the oligomerization domain of the second fusion protein comprises an amino acid sequence with at least 82% sequence identity to any one of SEQ ID NOs: 52-65. In some embodiments, the IFN polypeptide of the second fusion protein comprises an amino acid sequence with at least 83% sequence identity to any one of SEQ ID NOs: 1-7; the transmembrane polypeptide of the second fusion protein comprises an amino acid sequence with at least 83% sequence identity to any one of SEQ ID NOs: 66-74; and the oligomerization domain of the second fusion protein comprises an amino acid sequence with at least 83% sequence identity to any one of SEQ ID NOs: 52-65. In some embodiments, the IFN polypeptide of the second fusion protein comprises an amino acid sequence with at least 84% sequence identity to any one of SEQ ID NOs: 1-7; the transmembrane polypeptide of the second fusion protein comprises an amino acid sequence with at least 84% sequence identity to any one of SEQ ID NOs: 66-74; and the oligomerization domain of the second fusion protein comprises an amino acid sequence with at least 84% sequence identity to any one of SEQ ID NOs: 52-65. In some embodiments, the IFN polypeptide of the second fusion protein comprises an amino acid sequence with at least 85% sequence identity to any one of SEQ ID NOs: 1-7; the transmembrane polypeptide of the second fusion protein comprises an amino acid sequence with at least 85% sequence identity to any one of SEQ ID NOs: 66-74; and the oligomerization domain of the second fusion protein comprises an amino acid sequence with at least 85% sequence identity to any one of SEQ ID NOs: 52-65. In some embodiments, the IFN polypeptide of the second fusion protein comprises an amino acid sequence with at least 86% sequence identity to any one of SEQ ID NOs: 1-7; the transmembrane polypeptide of the second fusion protein comprises an amino acid sequence with at least 86% sequence identity to any one of SEQ ID NOs: 66-74; and the oligomerization domain of the second fusion protein comprises an amino acid sequence with at least 86% sequence identity to any one of SEQ ID NOs: 52-65. In some embodiments, the IFN polypeptide of the second fusion protein comprises an amino acid sequence with at least 87% sequence identity to any one of SEQ ID NOs: 1-7; the transmembrane polypeptide of the second fusion protein comprises an amino acid sequence with at least 87% sequence identity to any one of SEQ ID NOs: 66-74; and the oligomerization domain of the second fusion protein comprises an amino acid sequence with at least 87% sequence identity to any one of SEQ ID NOs: 52-65. In some embodiments, the IFN polypeptide of the second fusion protein comprises an amino acid sequence with at least 88% sequence identity to any one of SEQ ID NOs: 1-7; the transmembrane polypeptide of the second fusion protein comprises an amino acid sequence with at least 88% sequence identity to any one of SEQ ID NOs: 66-74; and the oligomerization domain of the second fusion protein comprises an amino acid sequence with at least 88% sequence identity to any one of SEQ ID NOs: 52-65. In some embodiments, the IFN polypeptide of the second fusion protein comprises an amino acid sequence with at least 89% sequence identity to any one of SEQ ID NOs: 1-7; the transmembrane polypeptide of the second fusion protein comprises an amino acid sequence with at least 89% sequence identity to any one of SEQ ID NOs: 66-74; and the oligomerization domain of the second fusion protein comprises an amino acid sequence with at least 89% sequence identity to any one of SEQ ID NOs: 52-65. In some embodiments, the IFN polypeptide of the second fusion protein comprises an amino acid sequence with at least 90% sequence identity to any one of SEQ ID NOs: 1-7; the transmembrane polypeptide of the second fusion protein comprises an amino acid sequence with at least 90% sequence identity to any one of SEQ ID NOs: 66-74; and the oligomerization domain of the second fusion protein comprises an amino acid sequence with at least 90% sequence identity to any one of SEQ ID NOs: 52-65. In some embodiments, the IFN polypeptide of the second fusion protein comprises an amino acid sequence with at least 91% sequence identity to any one of SEQ ID NOs: 1-7; the transmembrane polypeptide of the second fusion protein comprises an amino acid sequence with at least 91% sequence identity to any one of SEQ ID NOs: 66-74; and the oligomerization domain of the second fusion protein comprises an amino acid sequence with at least 91% sequence identity to any one of SEQ ID NOs: 52-65. In some embodiments, the IFN polypeptide of the second fusion protein comprises an amino acid sequence with at least 92% sequence identity to any one of SEQ ID NOs: 1-7; the transmembrane polypeptide of the second fusion protein comprises an amino acid sequence with at least 92% sequence identity to any one of SEQ ID NOs: 66-74; and the oligomerization domain of the second fusion protein comprises an amino acid sequence with at least 92% sequence identity to any one of SEQ ID NOs: 52-65. In some embodiments, the IFN polypeptide of the second fusion protein comprises an amino acid sequence with at least 93% sequence identity to any one of SEQ ID NOs: 1-7; the transmembrane polypeptide of the second fusion protein comprises an amino acid sequence with at least 93% sequence identity to any one of SEQ ID NOs: 66-74; and the oligomerization domain of the second fusion protein comprises an amino acid sequence with at least 93% sequence identity to any one of SEQ ID NOs: 52-65. In some embodiments, the IFN polypeptide of the second fusion protein comprises an amino acid sequence with at least 94% sequence identity to any one of SEQ ID NOs: 1-7; the transmembrane polypeptide of the second fusion protein comprises an amino acid sequence with at least 94% sequence identity to any one of SEQ ID NOs: 66-74; and the oligomerization domain of the second fusion protein comprises an amino acid sequence with at least 94% sequence identity to any one of SEQ ID NOs: 52-65. In some embodiments, the IFN polypeptide of the second fusion protein comprises an amino acid sequence with at least 95% sequence identity to any one of SEQ ID NOs: 1-7; the transmembrane polypeptide of the second fusion protein comprises an amino acid sequence with at least 95% sequence identity to any one of SEQ ID NOs: 66-74; and the oligomerization domain of the second fusion protein comprises an amino acid sequence with at least 95% sequence identity to any one of SEQ ID NOs: 52-65. In some embodiments, the IFN polypeptide of the second fusion protein comprises an amino acid sequence with at least 96% sequence identity to any one of SEQ ID NOs: 1-7; the transmembrane polypeptide of the second fusion protein comprises an amino acid sequence with at least 96% sequence identity to any one of SEQ ID NOs: 66-74; and the oligomerization domain of the second fusion protein comprises an amino acid sequence with at least 96% sequence identity to any one of SEQ ID NOs: 52-65. In some embodiments, the IFN polypeptide of the second fusion protein comprises an amino acid sequence with at least 97% sequence identity to any one of SEQ ID NOs: 1-7; the transmembrane polypeptide of the second fusion protein comprises an amino acid sequence with at least 97% sequence identity to any one of SEQ ID NOs: 66-74; and the oligomerization domain of the second fusion protein comprises an amino acid sequence with at least 97% sequence identity to any one of SEQ ID NOs: 52-65. In some embodiments, the IFN polypeptide of the second fusion protein comprises an amino acid sequence with at least 98% sequence identity to any one of SEQ ID NOs: 1-7; the transmembrane polypeptide of the second fusion protein comprises an amino acid sequence with at least 98% sequence identity to any one of SEQ ID NOs: 66-74; and the oligomerization domain of the second fusion protein comprises an amino acid sequence with at least 98% sequence identity to any one of SEQ ID NOs: 52-65. In some embodiments, the IFN polypeptide of the second fusion protein comprises an amino acid sequence with at least 99% sequence identity to any one of SEQ ID NOs: 1-7; the transmembrane polypeptide of the second fusion protein comprises an amino acid sequence with at least 99% sequence identity to any one of SEQ ID NOs: 66-74; and the oligomerization domain of the second fusion protein comprises an amino acid sequence with at least 99% sequence identity to any one of SEQ ID NOs: 52-65. In some embodiments, the IFN polypeptide of the second fusion protein comprises an amino acid sequence according to any one of SEQ ID NOs: 1-7; the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65.

In some embodiments, the antigen on the target cell comprises an antigen listed in Table 2, and the transmembrane polypeptide of the second fusion protein comprises a transmembrane domain of a Vesicular Stomatitis virus glycoprotein (VSV-G), a transmembrane domain and a cytosolic domain of VSV-G, a transmembrane domain of Influenza Neuraminidase (NA), a transmembrane domain and a cytosolic domain of Influenza Neuraminidase (NA), a transmembrane domain of influenza Hemagglutinin (HA), a transmembrane domain and a cytosolic domain of influenza Hemagglutinin (HA), a transmembrane domain of HIV surface glycoprotein GP120 or GP41, a transmembrane domain and a cytosolic domain of HIV surface glycoprotein GP120 or GP4, a transmembrane domain of Dengue E Protein, a transmembrane domain and a cytosolic domain of Dengue E Protein, a transmembrane domain of measles virus surface glycoprotein hemagglutinin (H) protein, or a transmembrane domain and a cytosolic domain of measles virus surface glycoprotein hemagglutinin (H) protein; and the oligomerization domain of the second fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, the antibody comprises an amino acid sequence from at least one complementarity determining region of BG10-19, 80R, 7D10, FI6, 1E01, H015, 2H5, ADRI-2F3, H004, H009, H007, H019, or H020; the transmembrane polypeptide of the second fusion protein comprises a transmembrane domain of a Vesicular Stomatitis virus glycoprotein (VSV-G), a transmembrane domain and a cytosolic domain of VSV-G, a transmembrane domain of Influenza Neuraminidase (NA), a transmembrane domain and a cytosolic domain of Influenza Neuraminidase (NA), a transmembrane domain of influenza Hemagglutinin (HA), a transmembrane domain and a cytosolic domain of influenza Hemagglutinin (HA), a transmembrane domain of HIV surface glycoprotein GP120 or GP41, a transmembrane domain and a cytosolic domain of HIV surface glycoprotein GP120 or GP4, a transmembrane domain of Dengue E Protein, a transmembrane domain and a cytosolic domain of Dengue E Protein, a transmembrane domain of measles virus surface glycoprotein hemagglutinin (H) protein, or a transmembrane domain and a cytosolic domain of measles virus surface glycoprotein hemagglutinin (H) protein; and the oligomerization domain of the second fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain.

In some embodiments, the antibody comprises a CDR-H1 according to SEQ ID NO: 75, a CDR-H2 according to SEQ ID NO: 76, a CDR-H3 according to SEQ ID NO: 77, a CDR-L1 according to SEQ ID NO: 114, a CDR-L2 according to SEQ ID NO: 115, and a CDR-L3 according to SEQ ID NO: 116; the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 78, a CDR-H2 according to SEQ ID NO: 79, a CDR-H3 according to SEQ ID NO: 80, a CDR-L1 according to SEQ ID NO: 117, a CDR-L2 according to SEQ ID NO: 118, and a CDR-L3 according to SEQ ID NO: 119, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 81, a CDR-H2 according to SEQ ID NO: 82, a CDR-H3 according to SEQ ID NO: 83, a CDR-L1 according to SEQ ID NO: 120, a CDR-L2 according to SEQ ID NO: 121, and a CDR-L3 according to SEQ ID NO: 122, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 84, a CDR-H2 according to SEQ ID NO: 85, a CDR-H3 according to SEQ ID NO: 86, a CDR-L1 according to SEQ ID NO: 123, a CDR-L2 according to SEQ ID NO: 124, and a CDR-L3 according to SEQ ID NO: 125, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 87, a CDR-H2 according to SEQ ID NO: 88, a CDR-H3 according to SEQ ID NO: 89, a CDR-L1 according to SEQ ID NO: 126, a CDR-L2 according to SEQ ID NO: 127, and a CDR-L3 according to SEQ ID NO: 128, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 90, a CDR-H2 according to SEQ ID NO: 91, a CDR-H3 according to SEQ ID NO: 92, a CDR-L1 according to SEQ ID NO: 129, a CDR-L2 according to SEQ ID NO: 130, and a CDR-L3 according to SEQ ID NO: 131, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 93, a CDR-H2 according to SEQ ID NO: 94, a CDR-H3 according to SEQ ID NO: 95, a CDR-L1 according to SEQ ID NO: 132, a CDR-L2 according to SEQ ID NO: 133, and a CDR-L3 according to SEQ ID NO: 134, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 96, a CDR-H2 according to SEQ ID NO: 97, a CDR-H3 according to SEQ ID NO: 98, a CDR-L1 according to SEQ ID NO: 135, a CDR-L2 according to SEQ ID NO: 136, and a CDR-L3 according to SEQ ID NO: 137, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 99, a CDR-H2 according to SEQ ID NO: 100, a CDR-H3 according to SEQ ID NO: 101, a CDR-L1 according to SEQ ID NO: 138, a CDR-L2 according to SEQ ID NO: 139, and a CDR-L3 according to SEQ ID NO: 140, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 102, a CDR-H2 according to SEQ ID NO: 103, a CDR-H3 according to SEQ ID NO: 104, a CDR-L1 according to SEQ ID NO: 141, a CDR-L2 according to SEQ ID NO: 142, and a CDR-L3 according to SEQ ID NO: 143, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 105, a CDR-H2 according to SEQ ID NO: 106, a CDR-H3 according to SEQ ID NO: 107, a CDR-L1 according to SEQ ID NO: 144, a CDR-L2 according to SEQ ID NO: 145, and a CDR-L3 according to SEQ ID NO: 146, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 108, a CDR-H2 according to SEQ ID NO: 109, a CDR-H3 according to SEQ ID NO: 110, a CDR-L1 according to SEQ ID NO: 147, a CDR-L2 according to SEQ ID NO: 148, and a CDR-L3 according to SEQ ID NO: 149, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65. In some embodiments, the antibody comprises a CDR-H1 according to SEQ ID NO: 111, a CDR-H2 according to SEQ ID NO: 112, a CDR-H3 according to SEQ ID NO: 113, a CDR-L1 according to SEQ ID NO: 150, a CDR-L2 according to SEQ ID NO: 151, and a CDR-L3 according to SEQ ID NO: 152, and the transmembrane polypeptide of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 66-74; and the oligomerization domain of the second fusion protein comprises an amino acid sequence with at least 75%, 80%, 85%, 90%, 91%, 92%, 93%, 94%, 95%, 96%, 97%, 98%, or at least 99% sequence identity to any one of SEQ ID NOs: 52-65.

In some embodiments, the antibody comprises a CDR-H1 according to SEQ ID NO: 75, a CDR-H2 according to SEQ ID NO: 76, a CDR-H3 according to SEQ ID NO: 77, a CDR-L1 according to SEQ ID NO: 114, a CDR-L2 according to SEQ ID NO: 115, and a CDR-L3 according to SEQ ID NO: 116; the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 78, a CDR-H2 according to SEQ ID NO: 79, a CDR-H3 according to SEQ ID NO: 80, a CDR-L1 according to SEQ ID NO: 117, a CDR-L2 according to SEQ ID NO: 118, and a CDR-L3 according to SEQ ID NO: 119, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 81, a CDR-H2 according to SEQ ID NO: 82, a CDR-H3 according to SEQ ID NO: 83, a CDR-L1 according to SEQ ID NO: 120, a CDR-L2 according to SEQ ID NO: 121, and a CDR-L3 according to SEQ ID NO: 122, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 84, a CDR-H2 according to SEQ ID NO: 85, a CDR-H3 according to SEQ ID NO: 86, a CDR-L1 according to SEQ ID NO: 123, a CDR-L2 according to SEQ ID NO: 124, and a CDR-L3 according to SEQ ID NO: 125, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 87, a CDR-H2 according to SEQ ID NO: 88, a CDR-H3 according to SEQ ID NO: 89, a CDR-L1 according to SEQ ID NO: 126, a CDR-L2 according to SEQ ID NO: 127, and a CDR-L3 according to SEQ ID NO: 128, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 90, a CDR-H2 according to SEQ ID NO: 91, a CDR-H3 according to SEQ ID NO: 92, a CDR-L1 according to SEQ ID NO: 129, a CDR-L2 according to SEQ ID NO: 130, and a CDR-L3 according to SEQ ID NO: 131, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 93, a CDR-H2 according to SEQ ID NO: 94, a CDR-H3 according to SEQ ID NO: 95, a CDR-L1 according to SEQ ID NO: 132, a CDR-L2 according to SEQ ID NO: 133, and a CDR-L3 according to SEQ ID NO: 134, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 96, a CDR-H2 according to SEQ ID NO: 97, a CDR-H3 according to SEQ ID NO: 98, a CDR-L1 according to SEQ ID NO: 135, a CDR-L2 according to SEQ ID NO: 136, and a CDR-L3 according to SEQ ID NO: 137, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 99, a CDR-H2 according to SEQ ID NO: 100, a CDR-H3 according to SEQ ID NO: 101, a CDR-L1 according to SEQ ID NO: 138, a CDR-L2 according to SEQ ID NO: 139, and a CDR-L3 according to SEQ ID NO: 140, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 102, a CDR-H2 according to SEQ ID NO: 103, a CDR-H3 according to SEQ ID NO: 104, a CDR-L1 according to SEQ ID NO: 141, a CDR-L2 according to SEQ ID NO: 142, and a CDR-L3 according to SEQ ID NO: 143, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 105, a CDR-H2 according to SEQ ID NO: 106, a CDR-H3 according to SEQ ID NO: 107, a CDR-L1 according to SEQ ID NO: 144, a CDR-L2 according to SEQ ID NO: 145, and a CDR-L3 according to SEQ ID NO: 146, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 108, a CDR-H2 according to SEQ ID NO: 109, a CDR-H3 according to SEQ ID NO: 110, a CDR-L1 according to SEQ ID NO: 147, a CDR-L2 according to SEQ ID NO: 148, and a CDR-L3 according to SEQ ID NO: 149, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65. In some embodiments, the antibody comprises a CDR-H1 according to SEQ ID NO: 111, a CDR-H2 according to SEQ ID NO: 112, a CDR-H3 according to SEQ ID NO: 113, a CDR-L1 according to SEQ ID NO: 150, a CDR-L2 according to SEQ ID NO: 151, and a CDR-L3 according to SEQ ID NO: 152, and the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65.

In some embodiments, the antibody comprises a VH comprising an amino acid sequence with at least 75% sequence identity to any one of SEQ ID Nos: 8-20 and a VL comprising an amino acid sequence with at least 75% sequence identity to any one of SEQ ID Nos: 21-33; the transmembrane polypeptide of the second fusion protein comprises an amino acid sequence with at least 75% sequence identity to any one of SEQ ID NOs: 66-74; and the oligomerization domain of the second fusion protein comprises an amino acid sequence with at least 75% sequence identity to any one of SEQ ID NOs: 52-65. In some embodiments, the antibody comprises a VH comprising an amino acid sequence with at least 76% sequence identity to any one of SEQ ID Nos: 8-20 and a VL comprising an amino acid sequence with at least 76% sequence identity to any one of SEQ ID Nos: 21-33: the transmembrane polypeptide of the second fusion protein comprises an amino acid sequence with at least 76% sequence identity to any one of SEQ ID NOs: 66-74; and the oligomerization domain of the second fusion protein comprises an amino acid sequence with at least 76% sequence identity to any one of SEQ ID NOs: 52-65. In some embodiments, the antibody comprises a VH comprising an amino acid sequence with at least 77% sequence identity to any one of SEQ ID Nos: 8-20 and a VL comprising an amino acid sequence with at least 77% sequence identity to any one of SEQ ID Nos: 21-33; the transmembrane polypeptide of the second fusion protein comprises an amino acid sequence with at least 77% sequence identity to any one of SEQ ID NOs: 66-74; and the oligomerization domain of the second fusion protein comprises an amino acid sequence with at least 77% sequence identity to any one of SEQ ID NOs: 52-65. In some embodiments, the antibody comprises a VH comprising an amino acid sequence with at least 78% sequence identity to any one of SEQ ID Nos: 8-20 and a VL comprising an amino acid sequence with at least 78% sequence identity to any one of SEQ ID Nos: 21-33: the transmembrane polypeptide of the second fusion protein comprises an amino acid sequence with at least 78% sequence identity to any one of SEQ ID NOs: 66-74; and the oligomerization domain of the second fusion protein comprises an amino acid sequence with at least 78% sequence identity to any one of SEQ ID NOs: 52-65. In some embodiments, the antibody comprises a VH comprising an amino acid sequence with at least 79% sequence identity to any one of SEQ ID Nos: 8-20 and a VL comprising an amino acid sequence with at least 79% sequence identity to any one of SEQ ID Nos: 21-33; the transmembrane polypeptide of the second fusion protein comprises an amino acid sequence with at least 79% sequence identity to any one of SEQ ID NOs: 66-74; and the oligomerization domain of the second fusion protein comprises an amino acid sequence with at least 79% sequence identity to any one of SEQ ID NOs: 52-65. In some embodiments, the antibody comprises a VH comprising an amino acid sequence with at least 80% sequence identity to any one of SEQ ID Nos: 8-20 and a VL comprising an amino acid sequence with at least 80% sequence identity to any one of SEQ ID Nos: 21-33: the transmembrane polypeptide of the second fusion protein comprises an amino acid sequence with at least 80% sequence identity to any one of SEQ ID NOs: 66-74; and the oligomerization domain of the second fusion protein comprises an amino acid sequence with at least 80% sequence identity to any one of SEQ ID NOs: 52-65. In some embodiments, the antibody comprises a VH comprising an amino acid sequence with at least 81% sequence identity to any one of SEQ ID Nos: 8-20 and a VL comprising an amino acid sequence with at least 81% sequence identity to any one of SEQ ID Nos: 21-33; the transmembrane polypeptide of the second fusion protein comprises an amino acid sequence with at least 81% sequence identity to any one of SEQ ID NOs: 66-74; and the oligomerization domain of the second fusion protein comprises an amino acid sequence with at least 81% sequence identity to any one of SEQ ID NOs: 52-65. In some embodiments, the antibody comprises a VH comprising an amino acid sequence with at least 82% sequence identity to any one of SEQ ID Nos: 8-20 and a VL comprising an amino acid sequence with at least 82% sequence identity to any one of SEQ ID Nos: 21-33; the transmembrane polypeptide of the second fusion protein comprises an amino acid sequence with at least 82% sequence identity to any one of SEQ ID NOs: 66-74; and the oligomerization domain of the second fusion protein comprises an amino acid sequence with at least 82% sequence identity to any one of SEQ ID NOs: 52-65. In some embodiments, the antibody comprises a VH comprising an amino acid sequence with at least 83% sequence identity to any one of SEQ ID Nos: 8-20 and a VL comprising an amino acid sequence with at least 83% sequence identity to any one of SEQ ID Nos: 21-33; the transmembrane polypeptide of the second fusion protein comprises an amino acid sequence with at least 83% sequence identity to any one of SEQ ID NOs: 66-74; and the oligomerization domain of the second fusion protein comprises an amino acid sequence with at least 83% sequence identity to any one of SEQ ID NOs: 52-65. In some embodiments, the antibody comprises a VH comprising an amino acid sequence with at least 84% sequence identity to any one of SEQ ID Nos: 8-20 and a VL comprising an amino acid sequence with at least 84% sequence identity to any one of SEQ ID Nos: 21-33: the transmembrane polypeptide of the second fusion protein comprises an amino acid sequence with at least 84% sequence identity to any one of SEQ ID NOs: 66-74; and the oligomerization domain of the second fusion protein comprises an amino acid sequence with at least 84% sequence identity to any one of SEQ ID NOs: 52-65. In some embodiments, the antibody comprises a VH comprising an amino acid sequence with at least 85% sequence identity to any one of SEQ ID Nos: 8-20 and a VL comprising an amino acid sequence with at least 85% sequence identity to any one of SEQ ID Nos: 21-33; the transmembrane polypeptide of the second fusion protein comprises an amino acid sequence with at least 85% sequence identity to any one of SEQ ID NOs: 66-74; and the oligomerization domain of the second fusion protein comprises an amino acid sequence with at least 85% sequence identity to any one of SEQ ID NOs: 52-65. In some embodiments, the antibody comprises a VH comprising an amino acid sequence with at least 86% sequence identity to any one of SEQ ID Nos: 8-20 and a VL comprising an amino acid sequence with at least 86% sequence identity to any one of SEQ ID Nos: 21-33; the transmembrane polypeptide of the second fusion protein comprises an amino acid sequence with at least 86% sequence identity to any one of SEQ ID NOs: 66-74; and the oligomerization domain of the second fusion protein comprises an amino acid sequence with at least 86% sequence identity to any one of SEQ ID NOs: 52-65. In some embodiments, the antibody comprises a VH comprising an amino acid sequence with at least 87% sequence identity to any one of SEQ ID Nos: 8-20 and a VL comprising an amino acid sequence with at least 87% sequence identity to any one of SEQ ID Nos: 21-33; the transmembrane polypeptide of the second fusion protein comprises an amino acid sequence with at least 87% sequence identity to any one of SEQ ID NOs: 66-74; and the oligomerization domain of the second fusion protein comprises an amino acid sequence with at least 87% sequence identity to any one of SEQ ID NOs: 52-65. In some embodiments, the antibody comprises a VH comprising an amino acid sequence with at least 88% sequence identity to any one of SEQ ID Nos: 8-20 and a VL comprising an amino acid sequence with at least 88% sequence identity to any one of SEQ ID Nos: 21-33; the transmembrane polypeptide of the second fusion protein comprises an amino acid sequence with at least 88% sequence identity to any one of SEQ ID NOs: 66-74; and the oligomerization domain of the second fusion protein comprises an amino acid sequence with at least 88% sequence identity to any one of SEQ ID NOs: 52-65. In some embodiments, the antibody comprises a VH comprising an amino acid sequence with at least 89% sequence identity to any one of SEQ ID Nos: 8-20 and a VL comprising an amino acid sequence with at least 89% sequence identity to any one of SEQ ID Nos: 21-33; the transmembrane polypeptide of the second fusion protein comprises an amino acid sequence with at least 89% sequence identity to any one of SEQ ID NOs: 66-74; and the oligomerization domain of the second fusion protein comprises an amino acid sequence with at least 89% sequence identity to any one of SEQ ID NOs: 52-65. In some embodiments, the antibody comprises a VH comprising an amino acid sequence with at least 90% sequence identity to any one of SEQ ID Nos: 8-20 and a VL comprising an amino acid sequence with at least 90% sequence identity to any one of SEQ ID Nos: 21-33: the transmembrane polypeptide of the second fusion protein comprises an amino acid sequence with at least 90% sequence identity to any one of SEQ ID NOs: 66-74; and the oligomerization domain of the second fusion protein comprises an amino acid sequence with at least 90% sequence identity to any one of SEQ ID NOs: 52-65. In some embodiments, the antibody comprises a VH comprising an amino acid sequence with at least 91% sequence identity to any one of SEQ ID Nos: 8-20 and a VL comprising an amino acid sequence with at least 91% sequence identity to any one of SEQ ID Nos: 21-33; the transmembrane polypeptide of the second fusion protein comprises an amino acid sequence with at least 91% sequence identity to any one of SEQ ID NOs: 66-74; and the oligomerization domain of the second fusion protein comprises an amino acid sequence with at least 91% sequence identity to any one of SEQ ID NOs: 52-65. In some embodiments, the antibody comprises a VH comprising an amino acid sequence with at least 92% sequence identity to any one of SEQ ID Nos: 8-20 and a VL comprising an amino acid sequence with at least 92% sequence identity to any one of SEQ ID Nos: 21-33: the transmembrane polypeptide of the second fusion protein comprises an amino acid sequence with at least 92% sequence identity to any one of SEQ ID NOs: 66-74; and the oligomerization domain of the second fusion protein comprises an amino acid sequence with at least 92% sequence identity to any one of SEQ ID NOs: 52-65. In some embodiments, the antibody comprises a VH comprising an amino acid sequence with at least 93% sequence identity to any one of SEQ ID Nos: 8-20 and a VL comprising an amino acid sequence with at least 93% sequence identity to any one of SEQ ID Nos: 21-33; the transmembrane polypeptide of the second fusion protein comprises an amino acid sequence with at least 93% sequence identity to any one of SEQ ID NOs: 66-74; and the oligomerization domain of the second fusion protein comprises an amino acid sequence with at least 93% sequence identity to any one of SEQ ID NOs: 52-65. In some embodiments, the antibody comprises a VH comprising an amino acid sequence with at least 94% sequence identity to any one of SEQ ID Nos: 8-20 and a VL comprising an amino acid sequence with at least 94% sequence identity to any one of SEQ ID Nos: 21-33: the transmembrane polypeptide of the second fusion protein comprises an amino acid sequence with at least 94% sequence identity to any one of SEQ ID NOs: 66-74; and the oligomerization domain of the second fusion protein comprises an amino acid sequence with at least 94% sequence identity to any one of SEQ ID NOs: 52-65. In some embodiments, the antibody comprises a VH comprising an amino acid sequence with at least 95% sequence identity to any one of SEQ ID Nos: 8-20 and a VL comprising an amino acid sequence with at least 95% sequence identity to any one of SEQ ID Nos: 21-33; the transmembrane polypeptide of the second fusion protein comprises an amino acid sequence with at least 95% sequence identity to any one of SEQ ID NOs: 66-74; and the oligomerization domain of the second fusion protein comprises an amino acid sequence with at least 95% sequence identity to any one of SEQ ID NOs: 52-65. In some embodiments, the antibody comprises a VH comprising an amino acid sequence with at least 96% sequence identity to any one of SEQ ID Nos: 8-20 and a VL comprising an amino acid sequence with at least 96% sequence identity to any one of SEQ ID Nos: 21-33: the transmembrane polypeptide of the second fusion protein comprises an amino acid sequence with at least 96% sequence identity to any one of SEQ ID NOs: 66-74; and the oligomerization domain of the second fusion protein comprises an amino acid sequence with at least 96% sequence identity to any one of SEQ ID NOs: 52-65. In some embodiments, the antibody comprises a VH comprising an amino acid sequence with at least 97% sequence identity to any one of SEQ ID Nos: 8-20 and a VL comprising an amino acid sequence with at least 97% sequence identity to any one of SEQ ID Nos: 21-33; the transmembrane polypeptide of the second fusion protein comprises an amino acid sequence with at least 97% sequence identity to any one of SEQ ID NOs: 66-74; and the oligomerization domain of the second fusion protein comprises an amino acid sequence with at least 97% sequence identity to any one of SEQ ID NOs: 52-65. In some embodiments, the antibody comprises a VH comprising an amino acid sequence with at least 98% sequence identity to any one of SEQ ID Nos: 8-20 and a VL comprising an amino acid sequence with at least 98% sequence identity to any one of SEQ ID Nos: 21-33: the transmembrane polypeptide of the second fusion protein comprises an amino acid sequence with at least 98% sequence identity to any one of SEQ ID NOs: 66-74; and the oligomerization domain of the second fusion protein comprises an amino acid sequence with at least 98% sequence identity to any one of SEQ ID NOs: 52-65. In some embodiments, the antibody comprises a VH comprising an amino acid sequence with at least 99% sequence identity to any one of SEQ ID Nos: 8-20 and a VL comprising an amino acid sequence with at least 99% sequence identity to any one of SEQ ID Nos: 21-33; the transmembrane polypeptide of the second fusion protein comprises an amino acid sequence with at least 99% sequence identity to any one of SEQ ID NOs: 66-74; and the oligomerization domain of the second fusion protein comprises an amino acid sequence with at least 99% sequence identity to any one of SEQ ID NOs: 52-65. In some embodiments, the antibody comprises a VH comprising an amino acid sequence according to any one of SEQ ID Nos: 8-20 and a VL comprising an amino acid sequence according to any one of SEQ ID Nos: 21-33; the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65.

In some embodiments, the homing polypeptide comprises an amino acid sequence with at least 75% sequence identity to any one of SEQ ID NOs: 34-51: the transmembrane polypeptide of the second fusion protein comprises an amino acid sequence with at least 75% sequence identity to any one of SEQ ID NOs: 66-74; and the oligomerization domain of the second fusion protein comprises an amino acid sequence with at least 75% sequence identity to any one of SEQ ID NOs: 52-65. In some embodiments, the homing polypeptide comprises an amino acid sequence with at least 76% sequence identity to any one of SEQ ID NOs: 34-51; the transmembrane polypeptide of the second fusion protein comprises an amino acid sequence with at least 76% sequence identity to any one of SEQ ID NOs: 66-74; and the oligomerization domain of the second fusion protein comprises an amino acid sequence with at least 76% sequence identity to any one of SEQ ID NOs: 52-65. In some embodiments, the homing polypeptide comprises an amino acid sequence with at least 77% sequence identity to any one of SEQ ID NOs: 34-51; the transmembrane polypeptide of the second fusion protein comprises an amino acid sequence with at least 77% sequence identity to any one of SEQ ID NOs: 66-74; and the oligomerization domain of the second fusion protein comprises an amino acid sequence with at least 77% sequence identity to any one of SEQ ID NOs: 52-65. In some embodiments, the homing polypeptide comprises an amino acid sequence with at least 78% sequence identity to any one of SEQ ID NOs: 34-51; the transmembrane polypeptide of the second fusion protein comprises an amino acid sequence with at least 78% sequence identity to any one of SEQ ID NOs: 66-74; and the oligomerization domain of the second fusion protein comprises an amino acid sequence with at least 78% sequence identity to any one of SEQ ID NOs: 52-65. In some embodiments, the homing polypeptide comprises an amino acid sequence with at least 79% sequence identity to any one of SEQ ID NOs: 34-51; the transmembrane polypeptide of the second fusion protein comprises an amino acid sequence with at least 79% sequence identity to any one of SEQ ID NOs: 66-74; and the oligomerization domain of the second fusion protein comprises an amino acid sequence with at least 79% sequence identity to any one of SEQ ID NOs: 52-65. In some embodiments, the homing polypeptide comprises an amino acid sequence with at least 80% sequence identity to any one of SEQ ID NOs: 34-51; the transmembrane polypeptide of the second fusion protein comprises an amino acid sequence with at least 80% sequence identity to any one of SEQ ID NOs: 66-74; and the oligomerization domain of the second fusion protein comprises an amino acid sequence with at least 80% sequence identity to any one of SEQ ID NOs: 52-65. In some embodiments, the homing polypeptide comprises an amino acid sequence with at least 81% sequence identity to any one of SEQ ID NOs: 34-51; the transmembrane polypeptide of the second fusion protein comprises an amino acid sequence with at least 81% sequence identity to any one of SEQ ID NOs: 66-74; and the oligomerization domain of the second fusion protein comprises an amino acid sequence with at least 81% sequence identity to any one of SEQ ID NOs: 52-65. In some embodiments, the homing polypeptide comprises an amino acid sequence with at least 82% sequence identity to any one of SEQ ID NOs: 34-51; the transmembrane polypeptide of the second fusion protein comprises an amino acid sequence with at least 82% sequence identity to any one of SEQ ID NOs: 66-74; and the oligomerization domain of the second fusion protein comprises an amino acid sequence with at least 82% sequence identity to any one of SEQ ID NOs: 52-65. In some embodiments, the homing polypeptide comprises an amino acid sequence with at least 83% sequence identity to any one of SEQ ID NOs: 34-51; the transmembrane polypeptide of the second fusion protein comprises an amino acid sequence with at least 83% sequence identity to any one of SEQ ID NOs: 66-74; and the oligomerization domain of the second fusion protein comprises an amino acid sequence with at least 83% sequence identity to any one of SEQ ID NOs: 52-65. In some embodiments, the homing polypeptide comprises an amino acid sequence with at least 84% sequence identity to any one of SEQ ID NOs: 34-51; the transmembrane polypeptide of the second fusion protein comprises an amino acid sequence with at least 84% sequence identity to any one of SEQ ID NOs: 66-74; and the oligomerization domain of the second fusion protein comprises an amino acid sequence with at least 84% sequence identity to any one of SEQ ID NOs: 52-65. In some embodiments, the homing polypeptide comprises an amino acid sequence with at least 85% sequence identity to any one of SEQ ID NOs: 34-51; the transmembrane polypeptide of the second fusion protein comprises an amino acid sequence with at least 85% sequence identity to any one of SEQ ID NOs: 66-74; and the oligomerization domain of the second fusion protein comprises an amino acid sequence with at least 85% sequence identity to any one of SEQ ID NOs: 52-65. In some embodiments, the homing polypeptide comprises an amino acid sequence with at least 86% sequence identity to any one of SEQ ID NOs: 34-51; the transmembrane polypeptide of the second fusion protein comprises an amino acid sequence with at least 86% sequence identity to any one of SEQ ID NOs: 66-74; and the oligomerization domain of the second fusion protein comprises an amino acid sequence with at least 86% sequence identity to any one of SEQ ID NOs: 52-65. In some embodiments, the homing polypeptide comprises an amino acid sequence with at least 87% sequence identity to any one of SEQ ID NOs: 34-51; the transmembrane polypeptide of the second fusion protein comprises an amino acid sequence with at least 87% sequence identity to any one of SEQ ID NOs: 66-74; and the oligomerization domain of the second fusion protein comprises an amino acid sequence with at least 87% sequence identity to any one of SEQ ID NOs: 52-65. In some embodiments, the homing polypeptide comprises an amino acid sequence with at least 88% sequence identity to any one of SEQ ID NOs: 34-51; the transmembrane polypeptide of the second fusion protein comprises an amino acid sequence with at least 88% sequence identity to any one of SEQ ID NOs: 66-74; and the oligomerization domain of the second fusion protein comprises an amino acid sequence with at least 88% sequence identity to any one of SEQ ID NOs: 52-65. In some embodiments, the homing polypeptide comprises an amino acid sequence with at least 89% sequence identity to any one of SEQ ID NOs: 34-51; the transmembrane polypeptide of the second fusion protein comprises an amino acid sequence with at least 89% sequence identity to any one of SEQ ID NOs: 66-74; and the oligomerization domain of the second fusion protein comprises an amino acid sequence with at least 89% sequence identity to any one of SEQ ID NOs: 52-65. In some embodiments, the homing polypeptide comprises an amino acid sequence with at least 90% sequence identity to any one of SEQ ID NOs: 34-51; the transmembrane polypeptide of the second fusion protein comprises an amino acid sequence with at least 90% sequence identity to any one of SEQ ID NOs: 66-74; and the oligomerization domain of the second fusion protein comprises an amino acid sequence with at least 90% sequence identity to any one of SEQ ID NOs: 52-65. In some embodiments, the homing polypeptide comprises an amino acid sequence with at least 91% sequence identity to any one of SEQ ID NOs: 34-51; the transmembrane polypeptide of the second fusion protein comprises an amino acid sequence with at least 91% sequence identity to any one of SEQ ID NOs: 66-74; and the oligomerization domain of the second fusion protein comprises an amino acid sequence with at least 91% sequence identity to any one of SEQ ID NOs: 52-65. In some embodiments, the homing polypeptide comprises an amino acid sequence with at least 92% sequence identity to any one of SEQ ID NOs: 34-51; the transmembrane polypeptide of the second fusion protein comprises an amino acid sequence with at least 92% sequence identity to any one of SEQ ID NOs: 66-74; and the oligomerization domain of the second fusion protein comprises an amino acid sequence with at least 92% sequence identity to any one of SEQ ID NOs: 52-65. In some embodiments, the homing polypeptide comprises an amino acid sequence with at least 93% sequence identity to any one of SEQ ID NOs: 34-51; the transmembrane polypeptide of the second fusion protein comprises an amino acid sequence with at least 93% sequence identity to any one of SEQ ID NOs: 66-74; and the oligomerization domain of the second fusion protein comprises an amino acid sequence with at least 93% sequence identity to any one of SEQ ID NOs: 52-65. In some embodiments, the homing polypeptide comprises an amino acid sequence with at least 94% sequence identity to any one of SEQ ID NOs: 34-51; the transmembrane polypeptide of the second fusion protein comprises an amino acid sequence with at least 94% sequence identity to any one of SEQ ID NOs: 66-74; and the oligomerization domain of the second fusion protein comprises an amino acid sequence with at least 94% sequence identity to any one of SEQ ID NOs: 52-65. In some embodiments, the homing polypeptide comprises an amino acid sequence with at least 95% sequence identity to any one of SEQ ID NOs: 34-51; the transmembrane polypeptide of the second fusion protein comprises an amino acid sequence with at least 95% sequence identity to any one of SEQ ID NOs: 66-74; and the oligomerization domain of the second fusion protein comprises an amino acid sequence with at least 95% sequence identity to any one of SEQ ID NOs: 52-65. In some embodiments, the homing polypeptide comprises an amino acid sequence with at least 96% sequence identity to any one of SEQ ID NOs: 34-51; the transmembrane polypeptide of the second fusion protein comprises an amino acid sequence with at least 96% sequence identity to any one of SEQ ID NOs: 66-74; and the oligomerization domain of the second fusion protein comprises an amino acid sequence with at least 96% sequence identity to any one of SEQ ID NOs: 52-65. In some embodiments, the homing polypeptide comprises an amino acid sequence with at least 97% sequence identity to any one of SEQ ID NOs: 34-51; the transmembrane polypeptide of the second fusion protein comprises an amino acid sequence with at least 97% sequence identity to any one of SEQ ID NOs: 66-74; and the oligomerization domain of the second fusion protein comprises an amino acid sequence with at least 97% sequence identity to any one of SEQ ID NOs: 52-65. In some embodiments, the homing polypeptide comprises an amino acid sequence with at least 98% sequence identity to any one of SEQ ID NOs: 34-51; the transmembrane polypeptide of the second fusion protein comprises an amino acid sequence with at least 98% sequence identity to any one of SEQ ID NOs: 66-74; and the oligomerization domain of the second fusion protein comprises an amino acid sequence with at least 98% sequence identity to any one of SEQ ID NOs: 52-65. In some embodiments, the homing polypeptide comprises an amino acid sequence with at least 99% sequence identity to any one of SEQ ID NOs: 34-51; the transmembrane polypeptide of the second fusion protein comprises an amino acid sequence with at least 99% sequence identity to any one of SEQ ID NOs: 66-74; and the oligomerization domain of the second fusion protein comprises an amino acid sequence with at least 99% sequence identity to any one of SEQ ID NOs: 52-65. In some embodiments, the homing polypeptide comprises an amino acid sequence according to any one of SEQ ID NOs: 34-51: the transmembrane polypeptide of the second fusion protein comprises an amino acid sequence according to any one of SEQ ID NOs: 66-74; and the oligomerization domain of the second fusion protein comprises an amino acid sequence according to any one of SEQ ID NOs: 52-65.

In some embodiments, the multivalent particle comprises a reporter, a therapeutic molecule, or combinations thereof. In some embodiments, the reporter protein comprises a fluorescent protein or an enzyme. Exemplary reporter genes include, but are not limited to, acetohydroxyacid synthase (AHAS), alkaline phosphatase (AP), beta galactosidase (LacZ), beta glucuronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination. In some embodiments, the reporter comprises a fluorescent protein. In some embodiments, the fluorescent protein comprises green fluorescent protein. In some embodiments, the reporter protein emits green fluorescence, yellow fluorescence, or red fluorescence. In some embodiments, the reporter comprises an enzyme. In some embodiments, the enzyme is β-galactosidase, alkaline phosphatase, β-lactamase, or luciferase. In some embodiments, the multivalent particle comprises a fluorophore expressed on a surface of the multivalent particle. In some embodiments, the fluorophore is conjugated to a membrane-intercalating polypeptide.

In some embodiments, the therapeutic molecule comprises an immune modulating protein, a cellular signal modulating molecule, a proliferation modulating molecule, or combinations thereof. In some embodiments, the therapeutic molecule comprises an immune checkpoint molecule. Exemplary immune checkpoint molecules include, but are not limited to cytotoxic T-lymphocyte associated protein 4 (CTLA4), PD-1, OX40, and CD28. In some embodiments, the therapeutic molecule comprises an inflammatory cytokine. In some embodiments, the inflammatory cytokine comprises IL-12, IL-18, TNF-alpha, or TNF-beta, or IFN-alpha, or IFN-beta. In some embodiments, the therapeutic molecule comprises a proliferation cytokine. In some embodiments, the proliferation cytokine comprises IL-7, or IL-15, or IL-21.

Various multivalent particles are contemplated herein. In some embodiments, the multivalent particle is synthetic. In some embodiments, the multivalent particle is recombinant. In some embodiments, the multivalent particle is recombinant and genetically encoded. In some embodiments, the multivalent particle does not comprise a lentiviral particle. In some embodiments, the multivalent particle comprises a lentiviral particle. In some embodiments, the multivalent particle does not comprise viral genetic material. In some embodiments, the multivalent particle comprises viral genetic material. In some embodiments, the multivalent particle comprises a bilayer. In some embodiments, the multivalent particle comprises a lipid bilayer. In some embodiments, the multivalent particle comprises a virus. In some embodiments, the multivalent particle comprises a replication incompetent virus. In some embodiments, the multivalent particle comprises a replication competent virus. In some embodiments, the multivalent particle comprises an enveloped particle. In some embodiments, the multivalent particle comprises a viral-like particle (VLP). In some embodiments, the multivalent particle comprises an extracellular vesicle. In some embodiments, the multivalent particle comprises an exosome. In some embodiments, the multivalent particle comprises an ectosome.

Multivalent particles as described herein, in some embodiments, comprise a first fusion protein, wherein the first fusion protein is expressed at multiple copies on a surface of the multivalent particle. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the multivalent particle. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the multivalent particle. In some embodiments, the first fusion protein is expressed at a valency of at least or about 10 copies on a surface of the multivalent particle. In some embodiments, the first fusion protein is expressed at a valency of at least or about 25 copies on a surface of the multivalent particle. In some embodiments, the first fusion protein is expressed at a valency of at least or about 50 copies on a surface of the multivalent particle. In some embodiments, the first fusion protein is expressed at a valency of at least or about 75 copies on a surface of the multivalent particle. In some embodiments, the first fusion protein is expressed at a valency of at least or about 100 copies on a surface of the multivalent particle. In some embodiments, the first fusion protein is expressed at a valency of at least or about 125 copies on a surface of the multivalent particle. In some embodiments, the first fusion protein is expressed at a valency of at least or about 150 copies on a surface of the multivalent particle. In some embodiments, the first fusion protein is expressed at a valency of at least or about 175 copies on a surface of the multivalent particle. In some embodiments, the first fusion protein is expressed at a valency of at least or about 200 copies on a surface of the multivalent particle. In some embodiments, the first fusion protein is expressed at a valency of at least or about 225 copies on a surface of the multivalent particle. In some embodiments, the first fusion protein is expressed at a valency of at least or about 250 copies on a surface of the multivalent particle. In some embodiments, the first fusion protein is expressed at a valency of at least or about 275 copies on a surface of the multivalent particle. In some embodiments, the first fusion protein is expressed at a valency of at least or about 300 copies on a surface of the multivalent particle. In some embodiments, the first fusion protein is expressed at a valency of at least or about 400 copies on a surface of the multivalent particle. In some embodiments, the first fusion protein is expressed at a valency of at least or about 500 copies on a surface of the multivalent particle. In some embodiments, the first fusion protein is expressed at a valency of at least or about 600 copies on a surface of the multivalent particle. In some embodiments, the first fusion protein is expressed at a valency of at least or about 700 copies on a surface of the multivalent particle. In some embodiments, the first fusion protein is expressed at a valency of at least or about 800 copies on a surface of the multivalent particle. In some embodiments, the first fusion protein is expressed at a valency of at least or about 900 copies on a surface of the multivalent particle. In some embodiments, the first fusion protein is expressed at a valency of at least or about 1000 copies on a surface of the multivalent particle. In some embodiments, the first fusion protein is expressed at a valency of at least or about 2000 copies on a surface of the multivalent particle.

In some embodiments, the multivalent particle is a viral-like particle. The viral-like particle as described herein, in some embodiments, comprise a first fusion protein, wherein the first fusion protein is expressed at multiple copies on a surface of the viral-like particle. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the viral-like particle. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the viral-like particle. In some embodiments, the first fusion protein is expressed at a valency of at least or about 10 copies on a surface of the viral-like particle. In some embodiments, the first fusion protein is expressed at a valency of at least or about 25 copies on a surface of the viral-like particle. In some embodiments, the first fusion protein is expressed at a valency of at least or about 50 copies on a surface of the viral-like particle. In some embodiments, the first fusion protein is expressed at a valency of at least or about 75 copies on a surface of the viral-like particle. In some embodiments, the first fusion protein is expressed at a valency of at least or about 100 copies on a surface of the viral-like particle. In some embodiments, the first fusion protein is expressed at a valency of at least or about 125 copies on a surface of the viral-like particle. In some embodiments, the first fusion protein is expressed at a valency of at least or about 150 copies on a surface of the viral-like particle. In some embodiments, the first fusion protein is expressed at a valency of at least or about 175 copies on a surface of the viral-like particle. In some embodiments, the first fusion protein is expressed at a valency of at least or about 200 copies on a surface of the viral-like particle. In some embodiments, the first fusion protein is expressed at a valency of at least or about 225 copies on a surface of the viral-like particle. In some embodiments, the first fusion protein is expressed at a valency of at least or about 250 copies on a surface of the viral-like particle. In some embodiments, the first fusion protein is expressed at a valency of at least or about 275 copies on a surface of the viral-like particle. In some embodiments, the first fusion protein is expressed at a valency of at least or about 300 copies on a surface of the viral-like particle. In some embodiments, the first fusion protein is expressed at a valency of at least or about 400 copies on a surface of the viral-like particle. In some embodiments, the first fusion protein is expressed at a valency of at least or about 500 copies on a surface of the viral-like particle. In some embodiments, the first fusion protein is expressed at a valency of at least or about 600 copies on a surface of the viral-like particle. In some embodiments, the first fusion protein is expressed at a valency of at least or about 700 copies on a surface of the viral-like particle. In some embodiments, the first fusion protein is expressed at a valency of at least or about 800 copies on a surface of the viral-like particle. In some embodiments, the first fusion protein is expressed at a valency of at least or about 900 copies on a surface of the viral-like particle. In some embodiments, the first fusion protein is expressed at a valency of at least or about 1000 copies on a surface of the viral-like particle. In some embodiments, the first fusion protein is expressed at a valency of at least or about 2000 copies on a surface of the viral-like particle.

In some embodiments, the multivalent particle is an extracellular vesicle. The extracellular vesicle as described herein, in some embodiments, comprise a first fusion protein, wherein the first fusion protein is expressed at multiple copies on a surface of the extracellular vesicle. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the extracellular vesicle. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the extracellular vesicle. In some embodiments, the first fusion protein is expressed at a valency of at least or about 10 copies on a surface of the extracellular vesicle. In some embodiments, the first fusion protein is expressed at a valency of at least or about 25 copies on a surface of the extracellular vesicle. In some embodiments, the first fusion protein is expressed at a valency of at least or about 50 copies on a surface of the extracellular vesicle. In some embodiments, the first fusion protein is expressed at a valency of at least or about 75 copies on a surface of the extracellular vesicle. In some embodiments, the first fusion protein is expressed at a valency of at least or about 100 copies on a surface of the extracellular vesicle. In some embodiments, the first fusion protein is expressed at a valency of at least or about 125 copies on a surface of the extracellular vesicle. In some embodiments, the first fusion protein is expressed at a valency of at least or about 150 copies on a surface of the extracellular vesicle. In some embodiments, the first fusion protein is expressed at a valency of at least or about 175 copies on a surface of the extracellular vesicle. In some embodiments, the first fusion protein is expressed at a valency of at least or about 200 copies on a surface of the extracellular vesicle. In some embodiments, the first fusion protein is expressed at a valency of at least or about 225 copies on a surface of the extracellular vesicle. In some embodiments, the first fusion protein is expressed at a valency of at least or about 250 copies on a surface of the extracellular vesicle. In some embodiments, the first fusion protein is expressed at a valency of at least or about 275 copies on a surface of the extracellular vesicle. In some embodiments, the first fusion protein is expressed at a valency of at least or about 300 copies on a surface of the extracellular vesicle. In some embodiments, the first fusion protein is expressed at a valency of at least or about 400 copies on a surface of the extracellular vesicle. In some embodiments, the first fusion protein is expressed at a valency of at least or about 500 copies on a surface of the extracellular vesicle. In some embodiments, the first fusion protein is expressed at a valency of at least or about 600 copies on a surface of the extracellular vesicle. In some embodiments, the first fusion protein is expressed at a valency of at least or about 700 copies on a surface of the extracellular vesicle. In some embodiments, the first fusion protein is expressed at a valency of at least or about 800 copies on a surface of the extracellular vesicle. In some embodiments, the first fusion protein is expressed at a valency of at least or about 900 copies on a surface of the extracellular vesicle. In some embodiments, the first fusion protein is expressed at a valency of at least or about 1000 copies on a surface of the extracellular vesicle. In some embodiments, the first fusion protein is expressed at a valency of at least or about 2000 copies on a surface of the extracellular vesicle.

In some embodiments, the multivalent particle is an exosome. The exosome as described herein, in some embodiments, comprise a first fusion protein, wherein the first fusion protein is expressed at multiple copies on a surface of the exosome. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the exosome. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the exosome. In some embodiments, the first fusion protein is expressed at a valency of at least or about 10 copies on a surface of the exosome. In some embodiments, the first fusion protein is expressed at a valency of at least or about 25 copies on a surface of the exosome. In some embodiments, the first fusion protein is expressed at a valency of at least or about 50 copies on a surface of the exosome. In some embodiments, the first fusion protein is expressed at a valency of at least or about 75 copies on a surface of the exosome. In some embodiments, the first fusion protein is expressed at a valency of at least or about 100 copies on a surface of the exosome. In some embodiments, the first fusion protein is expressed at a valency of at least or about 125 copies on a surface of the exosome. In some embodiments, the first fusion protein is expressed at a valency of at least or about 150 copies on a surface of the exosome. In some embodiments, the first fusion protein is expressed at a valency of at least or about 175 copies on a surface of the exosome. In some embodiments, the first fusion protein is expressed at a valency of at least or about 200 copies on a surface of the exosome. In some embodiments, the first fusion protein is expressed at a valency of at least or about 225 copies on a surface of the exosome. In some embodiments, the first fusion protein is expressed at a valency of at least or about 250 copies on a surface of the exosome. In some embodiments, the first fusion protein is expressed at a valency of at least or about 275 copies on a surface of the exosome. In some embodiments, the first fusion protein is expressed at a valency of at least or about 300 copies on a surface of the exosome. In some embodiments, the first fusion protein is expressed at a valency of at least or about 400 copies on a surface of the exosome. In some embodiments, the first fusion protein is expressed at a valency of at least or about 500 copies on a surface of the exosome. In some embodiments, the first fusion protein is expressed at a valency of at least or about 600 copies on a surface of the exosome. In some embodiments, the first fusion protein is expressed at a valency of at least or about 700 copies on a surface of the exosome. In some embodiments, the first fusion protein is expressed at a valency of at least or about 800 copies on a surface of the exosome. In some embodiments, the first fusion protein is expressed at a valency of at least or about 900 copies on a surface of the exosome. In some embodiments, the first fusion protein is expressed at a valency of at least or about 1000 copies on a surface of the exosome. In some embodiments, the first fusion protein is expressed at a valency of at least or about 2000 copies on a surface of the exosome.

In some embodiments, the multivalent particle is an ectosome. The ectosome as described herein, in some embodiments, comprise a first fusion protein, wherein the first fusion protein is expressed at multiple copies on a surface of the ectosome. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the ectosome. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the ectosome. In some embodiments, the first fusion protein is expressed at a valency of at least or about 10 copies on a surface of the ectosome. In some embodiments, the first fusion protein is expressed at a valency of at least or about 25 copies on a surface of the ectosome. In some embodiments, the first fusion protein is expressed at a valency of at least or about 50 copies on a surface of the ectosome. In some embodiments, the first fusion protein is expressed at a valency of at least or about 75 copies on a surface of the ectosome. In some embodiments, the first fusion protein is expressed at a valency of at least or about 100 copies on a surface of the ectosome. In some embodiments, the first fusion protein is expressed at a valency of at least or about 125 copies on a surface of the ectosome. In some embodiments, the first fusion protein is expressed at a valency of at least or about 150 copies on a surface of the ectosome. In some embodiments, the first fusion protein is expressed at a valency of at least or about 175 copies on a surface of the ectosome. In some embodiments, the first fusion protein is expressed at a valency of at least or about 200 copies on a surface of the ectosome. In some embodiments, the first fusion protein is expressed at a valency of at least or about 225 copies on a surface of the ectosome. In some embodiments, the first fusion protein is expressed at a valency of at least or about 250 copies on a surface of the ectosome. In some embodiments, the first fusion protein is expressed at a valency of at least or about 275 copies on a surface of the ectosome. In some embodiments, the first fusion protein is expressed at a valency of at least or about 300 copies on a surface of the ectosome. In some embodiments, the first fusion protein is expressed at a valency of at least or about 400 copies on a surface of the ectosome. In some embodiments, the first fusion protein is expressed at a valency of at least or about 500 copies on a surface of the ectosome. In some embodiments, the first fusion protein is expressed at a valency of at least or about 600 copies on a surface of the ectosome. In some embodiments, the first fusion protein is expressed at a valency of at least or about 700 copies on a surface of the ectosome. In some embodiments, the first fusion protein is expressed at a valency of at least or about 800 copies on a surface of the ectosome. In some embodiments, the first fusion protein is expressed at a valency of at least or about 900 copies on a surface of the ectosome. In some embodiments, the first fusion protein is expressed at a valency of at least or about 1000 copies on a surface of the ectosome. In some embodiments, the first fusion protein is expressed at a valency of at least or about 2000 copies on a surface of the ectosome.

In some embodiments, the multivalent particle is a virus. The virus as described herein, in some embodiments, comprise a first fusion protein, wherein the first fusion protein is expressed at multiple copies on a surface of the virus. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the virus. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the virus. In some embodiments, the first fusion protein is expressed at a valency of at least or about 10 copies on a surface of the virus. In some embodiments, the first fusion protein is expressed at a valency of at least or about 25 copies on a surface of the virus. In some embodiments, the first fusion protein is expressed at a valency of at least or about 50 copies on a surface of the virus. In some embodiments, the first fusion protein is expressed at a valency of at least or about 75 copies on a surface of the virus. In some embodiments, the first fusion protein is expressed at a valency of at least or about 100 copies on a surface of the virus. In some embodiments, the first fusion protein is expressed at a valency of at least or about 125 copies on a surface of the virus. In some embodiments, the first fusion protein is expressed at a valency of at least or about 150 copies on a surface of the virus. In some embodiments, the first fusion protein is expressed at a valency of at least or about 175 copies on a surface of the virus. In some embodiments, the first fusion protein is expressed at a valency of at least or about 200 copies on a surface of the virus. In some embodiments, the first fusion protein is expressed at a valency of at least or about 225 copies on a surface of the virus. In some embodiments, the first fusion protein is expressed at a valency of at least or about 250 copies on a surface of the virus. In some embodiments, the first fusion protein is expressed at a valency of at least or about 275 copies on a surface of the virus. In some embodiments, the first fusion protein is expressed at a valency of at least or about 300 copies on a surface of the virus. In some embodiments, the first fusion protein is expressed at a valency of at least or about 400 copies on a surface of the virus. In some embodiments, the first fusion protein is expressed at a valency of at least or about 500 copies on a surface of the virus. In some embodiments, the first fusion protein is expressed at a valency of at least or about 600 copies on a surface of the virus. In some embodiments, the first fusion protein is expressed at a valency of at least or about 700 copies on a surface of the virus. In some embodiments, the first fusion protein is expressed at a valency of at least or about 800 copies on a surface of the virus. In some embodiments, the first fusion protein is expressed at a valency of at least or about 900 copies on a surface of the virus. In some embodiments, the first fusion protein is expressed at a valency of at least or about 1000 copies on a surface of the virus. In some embodiments, the first fusion protein is expressed at a valency of at least or about 2000 copies on a surface of the virus.

In some embodiments, the multivalent particle is a replication incompetent virus. The replication incompetent virus as described herein, in some embodiments, comprise a first fusion protein, wherein the first fusion protein is expressed at multiple copies on a surface of the replication incompetent virus. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication incompetent virus. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication incompetent virus. In some embodiments, the first fusion protein is expressed at a valency of at least or about 10 copies on a surface of the replication incompetent virus. In some embodiments, the first fusion protein is expressed at a valency of at least or about 25 copies on a surface of the replication incompetent virus. In some embodiments, the first fusion protein is expressed at a valency of at least or about 50 copies on a surface of the replication incompetent virus. In some embodiments, the first fusion protein is expressed at a valency of at least or about 75 copies on a surface of the replication incompetent virus. In some embodiments, the first fusion protein is expressed at a valency of at least or about 100 copies on a surface of the replication incompetent virus. In some embodiments, the first fusion protein is expressed at a valency of at least or about 125 copies on a surface of the replication incompetent virus. In some embodiments, the first fusion protein is expressed at a valency of at least or about 150 copies on a surface of the replication incompetent virus. In some embodiments, the first fusion protein is expressed at a valency of at least or about 175 copies on a surface of the replication incompetent virus. In some embodiments, the first fusion protein is expressed at a valency of at least or about 200 copies on a surface of the replication incompetent virus. In some embodiments, the first fusion protein is expressed at a valency of at least or about 225 copies on a surface of the replication incompetent virus. In some embodiments, the first fusion protein is expressed at a valency of at least or about 250 copies on a surface of the replication incompetent virus. In some embodiments, the first fusion protein is expressed at a valency of at least or about 275 copies on a surface of the replication incompetent virus. In some embodiments, the first fusion protein is expressed at a valency of at least or about 300 copies on a surface of the replication incompetent virus. In some embodiments, the first fusion protein is expressed at a valency of at least or about 400 copies on a surface of the replication incompetent virus. In some embodiments, the first fusion protein is expressed at a valency of at least or about 500 copies on a surface of the replication incompetent virus. In some embodiments, the first fusion protein is expressed at a valency of at least or about 600 copies on a surface of the replication incompetent virus. In some embodiments, the first fusion protein is expressed at a valency of at least or about 700 copies on a surface of the replication incompetent virus. In some embodiments, the first fusion protein is expressed at a valency of at least or about 800 copies on a surface of the replication incompetent virus. In some embodiments, the first fusion protein is expressed at a valency of at least or about 900 copies on a surface of the replication incompetent virus. In some embodiments, the first fusion protein is expressed at a valency of at least or about 1000 copies on a surface of the replication incompetent virus. In some embodiments, the first fusion protein is expressed at a valency of at least or about 2000 copies on a surface of the replication incompetent virus.

In some embodiments, the multivalent particle is a replication competent virus. The replication competent virus as described herein, in some embodiments, comprise a first fusion protein, wherein the first fusion protein is expressed at multiple copies on a surface of the replication competent virus. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication competent virus. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication competent virus. In some embodiments, the first fusion protein is expressed at a valency of at least or about 10 copies on a surface of the replication competent virus. In some embodiments, the first fusion protein is expressed at a valency of at least or about 25 copies on a surface of the replication competent virus. In some embodiments, the first fusion protein is expressed at a valency of at least or about 50 copies on a surface of the replication competent virus. In some embodiments, the first fusion protein is expressed at a valency of at least or about 75 copies on a surface of the replication competent virus. In some embodiments, the first fusion protein is expressed at a valency of at least or about 100 copies on a surface of the replication competent virus. In some embodiments, the first fusion protein is expressed at a valency of at least or about 125 copies on a surface of the replication competent virus. In some embodiments, the first fusion protein is expressed at a valency of at least or about 150 copies on a surface of the replication competent virus. In some embodiments, the first fusion protein is expressed at a valency of at least or about 175 copies on a surface of the replication competent virus. In some embodiments, the first fusion protein is expressed at a valency of at least or about 200 copies on a surface of the replication competent virus. In some embodiments, the first fusion protein is expressed at a valency of at least or about 225 copies on a surface of the replication competent virus. In some embodiments, the first fusion protein is expressed at a valency of at least or about 250 copies on a surface of the replication competent virus. In some embodiments, the first fusion protein is expressed at a valency of at least or about 275 copies on a surface of the replication competent virus. In some embodiments, the first fusion protein is expressed at a valency of at least or about 300 copies on a surface of the replication competent virus. In some embodiments, the first fusion protein is expressed at a valency of at least or about 400 copies on a surface of the replication competent virus. In some embodiments, the first fusion protein is expressed at a valency of at least or about 500 copies on a surface of the replication competent virus. In some embodiments, the first fusion protein is expressed at a valency of at least or about 600 copies on a surface of the replication competent virus. In some embodiments, the first fusion protein is expressed at a valency of at least or about 700 copies on a surface of the replication competent virus. In some embodiments, the first fusion protein is expressed at a valency of at least or about 800 copies on a surface of the replication competent virus. In some embodiments, the first fusion protein is expressed at a valency of at least or about 900 copies on a surface of the replication competent virus. In some embodiments, the first fusion protein is expressed at a valency of at least or about 1000 copies on a surface of the replication competent virus. In some embodiments, the first fusion protein is expressed at a valency of at least or about 2000 copies on a surface of the replication competent virus.

Multivalent particles as described herein, in some embodiments, comprise a second fusion protein to further increase the valency of multivalent particle, wherein the second fusion protein is expressed at multiple copies on a surface of the multivalent particle. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the multivalent particle. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the multivalent particle. In some embodiments, the second fusion protein is expressed at a valency of at least or about 10 copies on a surface of the multivalent particle. In some embodiments, the second fusion protein is expressed at a valency of at least or about 25 copies on a surface of the multivalent particle. In some embodiments, the second fusion protein is expressed at a valency of at least or about 50 copies on a surface of the multivalent particle. In some embodiments, the second fusion protein is expressed at a valency of at least or about 75 copies on a surface of the multivalent particle. In some embodiments, the second fusion protein is expressed at a valency of at least or about 100 copies on a surface of the multivalent particle. In some embodiments, the second fusion protein is expressed at a valency of at least or about 125 copies on a surface of the multivalent particle. In some embodiments, the second fusion protein is expressed at a valency of at least or about 150 copies on a surface of the multivalent particle. In some embodiments, the second fusion protein is expressed at a valency of at least or about 175 copies on a surface of the multivalent particle. In some embodiments, the second fusion protein is expressed at a valency of at least or about 200 copies on a surface of the multivalent particle. In some embodiments, the second fusion protein is expressed at a valency of at least or about 225 copies on a surface of the multivalent particle. In some embodiments, the second fusion protein is expressed at a valency of at least or about 250 copies on a surface of the multivalent particle. In some embodiments, the second fusion protein is expressed at a valency of at least or about 275 copies on a surface of the multivalent particle. In some embodiments, the second fusion protein is expressed at a valency of at least or about 300 copies on a surface of the multivalent particle. In some embodiments, the second fusion protein is expressed at a valency of at least or about 400 copies on a surface of the multivalent particle. In some embodiments, the second fusion protein is expressed at a valency of at least or about 500 copies on a surface of the multivalent particle. In some embodiments, the second fusion protein is expressed at a valency of at least or about 600 copies on a surface of the multivalent particle. In some embodiments, the second fusion protein is expressed at a valency of at least or about 700 copies on a surface of the multivalent particle. In some embodiments, the second fusion protein is expressed at a valency of at least or about 800 copies on a surface of the multivalent particle. In some embodiments, the second fusion protein is expressed at a valency of at least or about 900 copies on a surface of the multivalent particle. In some embodiments, the second fusion protein is expressed at a valency of at least or about 1000 copies on a surface of the multivalent particle. In some embodiments, the second fusion protein is expressed at a valency of at least or about 2000 copies on a surface of the multivalent particle.

The viral-like particle as described herein, in some embodiments, comprises a second fusion protein, wherein the second fusion protein is expressed at multiple copies on a surface of the viral-like particle. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the viral-like particle. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the viral-like particle. In some embodiments, the second fusion protein is expressed at a valency of at least or about 10 copies on a surface of the viral-like particle. In some embodiments, the second fusion protein is expressed at a valency of at least or about 25 copies on a surface of the viral-like particle. In some embodiments, the second fusion protein is expressed at a valency of at least or about 50 copies on a surface of the viral-like particle. In some embodiments, the second fusion protein is expressed at a valency of at least or about 75 copies on a surface of the viral-like particle. In some embodiments, the second fusion protein is expressed at a valency of at least or about 100 copies on a surface of the viral-like particle. In some embodiments, the second fusion protein is expressed at a valency of at least or about 125 copies on a surface of the viral-like particle. In some embodiments, the second fusion protein is expressed at a valency of at least or about 150 copies on a surface of the viral-like particle. In some embodiments, the second fusion protein is expressed at a valency of at least or about 175 copies on a surface of the viral-like particle. In some embodiments, the second fusion protein is expressed at a valency of at least or about 200 copies on a surface of the viral-like particle. In some embodiments, the second fusion protein is expressed at a valency of at least or about 225 copies on a surface of the viral-like particle. In some embodiments, the second fusion protein is expressed at a valency of at least or about 250 copies on a surface of the viral-like particle. In some embodiments, the second fusion protein is expressed at a valency of at least or about 275 copies on a surface of the viral-like particle. In some embodiments, the second fusion protein is expressed at a valency of at least or about 300 copies on a surface of the viral-like particle. In some embodiments, the second fusion protein is expressed at a valency of at least or about 300 copies on a surface of the viral-like particle. In some embodiments, the second fusion protein is expressed at a valency of at least or about 400 copies on a surface of the viral-like particle. In some embodiments, the second fusion protein is expressed at a valency of at least or about 500 copies on a surface of the viral-like particle. In some embodiments, the second fusion protein is expressed at a valency of at least or about 600 copies on a surface of the viral-like particle. In some embodiments, the second fusion protein is expressed at a valency of at least or about 700 copies on a surface of the viral-like particle. In some embodiments, the second fusion protein is expressed at a valency of at least or about 800 copies on a surface of the viral-like particle. In some embodiments, the second fusion protein is expressed at a valency of at least or about 900 copies on a surface of the viral-like particle. In some embodiments, the second fusion protein is expressed at a valency of at least or about 1000 copies on a surface of the viral-like particle. In some embodiments, the second fusion protein is expressed at a valency of at least or about 2000 copies on a surface of the viral-like particle.

The extracellular vesicle, as described herein, in some embodiments, comprises a second fusion protein, wherein the second fusion protein is expressed at multiple copies on a surface of the extracellular vesicle. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the extracellular vesicle. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the extracellular vesicle. In some embodiments, the second fusion protein is expressed at a valency of at least or about 10 copies on a surface of the extracellular vesicle. In some embodiments, the second fusion protein is expressed at a valency of at least or about 25 copies on a surface of the extracellular vesicle. In some embodiments, the second fusion protein is expressed at a valency of at least or about 50 copies on a surface of the extracellular vesicle. In some embodiments, the second fusion protein is expressed at a valency of at least or about 75 copies on a surface of the extracellular vesicle. In some embodiments, the second fusion protein is expressed at a valency of at least or about 100 copies on a surface of the extracellular vesicle. In some embodiments, the second fusion protein is expressed at a valency of at least or about 125 copies on a surface of the extracellular vesicle. In some embodiments, the second fusion protein is expressed at a valency of at least or about 150 copies on a surface of the extracellular vesicle. In some embodiments, the second fusion protein is expressed at a valency of at least or about 175 copies on a surface of the extracellular vesicle. In some embodiments, the second fusion protein is expressed at a valency of at least or about 200 copies on a surface of the extracellular vesicle. In some embodiments, the second fusion protein is expressed at a valency of at least or about 225 copies on a surface of the extracellular vesicle. In some embodiments, the second fusion protein is expressed at a valency of at least or about 250 copies on a surface of the extracellular vesicle. In some embodiments, the second fusion protein is expressed at a valency of at least or about 275 copies on a surface of the extracellular vesicle. In some embodiments, the second fusion protein is expressed at a valency of at least or about 300 copies on a surface of the extracellular vesicle. In some embodiments, the second fusion protein is expressed at a valency of at least or about 400 copies on a surface of the extracellular vesicle. In some embodiments, the second fusion protein is expressed at a valency of at least or about 500 copies on a surface of the extracellular vesicle. In some embodiments, the second fusion protein is expressed at a valency of at least or about 600 copies on a surface of the extracellular vesicle. In some embodiments, the second fusion protein is expressed at a valency of at least or about 700 copies on a surface of the extracellular vesicle. In some embodiments, the second fusion protein is expressed at a valency of at least or about 800 copies on a surface of the extracellular vesicle. In some embodiments, the second fusion protein is expressed at a valency of at least or about 900 copies on a surface of the extracellular vesicle. In some embodiments, the second fusion protein is expressed at a valency of at least or about 1000 copies on a surface of the extracellular vesicle. In some embodiments, the second fusion protein is expressed at a valency of at least or about 2000 copies on a surface of the extracellular vesicle.

The exosome, as described herein, in some embodiments, comprises a second fusion protein, wherein the second fusion protein is expressed at multiple copies on a surface of the exosome. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the exosome. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the exosome. In some embodiments, the second fusion protein is expressed at a valency of at least or about 10 copies on a surface of the exosome. In some embodiments, the second fusion protein is expressed at a valency of at least or about 25 copies on a surface of the exosome. In some embodiments, the second fusion protein is expressed at a valency of at least or about 50 copies on a surface of the exosome. In some embodiments, the second fusion protein is expressed at a valency of at least or about 75 copies on a surface of the exosome. In some embodiments, the second fusion protein is expressed at a valency of at least or about 100 copies on a surface of the exosome. In some embodiments, the second fusion protein is expressed at a valency of at least or about 125 copies on a surface of the exosome. In some embodiments, the second fusion protein is expressed at a valency of at least or about 150 copies on a surface of the exosome. In some embodiments, the second fusion protein is expressed at a valency of at least or about 175 copies on a surface of the exosome. In some embodiments, the second fusion protein is expressed at a valency of at least or about 200 copies on a surface of the exosome. In some embodiments, the second fusion protein is expressed at a valency of at least or about 225 copies on a surface of the exosome. In some embodiments, the second fusion protein is expressed at a valency of at least or about 250 copies on a surface of the exosome. In some embodiments, the second fusion protein is expressed at a valency of at least or about 275 copies on a surface of the exosome. In some embodiments, the second fusion protein is expressed at a valency of at least or about 300 copies on a surface of the exosome. In some embodiments, the second fusion protein is expressed at a valency of at least or about 400 copies on a surface of the exosome. In some embodiments, the second fusion protein is expressed at a valency of at least or about 500 copies on a surface of the exosome. In some embodiments, the second fusion protein is expressed at a valency of at least or about 600 copies on a surface of the exosome. In some embodiments, the second fusion protein is expressed at a valency of at least or about 700 copies on a surface of the exosome. In some embodiments, the second fusion protein is expressed at a valency of at least or about 800 copies on a surface of the exosome. In some embodiments, the second fusion protein is expressed at a valency of at least or about 900 copies on a surface of the exosome. In some embodiments, the second fusion protein is expressed at a valency of at least or about 1000 copies on a surface of the exosome. In some embodiments, the second fusion protein is expressed at a valency of at least or about 2000 copies on a surface of the exosome.

The ectosome, as described herein, in some embodiments, comprises a second fusion protein, wherein the second fusion protein is expressed at multiple copies on a surface of the ectosome. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the ectosome. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the ectosome. In some embodiments, the second fusion protein is expressed at a valency of at least or about 10 copies on a surface of the ectosome. In some embodiments, the second fusion protein is expressed at a valency of at least or about 25 copies on a surface of the ectosome. In some embodiments, the second fusion protein is expressed at a valency of at least or about 50 copies on a surface of the ectosome. In some embodiments, the second fusion protein is expressed at a valency of at least or about 75 copies on a surface of the ectosome. In some embodiments, the second fusion protein is expressed at a valency of at least or about 100 copies on a surface of the ectosome. In some embodiments, the second fusion protein is expressed at a valency of at least or about 125 copies on a surface of the ectosome. In some embodiments, the second fusion protein is expressed at a valency of at least or about 150 copies on a surface of the ectosome. In some embodiments, the second fusion protein is expressed at a valency of at least or about 175 copies on a surface of the ectosome. In some embodiments, the second fusion protein is expressed at a valency of at least or about 200 copies on a surface of the ectosome. In some embodiments, the second fusion protein is expressed at a valency of at least or about 225 copies on a surface of the ectosome. In some embodiments, the second fusion protein is expressed at a valency of at least or about 250 copies on a surface of the ectosome. In some embodiments, the second fusion protein is expressed at a valency of at least or about 275 copies on a surface of the ectosome. In some embodiments, the second fusion protein is expressed at a valency of at least or about 300 copies on a surface of the ectosome. In some embodiments, the second fusion protein is expressed at a valency of at least or about 400 copies on a surface of the ectosome. In some embodiments, the second fusion protein is expressed at a valency of at least or about 500 copies on a surface of the ectosome. In some embodiments, the second fusion protein is expressed at a valency of at least or about 600 copies on a surface of the ectosome. In some embodiments, the second fusion protein is expressed at a valency of at least or about 700 copies on a surface of the ectosome. In some embodiments, the second fusion protein is expressed at a valency of at least or about 800 copies on a surface of the ectosome. In some embodiments, the second fusion protein is expressed at a valency of at least or about 900 copies on a surface of the ectosome. In some embodiments, the second fusion protein is expressed at a valency of at least or about 1000 copies on a surface of the ectosome. In some embodiments, the second fusion protein is expressed at a valency of at least or about 2000 copies on a surface of the ectosome.

The virus as described herein, in some embodiments, comprises a second fusion protein, wherein the second fusion protein is expressed at multiple copies on a surface of the virus. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the virus. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the virus. In some embodiments, the second fusion protein is expressed at a valency of at least or about 10 copies on a surface of the virus. In some embodiments, the second fusion protein is expressed at a valency of at least or about 25 copies on a surface of the virus. In some embodiments, the second fusion protein is expressed at a valency of at least or about 50 copies on a surface of the virus. In some embodiments, the second fusion protein is expressed at a valency of at least or about 75 copies on a surface of the virus. In some embodiments, the second fusion protein is expressed at a valency of at least or about 100 copies on a surface of the virus. In some embodiments, the second fusion protein is expressed at a valency of at least or about 125 copies on a surface of the virus. In some embodiments, the second fusion protein is expressed at a valency of at least or about 150 copies on a surface of the virus. In some embodiments, the second fusion protein is expressed at a valency of at least or about 175 copies on a surface of the virus. In some embodiments, the second fusion protein is expressed at a valency of at least or about 200 copies on a surface of the virus. In some embodiments, the second fusion protein is expressed at a valency of at least or about 225 copies on a surface of the virus. In some embodiments, the second fusion protein is expressed at a valency of at least or about 250 copies on a surface of the virus. In some embodiments, the second fusion protein is expressed at a valency of at least or about 275 copies on a surface of the virus. In some embodiments, the second fusion protein is expressed at a valency of at least or about 300 copies on a surface of the virus. In some embodiments, the second fusion protein is expressed at a valency of at least or about 300 copies on a surface of the virus. In some embodiments, the second fusion protein is expressed at a valency of at least or about 400 copies on a surface of the virus. In some embodiments, the second fusion protein is expressed at a valency of at least or about 500 copies on a surface of the virus. In some embodiments, the second fusion protein is expressed at a valency of at least or about 600 copies on a surface of the virus. In some embodiments, the second fusion protein is expressed at a valency of at least or about 700 copies on a surface of the virus. In some embodiments, the second fusion protein is expressed at a valency of at least or about 800 copies on a surface of the virus. In some embodiments, the second fusion protein is expressed at a valency of at least or about 900 copies on a surface of the virus. In some embodiments, the second fusion protein is expressed at a valency of at least or about 1000 copies on a surface of the virus. In some embodiments, the second fusion protein is expressed at a valency of at least or about 2000 copies on a surface of the virus.

The replication incompetent virus as described herein, in some embodiments, comprises a second fusion protein, wherein the second fusion protein is expressed at multiple copies on a surface of the replication incompetent virus. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication incompetent virus. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication incompetent virus. In some embodiments, the second fusion protein is expressed at a valency of at least or about 10 copies on a surface of the replication incompetent virus. In some embodiments, the second fusion protein is expressed at a valency of at least or about 25 copies on a surface of the replication incompetent virus. In some embodiments, the second fusion protein is expressed at a valency of at least or about 50 copies on a surface of the replication incompetent virus. In some embodiments, the second fusion protein is expressed at a valency of at least or about 75 copies on a surface of the replication incompetent virus. In some embodiments, the second fusion protein is expressed at a valency of at least or about 100 copies on a surface of the replication incompetent virus. In some embodiments, the second fusion protein is expressed at a valency of at least or about 125 copies on a surface of the replication incompetent virus. In some embodiments, the second fusion protein is expressed at a valency of at least or about 150 copies on a surface of the replication incompetent virus. In some embodiments, the second fusion protein is expressed at a valency of at least or about 175 copies on a surface of the replication incompetent virus. In some embodiments, the second fusion protein is expressed at a valency of at least or about 200 copies on a surface of the replication incompetent virus. In some embodiments, the second fusion protein is expressed at a valency of at least or about 225 copies on a surface of the replication incompetent virus. In some embodiments, the second fusion protein is expressed at a valency of at least or about 250 copies on a surface of the replication incompetent virus. In some embodiments, the second fusion protein is expressed at a valency of at least or about 275 copies on a surface of the replication incompetent virus. In some embodiments, the second fusion protein is expressed at a valency of at least or about 300 copies on a surface of the replication incompetent virus. In some embodiments, the second fusion protein is expressed at a valency of at least or about 300 copies on a surface of the replication incompetent virus. In some embodiments, the second fusion protein is expressed at a valency of at least or about 400 copies on a surface of the replication incompetent virus. In some embodiments, the second fusion protein is expressed at a valency of at least or about 500 copies on a surface of the replication incompetent virus. In some embodiments, the second fusion protein is expressed at a valency of at least or about 600 copies on a surface of the replication incompetent virus. In some embodiments, the second fusion protein is expressed at a valency of at least or about 700 copies on a surface of the replication incompetent virus. In some embodiments, the second fusion protein is expressed at a valency of at least or about 800 copies on a surface of the replication incompetent virus. In some embodiments, the second fusion protein is expressed at a valency of at least or about 900 copies on a surface of the replication incompetent virus. In some embodiments, the second fusion protein is expressed at a valency of at least or about 1000 copies on a surface of the replication incompetent virus. In some embodiments, the second fusion protein is expressed at a valency of at least or about 2000 copies on a surface of the replication incompetent virus.

The replication competent virus as described herein, in some embodiments, comprises a second fusion protein, wherein the second fusion protein is expressed at multiple copies on a surface of the replication competent virus. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication competent virus. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication competent virus. In some embodiments, the second fusion protein is expressed at a valency of at least or about 10 copies on a surface of the replication competent virus. In some embodiments, the second fusion protein is expressed at a valency of at least or about 25 copies on a surface of the replication competent virus. In some embodiments, the second fusion protein is expressed at a valency of at least or about 50 copies on a surface of the replication competent virus. In some embodiments, the second fusion protein is expressed at a valency of at least or about 75 copies on a surface of the replication competent virus. In some embodiments, the second fusion protein is expressed at a valency of at least or about 100 copies on a surface of the replication competent virus. In some embodiments, the second fusion protein is expressed at a valency of at least or about 125 copies on a surface of the replication competent virus. In some embodiments, the second fusion protein is expressed at a valency of at least or about 150 copies on a surface of the replication competent virus. In some embodiments, the second fusion protein is expressed at a valency of at least or about 175 copies on a surface of the replication competent virus. In some embodiments, the second fusion protein is expressed at a valency of at least or about 200 copies on a surface of the replication competent virus. In some embodiments, the second fusion protein is expressed at a valency of at least or about 225 copies on a surface of the replication competent virus. In some embodiments, the second fusion protein is expressed at a valency of at least or about 250 copies on a surface of the replication competent virus. In some embodiments, the second fusion protein is expressed at a valency of at least or about 275 copies on a surface of the replication competent virus. In some embodiments, the second fusion protein is expressed at a valency of at least or about 300 copies on a surface of the replication competent virus. In some embodiments, the second fusion protein is expressed at a valency of at least or about 300 copies on a surface of the replication competent virus. In some embodiments, the second fusion protein is expressed at a valency of at least or about 400 copies on a surface of the replication competent virus. In some embodiments, the second fusion protein is expressed at a valency of at least or about 500 copies on a surface of the replication competent virus. In some embodiments, the second fusion protein is expressed at a valency of at least or about 600 copies on a surface of the replication competent virus. In some embodiments, the second fusion protein is expressed at a valency of at least or about 700 copies on a surface of the replication competent virus. In some embodiments, the second fusion protein is expressed at a valency of at least or about 800 copies on a surface of the replication competent virus. In some embodiments, the second fusion protein is expressed at a valency of at least or about 900 copies on a surface of the replication competent virus. In some embodiments, the second fusion protein is expressed at a valency of at least or about 1000 copies on a surface of the replication competent virus. In some embodiments, the second fusion protein is expressed at a valency of at least or about 2000 copies on a surface of the replication competent virus.

Described herein, in some embodiments, are multivalent particles comprising improved binding properties. In some embodiments, the multivalent particles comprise a binding affinity (e.g., K_{D}) to the viral protein or target cell of less than 100 pM, less than 200 pM, less than 300 pM, less than 400 pM, less than 500 pM, less than 600 pM, less than 700 pM, less than 800 pM, or less than 900 pM In some embodiments, the multivalent particle comprises a K_{D} of less than 1 nM, less than 1.2 nM, less than 2 nM, less than 5 nM, or less than 10 nM. In some instances, the multivalent particle comprises a K_{D} of less than 1 nM. In some instances, the multivalent particle comprises a K_{D} of less than 1.2 nM. In some instances, the multivalent particle comprises a K_{D} of less than 2 nM. In some instances, the multivalent particle comprises a K_{D} of less than 5 nM. In some instances, the multivalent particle comprises a K_{D} of less than 10 nM.

In some embodiments, the multivalent particle comprises an IC₅₀ of less than 20 picomolar (pM) in a neutralization assay. In some embodiments, the multivalent particle comprises an IC₅₀ of less than 15 picomolar (pM) in a neutralization assay. In some embodiments, the multivalent particle comprises an IC₅₀ of less than 10 picomolar (pM) in a neutralization assay. In some embodiments, the multivalent particle comprises an IC₅₀ of less than 5 picomolar (pM) in a neutralization assay. In some embodiments, the multivalent particle comprises an IC₅₀ of less than 2.5 picomolar (pM) in a neutralization assay. In some embodiments, the multivalent particle comprises an IC₅₀ of less than 1 picomolar (pM) in a neutralization assay. In some embodiments, the multivalent particle comprises an IC₅₀ of less than 0.5 picomolar (pM) in a neutralization assay.

### Compositions and Methods for Generation of IFN-MVPs

Disclosed herein are compositions comprising a nucleic acid sequence that encodes any of the first fusion proteins disclosed herein. Disclosed herein are compositions comprising a nucleic acid sequence that encodes the first fusion proteins disclosed herein and any of the second fusion proteins disclosed herein. Disclosed herein are compositions comprising a DNA sequence that encodes the first fusion proteins disclosed herein. Disclosed herein are compositions comprising an mRNA sequence that encodes the first fusion proteins disclosed herein. Disclosed herein are compositions comprising a DNA sequence that encodes the first fusion proteins disclosed herein and any of the second fusion proteins disclosed herein. Disclosed herein are compositions comprising an mRNA sequence that encodes the first fusion proteins disclosed herein and any of the second fusion proteins disclosed herein.

In some embodiments, the composition further comprises a second nucleic acid sequence that encodes one or more packaging viral proteins. In some embodiments, the one or more packaging viral proteins comprises a lentiviral protein, a retroviral protein, an adenoviral protein, or combinations thereof. In some embodiments, the one or more packaging viral proteins comprises gag, pol, pre, tat, rev, or combinations thereof. In some embodiments, the composition further comprises a third nucleic acid sequence that encodes a reporter, a therapeutic molecule, or combinations thereof.

In some embodiments, the reporter protein comprises a fluorescent protein or an enzyme. Exemplary reporter genes include, but are not limited to, acetohydroxyacid synthase (AHAS), alkaline phosphatase (AP), beta galactosidase (LacZ), beta glucuronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), and derivatives thereof. Methods to determine modulation of a reporter gene are well known in the art, and include, but are not limited to, fluorometric methods (e.g., fluorescence spectroscopy, Fluorescence Activated Cell Sorting (FACS), fluorescence microscopy), and antibiotic resistance determination. In some embodiments, the reporter comprises a fluorescent protein. In some embodiments, the fluorescent protein comprises green fluorescent protein. In some embodiments, the reporter protein emits green fluorescence, yellow fluorescence, or red fluorescence. In some embodiments, the reporter comprises an enzyme. In some embodiments, the enzyme comprises β-galactosidase, alkaline phosphatase, β-lactamase, or luciferase.

In some embodiments, the therapeutic molecule comprises an immune modulating protein, a cellular signal modulating molecule, a proliferation modulating molecule, or combinations thereof. In some embodiments, the therapeutic molecule comprises an immune checkpoint molecule. Exemplary immune checkpoint molecules include, but are not limited to, CTLA4, PD1, OX40, and CD28. In some embodiments, the therapeutic molecule comprises an inflammatory cytokine. In some embodiments, the inflammatory cytokine comprises IL-12, IL-18, TNF-alpha, or TNF-beta. In some embodiments, the therapeutic molecule comprises a proliferation cytokine. In some embodiments, the proliferation cytokine comprises IL-7, or IL-15, IL-21.

In some embodiments, a nucleic acid sequence encoding the first fusion protein and a nucleic acid sequence encoding the second fusion protein are within a same vector. In some embodiments, a nucleic acid sequence encoding the first fusion protein and a nucleic acid sequence encoding the second fusion protein are within different vectors. In some embodiments, the first nucleic acid sequence, the second nucleic acid sequence, and the third nucleic acid sequence are within a same vector. In some embodiments, the first nucleic acid sequence, the second nucleic acid sequence, and the third nucleic acid sequence are within different vectors. In some embodiments, the first nucleic acid sequence, the second nucleic acid sequence, the third nucleic acid sequence, and the fourth nucleic acid sequence are within a same vector. In some embodiments, the first nucleic acid sequence, the second nucleic acid sequence, third nucleic acid sequence, and the fourth nucleic acid sequence are within different vectors. In some embodiments, the first nucleic acid sequence, the second nucleic acid sequence, the third nucleic acid sequence, and the fourth nucleic acid sequence are DNA sequences. In some embodiments, the first nucleic acid sequence, the second nucleic acid sequence, the third nucleic acid sequence, and the fourth nucleic acid sequence are mRNA sequences.

Various vectors, in some embodiments, are used herein. In some embodiments, the vector comprises a eukaryotic or prokaryotic vector. In some embodiments, the vector comprises a viral vector. In some embodiments, the vector comprises a lentivirus vector, an adenovirus vector, or an adeno-associated virus vector. Exemplary vectors include, without limitation, mammalian expression vectors: pSF-CMV-NEO-NH2-PPT-3XFLAG, pSF-CMV-NEO-COOH-3XFLAG, pSF-CMV-PURO-NH2-GST-TEV, pSF-OXB20-COOH-TEV-FLAG (R)-6His, pCEP4 pDEST27, pSF-CMV-Ub-KrYFP, pSF-CMV-FMDV-daGFP, pEF1a-mCherry-N1 Vector, pEF1a-tdTomato Vector, pSF-CMV-FMDV-Hygro, pSF-CMV-PGK-Puro, pMCP-tag (m), and pSF-CMV-PURO-NH2-CMYC; bacterial expression vectors: pSF-OXB20-BetaGal, pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8.

Described herein, in some embodiments, are methods of treating a cancer in a subject in need thereof comprising administering to the subject a composition, wherein the composition comprises a nucleic acid sequence that encodes a first fusion protein disclosed herein. Described herein, in some embodiments, are methods of treating a cancer in a subject in need thereof comprising administering to the subject a composition, wherein the composition comprises a nucleic acid sequence that encodes a first fusion protein disclosed herein and a second fusion protein disclosed herein. In some embodiments, the cancer comprises melanoma, leukemia, lymphoma, multiple myeloma, liver cancer, pancreatic cancer, lung cancer, breast cancer, prostate cancer, brain cancer, colorectal cancer, bladder cancer, kidney cancer, cervical cancer, ovarian cancer, esophageal cancer, mesothelioma, gastric cancer, and sarcoma. In some embodiments, the nucleic acid sequence comprises mRNA. In some embodiments, the nucleic acid sequence comprises DNA.

Described herein, in some embodiments, are methods of treating an infection in a subject in need thereof comprising administering to the subject a composition, wherein the composition comprises a nucleic acid sequence that encodes a first fusion protein disclosed herein. Described herein, in some embodiments, are methods of treating an infection in a subject in need thereof comprising administering to the subject a composition, wherein the composition comprises a nucleic acid sequence that encodes a first fusion protein disclosed herein and a second fusion protein disclosed herein. In some embodiments, the infection comprises infection by hepatitis B virus (HBV), SARS CoV-2, SARS CoV-1, MERS CoV, Influenza, respiratory syncytial virus, HIV, or measles. In some embodiments, the nucleic acid sequence comprises mRNA. In some embodiments, the nucleic acid sequence comprises DNA.

Described herein, in some embodiments, are methods of treating an autoimmune or an inflammatory disease in a subject in need thereof comprising administering to the subject a composition, wherein the composition comprises a nucleic acid sequence that encodes a first fusion protein. Described herein, in some embodiments, are methods of treating an autoimmune or an inflammatory disease in a subject in need thereof comprising administering to the subject a composition, wherein the composition comprises a nucleic acid sequence that encodes a first fusion protein disclosed herein and a second fusion protein disclosed herein. In some embodiments, the autoimmune disease comprises rheumatoid arthritis, systemic lupus erythematosus, multiple sclerosis, inflammatory bowel diseases, psoriasis, or aplastic anemia. In some embodiments, the nucleic acid sequence comprises mRNA. In some embodiments, the nucleic acid sequence comprises DNA.

In some embodiments, the composition is administered to the subject through inhalation. In some embodiments, the composition is administered to the subject through intranasal delivery. In some embodiments, the composition is administered to the subject through intratracheal delivery. In some embodiments, the composition is administered to the subject by an intraperitoneal injection. In some embodiments, the composition is administered to the subject by a subcutaneous injection. In some embodiments, the administering to the subject of the composition is sufficient to reduce or eliminate the cancer as compared to a baseline measurement of the cancer taken from the subject prior to the administering of the composition. In some embodiments, the administering to the subject of the composition is sufficient to reduce or eliminate the infection as compared to a baseline measurement of the infection taken from the subject prior to the administering of the composition. In some embodiments, the administering to the subject of the composition is sufficient to reduce or eliminate a symptom of the autoimmune disorder as compared to a baseline measurement of the symptom as measured from the subject prior to the administering of the composition. In some embodiments, the reduction is at least about 1-fold, 5-fold, 10-fold, 20-fold, 40-fold, 60-fold, 80-fold, or up to about 100-fold.

In some embodiments, the composition is administered with a liposome. In some embodiments, the liposome is a protamine liposome. In some embodiments, the liposome is a cationic polymer liposome. In some embodiments, the composition is administered with a lipid nanoparticle. In some embodiments, the composition is administered with a cationic lipid nanoparticle. In some embodiments, the composition is administered with a cationic lipid, cholesterol nanoparticle. In some embodiments, the composition is administered with a cationic lipid, cholesterol, PEG nanoparticle. In some embodiments, the composition is administered with a dendrimer nanoparticle. In some embodiments, the composition is administered as a naked nucleic acid sequence. In some embodiments, the composition is administered as a naked DNA sequence. In some embodiments, the composition is administered as a naked mRNA sequence.

In some embodiments, the composition is administered with an adeno-associated virus (AAV). In some embodiments, the composition is administered with a polymer. In some embodiments, the composition is administered with protamine. In some embodiments, the composition is administered with a polysaccharide particle. In some embodiments, the composition is administered with a cationic polymer. In some embodiments, the composition is administered with a cationic nano-emulsion. In some embodiments, the composition is administered with a transfection reagent. In some embodiments, the composition is administered with a dendritic cell.

### Pharmaceutical Compositions

Described herein, in some embodiments, are compositions comprising multivalent particles described herein. In some embodiments, the composition may be used to treat a disease disclosed herein, including but not limited to cancer, infection, or an autoimmune disease. For administration to a subject, the multivalent particles as disclosed herein, may be provided in a pharmaceutical composition together with one or more pharmaceutically acceptable carriers or excipients. In some embodiments, the multivalent particles as disclosed herein, may be provided in a composition together with one or more carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

Disclosed herein, in some embodiments, is a composition comprising a multivalent interferon particle (IFN-MVP) that comprises an enveloped particle displaying at least 10 copies of an interferon (IFN) on a surface of the IFN-MVP.

Disclosed herein, in some embodiments, is a composition comprising a guided IFN-MVP wherein the guided IFN-MVP comprises an enveloped particle that co-displays at least 10 copies of an IFN and at least 10 copies of a homing polypeptide on a surface of the IFN-MVP.

Disclosed herein, in some embodiments, is a composition comprising an antibody-guided IFN-MVP wherein the antibody-guided IFN-MVP comprises an enveloped particle that co-displays at least 10 copies of an IFN and at least 10 copies of an antibody on a surface of the IFN-MVP wherein the antibody binds specifically to an antigen on a target cell or a target viral protein.

Disclosed herein, in some embodiments, is a composition comprising a receptor-guided IFN-MVP wherein the receptor-guided IFN-MVP comprises an enveloped particle that co-displays at least 10 copies of an IFN and at least 10 copies of a receptor on a surface of the IFN-MVP wherein the receptor binds specifically to a target ligand.

Disclosed herein, in some embodiments, is a composition comprising a ligand-guided IFN-MVP wherein the ligand-guided IFN-MVP comprises an enveloped particle that co-displays at least 10 copies of an IFN and at least 10 copies of a ligand on a surface of the IFN-MVP wherein the ligand binds specifically to a target receptor.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, intravenous, or inhalation) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Methods of Treatment

Described herein are methods of treating cancer in a subject in need thereof comprising administering to the subject a multivalent particle described herein or a pharmaceutical composition comprising a multivalent particle disclosed herein. Described herein are methods of treating cancer in a subject in need thereof comprising expressing an IFN on a surface of a multivalent particle, wherein the multivalent particle has a binding affinity to an IFN receptor that is higher than the binding affinity of a soluble version of the IFN to the IFN receptor. In some embodiments, the cancer comprises melanoma, leukemia, lymphoma, multiple myeloma, liver cancer, pancreatic cancer, lung cancer, breast cancer, prostate cancer, brain cancer, colorectal cancer, bladder cancer, kidney cancer, cervical cancer, ovarian cancer, esophageal cancer, mesothelioma, gastric cancer, and sarcoma. In some embodiments, the multivalent particle is administered to the subject through inhalation. In some embodiments, the multivalent particle is administered to the subject through intranasal delivery. In some embodiments, the multivalent particle is administered to the subject through intratracheal delivery. In some embodiments, the multivalent particle is administered to the subject by an intraperitoneal injection. In some embodiments, the multivalent particle is administered to the subject by a subcutaneous injection. In some embodiments, the administering to the subject of the multivalent particle is sufficient to reduce or eliminate the cancer as compared to a baseline measurement of the cancer taken from the subject prior to the administering of the multivalent particle.

Described herein are methods of treating an infection in a subject in need thereof comprising administering to the subject a multivalent particle described herein or a pharmaceutical composition comprising a multivalent particle disclosed herein. Described herein are methods of treating hepatitis B virus (HBV), SARS CoV-2, SARS CoV-1, MERS CoV, Influenza, respiratory syncytial virus, HIV, or measles in a subject in need thereof comprising administering to the subject a multivalent particle described herein or a pharmaceutical composition comprising a multivalent particle disclosed herein. Described herein are methods of treating an infection in a subject in need thereof comprising expressing an IFN on a surface of a multivalent particle, wherein the multivalent particle has a binding affinity to an IFN receptor that is higher than the binding affinity of a soluble version of the IFN to the IFN receptor. Described herein are methods of treating hepatitis B virus (HBV), SARS CoV-2, SARS CoV-1, MERS CoV, Influenza, respiratory syncytial virus, HIV, or measles in a subject in need thereof comprising expressing an IFN on a surface of a multivalent particle, wherein the multivalent particle has a binding affinity to an IFN receptor that is higher than the binding affinity of a soluble version of the IFN to the IFN receptor. In some embodiments, the multivalent particle is administered to the subject intravenously. In some embodiments, the multivalent particle is administered to the subject through inhalation. In some embodiments, the multivalent particle is administered to the subject through intranasal delivery. In some embodiments, the multivalent particle is administered to the subject through intratracheal delivery. In some embodiments, the multivalent particle is administered to the subject by an intraperitoneal injection. In some embodiments, the multivalent particle is administered to the subject by a subcutaneous injection. In some embodiments, the multivalent particle induces T cell mediated cytotoxicity against viral infected cells. In some embodiments, the administering to the subject of the multivalent particle is sufficient to reduce or eliminate the hepatitis B virus (HBV), SARS CoV-2, SARS CoV-1, MERS CoV, Influenza, respiratory syncytial virus, HIV, or measles, as compared to a baseline measurement of the HBV taken from the subject prior to the administering of the multivalent particle. In some embodiments, the reduction is at least about 1-fold, 5-fold, 10-fold, 20-fold, 40-fold, 60-fold, 80-fold, or up to about 100-fold.

Described herein, in some embodiments, are methods of treating an autoimmune disease or an inflammatory disease in a subject in need thereof comprising administering to the subject a multivalent particle described herein or a pharmaceutical composition comprising a multivalent particle disclosed herein. Described herein are methods of treating an autoimmune disease or an inflammatory disease in a subject in need thereof comprising expressing an IFN on a surface of a multivalent particle, wherein the multivalent particle has a binding affinity to an IFN receptor that is higher than the binding affinity of a soluble version of the IFN to the IFN receptor. In some embodiments, the autoimmune disease comprises rheumatoid arthritis, systemic lupus erythematosus, multiple sclerosis, inflammatory bowel diseases, psoriasis, or aplastic anemia. In some embodiments, the multivalent particle is administered to the subject intravenously. In some embodiments, the multivalent particle is administered to the subject through inhalation. In some embodiments, the multivalent particle is administered to the subject through intranasal delivery. In some embodiments, the multivalent particle is administered to the subject through intratracheal delivery. In some embodiments, the multivalent particle is administered to the subject by an intraperitoneal injection. In some embodiments, the multivalent particle is administered to the subject by a subcutaneous injection. In some embodiments, the multivalent particle induces T cell mediated cytotoxicity against viral infected cells. In some embodiments, the administering to the subject of the multivalent particle is sufficient to reduce or eliminate a symptom of the autoimmune disease as compared to a baseline measurement of the symptom of the autoimmune disease measured from the subject prior to the administering of the multivalent particle. In some embodiments, the reduction is at least about 1-fold, 5-fold, 10-fold, 20-fold, 40-fold, 60-fold, 80-fold, or up to about 100-fold.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Materials and Methods

Antibody, IFN and receptor display constructs: Codon-optimized antibody (Ab), receptor and interferon (IFN) sequences were synthesized and cloned into specialized display constructs expressing fusion peptides consisting of the displayed protein ectodomain fused to a sequence encoding the transmembrane and cytoplasmic tail domains of VSV-G protein. To generate IFN-MVPs displaying oligomerized Abs and IEs, the extracellular domains of displayed peptides were fused to a synthetic VSV-G sequence encoding the D4 post-fusion trimerization domain and the transmembrane and cytoplasmic tail domains.

Production of IFN-MVPs as VLPs and EVs: IFN-MVPs based on VLPs and EVs were produced from transfected HEK 293T cells. To produce lentiviral VLP-based IFN-MVPs with viral genomes, HEK 293T cells were co-transfected with Ab and IE display constructs, along with a lentiviral packaging vector (i.e., psPAX2) and a lentiviral genome transfer vector. To produce lentiviral VLP-based IFN-MVPs without viral genomes, HEK 293T cells were co-transfected with Ab and IE display constructs, along with a lentiviral packaging vector (i.e., psPAX2). To produce extracellular vesicle-based IFN-MVPs, HEK 293T cells were transfected with Ab and IE display constructs alone.

Transfections were prepared by culturing 7.5×10⁶ HEK293T cells (ATCC CRL-3216) overnight in 10-cm dishes containing DMEM media with glucose, L-glutamine and sodium pyruvate supplemented with 10% fetal bovine serum and 1% Penicillin Streptomycin, referred to as “293T Growth Media.” The cells reach ≈90% confluence after 24 hours. A transfection DNA mixture was prepared with polyethylenimine (PEI) in OPTI-MEM reduced serum medium. The transfection mixture was incubated at room temperature for 15 minutes before being added to cells, which were then incubated at 37° C. in 5% CO₂. 6 hours post-transfection, 293T Growth Media was changed to 293T Growth Media supplemented with 0.1% sodium butyrate (referred to as “Transfection Media”) before being returned to incubation. After incubating for 24 hours at 37° C. with 5% CO₂ in Transfection Media, supernatant containing VLPs or EVs were collected, centrifuged at 1680 rpm for 5 minutes to remove cellular debris and mixed with 1× polyethylene glycol 8000 solution, before being stored at 4° C. for 24 hours to allow fractionation. Cells were replenished with fresh Transfection Media, and a second particle supernatant collection was performed at 48 hours. Supernatant collections were then pooled, PEG precipitated, and purified by size exclusion chromatography using Sephacryl S-300 High Resolution Beads.

Lentiviral and EV particle quantification by p24 ELISA and Tunable Resistive Pulse Sensing: P24 concentrations in VLP samples of IFN-MVPs were determined using an HIV p24 SimpleStep ELISA kit. Concentrations of lentiviral particles were extrapolated from the assumption that each lentiviral particle contains approximately 2000 molecules of p24, or 1.25×10⁴ pseudovirus particles per picogram of p24 protein.

EV-based IFN-MVPs were quantified via tunable resistive pulse sensing (TRPS). Purified EV collections were diluted in 0.2 μm-filtered PBS with 0.03% Tween-20 prior to qNano analysis. Concentration and size distributions of EV particles were then determined using an NP200 nanopore at a 45.5 mm stretch, and applied voltages between 0.5 and 0.7V were used to achieve a stable current of 130 nA through the nanopore. Measurements for each EV sample were taken at pressures of 3, 5 and 8 mbar, and considered valid if at least 500 events were recorded. The particle rate is linear and root mean squared signal noise was maintained below 10 pA. EV concentrations were then determined by comparison to a standardized multi-pressure calibration using CPC200 (mode diameter: 200 nm) carboxylated polystyrene beads diluted 1:200 in 0.2 μM-filtered PBS from an original concentration of 7.3×10¹¹ particles per/mL. Measurements were analyzed using IZON Control Suite 3.4 software to determine original sample concentrations.

Western blot analysis of IFN-MVPs: Expression of IFN, Ab and receptor fusion proteins on IFN-MVP VLPs and EVs was confirmed via western blot analysis of purified particles. Samples of purified IFN-MVPs were lysed at 4° C. for 10 minutes with cell lysis buffer before being mixed with NuPage LDS sample buffer and boiled at 95° C. for 5 minutes. Differences in oligomerization were determined by running samples in reducing and non-reducing conditions. Under reducing conditions, 5% 2-Mercaptoethanol was added to samples to dissociate oligomerized fusion display proteins on IFN-MVPs. Protein samples were then separated on NuPAGE 4-12% Bis-Tris gels and transferred onto a polyvinylidene fluoride (PVDF) membrane. PVDF membranes were blocked with TRIS-buffered saline with Tween-20 (TBST) and 5% skim milk for 1 hour, prior to overnight incubation with primary antibody diluted in 5% milk. For fusion display constructs expressing VSVG-tag, an anti-VSV-G epitope tag rabbit polyclonal antibody was used at a 1:2000 dilution. The following day, the PVDF membrane was washed 3 times with 1×TBST and stained with a goat-anti-rabbit secondary antibody at a 1:5000 dilution for 60 minutes in 5% milk. Post-secondary antibody staining, the PVDF membrane was again washed 3 times with TBST before imaging on a Licor Odyssey scanner.

Alternatively, western blot analyses were performed using an automated Simple Western size-based protein assay. Concentrated samples were lysed as described above, before being diluted 1:10 in 0.1× sample buffer for loading on capillaries. Ab, receptor and IFN fusion display protein expression levels were identified using the same primary rabbit polyclonal antibody at a 1:400 dilution and an HRP conjugated anti-rabbit secondary antibody. Chemiluminescence signal analysis and absolute quantitation were performed using Compass software.

Quantitative western blot analysis of IFN-MVPs: Quantitative western blot analyses were performed to determine the copies of displayed IFN, Ab and receptor fusion proteins displayed per particle. P24 ELISA or TRPS assays were used to determine IFN-MVP sample concentrations. Purified IFN-MVP samples were processed and analyzed via western blot under reducing conditions as described above. A reference decoy-Antivirus with a known display copy number or a synthetic protein standard of known concentration was used to generate a standard curve, from which copy numbers of displayed immune checkpoint on respective particles is determined.

In vitro IFN cellular reporter assay: HEK-Blue IFN reporter cells (Invivogen) were cultured in growth medium containing DMEM, 10% 60° C. heat inactivated FBS, 100U/mL penicillin streptomycin and 100 μg/mL Normocin. After 2 passages, 30 μg/mL Blasticidin and 100 μg/mL Zeocin were added to growth medium as selection antibiotics. Cells were passaged twice in selection medium before use in reporter assays. On the day of assay setup, cells were trypsinized, resuspended in growth medium without Blasticidin and Zeocin and seeded at a density of 5×10⁴ cells per well in 96 well plates. Serial dilutions of soluble IFNs, controls and IFN-MVPs were made using growth medium in PCR strip tubes before being added to cells and gently pipet mixed. Cells were then incubated overnight at 37° C., in 5% CO2 for 24 hours. Following incubation, QB SEAP reporter solution was prepared using 1 mL QB buffer (Invivogen), 1 mL QB reagent (Invivogen) and 98 mL DI H₂O. Solution was vortexed and incubated for 10 minutes at room temperature. 180 μL of QB SEAP reporter solution was then transferred to each well of a transparent 96 well plate. Finally, 20 μL of cell culture supernatants from cultured HEK-Blue IFN reporter cells was transferred to wells containing QB SEAP reporter solution and pipet mixed gently. After incubating at room temperature for 10 minutes, optical density of wells was determined via BMG Omega reader.

### Example 2: Design and Production of IFN-MVPs

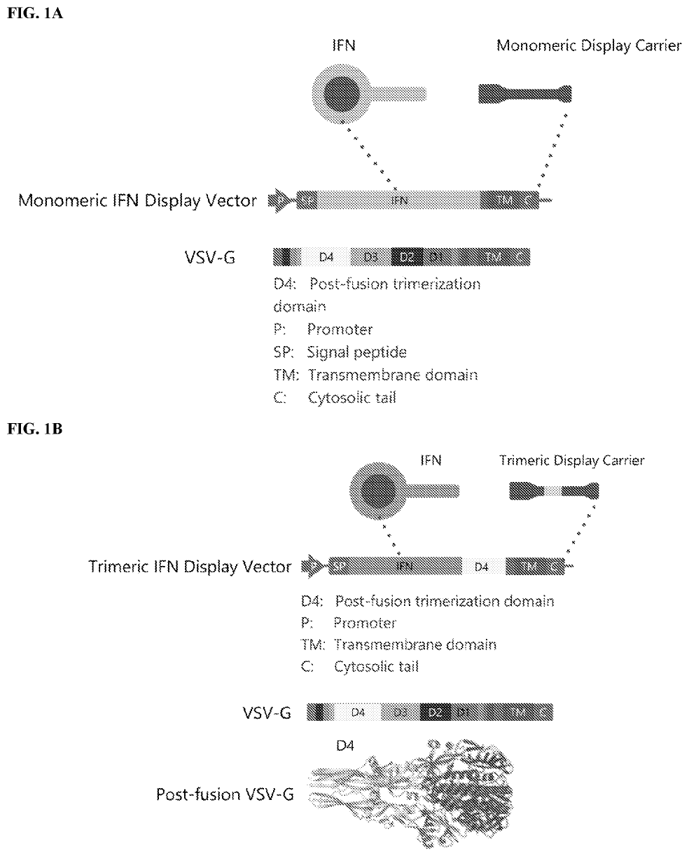

Design of IFN-MVPs: Two different forms of display vectors were designed for displaying IFNs, antibodies and receptors in monomeric and trimeric form on the surface of IFN-MVP vesicles. Monomeric display vectors express a fusion protein consisting of the protein of interest (IFN/Ab/receptor) linked to the transmembrane and intracellular domains of VSV-G protein (FIG. 1A). Trimeric antibody display constructs express a fusion protein consisting of the protein of interest linked to the D4 post-fusion trimerization, transmembrane and intracellular domains of VSV-G protein (FIG. 1B).

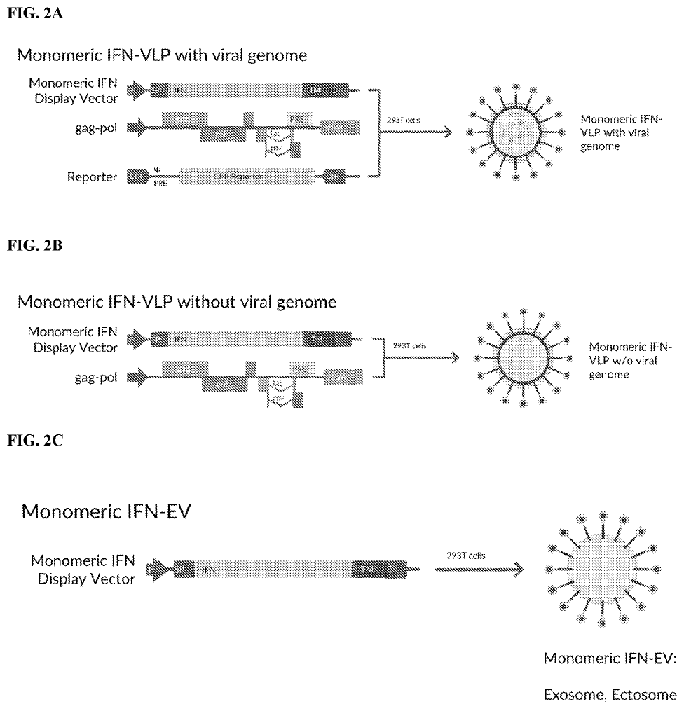

Production of monomeric IFN-MVPs: IFN-MVPs displaying a single type of IFN as monomers were produced as VLPs with or without genomes, and as extracellular vesicles (EVs) such as exosomes and ectosomes. To produce monomeric IFN-MVPs as VLPs containing RNA genomes, HEK 293T cells were co-transfected with monomeric IFN display constructs along with a lentiviral packaging construct expressing essential packaging components, such as Gag-Pol and Rev proteins, and a viral genome transfer vector encoding a GFP/luciferase reporter (FIG. 2A). To produce monomeric IFN-MVPs as VLPs without genomes, HEK 293T cells were co-transfected with monomeric IFN-display vectors and a lentiviral packaging construct, without a viral genome transfer vector (FIG. 2B). Finally, to produce monomeric IFN-MVPs as EVs, 293T cells were transfected with monomeric IFN display constructs alone (FIG. 2C).

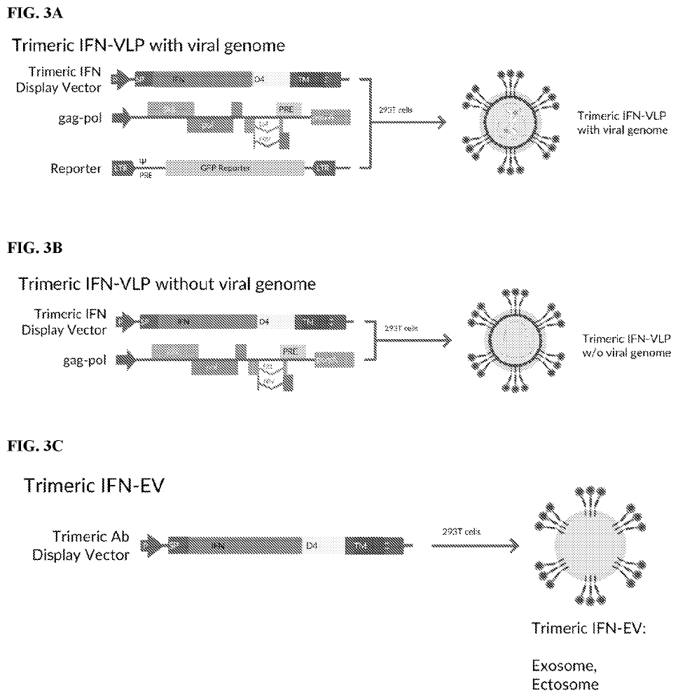

Production of trimeric IFN-MVPs: IFN-MVPs displaying a single type of IFN as trimers were produced as VLPs with or without genomes and as extracellular vesicles (EVs). To produce trimeric IFN-MVPs as VLPs containing RNA genomes, HEK 293T cells were co-transfected with trimeric IFN display constructs along with a lentiviral packaging construct expressing essential packaging components, such as Gag-Pol and Rev proteins, and a viral genome transfer vector encoding a GFP/luciferase reporter (FIG. 3A). To produce trimeric IFN-MVPs as VLPs without genomes, HEK 293T are were co-transfected with trimeric IFN-display vectors and a lentiviral packaging construct, without a viral genome transfer vector (FIG. 3B). Finally, to produce trimeric IFN-MVPs as EVs, 293T cells are transfected with trimeric IFN display constructs alone (FIG. 3C).

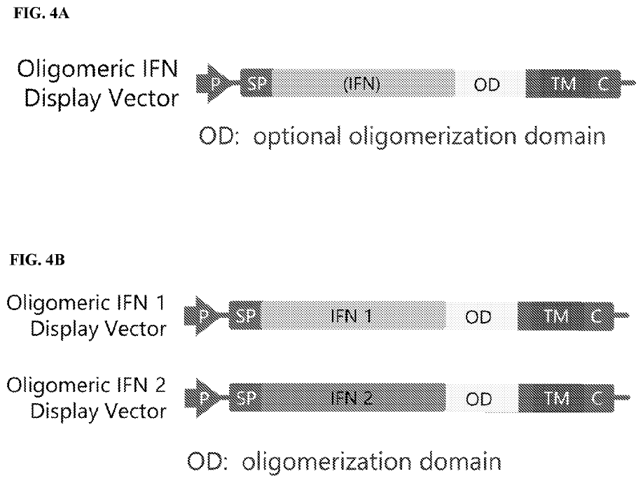

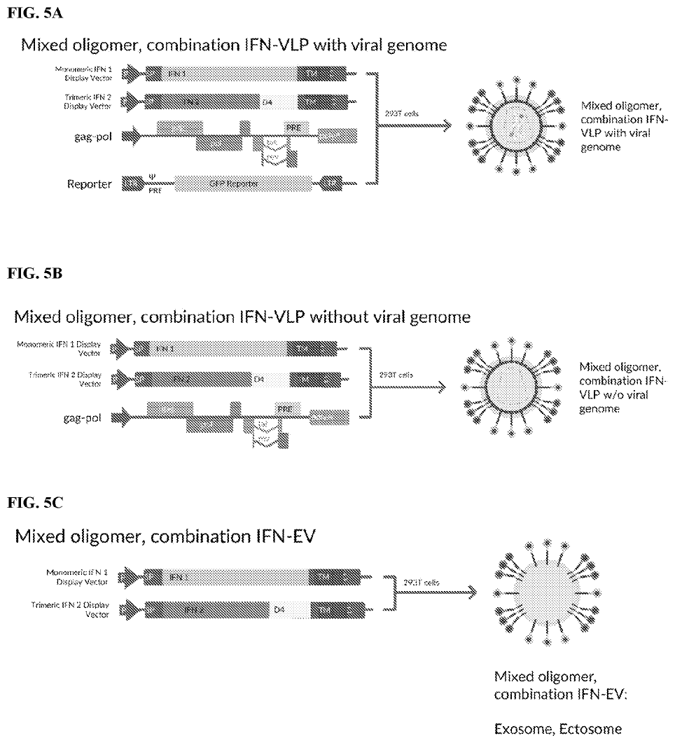

Production of mixed oligomer, multi-type IFN-MVPs: Mixed oligomer, multi-type IFN-MVPs co-displaying different types of IFNs in both monomeric and trimeric conformations are produced. The IFN-MVPs co-display combinations of distinct interferons (FIG. 4A & Table 8) in combinations of oligomeric display patterns (FIG. 4B & Table 9) and simultaneously stimulate multiple IFN signaling pathways within contacted cells. Like single-type IFN-MVPs, the mixed oligomer, multi-type IFN-MVPs can be designed atop VLPs and EVs. To produce multi-type IFN-MVPs as VLPs containing RNA genomes, HEK 293T cells are co-transfected with the desired combination of distinct IFN display constructs, as well as a lentiviral packaging construct expressing essential packaging components, such as Gag-Pol and Rev proteins, and a viral genome transfer vector encoding a GFP/luciferase reporter (FIG. 5A). To produce multi-type IFN-MVPs as VLPs without RNA genomes, HEK 293T cells are co-transfected with the desired combination of distinct IFN display constructs and a lentiviral packaging construct expressing essential packaging components, such as Gag-Pol and Rev proteins (FIG. 5B). Finally, to produce multi-type IFN-MVPs as EVs, 293T cells are transfected with desired combinations of distinct IFN display constructs, without structural or genome constructs (FIG. 5C).

TABLE 8
Exemplary IFNs

| IFN Type | Examples |
|---|---|
| Type I | α, β, ε, κ, ω |
| Type II | [symbol] |
| Type III | [symbol] 1-4 |

TABLE 9
Exemplary IFN-MVP Oligomerization Domains

| Oligomerization Domains | Valence |
|---|---|
| VSV-G protein D4 | Trimer |
| Dengue E protein fusion domain | Trimer |
| Foldon | Trimer |
| Leucine Zipper | Dimer |
| Influenza Neuraminidase stem | Tetramer |

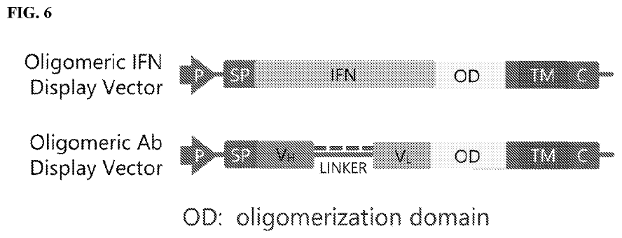

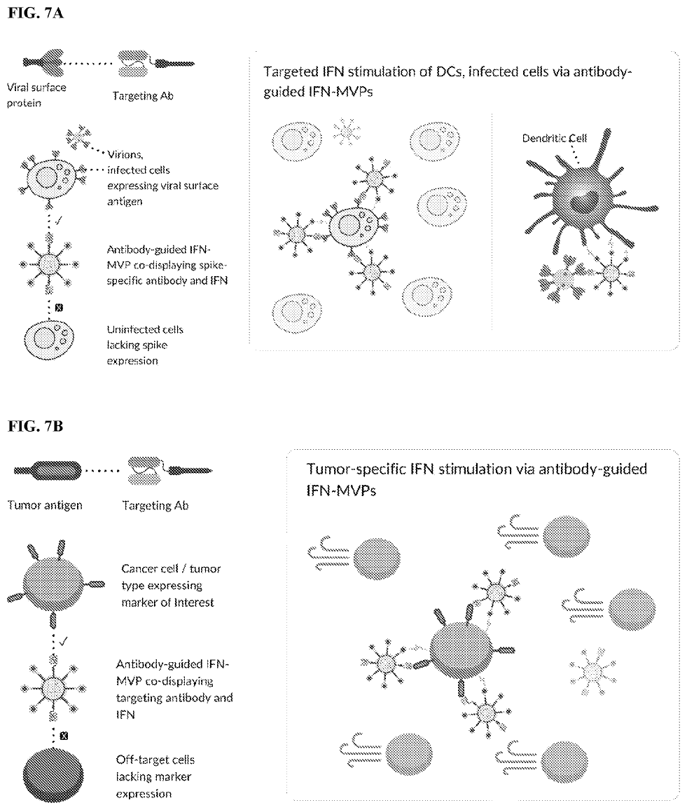

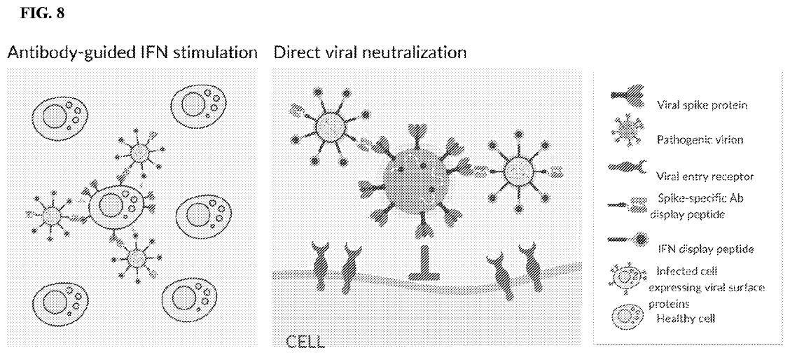

Production of antibody-guided IFN-MVPs: A variety of antibody-guided IFN-MVPs co-displaying IFNs and antibodies with various cellular and viral targets are designed and produced, in various oligomeric patterns (FIG. 6 & Table 9). Antibody-guided IFN-MVPs preferentially target cells and tissues expressing the matching antigen, thus restricting the effects of their displayed IFNs to target cells. Based on the nature of the displayed antibody (Table 10), antibody-guided IFN-MVPs can effectively serve as antigen-specific IFN signaling modulators. For example, an antibody-guided IFN-MVP displaying viral spike-specific antibodies preferentially bind to infected cells (FIG. 7A), while an antibody-guided IFN-MVP displaying tumor-associated antigen (TAA)-specific antibodies selectively target tumor cells expressing that tumor-associated antigen (FIG. 7B). Once bound to target cells, displayed IFNs induce IFN signaling pathways within the target cell. In the context of viruses, antibody-guided IFN-MVPs are dual-action, serving to both neutralize virions and engage IFN signaling in infected cells (FIG. 8). Similar to other formats of IFN-MVPs, antibody-guided IFN-MVPs can be designed atop VLPs and EVs.

TABLE 10
Exemplary Antibody-Guided IFN-MVPs
Antibody Specificity

Tumor Antigen
Viral Spike Protein
Immune Receptor/Ligand
Tissue/cell-specific Antigen

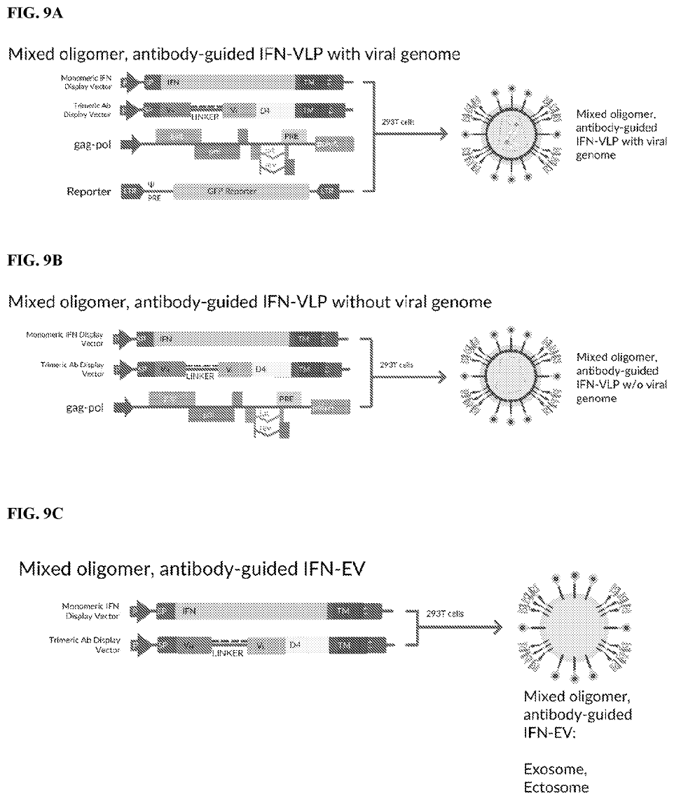

To produce antibody-guided IFN-MVPs as VLPs containing RNA genomes, HEK 293T cells are co-transfected with desired combinations of antibody and IFN display constructs, along with a lentiviral packaging construct expressing essential packaging components, such as Gag-Pol and Rev proteins, and a viral genome transfer vector encoding a GFP/luciferase reporter (FIG. 9A). To produce antibody-guided IFN-MVPs as VLPs without RNA genomes, HEK 293T cells are transfected with desired combinations of antibody and IFN display constructs, along with a lentiviral packaging construct expressing essential packaging components, such as Gag-Pol and Rev proteins (FIG. 9B). Finally, to produce antibody-guided IFN-MVPs as EVs, 293T cells are transfected with desired combinations of antibody and IFN display constructs, without structural or genome constructs (FIG. 9C).

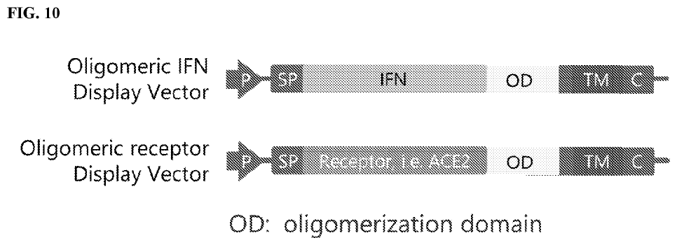

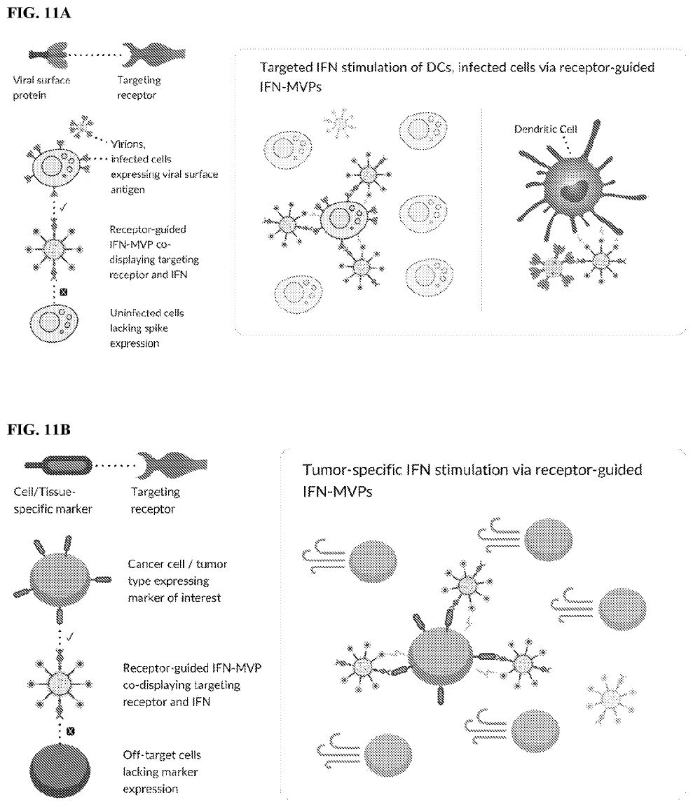

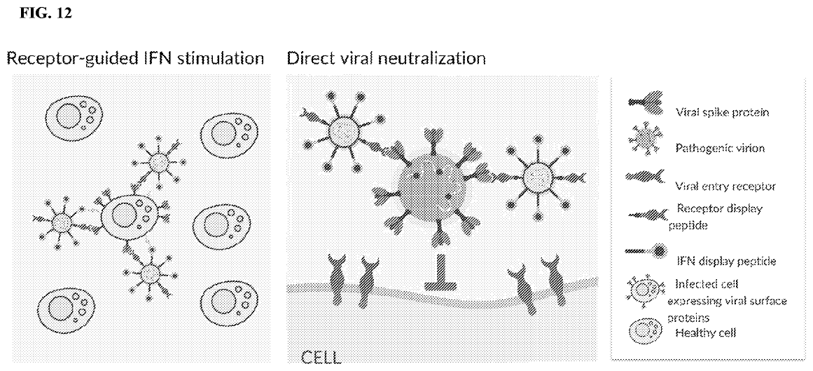

Production of receptor-guided IFN-MVPs: A variety of receptor-guided IFN-MVPs co-displaying IFNs and cellular receptors with various cellular and viral binding partners are designed and produced, in various oligomeric patterns (FIG. 10 & Table 7). Receptor-guided IFN-MVP selectively target cells and tissues expressing its cognate ligand (Table 11). For example, an IFN-MVP displaying a viral entry receptor targets infected cells bearing viral surface proteins (FIG. 11A), while an IFN-MVP displaying a cellular receptor for a tumor antigen targets tumor cells (FIG. 11B). In the context of viruses, receptor-guided IFN-MVPs are dual-action, serving to both neutralize virions and engage IFN signaling in infected cells (FIG. 12). Once localized, the displayed IFN induces IFN signaling pathways within the target cell. Like earlier types of IFN-MVPs, receptor-guided IFN-MVPs can be designed atop VLPs and EVs.

TABLE 11
Exemplary Receptor-Guided IFN-MVPs
Receptor Type

Viral entry receptor
Immune receptor/ligand
Tissue/cell specific receptor/ligand
Tumor specific receptor/ligand

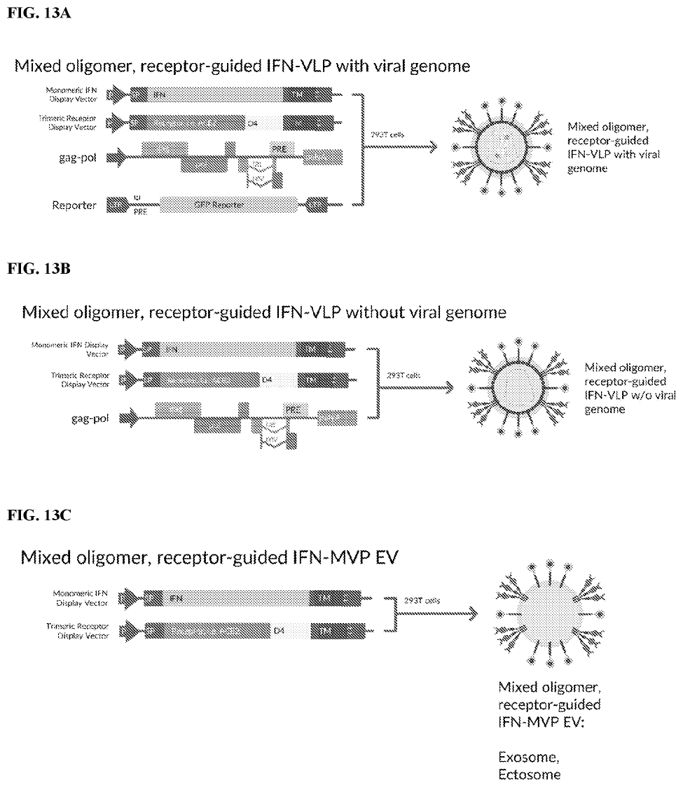

To produce receptor-guided IFN-MVPs as VLPs containing RNA genomes, HEK 293T cells are co-transfected with desired combinations of receptor and IFN display constructs, along with a lentiviral packaging construct expressing essential packaging components, such as Gag-Pol and Rev proteins, and a viral genome transfer vector encoding a GFP/luciferase reporter (FIG. 13A). To produce mixed receptor/IFN-MVPs as VLPs without RNA genomes, HEK 293T cells are transfected with desired combinations of receptor and IFN display constructs, along with a lentiviral packaging construct expressing essential packaging components, such as Gag-Pol and Rev proteins (FIG. 13B). To produce mixed receptor/IFN-MVPs as EVs, 293T cells are transfected with desired combinations of receptor and IFN display constructs, without structural or genome constructs (FIG. 13C).

### Example 3: Characterization of IFN, Antibody and Receptor Display on IFN-MVPs

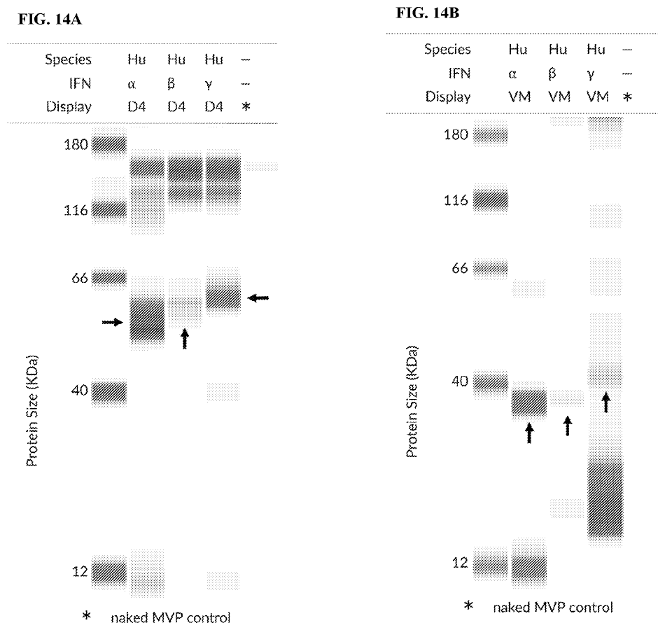

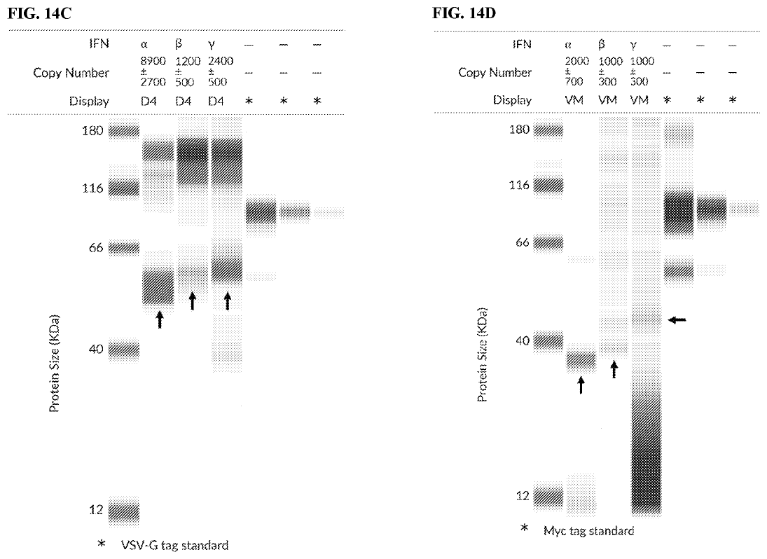

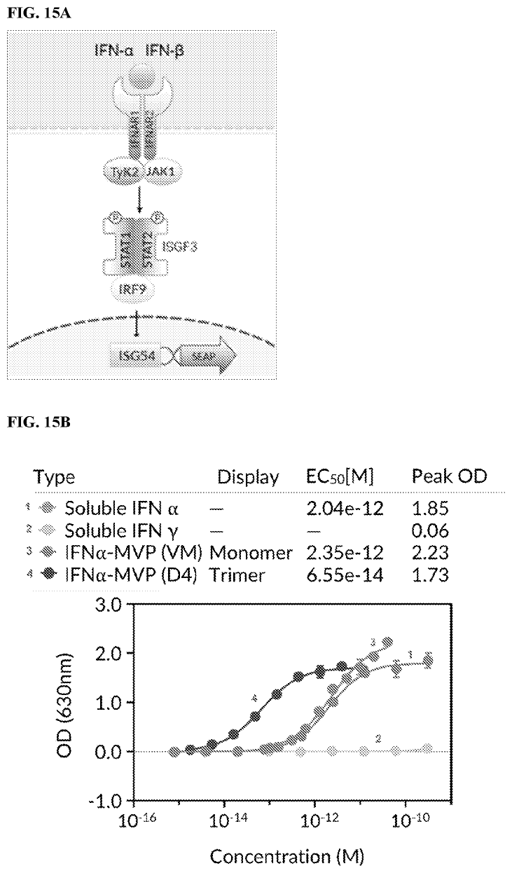

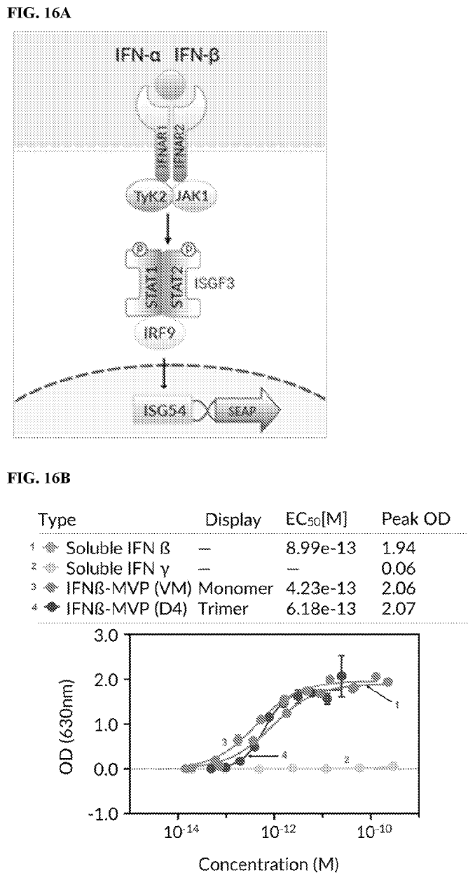

Concentrations of VLP- or EV-based IFN-MVPs were determined via P24 ELISA or tunable resistive pulse sensing (TRPS, qNano), respectively. Based on the concentrations, the copy numbers of displayed IFNs, antibodies and receptors on particles were determined by quantitative western-blot analysis (FIGS. 14A-14D, Table 12). The oligomerization patterns of displayed IFNs, antibodies and receptors were determined via non-reducing PAGE analysis. FIGS. 14A and 14B show reducing western blot analysis of three trimeric and monomeric IFN-MVPs displaying IFN α, β, and γ respectively. FIG. 14C shows reducing quantitative western blot analysis of three trimeric IFN-MVPs displaying IFN α, β, and γ respectively. The copy numbers of IFN α, β, and γ displayed on each type of trimeric IFN-MVP, as listed in Table 12, were 8900±2700, 1200±500, and 2400±500, respectively, which were determined based on a VSV-G tag synthetic standard curve. FIG. 14D shows reducing quantitative western blot analysis of three monomeric IFN-MVPs displaying IFN α, β, and γ respectively. The copy numbers of IFN α, β, and γ displayed on each type of monomeric IFN-MVP, as listed in Table 12, were 2000±700, 1000±300, 1000±300, respectively, which were determined based on a myc tag synthetic standard curve.

TABLE 12
Display Copy Number of IFN-MVP

| IFN-MVP | Display Copies Per Particle |
|---|---|
| IFNα/VM | 2000 ± 700 |
| IFNα/D4 | 8900 ± 2700 |
| IFNβ/VM | 1000 ± 300 |
| IFNβ/D4 | 1200 ± 500 |
| IFNγ/VM | 1000 ± 300 |
| IFNγ/D4 | 2400 ± 500 |

### Example 4: In Vitro Reporter Characterization of Monomeric and Trimeric IFN-MVPs

To determine whether IFN-MVPs display biologically active IFNs, IFN MVPs were tested in cellular reporter assays using HEK 293 cells expressing a secreted embryonic alkaline phosphatase reporter gene (SEAP) under the control of an IFN stimulated gene (ISG) promoter.

FIG. 15A illustrates Type I IFN reporter signaling pathway in HEK-Blue 293 reporter cells. IFN receptors IFNAR1 and IFNAR2 recognize functional type I IFNs, including IFNα, resulting in signal cascade via the JAK-STAT pathway and subsequent ISG-stimulated SEAP reporter expression. FIG. 15B illustrates in vitro reporter assay titration curves for monomeric and trimeric IFNα-MVPs, with soluble IFNα provided as a positive control and type II IFN γ provided as a negative control. Monomeric IFNα/VM and trimeric IFNα/D4 MVPs generated reporter responses at EC₅₀ concentrations of 2.4 pM and 0.06 pM, respectively, while soluble IFNα had an EC₅₀ of 2.0 pM (FIG. 15B).

FIG. 16A illustrates Type I IFN reporter signaling pathway in HEK-Blue 293 reporter cells. IFN receptors IFNAR1 and IFNAR2 recognize functional type I IFNs, including IFN β, resulting in signal cascade via the JAK-STAT pathway and subsequent ISG-stimulated SEAP reporter expression. FIG. 16B illustrates in vitro reporter assay titration curves for monomeric and trimeric IFNβ-MVPs, with soluble IFN β provided as a positive control and type II IFN Y provided as a negative control. Both monomeric and trimeric IFNβ-MVPs resulted in strong SEAP reporter expression and activity, demonstrating functional multivalent display of biologically active IFN β. Monomeric IFNβ/VM and trimeric IFNβ/D4 MVPs generated reporter responses at EC₅₀ concentrations of 0.42 pM and 0.62 pM, respectively, compared to 0.9 pM for soluble IFN β (FIG. 16B).

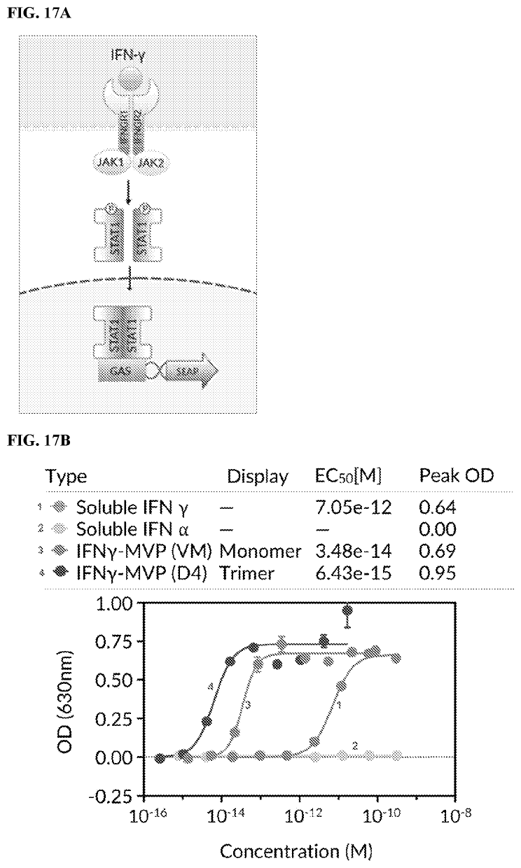

FIG. 17A Schematic illustrating Type II IFN reporter signaling pathway in HEK-Blue 293 reporter cells. Type II IFN receptors IFNGR1 and IFNGR2 recognize functional type II IFNs, including IFN γ, resulting in signal cascade via the JAK-STAT pathway and subsequent ISG-stimulated SEAP reporter expression. FIG. 17B illustrates in vitro reporter assay titration curves for monomeric and trimeric IFNγ-MVPs, with soluble IFN γ provided as a positive control and type I IFN α provided as a negative control. Both monomeric and trimeric IFNγ-MVPs resulted in strong SEAP reporter expression and activity, demonstrating functional multivalent display of biologically active IFN γ. Monomeric IFNγ/VM and trimeric and IFNγ/D4 MVPs generated reporter responses at EC₅₀ concentrations of 35 fM and 6.4 fM, respectively, more than 100-fold more potent than soluble IFN, which generated reporter responses at an EC₅₀ of 7.1 pM (FIG. 17B).

These results demonstrate that IFN-MVPs displaying various types of IFNs display biologically active IFNs that bind to canonical IFN receptors and trigger downstream ISGs.

### Example 5: In Vitro MHC Stimulation by Monomeric and Trimeric IFN-MVPs

To evaluate the function of IFN-MVPs in immune-modulation, the ability of monomeric and trimeric IFN-MVPs to upregulate MHC I expression was investigated in 293T cells. Cells were treated with IFN-MVPs or soluble IFN of the same type. Cells were cultured for 48-hours post-treatment and stained with an HLA I-specific primary antibody. Relative HLA I expression levels were then analyzed via FACS. The proportion of HLA I positive cells and the relative intensities of HLA I expression as determined by FACS were used to compare the efficacy of soluble IFN vs IFN-MVPs in inducing MHC I expression.

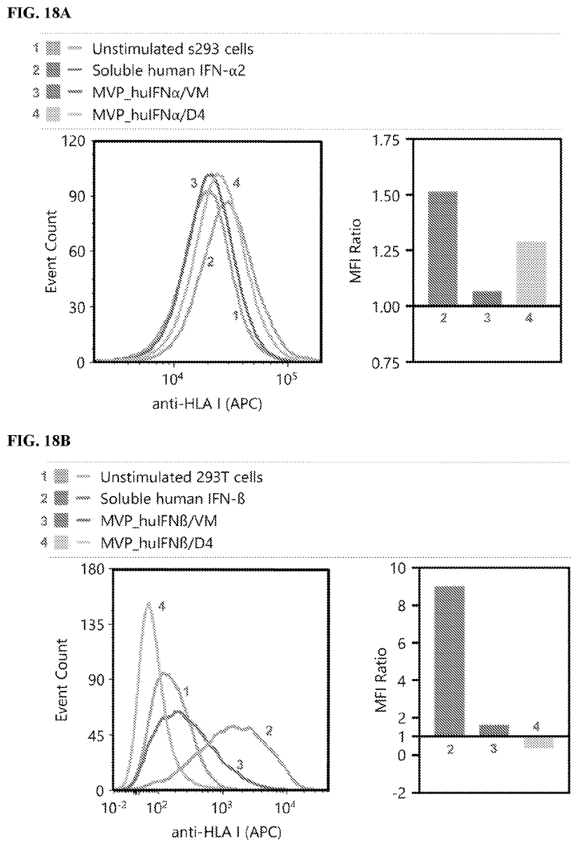

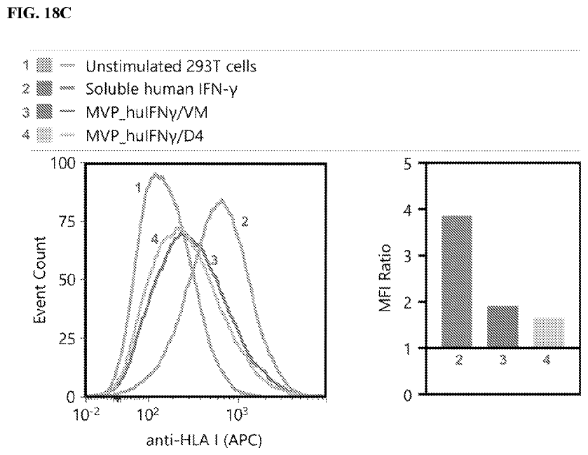

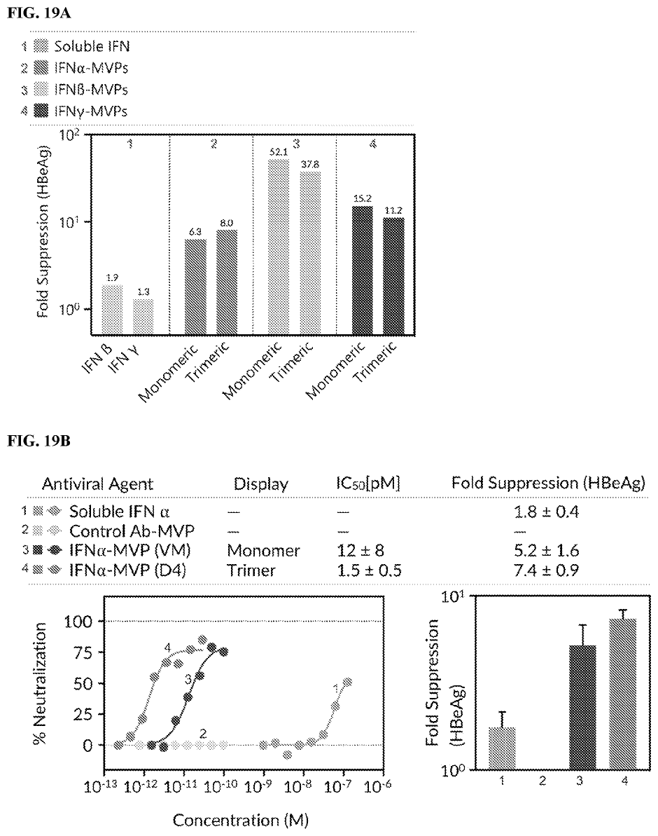

The in vitro MHC I-stimulating effects of monomeric IFNα-MVPs (MVP_huIFNα/VM) and trimeric IFNα-MVPs (MVP_huIFNα/D4) were investigated using s293 cells. Treatment with soluble IFNα resulted in a 1.51-fold increase in mean fluorescence intensity (MFI) compared to untreated s293 cells, while treatment with monomeric IFNα/VM and trimeric IFNα/D4 resulted in 1.07 and 1.29-fold increases, respectively (FIG. 18A). IFNα-MVPs displaying trimeric IFNα were significantly more effective than IFNα-MVPs displaying monomeric IFNα in stimulating MHC I expression, likely a result of increased local valency and binding affinity through trimeric IFNα display.

The in vitro MHC I-stimulating effects of monomeric IFNβ-MVPs (MVP_huIFNβ/VM) and trimeric IFNβ-MVPs (MVP_huIFNβ/D4) were investigated using 293T cells. Treatment with soluble IFNβ resulted in a 9-fold increase in MFI compared to untreated 293T cells, while treatment with monomeric IFNβ-MVPs resulted in 1.62 increase in MHC I expression (FIG. 18B). By contrast, trimeric IFNβ-MVP resulted in nearly three-fold decrease in MHC I expression (FIG. 18B). Trimeric IFNβ-MVP inhibited MHC I expression in 293T cells. The results demonstrated that signals mediated through trimeric, multivalent IFNβ-MVPs altered the function of IFNβ from immune-stimulating to inhibitory. Trimerized and multivalent IFNβ-MVPs can more effectively engage IFN receptors on the cell surface than the soluble IFNβ through avidity, therefore eliciting much stronger signals than the normal physiological signals from soluble IFNβ.

The in vitro MHC I-stimulating effects of monomeric IFNγ-MVPs (MVP_huIFNγ/VM) and trimeric IFNγ-MVPs (MVP_huIFNγ/D4) were investigated using 293T cells. Treatment with soluble IFNγ resulted in a 3.86-fold increase in MFI compared to untreated 293T cells, while treatment with monomeric or trimeric IFNγ-MVP resulted in 1.91 and 1.65-fold changes in MFI, respectively (FIG. 18C). The results demonstrate that IFN-MVPs display functional IFNs and were capable of inducing IFN-regulated pathways in target cells. Altering the oligomerization pattern of displayed IFNs dramatically changed the effects of the IFNs.

### Example 7: In Vitro Antiviral Effects of IFN-MVPs

Given that IFNs are known to induce antiviral cellular pathways in vitro, whether IFN-MVPs could inhibit HBV infection was investigated in culture. HepG2-NTCP cells were treated and cultured with serial dilutions of IFN-MVPs for 24 hours. Cells were then washed with 1×PBS to remove residual IFN-MVPs, before being infected with HBV. 9 days post-infection, supernatant from cell cultures was analyzed and the concentration of HBV e antigen (HBeAg) was determined and used to measure antiviral activity of IFN-MVPs.

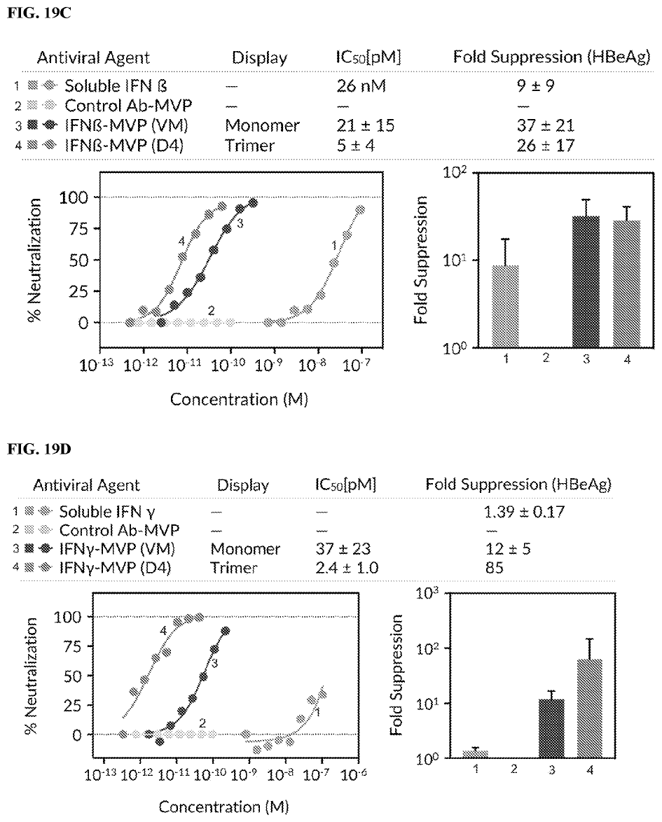

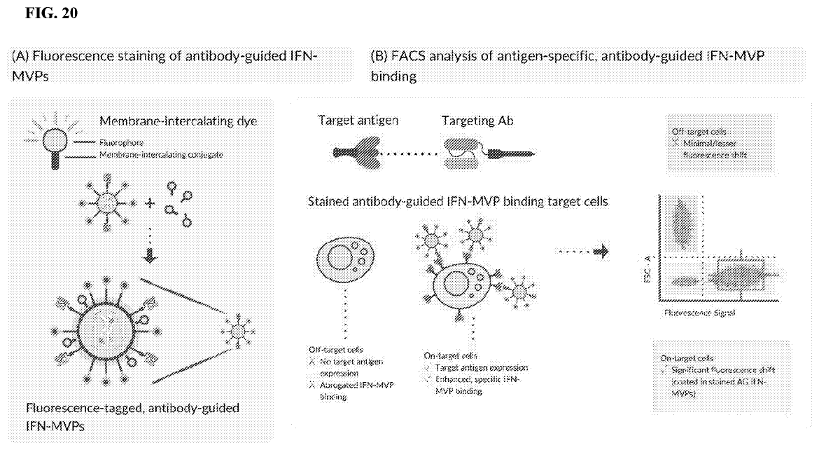

Six different IFN-MVPs displaying IFN α, β and γ in monomeric and trimeric forms were tested against HBV in vitro. Maximum fold of HBeAg suppression was determined relative to an untreated, HBV infection-only control condition. FIG. 19A compares suppression of HBeAg expression by various soluble IFNs and IFN-MVPs when used to inhibit HBV infection of HepG2-NTCP cells. Monomeric and trimeric IFNα-MVPs suppressed HBeAg expression 6.3-fold and 8.0-fold, respectively (FIG. 19A). Monomeric and trimeric IFNβ-MVPs suppressed HBeAg expression 52.1-fold and 37.8-fold, respectively (FIG. 19A). Monomeric and trimeric IFNγ-MVPs suppressed HBeAg expression 15.2-fold and 11.2-fold, respectively (FIG. 19A). The ability of soluble IFN α, β and γ to suppress HBeAg expression was also investigated. The results demonstrate that IFN-MVPs mediate antiviral IFN signaling pathways in target cells.

FIG. 19B illustrates in vitro replication inhibition of HBV by IFNα-MVPs. Soluble IFN α and a negative control MVP were provided for comparison. Fold suppression and percent neutralization were determined as a function of HBeAg detected in cell culture supernatants. Data indicate that trimeric IFNα-MVPs suppressed HBeAg expression more potently than monomeric IFNα-MVPs, and both were more than 1000-fold more potent than soluble IFN α in terms of IC₅₀. Non-specific control MVPs had no effect on HBeAg. FIG. 19C illustrates in vitro replication inhibition of HBV by IFNβ-MVPs. Soluble IFN β and a negative control MVP were provided for comparison. Fold suppression and percent neutralization were determined as a function of HBeAg detected in cell culture supernatants. Data indicate that trimeric IFNβ-MVPs suppressed HBeAg expression more potently than monomeric IFNβ-MVPs, and both were more than 1000-fold more potent than soluble IFN β in terms of IC₅₀. Non-specific control MVPs had no effect on HBeAg. FIG. 19D illustrates in vitro replication inhibition of HBV by IFNγ-MVPs. Soluble IFN γ and a negative control MVPs were provided for comparison. Fold suppression and percent neutralization were determined as a function of HBeAg detected in cell culture supernatants. Data indicate that trimeric IFNγ-MVPs suppressed HBeAg expression more potently than monomeric IFNγ-MVPs, and both are more than 1000-fold more potent than soluble IFN γ in terms of IC₅₀. Non-specific control MVPs have no effect on HBeAg.

### Example 8: Binding of Receptor-Guided and Antibody-Guided IFN-MVPs to Target Cells

To ascertain the specificity of receptor-guided and antibody-guided IFN-MVPs co-displaying cellular receptors or antibodies alongside IFNs, the ability of mixed IFN-MVPs to preferentially bind to target cells expressing the corresponding receptor/antibody target antigen is investigated. Target cell lines expressing the desired antigens are developed (stable cell lines, 293T transfection) and co-cultured with unrelated cells with distinct FACS profiles lacking target antigen expression. Receptor and antibody-guided IFN-MVPs are then labelled with CSFE or other compatible fluorescent dyes. Co-cultured target and off-target cells are then stained with dye-labelled IFN-MVPs before being analyzed via FACS to determine antigen-specific binding of IFN-MVPs. The analyses confirm expression of functionally specific receptors and antibodies on receptor-guided and antibody-guided IFN-MVPs. The data also provide insight on optimizing display copy numbers and oligomerization patterns to better tune antigen-specific mixed IFN-MVPs to target cells.

FIG. 20 illustrates antigen-specific binding of antibody-guided IFN-MVPs. The first step is to screen for binding of antibodies using phage display, yeast display, humanized mouse immunization against the target viral antigen, isolation of binding B cells from convalescent human patient B cells, and/or enrichment of binding antibodies. The second step is to design Ab-MVPs displaying the binder library. The second step can comprise designing Ab-MVPs with single displayed scFv, corresponding viral genome encoding displayed scFv; creating an Ab-MVP library of potential neutralizing Ab-MVPs; binding/infection assay enrichment of high affinity Ab-MVPs; iterating the steps until a suitable pool of the Ab-MVPs with strong binding remain; and validating and determining the best candidates in virus/pseudovirus neutralization assays. Step A of FIG. 20 illustrates a method of labeling antibody-guided IFN-MVPs with membrane intercalating fluorescent dyes. Step B of FIG. 20 illustrates analysis of antibody-guided IFN-MVP antigen-specific cell binding via FACS analysis.

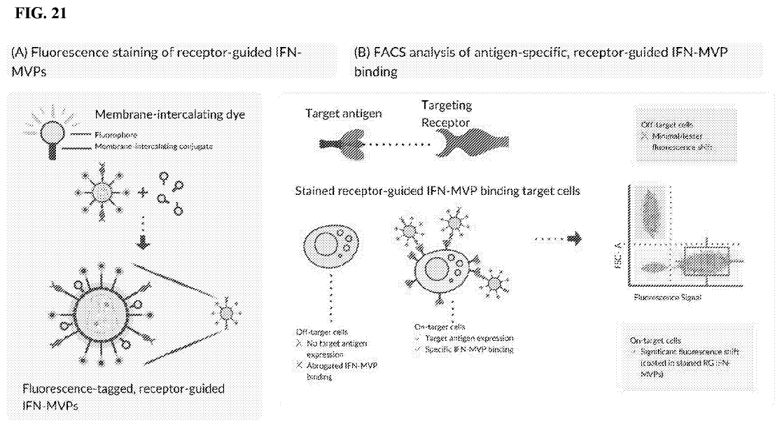

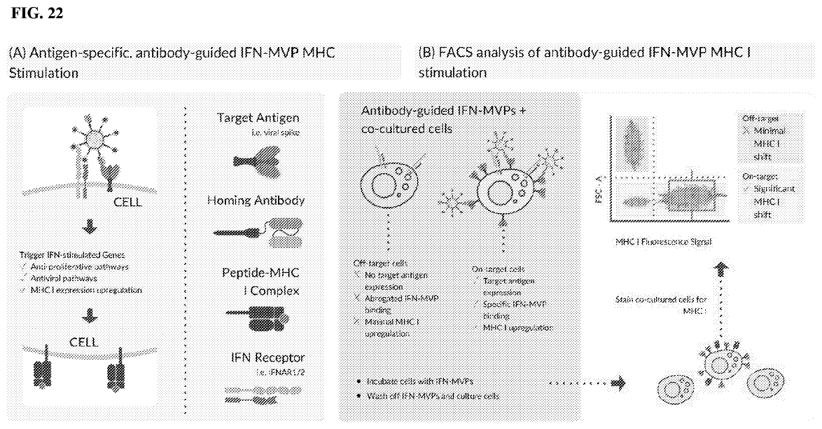

FIG. 21 illustrates antigen-specific binding of receptor-guided IFN-MVPs. The first step is to screen for binding of antibodies using phage display, yeast display, humanized mouse immunization against the target viral antigen, isolation of binding B cells from convalescent human patient B cells, and/or enrichment of binding antibodies. The second step is to design Ab-MVPs displaying the binder library. The second step can comprise designing Ab-MVPs with single displayed scFv, corresponding viral genome encoding displayed scFv; creating an Ab-MVP library of potential neutralizing Ab-MVPs; binding/infection assay enrichment of high affinity Ab-MVPs; iterating the steps until a suitable pool of the Ab-MVPs with strong binding remain; and validating and determining the best candidates in virus/pseudovirus neutralization assays. Step A of FIG. 21 illustrates a method of labeling receptor-guided IFN-MVPs with membrane intercalating fluorescent dyes. Step B of FIG. 21 illustrates analysis of receptor-guided IFN-MVP antigen-specific cell binding via FACS analysis.

### Example 9: In Vitro Antigen-Specific MHC Stimulation by Receptor and Antibody-Guided IFN-MVPs

To provide further validation for the concept of antigen-specific IFN-MVPs, the ability of bifunctional receptor or antibody-guided IFN-MVPs to preferentially upregulate HLA I expression is investigated in cells expressing target antigens. Target cell lines expressing the desired antigens are developed (stable cell lines, 293T transfection) and co-cultured with unrelated cells with distinct FACS profiles lacking target antigen expression. Co-cultured cells are treated with IFN-MVPs or an equivalent dosage of soluble IFN of the same type, based on the copies of displayed IFN per mixed IFN-MVP. After incubation, cells are then washed with 1×PBS to remove all soluble IFNs or IFN-MVPs, and cultured. Finally, cells are stained with an HLA I-specific antibody and relative HLA I expression levels across both cell types are analyzed via FACS. The proportion of HLA I positive cells within each co-cultured cell line and the relative fluorescence intensities of HLA I expression as determined by FACS are used to determine the antigen-specific effects of mixed receptor and antibody-guided IFN-MVPs.

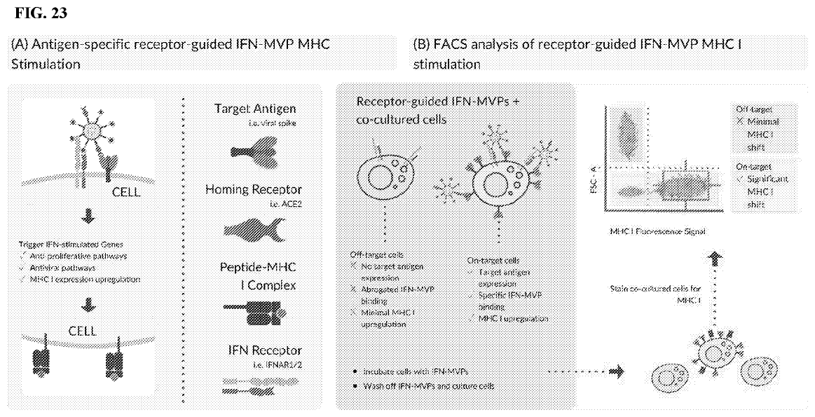

FIG. 22 illustrates antigen-specific MHC stimulation by antibody-guided IFN-MVPs. The first step is to screen for binding of antibodies using phage display, yeast display, humanized mouse immunization against the target viral antigen, isolation of binding B cells from convalescent human patient B cells, and/or enrichment of binding antibodies. The second step is to design Ab-MVPs displaying the binder library. The second step can comprise designing Ab-MVPs with single displayed scFv, corresponding viral genome encoding displayed scFv; creating an Ab-MVP library of potential neutralizing Ab-MVPs; binding/infection assay enrichment of high affinity Ab-MVPs; iterating the steps until a suitable pool of the Ab-MVPs with strong binding remain; and validating and determining the best candidates in virus/pseudovirus neutralization assays.

Step A of FIG. 22 illustrates antigen-specific cell targeting by antibody-guided IFN-MVPs co-displaying antibodies and IFNs. Displayed antibodies allow IFN-MVPs to preferentially bind to cells expressing target antigen, while displayed IFNs bind to cellular IFN receptors such as IFNAR1 to modulate downstream ISGs, including MHC I expression. Step B of FIG. 22 illustrates a method for determining antigen-specific MHC I expression modulation by antibody-guided IFN-MVPs via FACS analysis. IFN-MVPs are mixed with target cells expressing antibody-specific antigen and off-target cells lacking target antigen expression. Co-cultured cells are then stained for MHC I and expression differences analyzed via FACS.

FIG. 23 illustrates antigen-specific MHC stimulation with receptor-guided IFN MVPs. The first step is to screen for binding of antibodies using phage display, yeast display, humanized mouse immunization against the target viral antigen, isolation of binding B cells from convalescent human patient B cells, and/or enrichment of binding antibodies. The second step is to design Ab-MVPs displaying the binder library. The second step can comprise designing Ab-MVPs with single displayed scFv, corresponding viral genome encoding displayed scFv; creating an Ab-MVP library of potential neutralizing Ab-MVPs; binding/infection assay enrichment of high affinity Ab-MVPs; iterating the steps until a suitable pool of the Ab-MVPs with strong binding remain; and validating and determining the best candidates in virus/pseudovirus neutralization assays.

Step A of FIG. 23 illustrates antigen-specific cell targeting by receptor-guided IFN-MVPs co-displaying receptors and IFNs. Displayed receptors allow IFN-MVPs to preferentially bind to cells expressing the receptor cognate ligand, while displayed IFNs bind to cellular IFN receptors such as IFNAR1 to modulate downstream ISGs, including MHC I expression. Step B of FIG. 23 illustrates a method for determining antigen-specific MHC I expression modulation by antibody-guided IFN-MVPs via FACS analysis. IFN-MVPs are mixed with target cells expressing antibody-specific antigen and off-target cells lacking target antigen expression. Co-cultured cells are then stained for MHC I and expression differences analyzed via FACS.

### Example 10: Control of Tumor Development by TAA-Specific Receptor and Ab/IFN-MVPs

Syngeneic mouse tumor models for lung, breast, pancreatic and melanoma cancers are used to examine the effects of tumor-associated antigen (TAA)-specific mixed receptor and antibody-guided IFN-MVPs on tumor development. Purified receptor or antibody-guided IFN-MVPs are injected into mice after tumor implantation through tail-vein injection. Mice are repeatedly dosed with receptor or Ab/IFN-MVPs every 3 days for a total of 6 doses. Tumors are measured at various time points after treatment to determine whether mixed receptor or antibody-guided IFN-MVPs potentiate or inhibit tumor growth in vivo. The effects of receptor or antibody-guided IFN-MVPs on tumor control are compared to the positive control soluble forms of the IFNs displayed on corresponding IFN-MVPs. Tumor control functions of single and multi-type IFN-MVPs displaying IFNs alone are also investigated.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EMBODIMENTS

Embodiment 1. A multivalent particle comprising a first fusion protein that comprises an interferon (IFN) polypeptide and a transmembrane polypeptide wherein the first fusion protein is expressed at a valency of at least about 10 copies on a surface of the multivalent particle.

Embodiment 2. The multivalent particle of embodiment 1, wherein the multivalent particle further comprises a second fusion protein.

Embodiment 3. The multivalent particle of embodiment 2, wherein the second fusion protein comprises a transmembrane polypeptide and an IFN polypeptide that has less than 100% sequence identity to the IFN polypeptide of the first fusion protein and wherein the second fusion protein is expressed at least about 10 copies on a surface of the multivalent particle.

Embodiment 4. The multivalent particle of any of embodiments 1-3, wherein the IFN polypeptide of the first fusion protein comprises a human IFN polypeptide sequence.

Embodiment 5. The multivalent particle of any of embodiments 2-4, wherein the IFN polypeptide of the second fusion protein comprises a human IFN polypeptide sequence.

Embodiment 6. The multivalent particle of any of embodiments 1-5, wherein the IFN polypeptide comprises a Type I IFN, Type II IFN, or a Type III IFN.

Embodiment 7. The multivalent particle of embodiment 6, wherein the Type I IFN comprises IFN-alpha, IFN-beta, IFN-epsilon, IFN-kappa, or IFN-omega.

Embodiment 8. The multivalent particle of embodiment 6, wherein the Type II IFN comprises IFN-gamma.

Embodiment 9. The multivalent particle of embodiment 6, wherein the Type III IFN comprises IFN-lambda.

Embodiment 10. The multivalent particle of any of embodiments 1-9, wherein the IFN polypeptide of the first fusion protein comprises an amino acid sequence with at least 90% sequence identity to SEQ ID NOs: 1-7.

Embodiment 11. The multivalent particle of any of embodiments 2-10, wherein the IFN polypeptide of the second fusion protein comprises an amino acid sequence with at least 90% sequence identity to SEQ ID NOs: 1-7.

Embodiment 12. The multivalent particle of any of embodiments 2-10, wherein the second fusion protein comprises a transmembrane polypeptide and a homing polypeptide that targets the multivalent particle to a target cell or a target protein wherein the second fusion protein is expressed at least about 10 copies on a surface of the multivalent particle.

Embodiment 13. The multivalent particle of embodiment 12, wherein the target cell is a cancer cell.

Embodiment 14. The multivalent particle of embodiment 13, wherein the cancer cell is from a cancer comprising melanoma, leukemia, lymphoma, multiple myeloma, liver cancer, pancreatic cancer, lung cancer, breast cancer, prostate cancer, brain cancer, colorectal cancer, bladder cancer, kidney cancer, cervical cancer, ovarian cancer, esophageal cancer, mesothelioma, gastric cancer, and sarcoma.

Embodiment 15. The multivalent particle of embodiment 12, wherein the target protein is a viral surface protein from hepatitis B virus (HBV), SARS CoV-2, SARS CoV-1, MERS CoV, Influenza, respiratory syncytial virus, HIV, or measles.

Embodiment 16. The multivalent particle of embodiment 12, wherein the target protein comprises a viral spike protein.

Embodiment 17. The multivalent particle of embodiment 12, wherein the homing polypeptide comprises an antibody that binds specifically to an antigen on the target cell or the target viral protein.

Embodiment 18. The multivalent particle of embodiment 17, wherein the antigen on the target cell comprises an antigen listed in Table 2.

Embodiment 19. The multivalent particle of embodiment 17, wherein the antibody is a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.

Embodiment 20. The multivalent particle of any of embodiments 17-19, wherein the antibody comprises an amino acid sequence from at least one complementarity determining region of BG10-19, 80R, 7D10, FI6, 1E01, or H015.

Embodiment 21. The multivalent particle of any of embodiments 17-20, wherein the antibody is a multispecific antibody.

Embodiment 22. The multivalent particle of embodiment 12, wherein the homing polypeptide comprises a mammalian receptor that has binding specificity for the target viral protein.

Embodiment 23. The multivalent particle of embodiment 22, wherein the mammalian receptor comprises NTCP, ACE2, TRMPSS2, DPP4, CD4, CCR5, CXCR4, CD209, or CLEC4M.

Embodiment 24. The multivalent particle of embodiment 12, wherein the homing polypeptide comprises a mammalian ligand that recognizes a tumor-associated receptor.

Embodiment 25. The multivalent particle of embodiment 24, wherein the mammalian ligand recognizes EGF, VEGF, TGFbeta, IL-4, IL-11, IGF1, IL-6, or RGD peptide.

Embodiment 26. The multivalent particle of any of embodiments 1-25, wherein the first fusion protein is monomeric.

Embodiment 27. The multivalent particle of any of embodiments 1-25, wherein the first fusion protein comprises an oligomerization domain.

Embodiment 28. The multivalent particle of any of embodiments 2-27, wherein the second fusion protein is monomeric.

Embodiment 29. The multivalent particle of any of embodiments 2-27, wherein the second fusion protein comprises an oligomerization domain.

Embodiment 30. The multivalent particle of embodiment 27 or embodiment 29, wherein the oligomerization domain comprises a dimerization domain.

Embodiment 31. The multivalent particle of embodiment 30, wherein the dimerization domain comprises a leucine zipper dimerization domain.

Embodiment 32. The multivalent particle of embodiment 27 or embodiment 29, wherein the oligomerization domain comprises a trimerization domain.

Embodiment 33. The multivalent particle of embodiment 32, wherein the trimerization domain comprises a post-fusion oligomerization domain of viral surface protein.

Embodiment 34. The multivalent particle of embodiment 32, wherein the trimerization domain comprises a D4 post-fusion trimerization domain of VSV-G protein.

Embodiment 35. The multivalent particle of embodiment 32, wherein the trimerization domain comprises a Dengue E protein post-fusion trimerization domain.

Embodiment 36. The multivalent particle of embodiment 32, wherein the trimerization domain comprises a foldon trimerization domain.

Embodiment 37. The multivalent particle of embodiment 27 or embodiment 29, wherein the oligomerization domain comprises a tetramerization domain.

Embodiment 38. The multivalent particle of embodiment 37, wherein the tetramerization domain comprises an influenza neuraminidase stem domain.

Embodiment 39. The multivalent particle of embodiment 27 or embodiment 29, wherein the oligomerization domain comprises an amino acid sequence that has at least 95% sequence identity to an amino acid sequence according to SEQ ID NOs: 52-65.

Embodiment 40. The multivalent particle of any one of embodiments 1-39, wherein the first fusion protein comprises a signal peptide.

Embodiment 41. The multivalent particle of embodiment 40, wherein domains of the first fusion protein are arranged from the N-terminus to the C-terminus in the following orders:
(a) signal peptide, interferon polypeptide, oligomerization domain, and transmembrane polypeptide;
(b) signal peptide, interferon polypeptide, transmembrane polypeptide, and oligomerization domain; or
(c) signal peptide, oligomerization domain, display peptide, and transmembrane polypeptide.

Embodiment 42. The multivalent particle of any one of embodiments 1-41, wherein the first fusion protein further comprises a cytosolic domain.

Embodiment 43. The multivalent particle of embodiment 42, wherein domains of the first fusion protein are arranged from the N-terminus to the C-terminus in the following orders:
(a) signal peptide, interferon polypeptide, oligomerization domain, transmembrane polypeptide, and cytosolic domain;
(b) signal peptide, interferon polypeptide, transmembrane polypeptide, oligomerization domain, and cytosolic domain; or
(c) signal peptide, oligomerization domain, interferon polypeptide, transmembrane polypeptide, and cytosolic domain.

Embodiment 44. The multivalent particle of any of embodiments 2-43, wherein the second fusion protein comprises a signal peptide.

Embodiment 45. The multivalent particle of embodiment 3, wherein the second fusion protein comprises the IFN polypeptide that has less than 100% sequence identity to the IFN polypeptide of the first fusion protein.

Embodiment 46. The multivalent particle of embodiment 45, wherein domains of the second fusion protein are arranged from the N-terminus to the C-terminus in the following orders:
(a) signal peptide, interferon polypeptide, oligomerization domain, and transmembrane polypeptide;
(b) signal peptide, interferon polypeptide, transmembrane polypeptide, and oligomerization domain; or
(c) signal peptide, oligomerization domain, interferon polypeptide, and transmembrane polypeptide.

Embodiment 47. The multivalent particle of any one of embodiments 3, 45-46, wherein the second fusion protein further comprises a cytosolic domain.

Embodiment 48. The multivalent particle of embodiment 47, wherein domains of the second fusion protein are arranged from the N-terminus to the C-terminus in the following orders:
(a) signal peptide, interferon polypeptide, oligomerization domain, transmembrane polypeptide, and cytosolic domain;
(b) signal peptide, interferon polypeptide, transmembrane polypeptide, oligomerization domain, and cytosolic domain; or
(c) signal peptide, oligomerization domain, interferon polypeptide, transmembrane polypeptide, and cytosolic domain.

Embodiment 49. The multivalent particle any one of embodiments 12-44, wherein the second fusion protein comprises the homing polypeptide.

Embodiment 50. The multivalent particle of embodiment 49, wherein domains of the second fusion protein are arranged from the N-terminus to the C-terminus in the following orders:
(a) signal peptide, homing polypeptide, oligomerization domain, and transmembrane polypeptide;
(b) signal peptide, homing polypeptide, transmembrane polypeptide, and oligomerization domain; or
(c) signal peptide, oligomerization domain, homing polypeptide, and transmembrane polypeptide.

Embodiment 51. The multivalent particle of embodiment 50, wherein the second fusion protein further comprises a cytosolic domain.

Embodiment 52. The multivalent particle of embodiment 51, wherein domains of the second fusion protein are arranged from the N-terminus to the C-terminus in the following orders:
(a) signal peptide, homing polypeptide, oligomerization domain, transmembrane polypeptide, and cytosolic domain;
(b) signal peptide, homing polypeptide, transmembrane polypeptide, oligomerization domain, and cytosolic domain; or
(c) signal peptide, oligomerization domain, homing polypeptide, transmembrane polypeptide, and cytosolic domain.

Embodiment 53. The multivalent particle of any one of embodiments 1-52, wherein the transmembrane polypeptide anchors the first fusion protein to a bilayer of the multivalent particle.

Embodiment 54. The multivalent particle of any one of embodiments 2-53, wherein the transmembrane polypeptide anchors the second fusion protein to a bilayer of the multivalent particle.

Embodiment 55. The multivalent particle of any one of embodiments 1-54, wherein the transmembrane polypeptide comprises a transmembrane domain of a Vesicular Stomatitis virus glycoprotein (VSV-G).

Embodiment 56. The multivalent particle of any one of embodiments 1-54, wherein the transmembrane polypeptide comprises a transmembrane domain of influenza Hemagglutinin (HA).

Embodiment 57. The multivalent particle of any one of embodiments 1-54, wherein the transmembrane polypeptide comprises a transmembrane domain of HIV surface glycoprotein GP120 or GP41.

Embodiment 58. The multivalent particle of any one of embodiments 1-54, wherein the transmembrane polypeptide comprises a transmembrane domain of measles virus surface glycoprotein hemagglutinin (H) protein.

Embodiment 59. The multivalent particle of any one of embodiments 1-54, wherein the transmembrane polypeptide comprises an amino acid sequence at least about 90% identical to that set forth in SEQ ID NO: 66-74.

Embodiment 60. The multivalent particle of any one of embodiments 1-59, wherein the multivalent particle is synthetic.

Embodiment 61. The multivalent particle of any one of embodiments 1-59, wherein the multivalent particle is recombinant.

Embodiment 62. The multivalent particle of any one of embodiments 1-59, wherein the multivalent particle is an enveloped particle.

Embodiment 63. The multivalent particle of any one of embodiments 1-59, wherein the multivalent particle is a lentiviral particle.

Embodiment 64. The multivalent particle of any one of embodiments 1-59, wherein the multivalent particle does not comprise viral genetic material.

Embodiment 65. The multivalent particle of any one of embodiments 1-59, wherein the multivalent particle comprises a lipid bilayer.

Embodiment 66. The multivalent particle of any one of embodiments 1-59, wherein the multivalent particle comprises a virus.

Embodiment 67. The multivalent particle of any one of embodiments 1-59, wherein the multivalent particle comprises a replication incompetent virus.

Embodiment 68. The multivalent particle of any one of embodiments 1-59, wherein the multivalent particle comprises a replication competent virus.

Embodiment 69. The multivalent particle of any one of embodiments 1-59, wherein the multivalent particle comprises a viral-like particle.

Embodiment 70. The multivalent particle of any one of embodiments 1-59, wherein the multivalent particle comprises an extracellular vesicle.

Embodiment 71. The multivalent particle of any one of embodiments 1-59, wherein the extracellular vesicle comprises an ectosome.

Embodiment 72. The multivalent particle of any one of embodiments 1-59, wherein the extracellular vesicle comprises an exosome.

Embodiment 73. The multivalent particle of any one of embodiments 1-72, wherein the first fusion protein is expressed at a valency of about 10 copies on a surface of the multivalent particle.

Embodiment 74. The multivalent particle of any one of embodiments 1-72, wherein the first fusion protein is expressed at a valency of about 10 to 15 copies on a surface of the multivalent particle.

Embodiment 75. The multivalent particle of any one of embodiments 1-72, wherein the first fusion protein is expressed at a valency of at least about 25 copies on a surface of the multivalent particle.

Embodiment 76. The multivalent particle of any one of embodiments 1-72, wherein the first fusion protein is expressed at a valency of at least about 50 copies on a surface of the multivalent particle.

Embodiment 77. The multivalent particle of any one of embodiments 1-72, wherein the first fusion protein is expressed at a valency of at least about 100 copies on a surface of the multivalent particle.

Embodiment 78. The multivalent particle of any one of embodiments 1-72, wherein the first fusion protein is expressed at a valency of at least about 200 copies on a surface of the multivalent particle.

Embodiment 79. The multivalent particle of any one of embodiments 1-72, wherein the first fusion protein is expressed at a valency of at least about 400 copies on a surface of the multivalent particle.

Embodiment 80. The multivalent particle of any one of embodiments 1-72, wherein the first fusion protein is expressed at a valency of at least about 600 copies on a surface of the multivalent particle.

Embodiment 81. The multivalent particle of any one of embodiments 1-72, wherein the first fusion protein is expressed at a valency of at least about 1000 copies on a surface of the multivalent particle.

Embodiment 82. The multivalent particle of any one of embodiments 1-72, wherein the second fusion protein is expressed at a valency of about 10 copies on a surface of the multivalent particle.

Embodiment 83. The multivalent particle of any one of embodiments 2-82, wherein the second fusion protein is expressed at a valency of about 10 to 15 copies on a surface of the multivalent particle.

Embodiment 84. The multivalent particle of any one of embodiments 2-82, wherein the second fusion protein is expressed at a valency of at least about 25 copies on a surface of the multivalent particle.

Embodiment 85. The multivalent particle of any one of embodiments 2-82, wherein the second fusion protein is expressed at a valency of at least about 50 copies on a surface of the multivalent particle.

Embodiment 86. The multivalent particle of any one of embodiments 2-82, wherein the second fusion protein is expressed at a valency of at least about 100 copies on a surface of the multivalent particle.

Embodiment 87. The multivalent particle of any one of embodiments 2-82, wherein the second fusion protein is expressed at a valency of at least about 200 copies on a surface of the multivalent particle.

Embodiment 88. The multivalent particle of any one of embodiments 2-82, wherein the second fusion protein is expressed at a valency of at least about 400 copies on a surface of the multivalent particle.

Embodiment 89. The multivalent particle of any one of embodiments 2-82, wherein the second fusion protein is expressed at a valency of at least about 600 copies on a surface of the multivalent particle.

Embodiment 90. The multivalent particle of any one of embodiments 2-82, wherein the second fusion protein is expressed at a valency of at least about 1000 copies on a surface of the multivalent particle.

Embodiment 91. The multivalent particle of any one of embodiments 1-90, wherein the multivalent particle comprises a fluorophore expressed on a surface of the multivalent particle.

Embodiment 92. The multivalent particle of embodiment 91, wherein the fluorophore is conjugated to a membrane-intercalating polypeptide.

Embodiment 93. A composition comprising a nucleic acid sequence that encodes the first fusion protein of any of embodiments 1-92.

Embodiment 94. A composition comprising a nucleic acid sequence that encodes the first fusion protein of any one of embodiments 1-92 and the second fusion protein of any one of embodiments 2-92.

Embodiment 95. The composition of embodiment 93 or embodiment 94, wherein the composition further comprises a second nucleic acid sequence that encodes one or more packaging viral proteins.

Embodiment 96. The composition of embodiment 95, wherein the one or more packaging viral proteins is a lentiviral protein, a retroviral protein, an adenoviral protein, or combinations thereof.

Embodiment 97. The composition of embodiment 95, wherein the one or more packaging viral proteins comprises gag, pol, pre, tat, rev, or combinations thereof.

Embodiment 98. The composition of claim any one of embodiments 94-97, further comprising a third nucleic acid sequence that encodes a reporter, a therapeutic molecule, or combinations thereof.

Embodiment 99. The composition of embodiment 98, wherein the reporter is a fluorescent protein or luciferase.

Embodiment 100. The composition of embodiment 99, wherein the fluorescent protein is green fluorescent protein.

Embodiment 101. The composition of embodiment 98, wherein the therapeutic molecule is an immune modulating protein, a cellular signal modulating molecule, a proliferation modulating molecule, or combinations thereof.

Embodiment 102. The composition of any one of embodiments 98-101, wherein the nucleic acid sequence that encodes the first fusion protein and the second nucleic acid sequence and the third nucleic acid sequence are within a same vector.

Embodiment 103. The composition of any one of embodiments 98-101, wherein the nucleic acid sequence that encodes the first fusion protein and the second fusion protein and the second nucleic acid sequence and the third nucleic acid sequence are within a same vector.

Embodiment 104. The composition of any one of embodiments 98-101, wherein the nucleic acid sequence that encodes the first fusion protein and the second nucleic acid sequence and the third nucleic acid sequence are within different vectors.

Embodiment 105. The composition of any one of embodiments 98-101, wherein the nucleic acid sequence that encodes the first fusion protein and the second fusion protein and the second nucleic acid sequence and the third nucleic acid sequence are within different vectors.

Embodiment 106. The composition of any one of embodiments 102-105, wherein the vector is a lentivirus vector, an adenovirus vector, or an adeno-associated virus vector.

Embodiment 107. The composition of any one of embodiments 98-106, wherein the nucleic acid sequence that encodes the first fusion protein and the second fusion protein and the second nucleic acid sequence and the third nucleic acid sequence are DNAs.

Embodiment 108. The composition of any one of embodiments 98-106, wherein the nucleic acid sequence that encodes the first fusion protein and the second fusion protein and the second nucleic acid sequence and the third nucleic acid sequence are mRNAs.

Embodiment 109. A method of treating cancer in a subject in need thereof comprising administering to the subject the multivalent particle according to any one of embodiments 1-92.

Embodiment 110. The method of embodiment 109, wherein the cancer comprises melanoma, leukemia, lymphoma, multiple myeloma, liver cancer, pancreatic cancer, lung cancer, breast cancer, prostate cancer, brain cancer, colorectal cancer, bladder cancer, kidney cancer, cervical cancer, ovarian cancer, esophageal cancer, mesothelioma, gastric cancer, and sarcoma.

Embodiment 111. The method of embodiment 109 or embodiment 110, wherein the multivalent particle is administered to the subject through inhalation.

Embodiment 112. The method of embodiment 109 or embodiment 110, wherein the multivalent particle is administered to the subject through intranasal delivery.

Embodiment 113. The method of embodiment 109 or embodiment 110, wherein the multivalent particle is administered to the subject through intratracheal delivery.

Embodiment 114. The method of embodiment 109 or embodiment 110, wherein the multivalent particle is administered to the subject by an intraperitoneal injection.

Embodiment 115. The method of embodiment 109 or embodiment 110, wherein the multivalent particle is administered to the subject by a subcutaneous injection.

Embodiment 116. The method of any one of embodiments 109-115, wherein the administering to the subject of the multivalent particle is sufficient to reduce or eliminate the cancer as compared to a baseline measurement of the cancer taken from the subject prior to the administering of the multivalent particle.

Embodiment 117. A method of treating hepatitis B virus (HBV), SARS CoV-2, SARS CoV-1, MERS CoV, Influenza, respiratory syncytial virus, HIV, or measles in a subject in need thereof comprising administering to the subject the multivalent particle according to any one of embodiments 1-92.

Embodiment 118. The method of embodiment 117, wherein the multivalent particle is administered to the subject intravenously.

Embodiment 119. The method of embodiment 117, wherein the multivalent particle is administered to the subject through inhalation.

Embodiment 120. The method of embodiment 117, wherein the multivalent particle is administered to the subject through intranasal delivery.

Embodiment 121. The method of embodiment 117, wherein the multivalent particle is administered to the subject through intratracheal delivery.

Embodiment 122. The method of embodiment 117, wherein the multivalent particle is administered to the subject by an intraperitoneal injection.

Embodiment 123. The method of embodiment 117, wherein the multivalent particle is administered to the subject by a subcutaneous injection.

Embodiment 124. The method of any one of embodiments 117-123, wherein the multivalent particle induces T cell mediated cytotoxicity against viral infected cells.

Embodiment 125. The method of any one of embodiments 117-124, wherein the administering to the subject of the multivalent particle is sufficient to reduce or eliminate the hepatitis B virus (HBV), SARS CoV-2, SARS CoV-1, MERS CoV, Influenza, respiratory syncytial virus, HIV, or measles, eliminate the hepatitis B virus (HBV), SARS CoV-2, SARS CoV-1, MERS CoV, Influenza, respiratory syncytial virus, HIV, or measles, as compared to a baseline measurement taken of the hepatitis B virus (HBV), SARS CoV-2, SARS CoV-1, MERS CoV, Influenza, respiratory syncytial virus, HIV, or measles from the subject prior to the administering of the multivalent particle.

Embodiment 126. The method of embodiment 125, wherein the reduction is at least about 1-fold, 5-fold, 10-fold, 20-fold, 40-fold, 60-fold, 80-fold, or up to about 100-fold.

Embodiment 127. A method of treating an autoimmune disease or an inflammatory disease in a subject in need thereof comprising administering to the subject the multivalent particle according to any one of embodiments 1-92.

Embodiment 128. The method of embodiment 127, wherein the autoimmune disease comprises rheumatoid arthritis, systemic lupus erythematosus, multiple sclerosis, inflammatory bowel diseases, psoriasis, or aplastic anemia.

Embodiment 129. The method of embodiment 128, wherein the multivalent particle is administered to the subject intravenously.

Embodiment 130. The method of embodiment 128, wherein the multivalent particle is administered to the subject through inhalation.

Embodiment 131. The method of embodiment 128, wherein the multivalent particle is administered to the subject through intranasal delivery.

Embodiment 132. The method of embodiment 128, wherein the multivalent particle is administered to the subject through intratracheal delivery.

Embodiment 133. The method of embodiment 128, wherein the multivalent particle is administered to the subject by an intraperitoneal injection.

Embodiment 134. The method of embodiment 128, wherein the multivalent particle is administered to the subject by a subcutaneous injection.

Embodiment 135. The method of embodiment 128, wherein the multivalent particle induces T cell mediated cytotoxicity against viral infected cells.

Embodiment 136. The method of any one of embodiments 128-135, wherein the administering to the subject of the multivalent particle is sufficient to reduce or eliminate a symptom of the autoimmune disease as compared to a baseline measurement of the symptom of the autoimmune disease as measured from the subject prior to the administering of the multivalent particle.

Embodiment 137. The method of embodiment 136, wherein the reduction is at least about 1-fold, 5-fold, 10-fold, 20-fold, 40-fold, 60-fold, 80-fold, or up to about 100-fold.

Embodiment 138. A method of treating a cancer in a subject in need thereof comprising administering to the subject a composition of any one of embodiments 93-108.

Embodiment 139. The method of embodiment 138, wherein the cancer comprises melanoma, leukemia, lymphoma, multiple myeloma, liver cancer, pancreatic cancer, lung cancer, breast cancer, prostate cancer, brain cancer, colorectal cancer, bladder cancer, kidney cancer, cervical cancer, ovarian cancer, esophageal cancer, mesothelioma, gastric cancer, and sarcoma.

Embodiment 140. A method of treating a viral infection in a subject in need thereof comprising administering to the subject a composition of any one of embodiments 93-108.

Embodiment 141. The method of embodiment 140, wherein the infection comprises infection by hepatitis B virus (HBV), SARS CoV-2, SARS CoV-1, MERS CoV, Influenza, respiratory syncytial virus, HIV, or measles.

Embodiment 142. A method of treating an autoimmune disease or an inflammatory disease in a subject in need thereof comprising administering to the subject a composition of any one of embodiments 93-108.

Embodiment 143. The method of embodiment 142, wherein the autoimmune disease is rheumatoid arthritis, systemic lupus erythematosus, multiple sclerosis, inflammatory bowel diseases, psoriasis, or aplastic anemia.

Embodiment 144. The method of any one of embodiments 138-143, wherein the composition is administered to the subject through inhalation.

Embodiment 145. The method of any one of embodiments 138-143, wherein the composition is administered to the subject through intranasal delivery.

Embodiment 146. The method of any one of embodiments 138-143, wherein the composition is administered to the subject through intratracheal delivery.

Embodiment 147. The method of any one of embodiments 138-143, wherein the composition is administered to the subject by an intraperitoneal injection.

Embodiment 148. The method of any one of embodiments 138-143, wherein the composition is administered to the subject by a subcutaneous injection.

Embodiment 149. The method of embodiment 138, wherein the administering to the subject of the composition is sufficient to reduce or eliminate the cancer as compared to a baseline measurement of the cancer taken from the subject prior to the administering of the composition.

Embodiment 150. The method of embodiment 141, wherein the administering to the subject of the composition is sufficient to reduce or eliminate the hepatitis B virus (HBV), SARS CoV-2, SARS CoV-1, MERS CoV, Influenza, respiratory syncytial virus, HIV, or measles, as compared to a baseline measurement taken of the hepatitis B virus (HBV), SARS CoV-2, SARS CoV-1, MERS CoV, Influenza, respiratory syncytial virus, HIV, or measles from the subject prior to the administering of the composition.

Embodiment 151. The method of embodiment 142, wherein the administering to the subject of the composition is sufficient to reduce or eliminate a symptom of the autoimmune disease as compared to a baseline measurement of the symptom of the autoimmune disease as measured from the subject prior to the administering of the composition.

Embodiment 152. The method of any one of embodiments 138-151, wherein the composition is administered with a liposome.

Embodiment 153. The method of any one of embodiments 138-151, wherein the composition is administered with an adeno-associated virus (AAV).

Embodiment 154. The method of any one of embodiments 138-151, wherein the composition is administered with a lipid nanoparticle.

Embodiment 155. The method of any one of embodiments 138-151, wherein the composition is administered with a polymer.

Embodiment 156. The method of any one of embodiments 138-151, wherein the composition is administered as a naked nucleic acid sequence.

Embodiment 157. The method of any one of embodiments 138-151, wherein the composition is administered as a naked DNA sequence.

Embodiment 158. The method of any one of embodiments 138-151, wherein the composition is administered as a naked mRNA sequence.

Embodiment 159. A pharmaceutical composition comprising (a) the multivalent particle of any one of embodiments 1-92, and (b) a pharmaceutically acceptable excipient.

What is claimed is:

1. A multivalent particle comprising a first fusion protein that comprises an interferon (IFN) polypeptide and a transmembrane polypeptide wherein the first fusion protein is expressed on a surface of the multivalent particle.

2. The multivalent particle of claim 1, wherein the multivalent particle further comprises a second fusion protein.

3. The multivalent particle of claim 2, wherein the second fusion protein comprises a transmembrane polypeptide and an IFN polypeptide that has less than 100% sequence identity to the IFN polypeptide of the first fusion protein and wherein the second fusion protein is expressed on the surface of the multivalent particle.

4. The multivalent particle of claim 1, wherein the IFN polypeptide of the first fusion protein comprises a human IFN polypeptide sequence.

5. The multivalent particle of claim 2, wherein the IFN polypeptide of the second fusion protein comprises a human IFN polypeptide sequence.

6. The multivalent particle of claim 1, wherein the IFN polypeptide of the first fusion protein comprises a Type I IFN, Type II IFN, or a Type III IFN.

7. The multivalent particle of claim 6, wherein the Type I IFN comprises IFN-alpha, IFN-beta, IFN-epsilon, IFN-kappa, or IFN-omega.

8. The multivalent particle of claim 6, wherein the Type II IFN comprises IFN-gamma.

9. The multivalent particle of claim 6, wherein the Type III IFN comprises IFN-lambda.

10. The multivalent particle of claim 3, wherein the IFN polypeptide of the second fusion protein comprises a Type I IFN, Type II IFN, or a Type III IFN.

11. The multivalent particle of claim 10, wherein the Type I IFN comprises IFN-alpha, IFN-beta, IFN-epsilon, IFN-kappa, or IFN-omega.

12. The multivalent particle of claim 10, wherein the Type II IFN comprises IFN-gamma.

13. The multivalent particle of claim 10, wherein the Type III IFN comprises IFN-lambda.

14. The multivalent particle of claim 1, wherein the IFN polypeptide of the first fusion protein comprises an amino acid sequence with at least 90% sequence identity to any one of SEQ ID NOs: 1-7.

15. The multivalent particle of claim 3, wherein the IFN polypeptide of the second fusion protein comprises an amino acid sequence with at least 90% sequence identity to any one of SEQ ID NOs: 1-7.

16. The multivalent particle of claim 2, wherein the second fusion protein comprises a transmembrane polypeptide and a homing polypeptide that targets the multivalent particle to a target cell or a target protein wherein the second fusion protein is expressed on the surface of the multivalent particle.

17. The multivalent particle of claim 16, wherein the target cell comprises a cancer cell.

18. The multivalent particle of claim 17, wherein the cancer cell is from a cancer comprising melanoma, leukemia, lymphoma, multiple myeloma, liver cancer, pancreatic cancer, lung cancer, breast cancer, prostate cancer, brain cancer, colorectal cancer, bladder cancer, kidney cancer, cervical cancer, ovarian cancer, esophageal cancer, mesothelioma, gastric cancer, and sarcoma.

19. The multivalent particle of claim 16, wherein the target protein comprises a viral surface protein from hepatitis B virus (HBV), SARS CoV-2, SARS CoV-1, MERS CoV, Influenza, respiratory syncytial virus, HIV, or measles.

20. The multivalent particle of claim 16, wherein the target protein comprises a viral spike protein.

21. The multivalent particle of claim 16, wherein the homing polypeptide comprises an antibody that binds specifically to an antigen on the target cell or the target viral protein.

22. The multivalent particle of claim 21, wherein the antigen on the target cell comprises an antigen listed in Table 2.

23. The multivalent particle of claim 21, wherein the antibody comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.

24. The multivalent particle of claim 21, wherein the antibody comprises an amino acid sequence from at least one complementarity determining region of BG10-19, 80R, 7D10, FI6, 1E01, H015, 2H5, ADRI-2F3, H004, H009, H007, H019, or H020.

25. The multivalent particle of claim 21, wherein the antibody comprises a multi-specific antibody.

26. The multivalent particle of claim 16, wherein the homing polypeptide comprises a mammalian receptor that has binding specificity to the target viral protein.

27. The multivalent particle of claim 26, wherein the mammalian receptor comprises NTCP, ACE2, TMPRSS2, DPP4, CD4, HVEM, PD-1, CCR5, CXCR5, CD209, or CLEC4M.

28. The multivalent particle of claim 16, wherein the homing polypeptide comprises a mammalian ligand that recognizes a tumor-associated receptor.

29. The multivalent particle of claim 28, wherein the mammalian ligand comprises EGF, VEGF, TGFbeta, IL-4, IL-11, IGF1, IL-6, or RGD peptide.

30. The multivalent particle of claim 1, wherein the first fusion protein is monomeric.

31. The multivalent particle of claim 1, wherein the first fusion protein comprises an oligomerization domain.

32. The multivalent particle of claim 2, wherein the second fusion protein is monomeric.

33. The multivalent particle of claim 2, wherein the second fusion protein comprises an oligomerization domain.

34. The multivalent particle of claim 31, wherein the oligomerization domain of the first fusion protein comprises a dimerization domain.

35. The multivalent particle of claim 34, wherein the dimerization domain comprises a leucine zipper dimerization domain.

36. The multivalent particle of claim 31, wherein the oligomerization domain of the first fusion protein comprises a trimerization domain.

37. The multivalent particle of claim 36, wherein the trimerization domain comprises a post-fusion oligomerization domain of viral surface protein.

38. The multivalent particle of claim 36, wherein the trimerization domain comprises a D4 post-fusion trimerization domain of VSV-G protein.

39. The multivalent particle of claim 36, wherein the trimerization domain comprises a Dengue E protein post-fusion trimerization domain.

40. The multivalent particle of claim 36, wherein the trimerization domain comprises a foldon trimerization domain.

41. The multivalent particle of claim 31, wherein the oligomerization domain comprises a tetramerization domain.

42. The multivalent particle of claim 41, wherein the tetramerization domain comprises an influenza neuraminidase stem domain.

43. The multivalent particle of claim 33, wherein the oligomerization domain of the second fusion protein comprises a dimerization domain.

44. The multivalent particle of claim 43, wherein the dimerization domain comprises a leucine zipper dimerization domain.

45. The multivalent particle of claim 33, wherein the oligomerization domain of the second fusion protein comprises a trimerization domain.

46. The multivalent particle of claim 45, wherein the trimerization domain comprises a post-fusion oligomerization domain of viral surface protein.

47. The multivalent particle of claim 45, wherein the trimerization domain comprises a D4 post-fusion trimerization domain of VSV-G protein.

48. The multivalent particle of claim 45, wherein the trimerization domain comprises a Dengue E protein post-fusion trimerization domain.

49. The multivalent particle of claim 45, wherein the trimerization domain comprises a foldon trimerization domain.

50. The multivalent particle of claim 33, wherein the oligomerization domain of the second fusion protein comprises a tetramerization domain.

51. The multivalent particle of claim 50, wherein the tetramerization domain comprises an influenza neuraminidase stem domain.

52. The multivalent particle of claim 31, wherein the oligomerization domain of the first fusion protein comprises an amino acid sequence that has at least 90% sequence identity to an amino acid sequence of any one of SEQ ID NOs: 52-65.

53. The multivalent particle of claim 33, wherein the oligomerization domain of the second fusion protein comprises an amino acid sequence that has at least 90% sequence identity to an amino acid sequence of anyone of SEQ ID NOs: 52-65.

54. The multivalent particle of claim 31, wherein the first fusion protein comprises a signal peptide.

55. The multivalent particle of claim 54, wherein domains of the first fusion protein are arranged from the N-terminus to the C-terminus in the following orders:
(a) signal peptide, interferon polypeptide, oligomerization domain, and transmembrane polypeptide;
(b) signal peptide, interferon polypeptide, transmembrane polypeptide, and oligomerization domain; or
(c) signal peptide, oligomerization domain, display peptide, and transmembrane polypeptide.

56. The multivalent particle of claim 54, wherein the first fusion protein further comprises a cytosolic domain.

57. The multivalent particle of claim 56, wherein domains of the first fusion protein are arranged from the N-terminus to the C-terminus in the following orders:
(a) signal peptide, interferon polypeptide, oligomerization domain, transmembrane polypeptide, and cytosolic domain;
(b) signal peptide, interferon polypeptide, transmembrane polypeptide, oligomerization domain, and cytosolic domain; or
(c) signal peptide, oligomerization domain, interferon polypeptide, transmembrane polypeptide, and cytosolic domain.

58. The multivalent particle of claim 33, wherein the second fusion protein comprises a signal peptide.

59. The multivalent particle of claim 58, wherein domains of the second fusion protein are arranged from the N-terminus to the C-terminus in the following orders:
(a) signal peptide, interferon polypeptide, oligomerization domain, and transmembrane polypeptide;
(b) signal peptide, interferon polypeptide, transmembrane polypeptide, and oligomerization domain; or
(c) signal peptide, oligomerization domain, interferon polypeptide, and transmembrane polypeptide.

60. The multivalent particle of claim 58, wherein the second fusion protein further comprises a cytosolic domain.

61. The multivalent particle of claim 60, wherein domains of the second fusion protein are arranged from the N-terminus to the C-terminus in the following orders:
(a) signal peptide, interferon polypeptide, oligomerization domain, transmembrane polypeptide, and cytosolic domain;
(b) signal peptide, interferon polypeptide, transmembrane polypeptide, oligomerization domain, and cytosolic domain; or
(c) signal peptide, oligomerization domain, interferon polypeptide, transmembrane polypeptide, and cytosolic domain.

62. The multivalent particle of claim 16, wherein the second fusion protein comprises a signal peptide and an oligomerization domain.

63. The multivalent particle of claim 62, wherein domains of the second fusion protein are arranged from the N-terminus to the C-terminus in the following orders:
(a) signal peptide, homing polypeptide, oligomerization domain, and transmembrane polypeptide;
(b) signal peptide, homing polypeptide, transmembrane polypeptide, and oligomerization domain; or
(c) signal peptide, oligomerization domain, homing polypeptide, and transmembrane polypeptide.

64. The multivalent particle of claim 62, wherein the second fusion protein further comprises a cytosolic domain.

65. The multivalent particle of claim 64, wherein domains of the second fusion protein are arranged from the N-terminus to the C-terminus in the following orders:
(a) signal peptide, homing polypeptide, oligomerization domain, transmembrane polypeptide, and cytosolic domain;
(b) signal peptide, homing polypeptide, transmembrane polypeptide, oligomerization domain, and cytosolic domain; or
(c) signal peptide, oligomerization domain, homing polypeptide, transmembrane polypeptide, and cytosolic domain.

66. The multivalent particle of claim 1, wherein the transmembrane polypeptide of the first fusion protein anchors the first fusion protein to a lipid bilayer of the multivalent particle.

67. The multivalent particle of claim 3, wherein the transmembrane polypeptide of the second fusion protein anchors the second fusion protein to a lipid bilayer of the multivalent particle.

68. The multivalent particle of claim 16, wherein the transmembrane polypeptide of the second fusion protein anchors the second fusion protein to a lipid bilayer of the multivalent particle.

69. The multivalent particle of claim 1, wherein the transmembrane polypeptide of the first fusion protein comprises a transmembrane domain of a Vesicular Stomatitis virus glycoprotein (VSV-G).

70. The multivalent particle of claim 1, wherein the transmembrane polypeptide of the first fusion protein comprises a cytosolic domain of a Vesicular Stomatitis virus glycoprotein (VSV-G).

71. The multivalent particle of claim 1, wherein the transmembrane polypeptide of the first fusion protein comprises a transmembrane domain of Influenza Neuraminidase (NA).

72. The multivalent particle of claim 1, wherein the transmembrane polypeptide of the first fusion protein comprises a transmembrane domain of influenza Hemagglutinin (HA).

73. The multivalent particle of claim 1, wherein the transmembrane polypeptide of the first fusion protein comprises a transmembrane domain of HIV surface glycoprotein GP120 or GP41.

74. The multivalent particle of claim 1, wherein the transmembrane polypeptide of the first fusion protein comprises a transmembrane domain of Dengue E Protein.

75. The multivalent particle of claim 1, wherein the transmembrane polypeptide of the first fusion protein comprises a transmembrane domain of measles virus surface glycoprotein hemagglutinin (H) protein.

76. The multivalent particle of claim 1, wherein the transmembrane polypeptide of the first fusion protein comprises an amino acid sequence with at least about 90% sequence identity to any one of SEQ ID NOs: 66-74.

77. The multivalent particle of claim 3, wherein the transmembrane polypeptide of the second fusion protein comprises a transmembrane domain of a Vesicular Stomatitis virus glycoprotein (VSV-G).

78. The multivalent particle of claim 3, wherein the transmembrane polypeptide of the second fusion protein comprises a cytosolic domain of a Vesicular Stomatitis virus glycoprotein (VSV-G).

79. The multivalent particle of claim 3, wherein the transmembrane polypeptide of the second fusion protein comprises a transmembrane domain of Influenza Neuraminidase (NA).

80. The multivalent particle of claim 3, wherein the transmembrane polypeptide of the second fusion protein comprises a transmembrane domain of influenza Hemagglutinin (HA).

81. The multivalent particle of claim 3, wherein the transmembrane polypeptide of the second fusion protein comprises a transmembrane domain of HIV surface glycoprotein GP120 or GP41.

82. The multivalent particle of claim 3, wherein the transmembrane polypeptide of the second fusion protein comprises a transmembrane domain of Dengue E Protein.

83. The multivalent particle of claim 3, wherein the transmembrane polypeptide of the second fusion protein comprises a transmembrane domain of measles virus surface glycoprotein hemagglutinin (H) protein.

84. The multivalent particle of claim 3, wherein the transmembrane polypeptide of the second fusion protein comprises an amino acid sequence with at least about 90% sequence identity to any one of SEQ ID NOs: 66-74.

85. The multivalent particle of claim 16, wherein the transmembrane polypeptide of the second fusion protein comprises a transmembrane domain of a Vesicular Stomatitis virus glycoprotein (VSV-G).

86. The multivalent particle of claim 16, wherein the transmembrane polypeptide of the second fusion protein comprises a cytosolic domain of a Vesicular Stomatitis virus glycoprotein (VSV-G).

87. The multivalent particle of claim 16, wherein the transmembrane polypeptide of the second fusion protein comprises a transmembrane domain of Influenza Neuraminidase (NA).

88. The multivalent particle of claim 16, wherein the transmembrane polypeptide of the second fusion protein comprises a transmembrane domain of influenza Hemagglutinin (HA).

89. The multivalent particle of claim 16, wherein the transmembrane polypeptide of the second fusion protein comprises a transmembrane domain of HIV surface glycoprotein GP120 or GP41.

90. The multivalent particle of claim 16, wherein the transmembrane polypeptide of the second fusion protein comprises a transmembrane domain of Dengue E Protein.

91. The multivalent particle of claim 16, wherein the transmembrane polypeptide of the second fusion protein comprises a transmembrane domain of measles virus surface glycoprotein hemagglutinin (H) protein.

92. The multivalent particle of claim 16, wherein the transmembrane polypeptide of the second fusion protein comprises an amino acid sequence with at least about 90% sequence identity to any one of SEQ ID NOs: 66-74.

93. The multivalent particle of claim 1, wherein the multivalent particle is synthetic.

94. The multivalent particle of claim 1, wherein the multivalent particle is recombinant.

95. The multivalent particle of claim 1, wherein the multivalent particle comprises an enveloped particle.

96. The multivalent particle of claim 95, wherein the multivalent particle comprises a lentiviral particle.

97. The multivalent particle of claim 1, wherein the multivalent particle does not comprise viral genetic material.

98. The multivalent particle of claim 1, wherein the multivalent particle comprises a lipid bilayer.

99. The multivalent particle of claim 1, wherein the multivalent particle comprises a virus.

100. The multivalent particle of claim 1, wherein the multivalent particle comprises a replication incompetent virus.

101. The multivalent particle of claim 1, wherein the multivalent particle comprises a replication competent virus.

102. The multivalent particle of claim 1, wherein the multivalent particle comprises a viral-like particle.

103. The multivalent particle of claim 1, wherein the multivalent particle comprises an extracellular vesicle.

104. The multivalent particle of claim 1, wherein the extracellular vesicle comprises an ectosome.

105. The multivalent particle of claim 1, wherein the extracellular vesicle comprises an exosome.

106. The multivalent particle of claim 1, wherein the first fusion protein is expressed at a valency of about 10 copies on the surface of the multivalent particle.

107. The multivalent particle of claim 1, wherein the first fusion protein is expressed at a valency of about 10 to 15 copies on the surface of the multivalent particle.

108. The multivalent particle of claim 1, wherein the first fusion protein is expressed at a valency of at least about 25 copies on the surface of the multivalent particle.

109. The multivalent particle of claim 1, wherein the first fusion protein is expressed at a valency of at least about 50 copies on the surface of the multivalent particle.

110. The multivalent particle of claim 1, wherein the first fusion protein is expressed at a valency of at least about 100 copies on the surface of the multivalent particle.

111. The multivalent particle of claim 1, wherein the first fusion protein is expressed at a valency of at least about 200 copies on the surface of the multivalent particle.

112. The multivalent particle of claim 1, wherein the first fusion protein is expressed at a valency of at least about 400 copies on the surface of the multivalent particle.

113. The multivalent particle of claim 1, wherein the first fusion protein is expressed at a valency of at least about 600 copies on the surface of the multivalent particle.

114. The multivalent particle of claim 1, wherein the first fusion protein is expressed at a valency of at least about 1000 copies on the surface of the multivalent particle.

115. The multivalent particle of claim 2, wherein the second fusion protein is expressed at a valency of about 10 copies on the surface of the multivalent particle.

116. The multivalent particle of claim 2, wherein the second fusion protein is expressed at a valency of about 10 to 15 copies on the surface of the multivalent particle.

117. The multivalent particle of claim 2, wherein the second fusion protein is expressed at a valency of at least about 25 copies on the surface of the multivalent particle.

118. The multivalent particle of claim 2, wherein the second fusion protein is expressed at a valency of at least about 50 copies on the surface of the multivalent particle.

119. The multivalent particle of claim 2, wherein the second fusion protein is expressed at a valency of at least about 100 copies on the surface of the multivalent particle.

120. The multivalent particle of claim 2, wherein the second fusion protein is expressed at a valency of at least about 200 copies on the surface of the multivalent particle.

121. The multivalent particle of claim 2, wherein the second fusion protein is expressed at a valency of at least about 400 copies on the surface of the multivalent particle.

122. The multivalent particle of claim 2, wherein the second fusion protein is expressed at a valency of at least about 600 copies on the surface of the multivalent particle.

123. The multivalent particle of claim 2, wherein the second fusion protein is expressed at a valency of at least about 1000 copies on the surface of the multivalent particle.

124. The multivalent particle of claim 1, wherein the multivalent particle comprises a fluorophore expressed on a surface of the multivalent particle.

125. The multivalent particle of claim 124, wherein the fluorophore is conjugated to a membrane-intercalating polypeptide.

126. The multivalent particle of claim 21, wherein the antibody comprises a CDR-H1 according to SEQ ID NO: 75, a CDR-H2 according to SEQ ID NO: 76, a CDR-H3 according to SEQ ID NO: 77, a CDR-L1 according to SEQ ID NO: 114, a CDR-L2 according to SEQ ID NO: 115, and a CDR-L3 according to SEQ ID NO: 116.

127. The multivalent particle of claim 21, wherein the antibody comprises a CDR-H1 according to SEQ ID NO: 78, a CDR-H2 according to SEQ ID NO: 79, a CDR-H3 according to SEQ ID NO: 80, a CDR-L1 according to SEQ ID NO: 117, a CDR-L2 according to SEQ ID NO: 118, and a CDR-L3 according to SEQ ID NO: 119.

128. The multivalent particle of claim 21, wherein the antibody comprises a CDR-H1 according to SEQ ID NO: 81, a CDR-H2 according to SEQ ID NO: 82, a CDR-H3 according to SEQ ID NO: 83, a CDR-L1 according to SEQ ID NO: 120, a CDR-L2 according to SEQ ID NO: 121, and a CDR-L3 according to SEQ ID NO: 122.

129. The multivalent particle of claim 21, wherein the antibody comprises a CDR-H1 according to SEQ ID NO: 84, a CDR-H2 according to SEQ ID NO: 85, a CDR-H3 according to SEQ ID NO: 86, a CDR-L1 according to SEQ ID NO: 123, a CDR-L2 according to SEQ ID NO: 124, and a CDR-L3 according to SEQ ID NO: 125.

130. The multivalent particle of claim 21, wherein the antibody comprises a CDR-H1 according to SEQ ID NO: 87, a CDR-H2 according to SEQ ID NO: 88, a CDR-H3 according to SEQ ID NO: 89, a CDR-L1 according to SEQ ID NO: 126, a CDR-L2 according to SEQ ID NO: 127, and a CDR-L3 according to SEQ ID NO: 128.

131. The multivalent particle of claim 21, wherein the antibody comprises a CDR-H1 according to SEQ ID NO: 90, a CDR-H2 according to SEQ ID NO: 91, a CDR-H3 according to SEQ ID NO: 92, a CDR-L1 according to SEQ ID NO: 129, a CDR-L2 according to SEQ ID NO: 130, and a CDR-L3 according to SEQ ID NO: 131.

132. The multivalent particle of claim 21, wherein the antibody comprises a CDR-H1 according to SEQ ID NO: 93, a CDR-H2 according to SEQ ID NO: 94, a CDR-H3 according to SEQ ID NO: 95, a CDR-L1 according to SEQ ID NO: 132, a CDR-L2 according to SEQ ID NO: 133, and a CDR-L3 according to SEQ ID NO: 134.

133. The multivalent particle of claim 21, wherein the antibody comprises a CDR-H1 according to SEQ ID NO: 96, a CDR-H2 according to SEQ ID NO: 97, a CDR-H3 according to SEQ ID NO: 98, a CDR-L1 according to SEQ ID NO: 135, a CDR-L2 according to SEQ ID NO: 136, and a CDR-L3 according to SEQ ID NO: 137.

134. The multivalent particle of claim 21, wherein the antibody comprises a CDR-H1 according to SEQ ID NO: 99, a CDR-H2 according to SEQ ID NO: 100, a CDR-H3 according to SEQ ID NO: 101, a CDR-L1 according to SEQ ID NO: 138, a CDR-L2 according to SEQ ID NO: 139, and a CDR-L3 according to SEQ ID NO: 140.

135. The multivalent particle of claim 21, wherein the antibody comprises a CDR-H1 according to SEQ ID NO: 102, a CDR-H2 according to SEQ ID NO: 103, a CDR-H3 according to SEQ ID NO: 104, a CDR-L1 according to SEQ ID NO: 141, a CDR-L2 according to SEQ ID NO: 142, and a CDR-L3 according to SEQ ID NO: 143.

136. The multivalent particle of claim 21, wherein the antibody comprises a CDR-H1 according to SEQ ID NO: 105, a CDR-H2 according to SEQ ID NO: 106, a CDR-H3 according to SEQ ID NO: 107, a CDR-L1 according to SEQ ID NO: 144, a CDR-L2 according to SEQ ID NO: 145, and a CDR-L3 according to SEQ ID NO: 146.

137. The multivalent particle of claim 21, wherein the antibody comprises a CDR-H1 according to SEQ ID NO: 108, a CDR-H2 according to SEQ ID NO: 109, a CDR-H3 according to SEQ ID NO: 110, a CDR-L1 according to SEQ ID NO: 147, a CDR-L2 according to SEQ ID NO: 148, and a CDR-L3 according to SEQ ID NO: 149.

138. The multivalent particle of claim 21, wherein the antibody comprises a CDR-H1 according to SEQ ID NO: 111, a CDR-H2 according to SEQ ID NO: 112, a CDR-H3 according to SEQ ID NO: 113, a CDR-L1 according to SEQ ID NO: 150, a CDR-L2 according to SEQ ID NO: 151, and a CDR-L3 according to SEQ ID NO: 152.

139. The multivalent particle of claim 21, wherein the antibody comprises:
(a) a heavy chain variable region (VH) comprising an amino acid sequence that has at least 90% sequence identity to any one of SEQ ID Nos: 8-20; and
(b) a light chain variable region (VL) comprising an amino acid sequence that has at least 90% sequence identity to any one of SEQ ID Nos: 21-33.

140. The multivalent particle of claim 26, wherein the mammalian receptor comprises an amino acid sequence that has at least 90% sequence identity to any one of SEQ ID NOs: 34-43.

141. The multivalent particle of claim 28, wherein the mammalian ligand comprises an amino acid sequence that has at least 90% sequence identity to any one of SEQ ID NOs: 44-51.

142. The multivalent particle of claim 1, wherein:
(a) the IFN polypeptide of the first fusion protein comprises an amino acid sequence that has at least 90% sequence identity to any one of SEQ ID NOs: 1-7; and
(b) the transmembrane polypeptide of the first fusion protein comprises an amino acid sequence that has at least about 90% sequence identity to any one of SEQ ID NOS: 66-74.

143. The multivalent particle of claim 3, wherein:
(a) the IFN polypeptide of the second fusion protein comprises an amino acid sequence that has at least 90% sequence identity to any one of SEQ ID NOs: 1-7; and
(b) the transmembrane polypeptide of the second fusion protein comprises an amino acid sequence that has at least about 90% sequence identity to any one of SEQ ID NOS: 66-74.

144. The multivalent particle of claim 21, wherein:
(a) the antibody comprises:
(i) a heavy chain variable region (VH) comprising an amino acid sequence that has at least 90% sequence identity to any one of SEQ ID Nos: 8-20; and
(ii) a light chain variable region (VL) comprising an amino acid sequence that has at least 90% sequence identity to any one of SEQ ID Nos: 21-33; and
(b) the transmembrane polypeptide of the second fusion protein comprises an amino acid sequence that has at least about 90% sequence identity to any one of SEQ ID NOS: 66-74.

145. The multivalent particle of claim 26, wherein:
(a) the mammalian receptor comprises an amino acid sequence that has at least 90% sequence identity to any one of SEQ ID NOs: 34-43; and
(b) the transmembrane polypeptide of the second fusion protein comprises an amino acid sequence that has at least about 90% sequence identity to any one of SEQ ID NOS: 66-74.

146. The multivalent particle of claim 28, wherein:
(a) the mammalian ligand comprises an amino acid sequence that has at least 90% sequence identity to any one of SEQ ID NOs: 44-51; and
(b) the transmembrane polypeptide of the second fusion protein comprises an amino acid sequence that has at least about 90% sequence identity to any one of SEQ ID NOs: 66-74.

147. The multivalent particle of claim 31, wherein:
(a) the IFN polypeptide of the first fusion protein comprises an amino acid sequence that has at least 90% sequence identity to any one of SEQ ID NOs: 1-7;
(b) the transmembrane polypeptide of the first fusion protein comprises an amino acid sequence that has at least about 90% sequence identity to any one of SEQ ID NOs: 66-74; and
(c) the oligomerization domain of the first fusion protein comprises an amino acid sequence that has at least 90% sequence identity to any one of SEQ ID NOs: 52-65.

148. The multivalent particle of claim 31, wherein the first fusion protein comprises an amino acid sequence that has at least 90% sequence identity to any one of SEQ ID NOs: 153-158.

149. The multivalent particle of claim 3, wherein the second fusion protein comprises an oligomerization domain, wherein:
(a) the IFN polypeptide of the second fusion protein comprises an amino acid sequence that has at least 90% sequence identity to any one of SEQ ID NOs: 1-7;
(b) the transmembrane polypeptide of the second fusion protein comprises an amino acid sequence that has at least about 90% sequence identity to any one of SEQ ID NOS: 66-74; and
(c) the oligomerization domain of the second fusion protein comprises an amino acid sequence that has at least 90% sequence identity to any one of SEQ ID NOs: 52-65.

150. The multivalent particle of claim 21, wherein the second fusion protein comprises an oligomerization domain, wherein:
(a) the antibody comprises:
(i) a heavy chain variable region (VH) comprising an amino acid sequence that has at least 90% sequence identity to any one of SEQ ID Nos: 8-20; and
(ii) a light chain variable region (VL) comprising an amino acid sequence that has at least 90% sequence identity to any one of SEQ ID Nos: 21-33;
(b) the transmembrane polypeptide of the second fusion protein comprises an amino acid sequence that has at least about 90% sequence identity to any one of SEQ ID NOs: 66-74; and
(c) the oligomerization domain of the second fusion protein comprises an amino acid sequence that has at least 90% sequence identity to any one of SEQ ID NOs: 52-65.

151. The multivalent particle of claim 26, wherein the second fusion protein comprises an oligomerization domain, wherein:
(a) the mammalian receptor comprises an amino acid sequence that has at least 90% sequence identity to any one of SEQ ID NOs: 34-43;
(b) the transmembrane polypeptide of the second fusion protein comprises an amino acid sequence that has at least about 90% sequence identity to any one of SEQ ID NOS: 66-74; and
(c) the oligomerization domain of the second fusion protein comprises an amino acid sequence that has at least 90% sequence identity to any one of SEQ ID NOs: 52-65.

152. The multivalent particle of claim 28, wherein the second fusion protein comprises an oligomerization domain, wherein:
(a) the mammalian ligand comprises an amino acid sequence that has at least 90% sequence identity to any one of SEQ ID NOs: 44-51;
(b) the transmembrane polypeptide of the second fusion protein comprises an amino acid sequence that has at least about 90% sequence identity to any one of SEQ ID NOs: 66-74; and
(c) the oligomerization domain of the second fusion protein comprises an amino acid sequence that has at least 90% sequence identity to any one of SEQ ID NOs: 52-65.

153. A method of treating a disease in a subject in need thereof comprising expressing an IFN on a surface of a multivalent particle, wherein the multivalent particle has a binding affinity to an IFN receptor that is higher than the binding affinity of a soluble version of the IFN to the IFN receptor.

154. A composition comprising a multivalent interferon particle (IFN-MVP) that comprises an enveloped particle displaying at least 10 copies of an interferon (IFN) on a surface of the IFN-MVP.

155. A composition comprising a guided IFN-MVP wherein the guided IFN-MVP comprises an enveloped particle that co-displays at least 10 copies of an IFN and at least 10 copies of a homing polypeptide on a surface of the IFN-MVP.

156. A composition comprising an antibody-guided IFN-MVP wherein the antibody-guided IFN-MVP comprises an enveloped particle that co-displays at least 10 copies of an IFN and at least 10 copies of an antibody on a surface of the IFN-MVP wherein the antibody binds specifically to an antigen on a target cell or a target viral protein.

157. A composition comprising a receptor-guided IFN-MVP wherein the receptor-guided IFN-MVP comprises an enveloped particle that co-displays at least 10 copies of an IFN and at least 10 copies of a receptor on a surface of the IFN-MVP wherein the receptor binds specifically to a target ligand.

158. A composition comprising a ligand-guided IFN-MVP wherein the ligand-guided IFN-MVP comprises an enveloped particle that co-displays at least 10 copies of an IFN and at least 10 copies of a ligand on a surface of the IFN-MVP wherein the ligand binds specifically to a target receptor.
